(19) United States
(12) Patent Application Publication
(10) Pub. No.: US 2006/0135773 A1
(43) Pub. Date: Jun. 22, 2006

(54) Trisubstituted nitrogen modulators of tyrosine phosphatases

(76) Inventors: Joseph E. Semple, San Diego, CA (US); Darryl Rideout, San Diego, CA (US); Ruth F. Nutt, Santa Fe, NM (US); Mark Shenderovich, San Diego, CA (US); Jing Wang, San Diego, CA (US); Shankari Mylvaganam, San Diego, CA (US); Feiyue Wu, Poway, CA (US); Chung Ying Tsai, San Diego, CA (US); Venkatachalapathi V. Yalamoori, San Diego, CA (US); Colin J. Loweth, San Diego, CA (US)

Correspondence Address: JONES DAY, 222 EAST 41ST ST, NEW YORK, NY, 10017, US

(21) Appl. No.: 11/156,230

(22) Filed: Jun. 17, 2005

Related U.S. Application Data
(60) Provisional application No. 60/581,251, filed on Jun. 17, 2004.
(60) Provisional application No. 60/634,200, filed on Dec. 7, 2004.
(60) Provisional application No. 60/638,419, filed on Dec. 22, 2004.

(51) Int. Cl. C07F 9/06; C07D 207/30; C07D 257/04

(52) U.S. Cl. 546/22; 562/10; 562/450; 548/561; 548/253; 558/410

(57) ABSTRACT

Compounds, compositions and methods are provided for modulating the activity of protein tyrosine phosphatases, including PTP-1B. In one embodiment, the compounds are N,N-dibenzylarylsulfonamides.

### RELATED APPLICATIONS

Priority is claimed herein under 35 U.S.C. § 119(e) to U.S. Provisional Patent Application Ser. Nos. 60/581,251, 60/634,200, and 60/638,419, filed Jun. 17, 2004, Dec. 7, 2004, and Dec. 22, 2004 respectively. The disclosures of the above-referenced applications are incorporated by reference herein in their entirety.

### TECHNICAL FIELD

Provided herein are methods of inhibiting the activity of tyrosine phosphatases that regulate signal transduction, and, more particularly, to the use of trisubstituted nitrogen compounds and compositions as tyrosine phosphatase inhibitors for the treatment of conditions and diseases which respond to phosphatase inhibition.

### BACKGROUND

Cellular signal transduction is a fundamental mechanism whereby external stimuli that regulate cellular processes are relayed to the interior of cells. The biochemical pathways through which signals are transmitted within cells comprise a circuitry of directly or functionally connected interactive proteins. One of the key biochemical mechanisms of signal transduction involves the reversible phosphorylation of tyrosine residues on proteins. The phosphorylation state of a protein may affect its conformation and/or enzymatic activity as well as its cellular location. The phosphorylation state of a protein is modified through the reciprocal actions of protein tyrosine kinases (PTKs) and protein tyrosine phosphatases (PTPs) at various specific tyrosine residues.

A common mechanism by which receptors regulate cell function is through an inducible tyrosine kinase activity which is either endogenous to the receptor or is imparted by other proteins that become associated with the receptor (Darnell et al., 1994, Science 264:1415-1421; Heldin, 1995, Cell 80:213-223; Pawson, 1995, Nature 373:573-580).

Protein tyrosine kinases comprise a large family of transmembrane receptor and intracellular enzymes with multiple functional domains (Taylor et al., 1992 Ann. Rev. Cell Biol. 8:429-62). The binding of ligand allosterically transduces a signal across the cell membrane where the cytoplasmic portion of the PTKs initiates a cascade of molecular interactions that disseminate the signal throughout the cell and into the nucleus. Many receptor protein tyrosine kinase (RPTKs), such as epidermal growth factor receptor (EGFR) and platelet-derived growth factor receptor (PDGFR) undergo oligomerization upon ligand binding, and the receptors self-phosphorylate (via autophosphorylation or transphosphorylation) on specific tyrosine residues in the cytoplasmic portions of the receptor (Schlessinger and Ullrich, 1992, Neuron, 9:383-91, Heldin, 1995, Cell 80:213-223). Cytoplasmic protein tyrosine kinases (CPTKs), such as Janus kinases (e.g., JAK1, JAK2, TYK2) and Src kinases (e.g., src, lck, fyn), are associated with receptors for cytokines (e.g., IL-2, IL-3, IL-6, erythropoietin) and interferons, and antigen receptors. These receptors also undergo oligomerization and have tyrosine residues that become phosphorylated during activation, but the receptor polypeptides themselves do not possess kinase activity.

Like the PTKs, the protein tyrosine phosphatases (PTPs) comprise a family of transmembrane and cytoplasmic enzymes, possessing at least an approximately 230 amino acid catalytic domain containing a highly conserved active site with the consensus motif >I/V!HCXAGXXR>S/T!G. The substrates of PTPs may be PTKs which possess phosphotyrosine residues or the substrates of PTKs (Hunter, 1989, Cell 58:1013-16; Fischer et al., 1991, Science 253:401-6; Saito & Streuli, 1991, Cell Growth and Differentiation 2:59-65; Pot and Dixon, 1992, Biochem. Biophys. Acta 1136:35-43). Among these, Protein Tyrosine Phosphatase-1B (PTP-1B) is an intracellular protein found in various human tissues (Charbonneau et al., 1989, Proc. Natl. Acad. Sci. USA 86:5252-5256; Goldstein, 1993, Receptor 3:1-15).

Transmembrane or receptor-like PTPs (RPTPs) possess an extracellular domain, a single transmembrane domain, and one or two catalytic domains followed by a short cytoplasmic tail. The extracellular domains of these RPTPs are highly divergent, with small glycosylated segments (e.g., RPTPα, RPTPε), tandem repeats of immunoglobulin-like and/or fibronectin type III domains (e.g., LAR) or carbonic anhydrase like domains (e.g., RPTPγ, RPTPβ). These extracellular features might suggest that these RPTPs function as a receptor on the cell surface, and their enzymatic activity might be modulated by ligands. Intracellular or cytoplasmic PTPs (CPTPs), such as PTP1C, PTP1D, typically contain a single catalytic domain flanked by several types of modular conserved domains. For example, PTP1C, a hemopoietic cell CPTP is characterized by two Src-homology homology 2 (SH2) domains that recognize short peptide motifs bearing phosphotyrosine (pTyr).

In general, these modular conserved domains influence the intracellular localization of the protein. SH2-containing proteins are able to bind pTyr sites in activated receptors and cytoplasmic phosphoproteins. Another conserved domain known as SH3 binds to proteins with proline-rich regions. A third type known as pleckstrin-homology (PH) domain has also been identified. These modular domains have been found in both CPTKs and CPTPs as well as in non-catalytic adapter molecules, such as Grbs (Growth factor Receptor Bound), which mediate protein-protein interactions between components of the signal transduction pathway (Skolnik et al., 1991, Cell 65:83-90; Pawson, 1995, Nature 373:573-580).

Multiprotein signaling complexes comprising receptor subunits, kinases, phosphatases and adapter molecules are assembled in subcellular compartments through the specific and dynamic interactions between these domains with their binding motifs. Such signaling complexes integrate the extracellular signal from the ligand-bound receptor and relay the signal to other downstream signaling proteins or complexes in other locations inside the cell or in the nucleus (Koch et al., 1991, Science 252:668-674; Pawson, 1994, Nature 373:573-580; Mauro et al., 1994, Trends Biochem Sci 19:151-155; Cohen et al., 1995, Cell 80:237-248).

The levels of tyrosine phosphorylation required for normal cell growth and differentiation at any time are achieved through the coordinated action of PTKs and PTPS. Depending on the cellular context, these two types of enzymes may either antagonize or cooperate with each other during signal transduction. An imbalance between these enzymes may impair normal cell functions leading to metabolic disorders and cellular transformation.

For example, insulin binding to the insulin receptor, which is a PTK, triggers a variety of metabolic and growth promoting effects such as glucose transport, biosynthesis of glycogen and fats, DNA synthesis, cell division and differentiation. Diabetes mellitus, which is characterized by insufficient or a lack of insulin signal transduction, can be caused by any abnormality at any step along the insulin signaling pathway (Olefsky, 1988, in “Cecil Textbook of Medicine,” 18th Ed., 2:1360-81).

It is also well known, for example, that the overexpression of PTKs, such as HER2, can play a decisive role in the development of cancer (Slamon et al., 1987, Science 235:77-82) and that antibodies capable of blocking the activity of this enzyme can abrogate tumor growth (Drebin et al., 1988, Oncogene 2:387-394). Blocking the signal transduction capability of tyrosine kinases such as Flk-1 and the PDGF receptor have been shown to block tumor growth in animal models (Millauer et al., 1994, Nature 367:577; Ueno et al., Science 252:844-848).

Tyrosine phosphatases also play a role in signal transduction. For example, ectopic expression of RPTPα produces a transformed phenotype in embryonic fibroblasts (Zheng et al., Nature 359:336-339), and overexpression of RPTPα in embryonal carcinoma cells causes the cells to differentiate into a cell type with neuronal phenotype (den Hertog et al., EMBO J 12:3789-3798). The gene for human RPTPγ has been localized to chromosome 3p21 which is a segment frequently altered in renal and small lung carcinoma. Mutations may occur in the extracellular segment of RPTPγ which result in RPTPs that no longer respond to external signals (LaForgia et al., Wary et al., 1993, Cancer Res 52:478-482). Mutations in the gene encoding PTP1C (also known as HCP, SHP) are the cause of the motheaten phenotype in mice which suffer severe immunodeficiency, and systemic autoimmune disease accompanied by hyperproliferation of macrophages (Schultz et al., 1993, Cell 73:1445-1454). PTP1D (also known as Syp or PTP2C) has been shown to bind through SH2 domains to sites of phosphorylation in PDGFR, EGFR and insulin receptor substrate 1 (IRS-1). Reducing the activity of PTP1D by microinjection of anti-PTP1D antibody has been shown to block insulin or EGF-induced mitogenesis (Xiao et al., 1994, J Biol Chem 269:21244-21248).

Much effort has been devoted to determining which proteins are substrates of PTP-1B. One such identified substrate is the insulin receptor. The binding of insulin to its receptor results in autophosphorylation of the receptor, most notably on tyrosines 1146, 1150, and 1151 in the kinase catalytic domain (White & Kahn, 1994, J Biol. Chem. 269:1-4). This activates the insulin receptor tyrosine kinase, and phosphorylates the insulin receptor substrate proteins that propagate the insulin-signaling event to mediate insulin's various biological effects.

A glutathione S-transferase (GST) fusion protein of PTP-1B that had a point mutation in the PTP-1B catalytic domain was constructed by Seely et al., 1996, Diabetes 45:1379-1385. Although catalytically inactive, this fusion protein was able to bind to the insulin receptor, as demonstrated by its ability to precipitate the insulin receptor from purified receptor preparations and from whole cell lysates derived from cells expressing the insulin receptor.

Recently, it was reported that PTP-1B is a negative regulator of the insulin signalling pathway (Kennedy et al., 1999, Science 283:1544-1548). It is also known that mice lacking PTP-1B are resistant to both diabetes and obesity. These data suggest that inhibitors of PTP-1B may be beneficial in the treatment of Type 2 diabetes.

Thus, inhibitors of PTP-1B improve insulin-sensitivity, and demonstrate utility in controlling or treating Type 1 and Type 2 diabetes, in improving insulin sensitivity, and in improving glucose tolerance. Such inhibitor compounds and compositions may also prove useful in treating or preventing cancer, neurodegenerative diseases and the like.

### SUMMARY

Provided herein are compounds, compositions and methods for the modulation of tyrosine phosphatase activity. Such compounds, compositions and methods will find use in the treatment of conditions and diseases caused by dysfunctional signal transduction.

In one embodiment, provided is a method for inhibiting protein tyrosine phosphatase activity, which comprises administering a compound having the formula I: 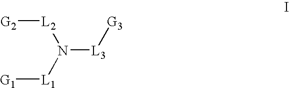
or a pharmaceutically acceptable derivative thereof, wherein:

L₁, L₂ and L₃ are linkers as hereinafter more fully defined, including the following;

L₁, L₂ and L₃ are independently selected from:

N—C single bond (i.e. G₁, G₂, or G₃ are directly bonded to N by a single bond), alkylene, alkenylene, alkynylene, cycloalkylene, oxocycloalkylene, amidocycloalkylene, heterocyclylene, heteroarylene, oxo, sulfonyl, alkylsulfonyl, alkenylsulfonyl, alkynylsulfonyl, amido, carboxamido, alkylamido, alkylcarboxamido, and alkoxyoxo; and

where each of the above substituents are unsubstituted or substituted with one or more substituents, in one embodiment, one, two, or three substituents, each independently selected from D¹, where D¹ is H, alkyl, alkenyl, alkynyl, aryl, cycloalkyl, heterocyclyl, heteroaryl, amido, cyano, cyanoalkenyl, amidoalkyl, amidoalkenyl, and oxo;

G₁, G₂ and G₃ are substituent moieties as hereinafter more fully defined, including the following;

G₁, G₂, and G₃ are independently selected from:

(i) alkyl, alkenyl, alkynyl, aryl, alkaryl, arylalkyl, alkarylalkyl, alkenylaryl, alkylsulfonyl, alkenylsulfonyl, alkynylsulfonyl, amido, alkylamino, alkylaminoaryl, arylamino, aminoalkyl, aminoaryl, alkoxy, alkoxyaryl, aryloxy, alkylamido, alkylcarboxamido, arylcarboxamido, alkoxyoxo, biaryl, alkoxyoxoaryl, amidocycloalkyl, carboxyalkylaryl, carboxyaryl, carboxyamidoaryl, carboxamido, cyanoalkyl, cyanoalkenyl, cyanobiaryl, cycloalkyl, cycloalkyloxo, cycloalkylaminoaryl, haloalkyl, haloalkylaryl, haloaryl, heterocyclyl, heteroaryl, hydroxyalkylaryl, and sulfonyl; and

(ii) G₁ and G₂ can be linked together to form a cycloalkyl, oxocycloalkyl, cycloalkyloxo, amidocycloalkyl, cycloalkylamido, alkenylaryl, amidoalkenylaryl, and the following groups, 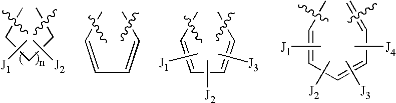
where J₁, J₂, J₃, and J₄ are selected from H, alkyl, alkenyl, alkynyl, aryl, cycloalkyl, heterocyclyl, heteroaryl, amido, cyano, cyanoalkenyl, amidoalkyl, amidoalkenyl, oxo, and CH═C(CN)C(O)NH; and

where each of the above substituents are unsubstituted or substituted with one or more substituents, in one embodiment, one, two, or three substituents, each independently selected from M¹, where M¹ is H, alkyl, alkenyl, alkynyl, aryl, arylalkyl, alkoxy, alkoxyoxo, alkylthia, amino, amido, arylamino, aryloxy, alkylamino, alkylsulfonyl, alkylcarboxyalkylphosphonato, arylcarboxamido, carboxy, carboxyoxo, carboxyalkyl, carboxyalkyloxa, carboxyalkenyl, carboxyamido, carboxyhydroxyalkyl, cycloalkyl, amido, cyano, cyanoalkenyl, cyanoaryl, amidoalkyl, amidoalkenyl, halo, haloalkyl, haloalkylsulfonyl, heterocyclyl, heteroaryl, heteroarylalkyl, heteroarylalkoxy, hydroxy, hydroxyalkyl, hydroxyamino, hydroxyimino, heteroarylalkyloxa, nitro, phosphonato, phosphonatoalkyl, and phosphonatohaloalkyl. 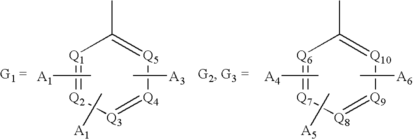
Q₁ through Q₁₇ are independently selected from no bond (direct link), C, N, S, and O, with the proviso that the resulting combination of atoms is a chemically stable cyclic and/or (hetero)aromatic ring system; and
Appended A₁ through A₆ substituent groups can be combined to form stable mono- or bicyclic-fused alicyclic, heterocyclic and/or (hetero)aromatic rings.

Also provided are compounds of the formulae shown above and elsewhere herein and compositions useful for modulating protein tyrosine phosphatase activity.

### DETAILED DESCRIPTION

A. Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, protein tyrosine phosphatase (PTP) refers to an enzyme of the PTP class, including enzymes that are both tyrosine-specific and dual-specific in their phoshpatase activity. In one embodiment, such phosphatases encompass both transmembrane receptor-like PTPs (RPTPs) as well as soluble cytosolic proteins. RPTPs include small glycosylated segments (e.g., RPTPa, RPTPe), tandem repeats of immunoglobulin-like and/or fibronectin type III domains (e.g., LAR) or carbonic anhydrase like domains (e.g., RPTPg, RPTPb). Intracellular or cytoplasmic PTPs (CPTPs), include PTP1B or PTP-1B, PTP1C and PTP1D, and typically contain a single catalytic domain flanked by several types of modular conserved domains.

As used herein, protein tyrosine phosphatase 1B (PTP-1B) refers to a 37-kD protein comprised of a single domain, is topologically organized into 8 alpha helices and 12 beta sheets. See, e.g., Jia, Z., Barford, D., Flint, A. J., and N. K. Tonks (1995) Science 268:1754-1758; Pannifer A., Flint A., Tonks N., and Barford D.(1998) The Journal of Biological Chemistry 273:10454-10462; Charbonneau et al., 1989, Proc. Natl. Acad. Sci. USA 86:5252-5256; Goldstein, 1993, Receptor 3:1-15.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl or cycloalkyl. Pharmaceutically acceptable enol esters include, but are not limited to, derivatives of formula C═C(OC(O)R) where R is hydrogen, alkyl, alkenyl, alkynyl or cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating diseases or disorders in which PTP-1B activity is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response, such as modulation of PTP-1B activity, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392). Other prodrugs are as defined elsewhere herein.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are independently alkyl, including lower alkyl; “carboxamide” refers to groups of formula —NR′COR in which R′ and R are independently alkyl, including lower alkyl.

As used herein, “diarylaminocarbonyl” refers to —C(O)NRR′ in which R and R′ are independently selected from aryl, including lower aryl, such as phenyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein, “oxaalkylene” refers to —(CRR)ₙ—O—(CRR)ₘ—, where n and m are each independently an integer from 0 to 4. As used herein, “thiaalkylene” refers to —(CRR)ₙ—S—(CRR)ₘ—, —(CRR)ₙ—S(═O)—(CRR)ₘ—, and —(CRR)ₙ—S(═O)₂—(CRR)ₘ—, where n and m are each independently an integer from 0 to 4.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 6 carbons.

As used herein, “heteroarylene” refers to a divalent monocyclic or multicyclic aromatic ring system, in one embodiment of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur. The term “lower heteroarylene” refers to heteroarylene groups having 5 or 6 atoms in the ring.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted alkyl,” “substituted alkenyl,” “substituted alkynyl,” “substituted cycloalkyl,” “substituted cycloalkenyl,” “substituted cycloalkynyl,” “substituted aryl,” “substituted heteroaryl,” “substituted heterocyclyl,” “substituted alkylene,” “substituted alkenylene,” “substituted alkynylene,” “substituted cycloalkylene,” “substituted cycloalkenylene,” “substituted cycloalkynylene,” “substituted arylene,” “substituted heteroarylene” and “substituted heterocyclylene” refer to alkyl, alkenyl, alkynyl, cycloalkyl, cycloalkenyl, cycloalkynyl, aryl, heteroaryl, heterocyclyl, alkylene, alkenylene, alkynylene, cycloalkylene, cycloalkenylene, cycloalkynylene, arylene, heteroarylene and heterocyclylene groups, respectively, that are substituted with one or more substituents, in certain embodiments one, two, three or four substituents, where the substituents are as defined herein, in one embodiment selected from Q1.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylid-ene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the divalent group —SO₂NHNH—. “Hydrazide” refers to the divalent group —C(O)NHNH—. “Azo” refers to the divalent group —N═N—. “Hydrazinyl” refers to the divalent group —NH—NH—.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

B. Compounds, Compositions and Methods

In one embodiment, provided is a method for inhibiting protein tyrosine phosphatase activity, which comprises administering a compound having the formula I: 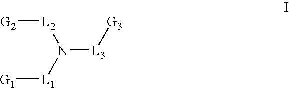
or a pharmaceutically acceptable derivative thereof, wherein:

L₁, L₂ and L₃ are independently selected from:

N—C single bond (i.e. G₁, G₂, or G₃ are directly bonded to N by a single bond), alkylene, alkenylene, alkynylene, cycloalkylene, oxocycloalkylene, amidocycloalkylene, heterocyclylene, heteroarylene, oxo, sulfonyl, alkylsulfonyl, alkenylsulfonyl, alkynylsulfonyl, amido, carboxamido, alkylamido, alkylcarboxamido, and alkoxyoxo; and

where each of the above substituents are unsubstituted or substituted with one or more substituents, in one embodiment, one, two, or three substituents, each independently selected from D¹, where D¹ is H, alkyl, alkenyl, alkynyl, aryl, cycloalkyl, heterocyclyl, heteroaryl, amido, cyano, cyanoalkenyl, amidoalkyl, amidoalkenyl, and oxo;

G₁, G₂, and G₃ are independently selected from:

(i) alkyl, alkenyl, alkynyl, aryl, alkaryl, arylalkyl, alkarylalkyl, alkenylaryl, alkylsulfonyl, alkenylsulfonyl, alkynylsulfonyl, amido, alkylamino, alkylaminoaryl, arylamino, aminoalkyl, aminoaryl, alkoxy, alkoxyaryl, aryloxy, alkylamido, alkylcarboxamido, arylcarboxamido, alkoxyoxo, biaryl, alkoxyoxoaryl, amidocycloalkyl, carboxyalkylaryl, carboxyaryl, carboxyamidoaryl, carboxamido, cyanoalkyl, cyanoalkenyl, cyanobiaryl, cycloalkyl, cycloalkyloxo, cycloalkylaminoaryl, haloalkyl, haloalkylaryl, haloaryl, heterocyclyl, heteroaryl, hydroxyalkylaryl, and sulfonyl; and

(ii) G₁ and G₂ can be linked together to form a cycloalkyl, oxocycloalkyl, cycloalkyloxo, amidocycloalkyl, cycloalkylamido, alkenylaryl, amidoalkenylaryl, and the following groups, 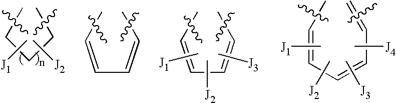
where J₁, J₂, J₃, and J₄ are selected from H, alkyl, alkenyl, alkynyl, aryl, cycloalkyl, heterocyclyl, heteroaryl, amido, cyano, cyanoalkenyl, amidoalkyl, amidoalkenyl, oxo, and CH═C(CN)C(O)NH; and

where each of the above substituents are unsubstituted or substituted with one or more substituents, in one embodiment, one, two, or three substituents, each independently selected from M¹, where M¹ is H, alkyl, alkenyl, alkynyl, aryl, arylalkyl, alkoxy, alkoxyoxo, alkylthia, amino, amido, arylamino, aryloxy, alkylamino, alkylsulfonyl, alkylcarboxyalkylphosphonato, arylcarboxamido, carboxy, carboxyoxo, carboxyalkyl, carboxyalkyloxa, carboxyalkenyl, carboxyamido, carboxyhydroxyalkyl, cycloalkyl, amido, cyano, cyanoalkenyl, cyanoaryl, amidoalkyl, amidoalkenyl, halo, haloalkyl, haloalkylsulfonyl, heterocyclyl, heteroaryl, heteroarylalkyl, heteroarylalkoxy, hydroxy, hydroxyalkyl, hydroxyamino, hydroxyimino, heteroarylalkyloxa, nitro, phosphonato, phosphonatoalkyl, and phosphonatohaloalkyl.

In one embodiment, L₁, L₂ and L₃ are independently selected from:

N—C single bond (i.e. G1, G2, or G3 are directly bonded to N by a single bond), (CRR1)ₘ, CF₂, CF₂CF₂, C(═O), C(═O)C(═O), C(═O)(CRR1)ₘ, (CRR1)ₘC(═O)(CRR1)ₘ, C(═O)O(CRR1)ₘ, (CRR1)ₘC(═O)O, N(R), —C(═O)N(R)N(R1), N(R)SO₂N(R1), C(═O)N(R), N(R)C(═O)N(R1), O, OC(═O)N(R), P(═O)(OR), P(═O)(NR), P(═S)(OR), P(═S)(NR), SO₂, S(═O)ₙ(CRR1)ₘ, (CRR1)ₘS(═O)ₙ(CRR1)ₘ, where m=0-6 and n=0-2, S(═O)(═NR), S(═NR)(═NR1), SO₂NR, wherein R and R1 are independently selected from hydrogen, C1-C6 alkyl which is optionally substituted with 1 to 3 substituents selected from the group consisting of Y₁, Y₂, and Y₃, —OC(R2R3)OC(═O)R4, —OC(R2R3)OC(═O)OR4, where R2, R3 and R4 are independently selected from H, C₁-C₇ alkyl, R2, R3 and R4 can be combined to form a 5-7-membered ring, C2-C6 alkenyl which is optionally substituted with 1 to 3 substituents selected from the group consisting of Y₁, Y₂, and Y₃, C2-C6 alkynyl which is optionally substituted with 1 to 3 substituents selected from the group consisting of Y₁, Y₂, and Y₃, C3-C8 cycloalkyl which is optionally substituted with 1 to 3 substituents selected from the group consisting of Y₁, Y₂, and Y₃, C6-C14 aryl which is optionally substituted with 1 to 3 substituents selected from the group consisting of Y₁, Y₂, and Y₃, C10-C20 linked biaryl and heterobiaryl, which is optionally substituted with 1 to 3 substituents selected from the group consisting of Y₁, Y₂, and Y₃, C7-C16 aralkyl, which is optionally substituted with 1 to 3 substituents selected from the group consisting of Y₁, Y₂, and Y₃,

C5-C14 monocyclic-heteroaryl and bicyclic-heteroaryl, which is optionally substituted with 1 to 3 substituents selected from the group consisting of Y₁, Y₂, and Y₃, and C5-C14 heteroaralkyl which is optionally substituted on the alkyl chain and on the ring with 1 to 3 substituents selected from the group consisting of Y₁, Y₂, and Y₃, R and R1 can be joined together to form an alicyclic or heterocyclic ring;

R and R1 are independently and optionally substituted with 1 to 3 substituents Y₁, Y₂, and Y₃.

In another embodiment, L₁, L₂ and L₃ are independently selected from:

N—C single bond (i.e. G1, G2, or G3 are directly bonded to N by a single bond), alkylene, alkenylene, alkynylene, cycloalkylene, oxocycloalkylene, amidocycloalkylene, heterocyclylene, heteroarylene, oxo, sulfonyl, alkylsulfonyl, alkenylsulfonyl, alkynylsulfonyl, amido, carboxamido, alkylamido, alkylcarboxamido, and alkoxyoxo; where each of the above substituents are unsubstituted or substituted with one or more substituents, in one embodiment, one, two, or three substituents, each independently selected from D¹, where D¹ is H, alkyl, alkenyl, alkynyl, cycloalkyl, heterocyclyl, heteroaryl, amido, cyano, cyanoalkenyl, amidoalkyl, amidoalkenyl, and oxo.

In another embodiment, L₁, L₂ and L₃ are independently selected from:

N—C single bond, C1-C5 alkylene, C1-C5 alkenylene, C1-C5 alkynylene, C3-C15 cycloalkylene, C3-C15 oxocycloalkylene, C3-C15 amidocycloalkylene, C3-C15 heterocyclylene, C3-C15 heteroarylene, oxo, sulfonyl, alkylsulfonyl, alkenylsulfonyl, alkynylsulfonyl, amido, carboxamido, alkylamido, alkylcarboxamido, and alkoxyoxo; where each of the above substituents are unsubstituted or substituted with one or more substituents, in one embodiment, one, two, or three substituents, each independently selected from D¹, where D¹ is H, alkyl, alkenyl, alkynyl, cycloalkyl, heterocyclyl, heteroaryl, amido, cyano, cyanoalkenyl, amidoalkyl, amidoalkenyl, and oxo.

In another embodiment, L₁, L₂ and L₃ are independently selected from:

N—C single bond, C1-C5 alkylene, C1-C5 alkenylene, C1-C5 alkynylene, C3-C15 cycloalkylene, C3-C15 oxocycloalkylene, C3-C15 amidocycloalkylene, C3-C15 heterocyclylene, C3-C15 heteroarylene, oxo, sulfonyl, alkylsulfonyl, alkenylsulfonyl, alkynylsulfonyl, amido, carboxamido, alkylamido, alkylcarboxamido, and alkoxyoxo.

In another embodiment, L₁, L₂ and L₃ are independently selected from:

N—C single bond, —CH₂, —C(═O), —CH₂CH₂, —SO₂, —S(═O)₂CH₂, —S(═O)₂CH₂CH₂, —C(═O)NHCH₂, —C(═O)OCH₂, and —S(═O)₂CH═CH.

In another embodiment, L₁, L₂ or L₃ is independently N—C single bond.

In another embodiment, L₁, L₂ or L₃ is independently —CH₂. In another embodiment, L₁, L₂ or L₃ is independently —CH₂CH₂. In another embodiment, L₁, L₂ or L₃ is independently —C(═O)NHCH₂. In another embodiment, L₁, L₂ or L₃ is independently —C(═O). In another embodiment, L₁, L₂ or L₃ is independently —C(═O)OCH₂. In another embodiment, L₁, L₂ or L₃ is independently —SO₂. In another embodiment, L₁, L₂ or L₃ is independently —S(═O)₂CH₂. In another embodiment, L₁, L₂ or L₃ is independently —S(═O)₂CH₂CH₂. In another embodiment, L₁, L₂ or L₃ is independently —S(═O)₂CH═CH.

In another embodiment, G₁, G₂ and G₃ are independently selected from: H, C1-6 alkyl and which is optionally substituted with 1 to 3 substituents selected from the group consisting of Y₁, Y₂, and Y₃, C2-C6 alkenyl which is optionally substituted with 1 to 3 substituents selected from the group consisting of Y₁, Y₂, and Y₃, C2-C6 alkynyl which is optionally substituted with 1 to 3 substituents selected from the group consisting of Y₁, Y₂, and Y₃; C3-C8 cycloalkyl which is optionally substituted with 1 to 3 substituents selected from the group consisting of Y₁, Y₂, and Y₃; C6-C14 aryl which is optionally substituted with 1 to 3 substituents selected from the group consisting of Y₁, Y₂, and Y₃; C7-C16 aralkyl which is optionally substituted with 1 to 3 substituents selected from the group consisting of Y₁, Y₂, and Y₃; C5-C14 heteroaryl, which is optionally substituted with 1 to 3 substituents selected from the group consisting of Y₁, Y₂, and Y₃, and C5-C14 heteroaralkyl, which is optionally substituted on the ring and the alkyl chain with 1 to 3 substituents selected from the group consisting of Y₁, Y₂, and Y₃; and

Y₁, Y₂, and Y₃ are selected from R, (CRR1)ₙOR, OH, (CRR1)ₙNRR1, C(═NR)NRR1, C(═NOR)NRR1, halogen (F, Cl, Br, I), cyano, nitro, CF₃, CF₂CF₃, CH₂CF₃, CH(CF₃)₂, C(OH)(CF₃)₂, OCHCl₂, OCF₃, OCF₂H, OCF₂CF₃, OCH₂CF₃, (CRR1)ₙOC(═O)NRR1, (CRR1)ₙNHC(═O)C(═O)OR, (CRR1)ₙNHC(═O)NRSO₂(Me, CF₃), (CRR1)ₙNHSO₂(Me, CF₃), (CRR1)ₙNHSO₂NRR1, NHSO₂NRC(═O)(Me, CF₃), (CRR1)ₙNHC(═O)R, (CRR1)ₙNHC(═O)NRR1, C(═O)OH, (CRR1)ₙC(═O)OH, C(═O)OR, C(═O)O(CRR1)OC(═O)R, C(═O)O(CRR1)OC(═O)OR, C(═O)R, —(CRR1)ₙC(═O)R, (CF₂)ₙC(═O)R, (CFR)ₙC(═O)R, tetrazolyl (Tzl), (CRR1)ₙTzl, (CF₂)ₙTzl, (CFR)ₙTzl, (CRR1)ₙC(═O)OR, (CRR1)ₙC(═O)NH₂, (CRR1)ₙC(═O)NRR1, (CRR1)ₙC(═O)C(═O)OR, (CRR1)ₙCH(OR)C(═O)OR, (CF₂)ₙC(═O)OH, (CF₂)ₙC(═O)OR, (CF₂)ₙC(═O)NH₂, (CF₂)ₙC(═O)NRR1, (CRR1)ₙC(═O)C(═O)OR, (CRR1)ₙCH(OR)C(═O)OR, C(R)═C(R1), C(═O)OR, C(R)═C(R1)-Tzl, (CRR1)ₙP(═O)(OH)₂, (CRR1)ₙP(═O)(OR)(OR1), P(═O)(OR)[(OCRR1)OC(═O)R], P(═O)(OR)[(OCRR1)OC(═O)OR], P(═O)[(OCRR1)OC(═O)R)][(OCRR1)OC(═O)R], P(═O)[(OCRR1)OC(═O)OR)][(OCRR1)OC(═O)OR], (CRR1)ₙP(═O)(Me)(OR), (CRR1)ₙP(═O)(CF₃)(OR), (CF₂)ₙP(═O)(OR)(OR1), (CF₂)ₙP(═O)(Me)(OR), (CF₂)ₙP(═O)(CF₃)(OR), (CFR)_{q}P(═O)(OR)(OR1), CR═CR—P(═O)(OR)(OR1), CR═CR—P(═O)(Me)(OR), CC—P(═O)(OR)(OR1), (C═O)P(═O)(OR)(OR1), (C═O)P(═O)(Me)(OR), (C═O)P(═O)(CF₃)(OR), (CROR1)ₙP(═O)(OR)(OR1), (CROR1)ₙP(═O)(Me)(OR), (CROR1)ₙP(═O)(CF₃)(OR), O(CRR1)ₙC(═O)OR, O(CF₂)ₙC(═O)OR, OCH[C(═O)OR]₂, O(CRR1)ₙCH[C(═O)OR]₂, OCF[C(═O)OR]₂, O(CRR1)ₙC(═O)C(═O)OR, O(CF₂)ₙC(═O)C(═O)OR, O(CRR1)ₙTzl, O(CF₂)ₙTzl, OCH(Tzl)₂, O(CF₂)ₙP(═O)(OR)(OR1), O(CF₂)ₙP(═O)(Me)(OR), O(CF₂)ₙP(═O)(CF₃)(OR), O(CFR)ₙP(═O)(OR)(OR1), O(CFR)ₙP(═O)(Me)(OR), O(CFR)ₙP(═O)(CF₃)(OR), (CRR1)ₙP(═O)(OR)(OR1), O(CRR1)ₙP(═O)(Me)(OR), O(CRR1)ₙP(═O)(CF₃)(OR), OCF[P(═O)(Me)(OR)]₂, SO₃H, —(CRR1)ₙSO₃H, S(O)ₙR, SCF₃, SCHF₂, SO₂CF₃, SO₂Ph, (CRR1)ₙS(O)ₙR, (CRR1)ₙS(O)₂CF₃, (CRR1)ₙSO₂NRR1, (CRR1)ₙSO₂NRC(═O)(Me, CF₃), (CF₂ₙSO₃H, (CFR)ₙSO₃H, (CF₂)ₙSO₂NRR1, wherein n=0-2, and R and R1 are as defined above; Y₁, Y₂ and/or Y₃ may also be selected together to be (CRR1)₂₋₆ and substituted variants thereof, —O[C(R2)(R3)]ᵣO— or —O[C(R2)(R3)]ᵣ₊₁—, wherein r is an integer from 1 to 4 and R2 and R3 are independently selected from the group consisting of hydrogen, C1-C12 alkyl, C6-14 aryl, C5-C14 heteroaryl, C7-C15 aralkyl, and C5-C14 heteroarylalkyl.

In another embodiment, G₁, G₂, and G₃ are independently selected from:

alkyl, alkenyl, alkynyl, aryl, alkaryl, arylalkyl, alkarylalkyl, alkenylaryl, alkylsulfonyl, alkenylsulfonyl, alkynylsulfonyl, amido, alkylamino, alkylaminoaryl, arylamino, aminoalkyl, aminoaryl, alkoxy, alkoxyaryl, aryloxy, alkylamido, alkylcarboxamido, arylcarboxamido, alkoxyoxo, biaryl, alkoxyoxoaryl, amidocycloalkyl, carboxyalkylaryl, carboxyaryl, carboxyamidoaryl, carboxamido, cyanoalkyl, cyanoalkenyl, cyanobiaryl, cycloalkyl, cycloalkyloxo, cycloalkylaminoaryl, haloalkyl, haloalkylaryl, haloaryl, heterocyclyl, heteroaryl, hydroxyalkylaryl, and sulfonyl; where each of the above substituents are unsubstituted or substituted with one or more substituents, in one embodiment, one, two, or three substituents, each independently selected from M¹, where M¹ is H, alkyl, alkenyl, alkynyl, aryl, arylalkyl, alkoxy, alkoxyoxo, alkylthia, amino, amido, arylamino, aryloxy, alkylamino, alkylsulfonyl, alkylcarboxyalkylphosphonato, arylcarboxamido, carboxy, carboxyoxo, carboxyalkyl, carboxyalkyloxa, carboxyalkenyl, carboxyamido, carboxyhydroxyalkyl, cycloalkyl, amido, cyano, cyanoalkenyl, cyanoaryl, amidoalkyl, amidoalkenyl, halo, haloalkyl, haloalkylsulfonyl, heterocyclyl, heteroaryl, heteroarylalkyl, heteroarylalkoxy, hydroxy, hydroxyalkyl, hydroxyamino, hydroxyimino, heteroarylalkyloxa, nitro, phosphonato, phosphonatoalkyl, and phosphonatohaloalkyl.

In another embodiment, G₁ and G₂ can be linked together to form a cycloalkyl, oxocycloalkyl, cycloalkyloxo, amidocycloalkyl, cycloalkylamido, alkenylaryl, amidoalkenylaryl, and the following groups, 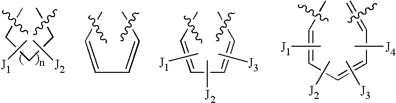
where J₁, J₂, J₃, and J₄ are selected from H, alkyl, alkenyl, alkynyl, cycloalkyl, heterocyclyl, heteroaryl, amido, cyano, cyanoalkenyl, amidoalkyl, amidoalkenyl, oxo, and CH═C(CN)C(O)N.

In another embodiment, G₁ and G₂ can be linked together to form the following groups,

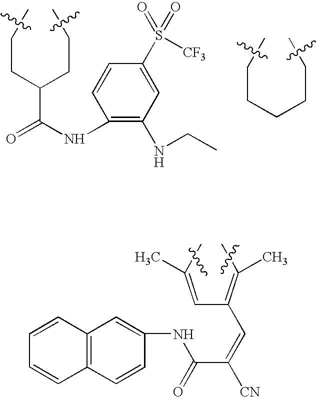

In another embodiment, G₁ and G₂ can be linked together to form

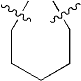

In another embodiment, G₁ and G₂ can be linked together to form.

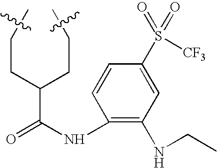

In another embodiment, G₁ and G₂ can be linked together to form

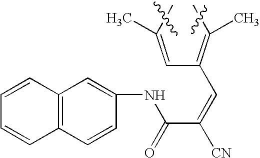

In another embodiment, G₁, G₂, and G₃ are independently selected from, —CH(E¹)₂, —CH═CH(E¹), —CH(E¹)CH₂(E¹), —CH═C(E¹)C(O)NHE¹, 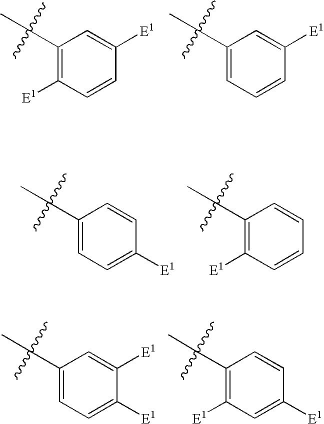
where E¹ at each instance is independently selected from —CN, —OCH₃, —COOH, Cl, F, Br, CH₃,

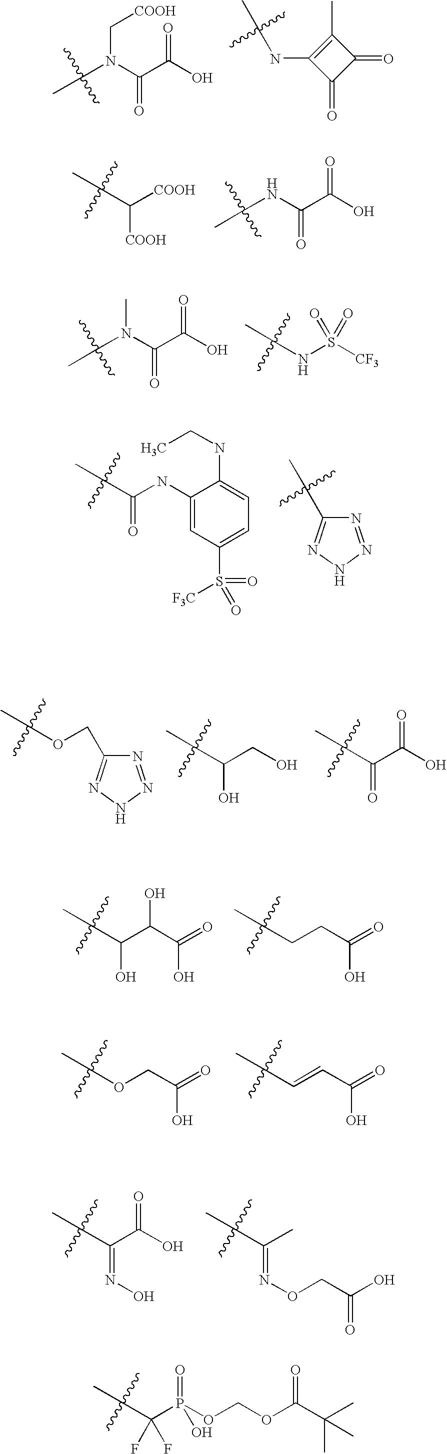

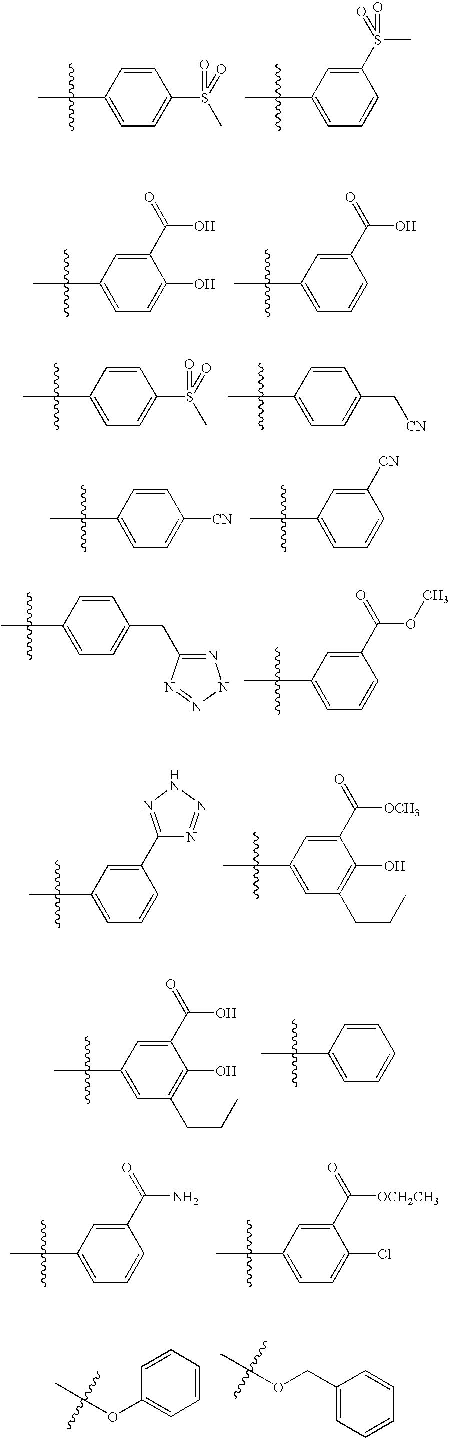

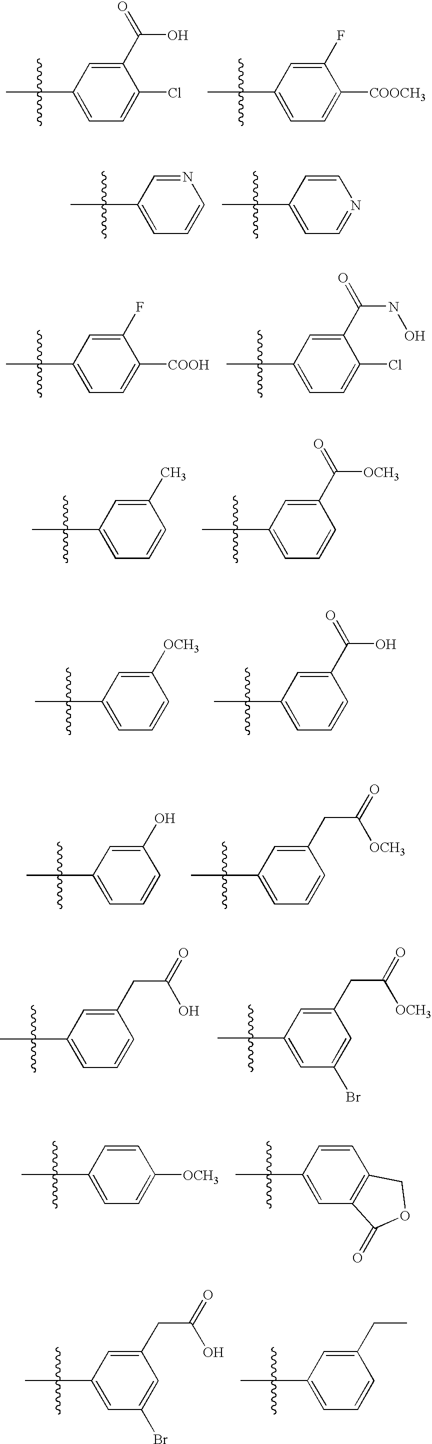

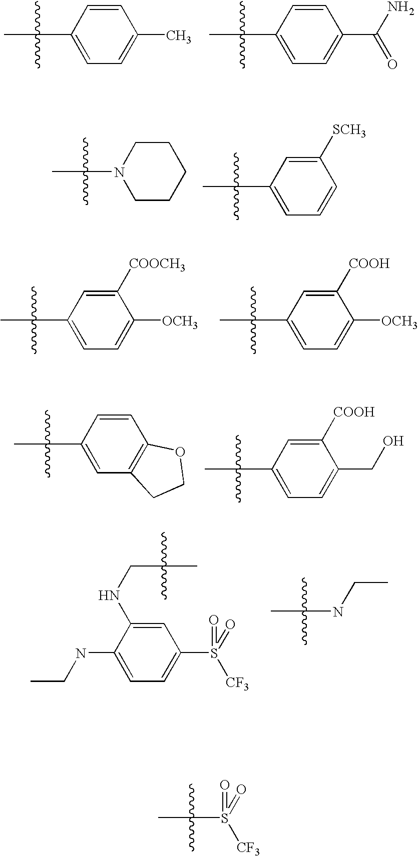

In another embodiment, G₁, G₂, and G₃ are independently selected from, CH₃, C(═O)CH₃, CH(CH₃)CH₃,

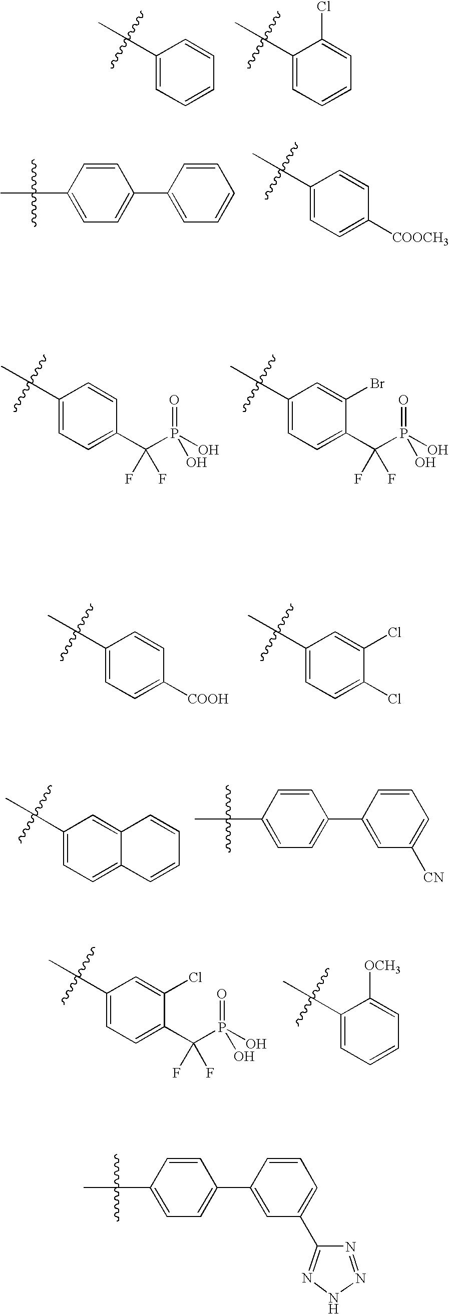

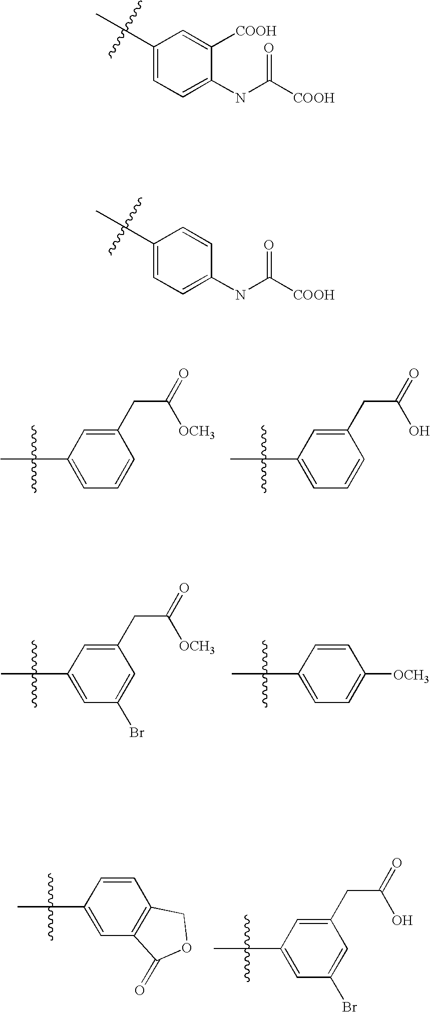

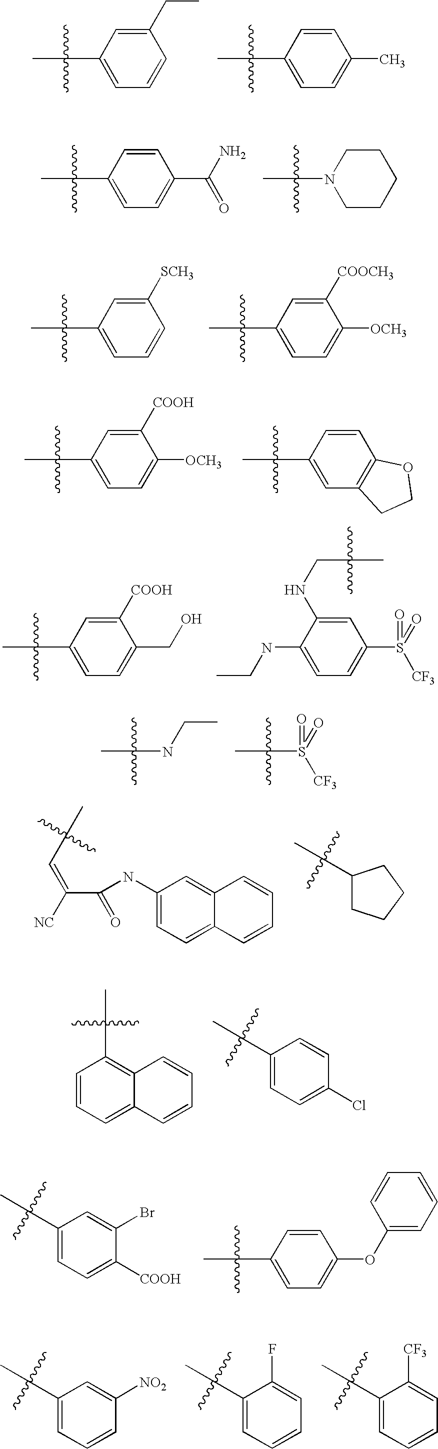

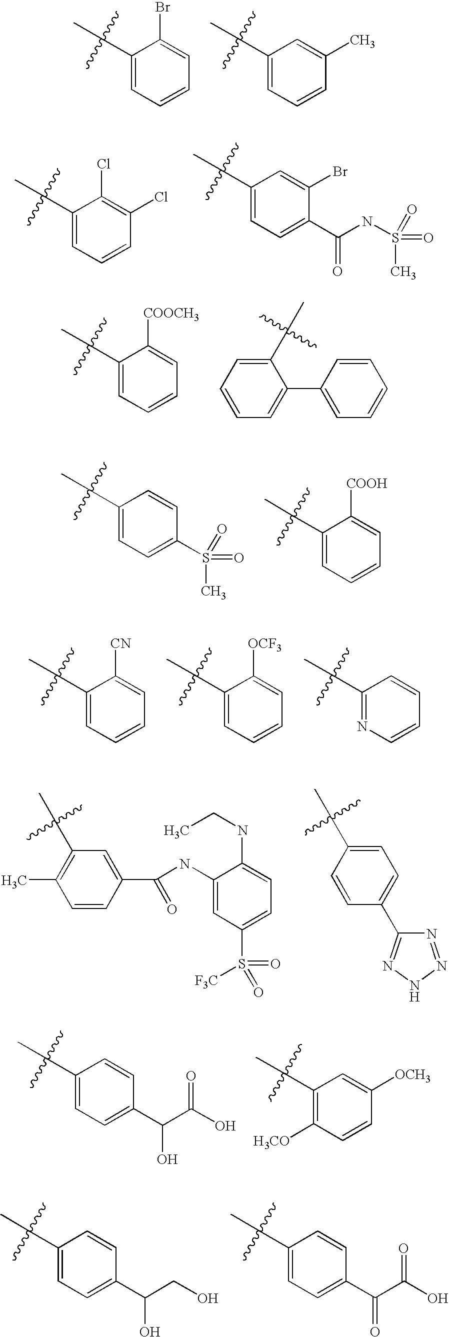

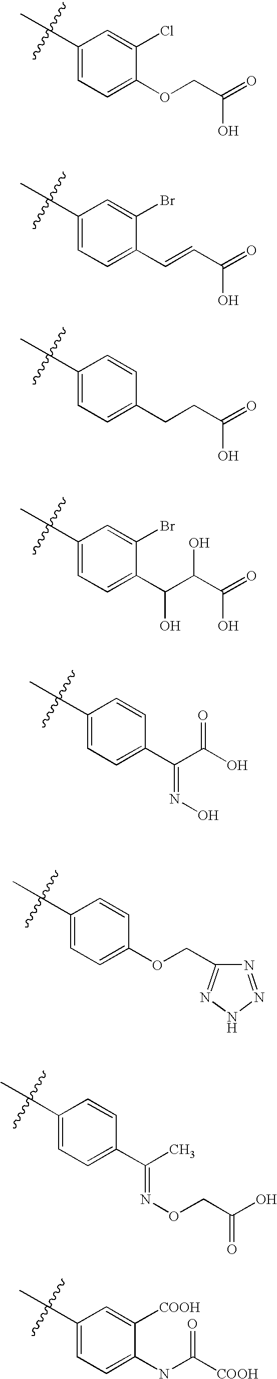

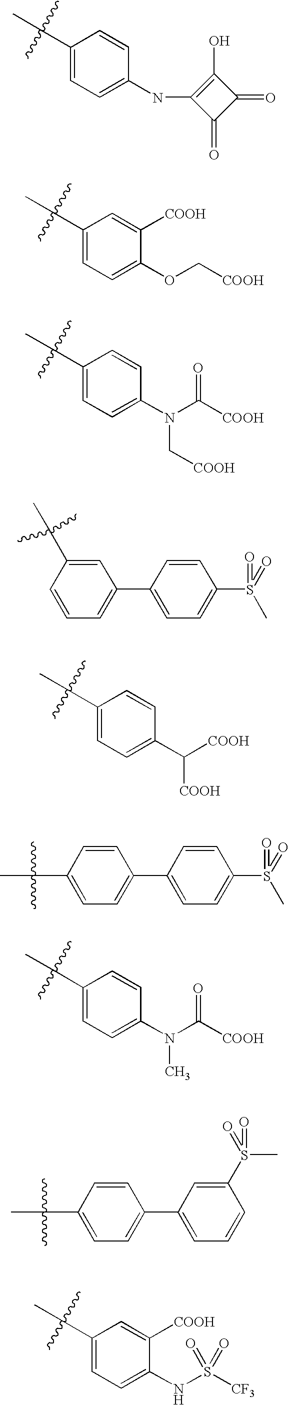

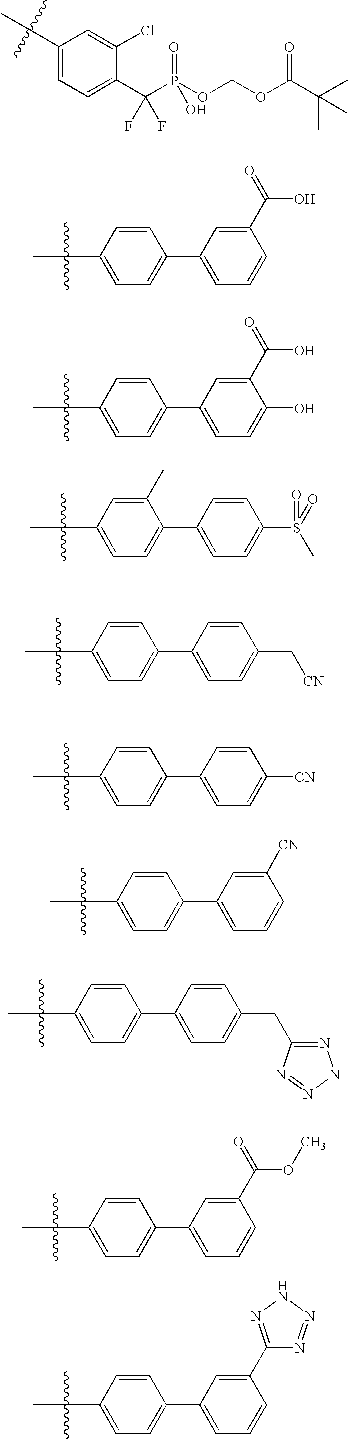

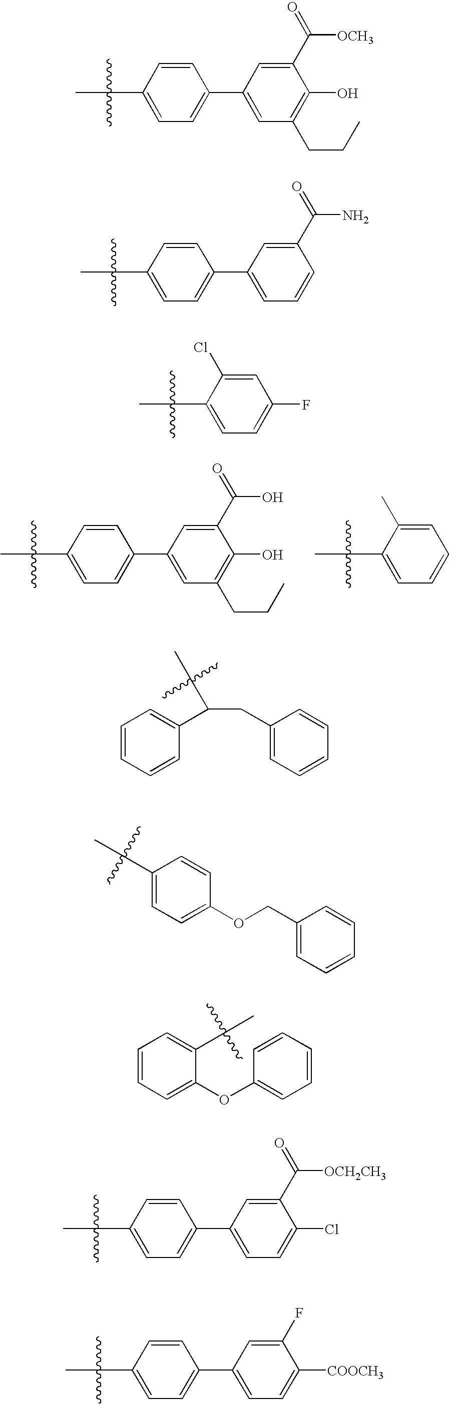

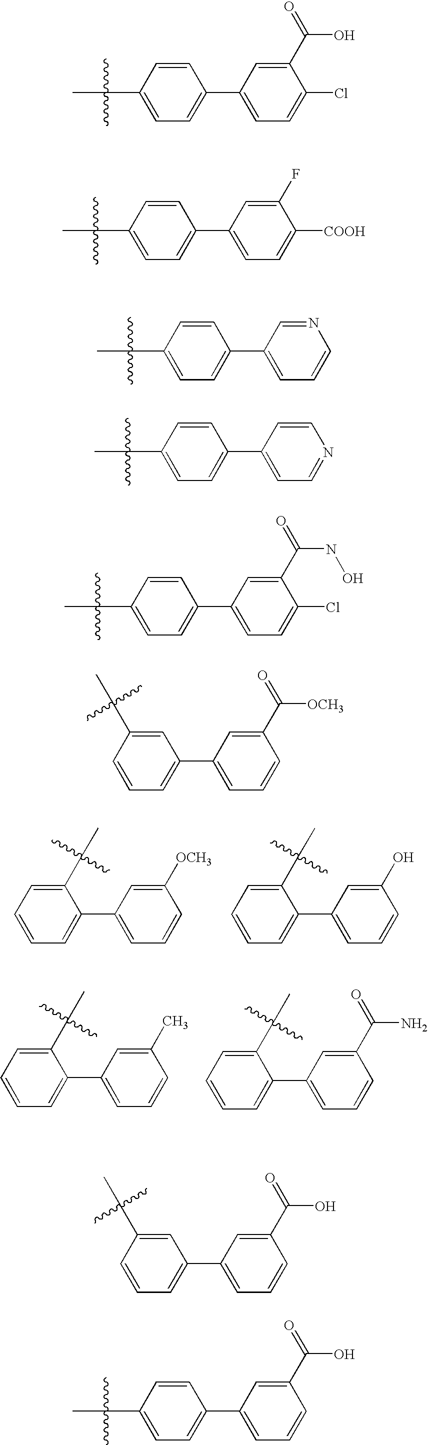

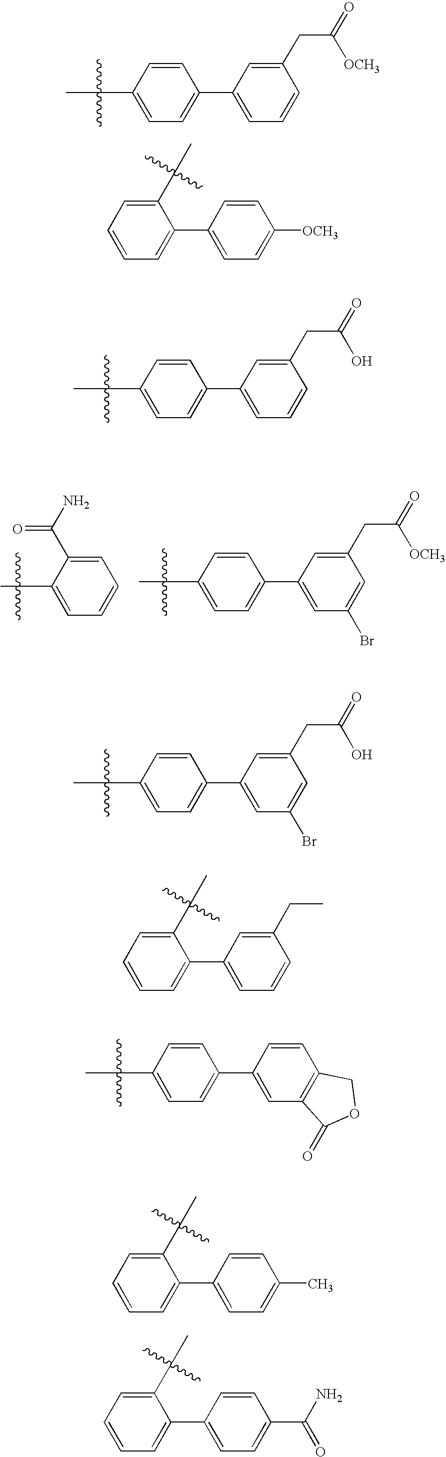

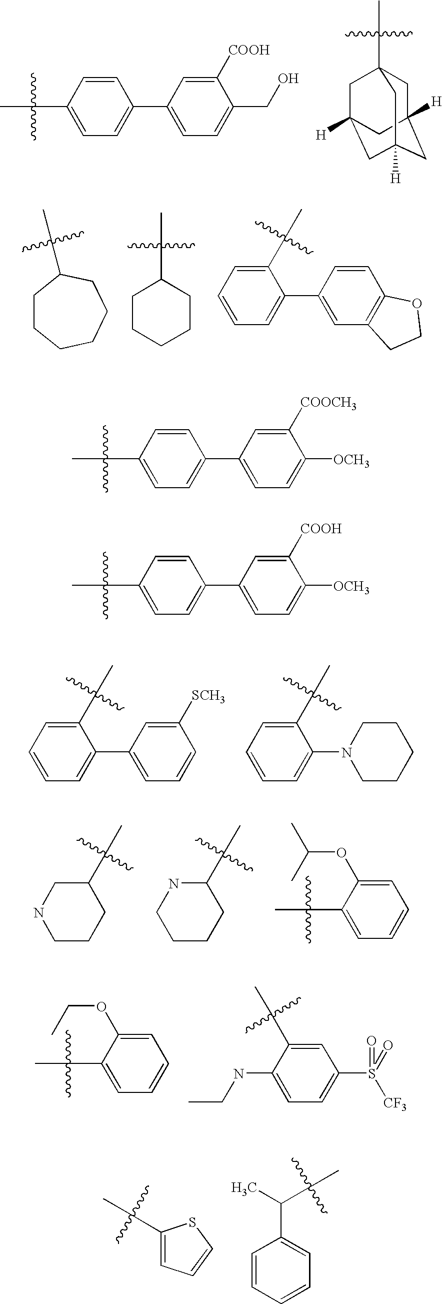

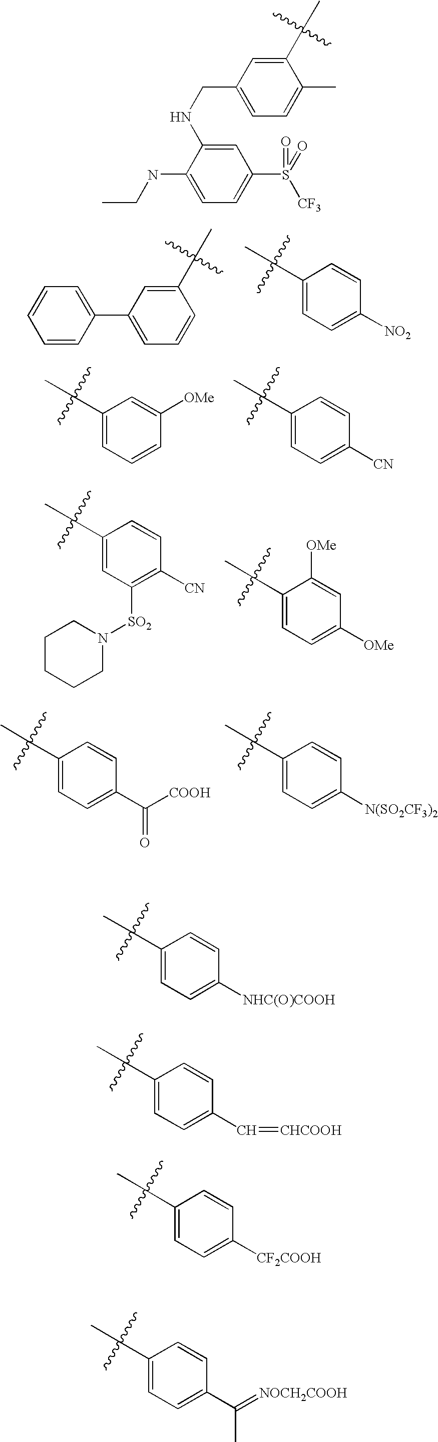

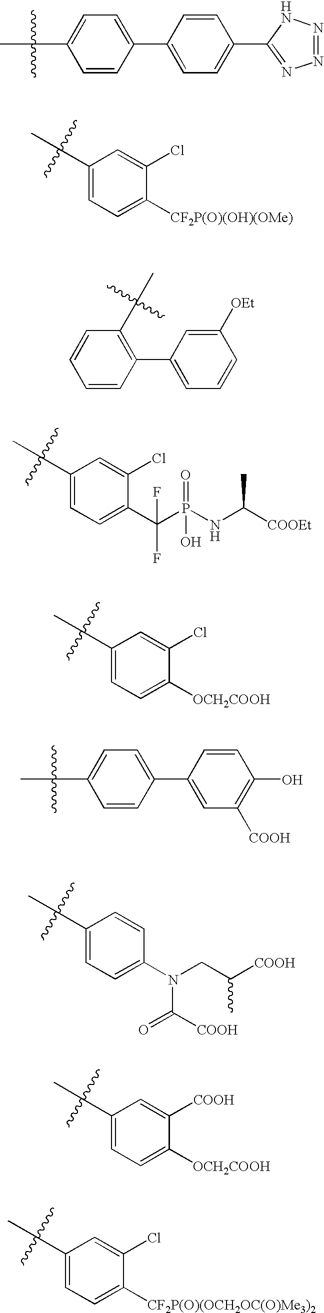

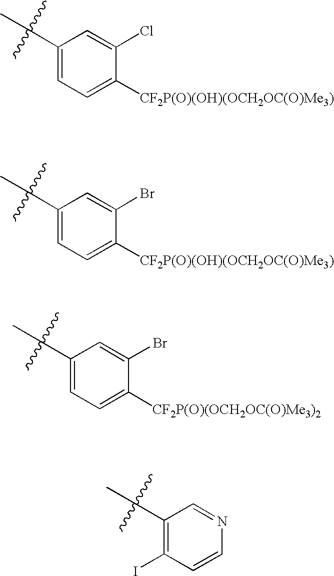

In another embodiment, G₁, G₂, or G₃ are independently selected from,

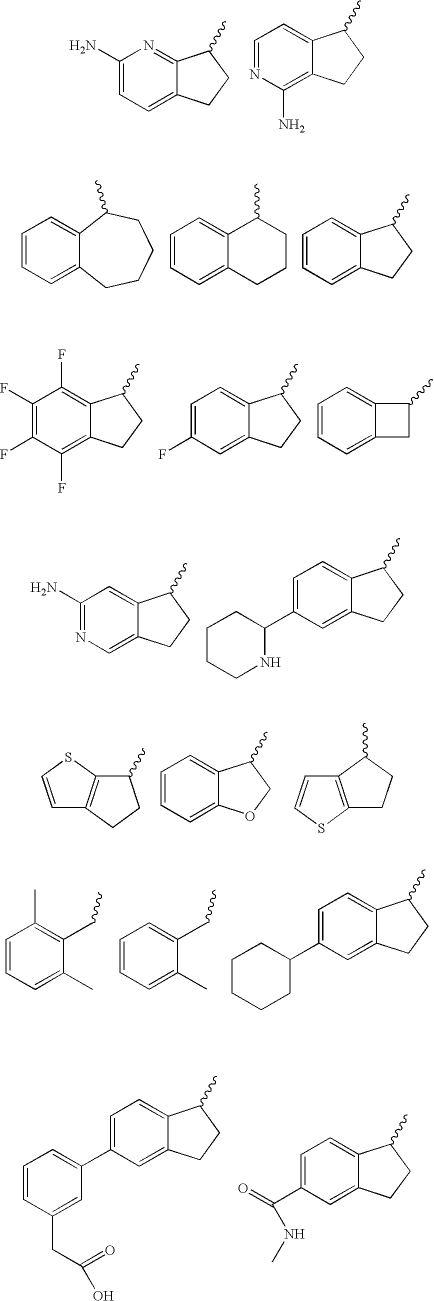

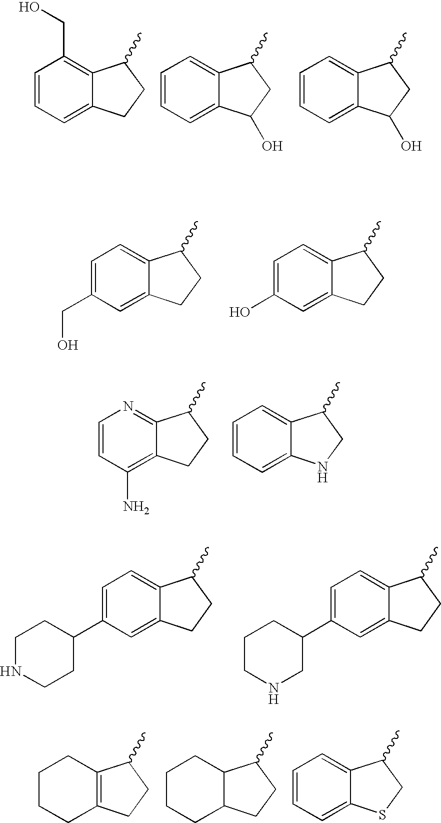

In another embodiment, G₁, G₂, or G₃ are independently selected from,

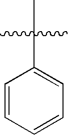

In another embodiment, G₁, G₂, or G₃ are independently selected from,

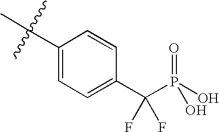

In the above formula I, the defined linkers and groups can also be in accordance with the following description, where bonds are shown only where required for clarity. All combinations of the following groups are to be considered within the scope of the instant disclosure.

Linkers:

L₁, L₂ and L3 are independently selected from the following: no bond (i.e. direct link from N to G₁, G₂, or G₃), (CRR1)ₘ, CF₂, CF₂CF₂, C(═O), C(═O)C(═O), C(═O)(CRR1)ₘ, (CRR1)ₘC(═O)(CRR1)ₘ, C(═O)O(CRR1)ₘ, (CRR1)ₘC(═O)O, N(R), —C(═O)N(R)N(R1), N(R)SO₂N(R1), C(═O)N(R), N(R)C(═O)N(R1), O, OC(═O)N(R), P(═O)(OR), P(═O)(NR), P(═S)(OR), P(═S)(NR), SO₂, S(═O)ₙ(CRR1)ₘ, (CRR1)ₘS(═O)ₙ(CRR)ₘ, where m=0-6 and n=0-2, S(═O)(═NR), S(═NR)(═NR1), SO₂NR, C1-C₇ alkyl, C₃-C₇-alkenyl, C₃-C₇-alkynyl, C₃-C₇ cycloalkyl, aryl of from about 6 to about 10 carbon atoms, heteroaryl containing from about 5 to about 10 atoms (selected from C, N, O),

wherein R and R1 are independently selected from hydrogen, alkyl of 1 to about 6 carbon atoms and which is unsubstituted or mono-, di- or tri-substituted with 1 to 3 substituents selected from the group consisting of Y₁, Y₂, and Y₃, —OC(R2R3)OC(═O)R4, —OC(R2R3)OC(═O)OR4,

where R2, R3 and R4 are independently selected from H, C₁-C₇ alkyl, R2, R3 and R4 can be combined to form a 5-7-membered ring, alkenyl of 2 to about 6 carbon atoms and which is unsubstituted or mono-, di- or tri-substituted with 1 to 3 substituents selected from the group consisting of Y₁, Y₂, and Y₃, alkynyl of 2 to about 6 carbon atoms and which is unsubstituted or mono-, di- or tri-substituted with 1 to 3 substituents selected from the group consisting of Y₁, Y₂, and Y₃, cycloalkyl of 3 to about 8 carbon atoms and which is unsubstituted or mono-, di- or tri-substituted with 1 to 3 substituents selected from the group consisting of Y₁, Y₂, and Y₃, aryl of about 6 to about 14 carbon atoms and which is unsubstituted or mono-, di- or tri-substituted with 1 to 3 substituents selected from the group consisting of Y₁, Y₂, and Y₃, linked biaryl and heterobiaryl of about 10 to 20 atoms featuring two (hetero)aromatic ring systems linked through a single bond, with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, aralkyl of about 7 to about 16 carbon atoms which is unsubstituted or mono-, di- or tri-substituted with 1 to 3 substituents selected from the group consisting of Y₁, Y₂, and Y₃,

monocyclic-heteroaryl and bicyclic-heteroaryl, each of about 5 to about 14 ring atoms with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, and heteroaralkyl of about 5 to about 14 ring atoms with the ring atoms selected from carbon and heteroatoms, wherein the heteroatoms are selected from oxygen, nitrogen, and sulfur, which is unsubstituted or substituted on the alkyl chain and which is unsubstituted on the ring or mono-, di- or tri-substituted on the ring with 1 to 3 substituents selected from the group consisting of Y₁, Y₂, and Y₃. R and R1 can be joined together to form an alicyclic or heterocyclic ring;

Examples of aryl groups include phenyl, naphthyl, tetrahydronaphthyl, indenyl, indanyl, anthracenyl and fluorenyl ring systems.

Examples of monocyclic heteroaryl, e.g. heteroaryl of about 5 to 6 ring atoms include furyl, thienyl, pyrrolyl, pyrazolyl, imidazolyl, triazolyl, oxazolyl, isoxazolyl, oxadiazolyl, thiazolyl, isothiazolyl, thiadiazolyl, pyridyl, pyridazinyl, pyrimidinyl, pyrazinyl, triazinyl (1,3,5- and 1,2,4-isomers) and tetrazinyl ring systems.

Examples of bicyclic heteroaryl, e.g. heteroaryl of about 8 to 10 ring atoms, include benzothienyl, benzofuranyl, indolyl, benzimidazoyl, indazolyl, benzotriazolyl, benzothiazolyl, isobenzothiazolyl, benzoxazolyl, isobenzoxazolyl, quinolinyl, isoquinolinyl, cinnolinyl, phthalazinyl, quinazolinyl, quinoxalinyl, and stable partially reduced congeners, such as, e.g., dihydrobenzofuranyl, indolinyl, dihydrobenzothienyl, dihydrobenzopyranyl (chromane), iso-dihydro-benzopyranyl (isochromane), dihydrobenzothiopyranyl (thiochroman), iso-dihydrobenzothiopyranyl (isothiochroman), tetrahydroquinolinyl, tetrahydroisoquinolinyl and similar ring systems.

Examples of linked biaryl and heterobiaryl include 2-phenylphenyl, 3-phenylphenyl, 4-phenylphenyl, phenylnaphthyl, bithienyl, thienyloxazolyl, phenylpyridyl, thiazolylpyridyl, phenylpyrimidinyl, phenyltriazinyl, phenylthienyl, naphthylfuranyl and heterocyclic analogs of these in which C is replaced by N, C═C is replaced by S, and/or C═C is replaced by O.

R and R1 are independently and optionally substituted with 1 to 3 substituents Y₁, Y₂, and Y₃ selected from the group consisting of R, (CRR1)ₙOR, OH, (CRR1)ₙNRR1, C(═NR)NRR1, C(═NOR)NRR1, halogen (F, Cl, Br, I), cyano, nitro, CF₃, CF₂CF₃, CH₂CF₃, CH(CF₃)₂, C(OH)(CF₃)₂, OCHCl₂, OCF₃, OCF₂H, OCF₂CF₃, OCH₂CF₃, (CRR1)ₙOC(═O)NRR1, (CRR1)ₙNHC(═O)C(═O)OR, (CRR1)ₙNHC(═O)NRSO₂(Me, CF₃), (CRR1)ₙNHSO₂(Me, CF₃), (CRR1)ₙNHSO₂NRR1, NHSO₂NRC(═O)(Me, CF₃), (CRR1)ₙNHC(═O)R, (CRR1)ₙNHC(═O)NRR1, C(═O)OH, (CRR1)ₙC(═O)OH, C(═O)OR, C(═O)O(CRR1)OC(═O)R, C(═O)O(CRR1)OC(═O)OR, C(═O)R, —(CRR1)ₙC(═O)R, (CF₂)ₙC(═O)R, (CFR)ₙC(═O)R, tetrazolyl (Tzl), (CRR1)ₙTzl, (CF₂)ₙTzl, (CFR)ₙTzl, (CRR1)ₙC(═O)OR, (CRR1)ₙC(═O)NH₂, (CRR1)ₙC(═O)NRR1, (CRR1)ₙC(═O)C(═O)OR, (CRR1)ₙCH(OR)C(═O)OR, (CF₂)ₙC(═O)OH, (CF₂)ₙC(═O)OR, (CF₂)ₙC(═O)NH₂, (CF₂)ₙC(═O)NRR1, (CRR1)ₙC(═O)C(═O)OR, (CRR1)ₙCH(OR)C(═O)OR, C(R)═C(R1), C(═O)OR, C(R)═C(R1)-Tzl, (CRR1)ₙP(═O)(OH)₂, (CRR1)ₙP(═O)(OR)(OR1), P(═O)(OR)[(OCRR1)OC(═O)R], P(═O)(OR)[(OCRR1)OC(═O)OR], P(═O)[(OCRR1)OC(═O)R)][(OCRR1)OC(═O)R], P(═O)[(OCRR1)OC(═O)OR)][(OCRR1)OC(═O)OR], (CRR1)ₙP(═O)(Me)(OR), (CRR1)ₙP(═O)(CF₃)(OR), (CF₂)ₙP(═O)(OR)(OR1), (CF₂)ₙP(═O)(Me)(OR), (CF₂)ₙP(═O)(CF₃)(OR), (CFR)_{q}P(═O)(OR)(OR1), CR═CR—P(═O)(OR)(OR1), CR═CR—P(═O)(Me)(OR), CC—P(═O)(OR)(OR1), (C═O)P(═O)(OR)(OR1), (C═O)P(═O)(Me)(OR), (C═O)P(═O)(CF₃)(OR), (CROR1)ₙP(═O)(OR)(OR1), (CROR1)ₙP(═O)(Me)(OR), (CROR1)ₙP(═O)(CF₃)(OR), O(CRR1)ₙC(═O)OR, O(CF₂)ₙC(═O)OR, OCH[C(═O)OR]₂, O(CRR1)ₙCH[C(═O)OR]₂, OCF[C(═O)OR]₂, O(CRR1)ₙC(═O)C(═O)OR, O(CF₂)ₙC(═O)C(═O)OR, O(CRR1)ₙTzl, O(CF₂)ₙTzl, OCH(Tzl)₂, O(CF₂)ₙP(═O)(OR)(OR1), O(CF₂)ₙP(═O)(Me)(OR), O(CF₂)ₙP(═O)(CF₃)(OR), O(CFR)ₙP(═O)(OR)(OR1), O(CFR)ₙP(═O)(Me)(OR), O(CFR)ₙP(═O)(CF₃)(OR), (CRR1)ₙP(═O)(OR)(OR1), O(CRR1)ₙP(═O)(Me)(OR), O(CRR1)ₙP(═O)(CF₃)(OR), OCF[P(═O)(Me)(OR)]₂, SO₃H, —(CRR1)ₙSO₃H, S(O)ₙR, SCF₃, SCHF₂, SO₂CF₃, SO₂Ph, (CRR1)ₙS(O)ₙR, (CRR1)ₙS(O)₂CF₃, (CRR1)ₙSO₂NRR1, (CRR1)ₙSO₂NRC(═O)(Me, CF₃), (CF₂)ₙSO₃H, (CFR)ₙSO₃H, (CF₂)ₙSO₂NRR1, wherein n=0-2, and R and R1 are as defined above;

Y₁, Y₂ and/or Y₃ may also be selected together to be (CRR1)₂₋₆ and substituted variants thereof, —O[C(R)(R3)]ᵣO— or —O[C(R2)(R3)]ᵣ₊₁—, wherein r is an integer from 1 to 4 and R2 and R3 are independently selected from the group consisting of hydrogen, alkyl of 1 to about 12 carbon atoms, aryl of about 6 to about 14 carbon atoms, heteroaryl of about 5 to about 14 ring atoms, aralkyl of about 7 to about 15 carbon atoms, and heteroarylalkyl of about 5 to about 14 ring atoms;

G₁, G₂, and G₃ are selected from the following:

(i) H, alkyl of 1 to about 6 carbon atoms and which is unsubstituted or mono-, di- or tri-substituted with 1 to 3 substituents selected from the group consisting of Y₁, Y₂, and Y₃, alkenyl of 2 to about 6 carbon atoms and which is unsubstituted or mono-, di- or tri-substituted with 1 to 3 substituents selected from the group consisting of Y₁, Y₂, and Y₃, alkynyl of 2 to about 6 carbon atoms and which is unsubstituted or mono-, di- or tri-substituted with 1 to 3 substituents selected from the group consisting of Y₁, Y₂, and Y₃, cycloalkyl of 3 to about 8 carbon atoms and which is unsubstituted or mono-, di- or tri-substituted with 1 to 3 substituents selected from the group consisting of Y₁, Y₂, and Y₃, aryl of about 6 to about 14 carbon atoms and which is unsubstituted or mono-, di- or tri-substituted with 1 to 3 substituents selected from the group consisting of Y₁, Y₂, and Y₃, aralkyl of about 7 to about 16 carbon atoms which is unsubstituted or mono-, di- or tri-substituted with 1 to 3 substituents selected from the group consisting of Y₁, Y₂, and Y₃, heteroaryl of about 5 to about 14 ring atoms with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, and heteroaralkyl of about 5 to about 14 ring atoms with the ring atoms selected from carbon and heteroatoms, wherein the heteroatoms are selected from oxygen, nitrogen, and sulfur, which is unsubstituted or substituted on the alkyl chain and which is unsubstituted on the ring or mono-, di- or tri-substituted on the ring with 1 to 3 substituents selected from the group consisting of Y₁, Y₂, and Y₃;

(ii) P(═O)(OR)(OR1), including P(═O)(OH)₂, P(═O)(OH)(OCH₃), P(═O)(OH)(OC₂H₅), P(═O)(OR)(OR1), P(═O)(OR)[(OCRR1)OC(═O)R], P(═O)(OR)[(OCRR1)OC(═O)OR], P(═O)[(OCRR1OC(═O)R)][(OCRR1)OC(═O)R], P(═O)[(OCRR1)OC(═O)OR)][(OCRR1)OC(═O)OR], P(═O)(Me)(OR), P(═O)(CF₃)(OR), P(═O)(Me)(NHR), P(═O)(NHR)(OR), P(═O)(NHR)(NHR1), CR═CR—P(═O)(OR)(OR1), CR═CR—P(═O)(Me)(OR), CR═CR—P(═O)(CF₃)(OR), CR═CR—P(═O)(Me)(NHR), CR═CR—P(═O)(NHR)(OR), CR═CR—P(═O)(NHR)(NHR1), [CH(OH)]_{q}P(═O)(OR)(OR1), [CH(OH)]_{q}P(═O)(Me)(OR1), [CH(OH)]_{q}P(═O)(CF₃)(OR1), CC—P(═O)(OR)(OR1), CC—P(═O)(Me)(OR), CC—P(═O)(CF₃)(OR), CC—(CF₂)_{q}—P(═O)(OR)(OR1), CC—(CF₂)_{q}—P(═O)(Me)(OR1), CC—(CF₂)_{q}—P(═O)(CF₃)(OR1), [CH(OH)]_{q}CF₂P(═O)(OR)(OR1), [CH(OH)]_{q}(CF₂)_{q}P(═O)(Me)(OR1), [CH(OH)]_{q}(CF₂)_{q}P(═O)(CF₃)(OR1), (CF2)_{q}P(═O)(OR)(OR1), (CF₂)_{q}P(═O)(Me)(OR), (CF₂)_{q}P(═O)(CF₃)(OR), (CF₂)_{q}P(═O)(Me)NHR, (CF2)_{q}P(═O)(NHR)(OR), (CFR)_{q}P(═O)(OR)(OR1), (CFR)_{q}P(═O)(Me)(OR), (CFR)_{q}P(═O)(CF₃)(OR), (CFR)_{q}P(═O)(Me)NHR, (CF₂)_{q}P(═O)(NHR)(OR), CF═CF—P(═O)(OR)(OR1), CF═CF—P(═O)(Me)(OR), CF═CF—P(═O)(CF₃)(OR), CF═CF—P(═O)(Me)(NHR), CF═CF—P(═O)(NHR)(OR), CH═C[P(═O)(OR)₂]₂, CF═C[P(═O)(OR)₂]₂, CH[P(═O)(OR)₂]₂, CH[P(═O)(OR)(OR1)]₂, CH[P(═O)(Me)(OR)]₂, CH[P(═O)(CF₃)(OR)]₂, CH[P(═O)(Me)NHR]₂, CH[P(═O)(NHR)(OR)]₂, CF[P(═O)(OR)₂]₂, CF[P(═O)(OR)(OR1)]₂, CF[P(═O)(Me)(OR)]₂, CF[P(═O)(CF₃)(OR)]₂, CF[P(═O)(Me)(NHR)]₂, CF[P(═O)(NHR)(OR)]₂, C(OH)[P(═O)(OR)(OR1)]₂, C(OH)[P(═O)(Me)(OR)]₂, C(OH)[P(═O)(CF₃)(OR)]₂, C(OH)[P(═O)(Me)NHR]₂, C(OH)[P(═O)(NHR)(OR)]₂, wherein q =1 to 3

(iii) SO₃H, SO₂NH₂, SO₂NHTzl, SO₂NHC(═O)(Me, CF₃), SO₂NHC(═O)NH₂, (CRR1)_{q}SO₃H, (CRR1)_{q}SO₂NH₂, (CRR1)_{q}SO₂NHTzl, (CRR1)_{q}SO₂NHC(═O)(Me, CF₃), (CRR1)_{q}SO₂NHC(═O)NH₂, SO₂NHCRR1C(═O)C(═O)OR, SO₂CF₃, CH(SO₂Me)₂, CH(SO₂CF₃)₂, SO₂CRR1C(═O)OR, SO₂CH[C(═O)OR]₂, (CRR1)_{q}SO₂NHCRR1C(═O)C(═O)OR, (CRR1)_{q}SO₂CF₃, (CRR1)_{q}CH(SO₂Me)₂, (CRR1)_{q}CH(SO₂CF₃)₂, (CRR1)_{q}SO₂CRR1C(═O)OR, (CRR1)_{q}SO₂CH[C(═O)OR]₂, SO₂(CRR1)_{q}C(═O)(Me, CF₃), SO₂(CRR1)_{q}SO₂(Me, CF₃), SO₂(CRR1)_{q}Tzl, SO₂(CRR1)_{q}P(═O)(OR)₂, SO₂(CF₂)_{q}C(═O)OR, SO₂(CF₂)_{q}Tzl, SO₂(CF₂)_{q}P(═O)(OR)₂, SO₂NHSO₂(CF₃, Me), (CF₂)_{q}SO₂(OH, NH₂), (CF₂)_{q}SO₂NHC(═O)(CF₃, Me), (CFR)_{q}SO₂(OH, NH₂), (CFR)_{q}SO₂NHC(═O)(CF₃, Me), CR═CRSO₂(OR, NHR), CR═CRSO₂NH₂, CR═CRSO₂NHC(═O)(Me, CF₃), C(═NSO₂CF₃)(NHSO₂CF₃),

(iv) NHC(═O)C(═O)OR, NHC(═O)C(═O)O(CRR1)OC(═O)R, NHC(═O)CC(═O)O(CRR1)OC(═O)OR, NHC(═O)NRSO₂(Me, CF₃), NHSO₂(Me, CF₃), NHSO₂NRR1, NHSO₂NRC(═O)(Me, CF₃), NH(CRR1)_{q}C(═O)OR, NH(CF₂)_{q}C(═O)OR, NHTzl, NHC(═O)Tzl, NHSO₂Tzl, NH(CF₂)_{q}Tzl, NHSO₂(CRR1)_{q}C(═O)OR, NHSO₂(CF₂)_{q}C(═O)OR, (CRR1)_{q}NO₂, (CF₂)_{q}NO₂, CR═CRNO₂, CF═CFNO₂, (CRR1)_{q}NHSO₂(Me/CF₃), (CRR1)_{q}NHC(═O)(Me/CF₃), N(OCRR1C(═O)OR)CRR1C(═O)OR, NHCH[C(═O)OR]CH(OH)C(═O)OR, NHC(═O)[CH(OH)]_{q}C(═O)OR, NH(CRR1)_{q}P(═O)(OR)(OR1), NH(CRR1)_{q}P(═O)(Me)(OR), NH(CRR1)_{q}P(═O)(CF₃)(OR), NH(CF₂)_{q}P(═O)(OR)(OR1), NH(CF₂)_{q}P(═O)(Me)(OR), NH(CF₂)_{q}P(═O)(CF₃)(OR), NH(CFR)_{q}P(═O)(OR)(OR1), NH(CFR)_{q}P(═O)(Me)(OR), NH(CFR)_{q}P(═O)(CF₃)(OR),

(v) C(═O)OR, C(═O)O(CRR1)OC(═O)R, C(═O)O(CRR1)OC(═O)OR, C(═O)NHR, (CF₂)_{q}C(═O)OR, (CFR)_{q}C(═O)OR, CH[C(═O)OR]₂, CF[C(═O)OR]₂, CH═C[C(═O)OR]₂, CF═C[C(═O)OR]₂, C(R4)═C(R5)(R6), (where R4, R5=H, Me, anionic groups, including OH, SO₃H, carboxyl, tetrazole, 3-hydroxy-isoxazol-5-yl, C(═O)NHSO₂(Me, CF₃), C(═O)NHC(═O)(Me, CF₃), SO₂NHC(═O)(Me, CF₃), R6=H, F), C(═O)C(═O)OR, C(═O)CH[C(═O)OR]₂, C(═O)CH(Tzl)₂, C(═O)CRR1C(═O)(Me, CF₃, Ph), C(═O)CRR1SO₂(Me, CF₃, Ph), (CRR1)_{q}C(═O)C(═O)OR, (CF₂)_{q}C(═O)C(═O)OR, [CH(OR)]_{q}C(═O)OR, (CRR1)_{q}[CH(OR)]_{q}C(═O)OR, CR═CRCH(OR)C(═O)OR, C(OR)(CF₃)C(═O)OR, (CF₂)_{q}C(═O)CF₃, (CF₂)_{q}C(OH)₂CF₃, (CHF)_{q}C(═O)CF₃, (CF₂)_{q}C(═O)CF₃, (CHF)_{q}C(OR)₂CF₃, (CF₂)_{q}C(OR)₂CF₃, CH(OR)CH[C(═O)OR]₂, C(OR)[CRR1C(═O)OR]₂, (CF₂)_{q}C(OR)C(═O)OR, C(═O)C(═NOR)C(═O)(CH₃, OR), C(═O)CRR1C(═O)C(═O)OR, C(═NOR)C(═O)OR, CH═NOCRR1C(═O)OR, C[C(═O)OH]═NOCRR1C(═O)OR, CH(CN)NHC(═O)C(═O)OR, CH(NHCHO)C(═O)C(═O)OR, CH(NHCHO)C(OR)C(═O)OR, C(═O)N[CRR1C(═O)OR]OCRR1C(═O)OR, C(═O)N[CRR1C(═O)OR]₂, C(═O)N(CRR1Tzl)₂, C(═O)N[CRR1P(═O)(OR)₂]₂, C(═O)NHC(CRR1OR)₃,

(vi) Tzl, CR(Tzl)₂, (CRR1)_{q}Tzl, (CF₂)_{q}Tzl, (CFR)_{q}Tzl, CF(Tzl)₂, (CF₂)_{q}CF(Tzl)₂, (CF₂)_{q}CR(Tzl)₂, CR═CR-Tzl, CF═CH-Tzl, CH═CF-Tzl, CF═CF-Tzl, CH═C(Tzl)₂, CF═C(Tzl)₂, C(H, F)═C(Tzl)[P(═O)(OR)(OR1), P(═O)(Me)(OR), P(═O)(CF₃)(OR), P(═O)(Me)(NHR), P(═O)(NHR)(OR), C(═O)OR],

(vii) OH, OR, O(CRR1)_{q}C(═O)OR, O(CF₂)_{q}C(═O)OR, OCH[C(═O)OR]₂, O(CRR1)_{q}CH[C(═O)OR]₂, OCF[C(═O)OR]₂, O(CRR1)_{q}CF[C(═O)OR]₂, O(CRR1)_{q}C(═O)C(═O)OR, O(CF₂)_{q}C(═O)C(═O)OR, O(CRR1)_{q}[CH(OR)]_{q}C(═O)OR, OCH[CRR1C(═O)OR]₂, OCF[CRR1C(═O)OR]₂, O(CF₂)_{q}CR(OR1)C(═O)OR, OTzl, O(CRR1)_{q}Tzl, O(CF₂)_{q}Tzl, OCH(Tzl)₂, O(CF₂)_{q}CF(Tzl)₂, O(CF₂)_{q}CR(Tzl)₂, OCF(Tzl)₂, O(CF₂)_{q}P(═O)(OR)(OR1), O(CF₂)_{q}P(═O)(Me)(OR), O(CF₂)_{q}P(═O)(CF₃)(OR), O(CF₂)_{q}P(═O)(Me)(NHR), O(CF₂)_{q}P(═O)(NHR)(OR), O(CF₂)_{q}P(═O)(NHR)(NHR1), O(CFR)_{q}P(═O)(OR)(OR1), O(CFR)_{q}P(═O)(Me)(OR), O(CFR)_{q}P(═O)(CF₃)(OR), O(CFR)_{q}P(═O)(Me)(NHR), O(CFR)_{q}P(═O)(NHR)(OR), O(CFR)_{q}P(═O)(NHR)(NHR1), O(CRR1)_{q}P(═O)(OR)(OR1), O(CRR1)_{q}P(═O)(Me)(OR), O(CRR1)_{q}P(═O)(CF₃)(OR), O(CRR1)_{q}P(═O)(Me)(NHR), O(CRR1)_{q}P(═O)(NHR)(OR), O(CRR1)_{q}P(═O)(Me)(OR), OCH[P(═O)(OR)(OR1)]₂, OCH[P(═O)(Me)(OR)]₂, OCH[P(═O)(Me)(OR)]₂, OCH[P(═O)(CF₃)(NHR)]₂, OCH[P(═O)(NHR)(OR)]₂, OCF[P(═O)(OR)(OR1)]₂, OCF[P(═O)(Me)(OR)]₂, OCF[P(═O)(CF₃)(NHR)]₂, OCF[P(═O)(NHR)(OR)]₂, O(CRR1)_{q}(CF₂)_{q}P(═O)(OR)(OR1), O(CRR1)_{q}(CF₂)_{q}P(═O)(Me)(OR), O(CRR1)_{q}(CF₂)_{q}P(═O)(CF₃)(OR), O(CRR1)_{q}(CF₂)_{q}P(═O)(Me)(NHR), O(CRR1)_{q}(CF₂)_{q}P(═O)(NHR)(OR), ON═CH—C(═O)OR, ON═C[C(═O)OR]CRR1C(═O)OR,

(viii) Heteroaryl, squarate, and related derivatives, including:

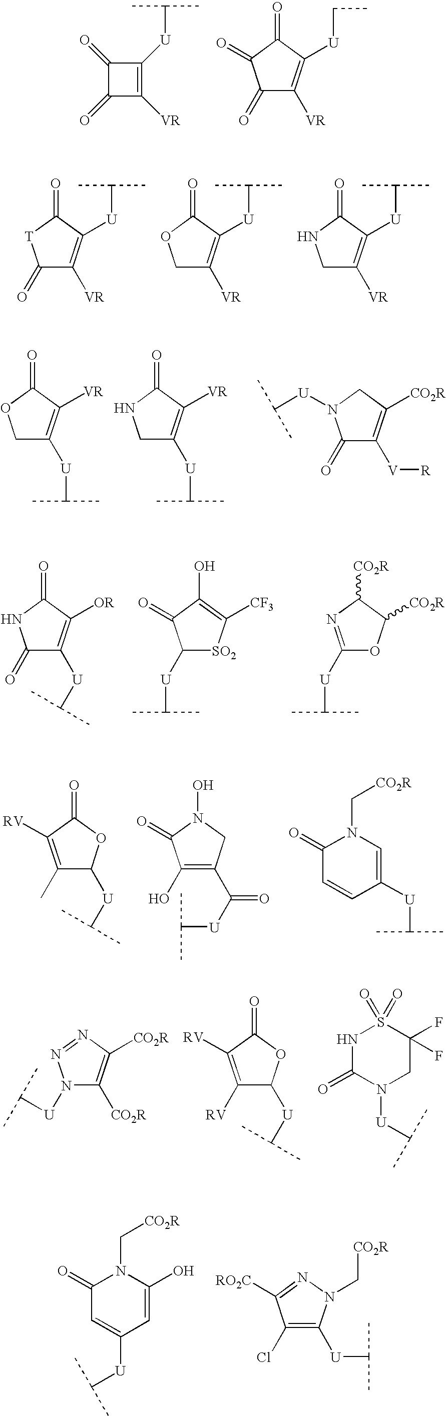

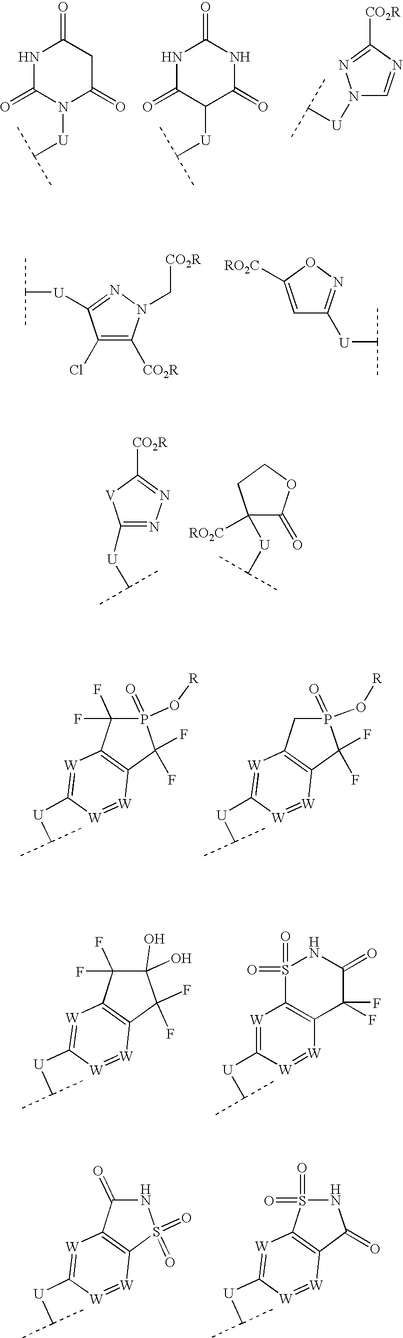

wherein T=O, NR1, CR; U and V are chosen from direct link, (CRR1)_{q}, O, S, NR1; W=CR, N R and R1 are as defined above.

Other compounds provided herein contain linkers L₁ and L₂ each containing 1 to 2 atoms and G₁ groups with optionally substituted aromatic and heteroaromatic groups of the generic formula:

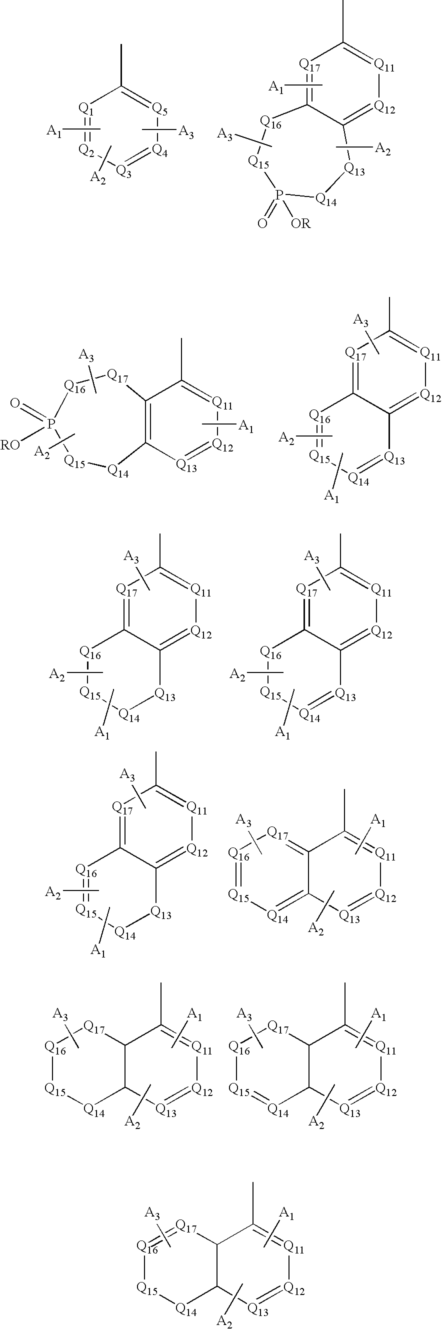

G₂ is selected from optionally substituted aromatic and heteroaromatic groups of the generic formula:

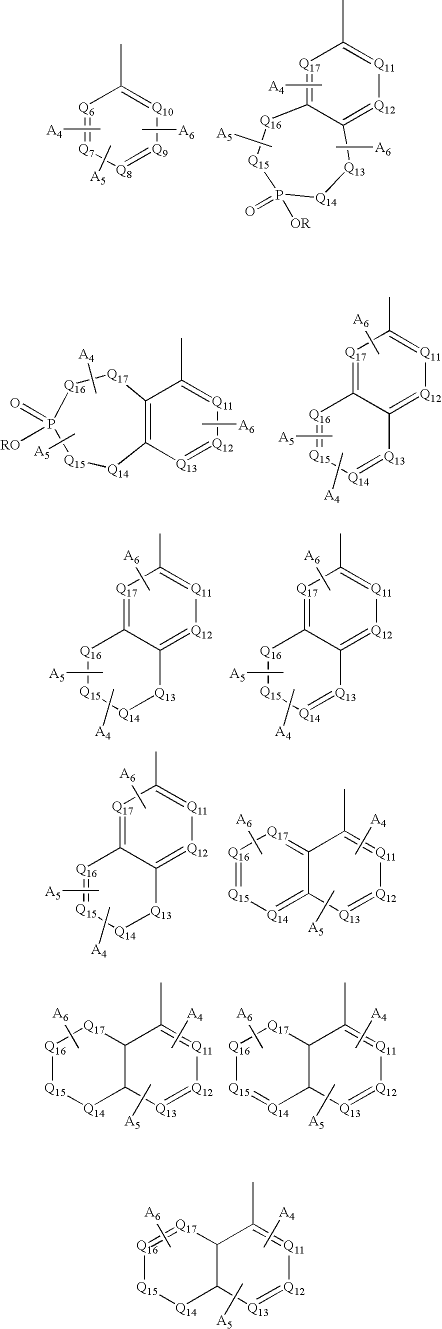

wherein A1-A6 are independently selected from

(i) H, F, Cl, Br, I, CF₃, OH, OCF₃, OCHCl₂, CN, NO₂, C₁-C₆-alkyl which is unsubstituted or mono-, di- or tri-substituted with 1 to 3 substituents selected from the group consisting of Y₁, Y₂, and Y₃, C₂-C₆-alkenyl which is unsubstituted or mono-, di- or tri-substituted with 1 to 3 substituents selected from the group consisting of Y₁, Y₂, and Y₃, C₂-C₆-alkynyl which is unsubstituted or mono-, di- or tri-substituted with 1 to 3 substituents selected from the group consisting of Y₁, Y₂, and Y₃, C₁-C₆ alkoxy which is unsubstituted or mono-, di- or tri-substituted with 1 to 3 substituents selected from the group consisting of Y₁, Y₂, and Y₃, C₃-C₆ alkenyloxy which is unsubstituted or mono-, di- or tri-substituted with 1 to 3 substituents selected from the group consisting of Y₁, Y₂, and Y₃, C₃-C₆ alkynyloxy which is unsubstituted or mono-, di- or tri-substituted with 1 to 3 substituents selected from the group consisting of Y₁, Y₂, and Y₃, wherein Y₁, Y₂, and Y₃ are defined above, C₃-C₈-cycloalkyl which is unsubstituted or mono-, di- or tri-substituted with 1 to 3 substituents selected from the group consisting of Y₁, Y₂, and Y₃, aryl of about 6 to about 14 carbon atoms and which is unsubstituted or mono-, di- or tri-substituted with 1 to 3 substituents selected from the group consisting of Y₁, Y₂, and Y₃, aralkyl of about 7 to about 16 carbon atoms which is unsubstituted or mono-, di- or tri-substituted with 1 to 3 substituents selected from the group consisting of Y₁, Y₂, and Y₃, heteroaryl of about 5 to about 14 ring atoms with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, and heteroaralkyl of about 5 to about 14 ring atoms with the ring atoms selected from carbon and heteroatoms, wherein the heteroatoms are selected from oxygen, nitrogen, and sulfur, which is unsubstituted or substituted on the alkyl chain and which is unsubstituted on the ring or mono-, di- or tri-substituted on the ring with 1 to 3 substituents selected from the group consisting of Y₁, Y₂, and Y₃; two adjacent A groups (e.g. A₁, A₂) may be joined together to form a fused alicyclic, heteroaromatic or aromatic ring. R, R1=H, C₁-C₆-alkyl, C₂-C₆-alkenyl, C₂-C₆-alkynyl. R, R1 may be joined together to form an alicyclic or heterocyclic ring. One or more of A₁-A₆ may serve as a linking atom, such as O, S(O)₀₋₂, C(RR1), P(═O), P(═S), or N(R).

Other A₁-A₆ phosphorous-containing moieties include the following:

P(═O)(OR)(OR1), especially P(═O)(OH)₂, P(═O)(OH)(OCH₃), P(═O)(OH)(OC₂H₅), P(═O)(OR)(OR1), P(═O)(OR)[(OCRR1)OC(═O)R], P(═O)(OR)[(OCRR1OC(═O)OR], P(═O)[(OCRR1)OC(═O)R)][(OCRR1)OC(═O)R], P(═O)[(OCRR1)OC(═O)OR)][(OCRR1)OC(═O)OR], P(═O)(OR)(OR1), P(═O)(Me)(OR), P(═O)(CF₃)(OR), P(═O)(Me)(NHR), P(═O)(NHR)(OR), P(═O)(NHR)(NHR1), CR═CR—P(═O)(OR)(OR1), CR═CR—P(═O)(Me)(OR), CR═CR—P(═O)(CF₃)(OR), CR═CR—P(═O)(Me)(NHR), CR═CR—P(═O)(NHR)(OR), CR═CR—P(═O)(NHR)(NHR1), [CH(OH)]_{q}P(═O)(OR)(OR1), [CH(OH)]_{q}P(═O)(Me)(OR1), [CH(OH)]_{q}P(═O)(CF₃)(OR1), CC—P(═O)(OR)(OR1), CC—P(═O)(Me)(OR), CC—P(═O)(CF₃)(OR), CC—(CF₂)_{q}—P(═O)(OR)(OR1), CC—(CF₂)_{q}—P(═O)(Me)(OR1), CC—(CF₂)_{q}—P(═O)(CF₃)(OR1), [CH(OH)]_{q}CF₂P(═O)(OR)(OR1), [CH(OH)]_{q}(CF₂)_{q}P(═O)(Me)(OR1), [CH(OH)]_{q}(CF₂)_{q}P(═O)(CF₃)(OR1), (CF₂)_{q}P(═O)(OR)(OR1), (CF₂)_{q}P(═O)(Me)(OR), (CF₂)_{q}P(═O)(CF₃)(OR), (CF₂)_{q}P(═O)(Me)NHR, (CF₂)_{q}P(═O)(NHR)(OR), (CFR)_{q}P(═O)(OR)(OR1), (CFR)_{q}P(═O)(Me)(OR), —(CFR)_{q}P(═O)(CF₃)(OR), (CFR)_{q}P(═O)(Me)NHR, (CF₂)_{q}P(═O)(NHR)(OR), CF═CF—P(═O)(OR)(OR1), CF═CF—P(═O)(Me)(OR), CF═CF—P(═O)(CF₃)(OR), CF═CF—P(═O)(Me)(NHR), CF═CF—P(═O)(NHR)(OR), CH═C[P(═O)(OR)₂]₂, CF═C[P(═O)(OR)₂]₂,CH[P(═O)(OR)₂]₂, CH[P(═O)(OR)(OR1)]₂, CH[P(═O)(Me)(OR)]₂, CH[P(═O)(CF₃)(OR)]₂, CH[P(═O)(Me)NHR]₂, CH[P(═O)(NHR)(OR)]₂, CF[P(═O)(OR)₂]₂, CF[P(═O)(OR)(OR1)]₂, CF[P(═O)(Me)(OR)]₂, CF[P(═O)(CF₃)(OR)]₂, CF[P(═O)(Me)(NHR)]₂, CF[P(═O)(NHR)(OR)]₂, C(OH)[P(═O)(OR)(OR1)]₂, C(OH)[P(═O)(Me)(OR)]₂, C(OH)[P(═O)(CF₃)(OR)]₂, C(OH)[P(═O)(Me)NHR]₂, C(OH)[P(═O)(NHR)(OR)]₂, wherein q=1 to 3 throughout

Other A₁-A₆ sulfur-containing moieties include the following:

SO₃H, SO₂NH₂, SO₂NHTzl, SO₂NHC(═O)(Me, CF₃), SO₂NHC(═O)NH₂, (CRR1)_{q}SO₃H, (CRR1)_{q}SO₂NH₂, (CRR1)_{q}SO₂NHTzl, (CRR1)_{q}SO₂NHC(═O)(Me, CF₃), CH(SO₂Me)₂, CH(SO₂CF₃)₂, SO₂CRR1C(═O)OR, SO₂CH[C(═O)OR]₂, (CRR1)_{q}SO₂NHCRR1C(═O)C(═O)OR, (CRR1)_{q}SO₂CF₃, (CRR1)_{q}CH(SO₂Me)₂, (CRR1)_{q}CH(SO₂CF₃)₂, (CRR1)_{q}SO₂CRR1C(═O)OR, (CRR1)_{q}SO₂CH[C(═O)OR]₂, SO₂(CRR1)_{q}C(═O)(Me, CF₃), SO₂(CRR1)_{q}SO₂(Me, CF₃), SO₂(CRR1)_{q}Tzl, SO₂(CRR1)_{q}P(═O)(OR)₂, SO₂(CF₂)_{q}C(═O)OR, SO₂(CF₂)_{q}Tzl, SO₂(CF₂)_{q}P(═O)(OR)₂, SO₂NHSO₂(CF₃, Me), (CF₂)_{q}SO₂(OH, NH₂), (CF₂)_{q}SO₂NHC(═O)(CF₃, Me), (CFR)_{q}SO₂(OH, NH₂), (CFR)_{q}SO₂NHC(═O)(CF₃, Me), CR═CRSO₂(OR, NHR), CR═CRSO₂NH₂, CR═CRSO₂NHC(═O)(Me, CF₃), C(═NSO₂CF₃)(NHSO₂CF₃),

Other A₁-A₆ nitrogen-containing moieties include the following:

NHC(═O)C(═O)OR, NHC(═O)C(═O)O(CRR1)OC(═O)R, NHC(═O)CC(═O)O(CRR1OC(═O)OR, NHC(═O)NRSO₂(Me, CF₃), NHSO₂(Me, CF₃), NHSO₂NRR1, NHSO₂NRC(═O)(Me, CF₃), NH(CRR1)_{q}C(═O)OR, NH(CF₂)_{q}C(═O)OR, NHTzl, NHC(═O)Tzl, NHSO₂Tzl, NH(CF₂)_{q}Tzl, NHSO₂(CRR1)_{q}C(═O)OR, NHSO₂(CF₂)_{q}C(═O)OR, (CRR1)_{q}NO₂, (CF₂)_{q}NO₂, CR═CRNO₂, CF═CFNO₂, (CRR1)_{q}NHSO₂(Me/CF₃), (CRR1)_{q}NHC(═O)(Me/CF₃), N(OCRR1C(═O)OR)CRR1C(═O)OR, NHCH[C(═O)OR]CH(OH)C(═O)OR, NHC(═O)[CH(OH)]_{q}C(═O)OR, NH(CRR1)_{q}P(═O)(OR)(OR1), NH(CRR1)_{q}P(═O)(Me)(OR), NH(CRR1)_{q}P(═O)(CF₃)(OR), NH(CF₂)_{q}P(═O)(OR)(OR1), NH(CF₂)_{q}P(═O)(Me)(OR), NH(CF₂)_{q}P(═O)(CF₃)(OR), NH(CFR)_{q}P(═O)(OR)(OR1), NH(CFR)_{q}P(═O)(Me)(OR), NH(CFR)_{q}P(═O)(CF₃)(OR),

Other A₁-A₆ carbonyl-containing moieties include the following:

C(═O)OR, C(═O)O(CRR1)OC(═O)R, C(═O)O(CRR1)OC(═O)OR, C(═O)NHR, (CF₂)_{q}C(═O)OR, (CFR)_{q}C(═O)OR, CH[C(═O)OR]₂, CF[C(═O)OR]₂, CH═C[C(═O)OR]₂, CF═C[C(═O)OR]₂, C(R4)═C(R5)(R6), (where R4, R5=H, Me, anionic groups, including OH, SO₃H, carboxyl, tetrazole, 3-hydroxy-isoxazol-5-yl, C(═O)NHSO₂(Me, CF₃), C(═O)NHC(═O)(Me, CF₃), SO₂NHC(═O)(Me, CF₃), R6=H, F), C(═O)C(═O)OR, C(═O)CH[C(═O)OR]₂, C(═O)CH(Tzl)₂, C(═O)CRR1C(═O)(Me, CF₃, Ph), C(═O)CRR1SO₂(Me, CF₃, Ph), (CRR1)_{q}C(═O)C(═O)OR, (CF₂)_{q}C(═O)C(═O)OR, [CH(OR)]_{q}C(═O)OR, (CRR1)_{q}[CH(OR)]_{q}C(═O)OR, CR═CRCH(OR)C(═O)OR, C(OR)(CF₃)C(═O)OR, (CF₂)_{q}C(═O)CF₃, (CF₂)_{q}C(OH)₂CF₃, CHFC(═O)CF₃, CHFC(OR)₂CF₃, CH(OR)CH[C(═O)OR]₂, C(OR)[CRR1C(═O)OR]₂, (CF₂)_{q}C(OR)C(═O)OR, C(═O)C(═NOR)C(═O)(CH₃, OR), C(═O)CRR1C(═O)C(═O)OR, C(═NOR)C(═O)OR, CH═NOCRR1C(═O)OR, C[C(═O)OH]═NOCRR1C(═O)OR, CH(CN)NHC(═O)C(═O)OR, CH(NHCHO)C(═O)C(═O)OR, CH(NHCHO)C(OR)C(═O)OR, C(═O)N[CRR1C(═O)OR]OCRR1C(═O)OR, C(═O)N[CRR1C(═O)OR]₂, C(═O)N(CRR1Tzl)₂, C(═O)N[CRR1P(═O)(OR)₂]₂, C(═O)NHC(CRR1OR)₃,

Other A₁-A₆ tetrazole (Tzl)-containing moieties include the following:

Tzl, CR(Tzl)₂, (CRR1)_{q}Tzl, (CF₂)_{q}Tzl, (CFR)_{q}Tzl, CF(Tzl)₂, (CF₂)_{q}CF(Tzl)₂, (CF₂)_{q}CR(Tzl)₂, CR═CR-Tzl, CF═CH-Tzl, CH═CF-Tzl, CF═CF-Tzl, CH═C(Tzl)₂, CF═C(Tzl)₂, C(H, F)═C(Tzl)[P(═O)(OR)(OR1), P(═O)(Me)(OR), P(═O)(CF₃)(OR), P(═O)(Me)(NHR), P(═O)(NHR)(OR), C(═O)OR],

Other A₁-A₆ oxygen-containing or oxygen-linked moieties include the following:

OH, OR, O(CRR1)_{q}C(═O)OR, O(CF₂)_{q}C(═O)OR, OCH[C(═O)OR]₂, O(CRR1)_{q}CH[C(═O)OR]₂, OCF[C(═O)OR]₂, O(CRR1)_{q}CF[C(═O)OR]₂, O(CRR1)_{q}C(═O)C(═O)OR, O(CF₂)_{q}C(═O)C(═O)OR, O(CRR1)_{q}[CH(OR)]_{q}C(═O)OR, OCH[CRR1C(═O)OR]₂, OCF[CRR1C(═O)OR]₂, O(CF₂)_{q}CR(OR1)C(═O)OR, OTzl, O(CRR1)_{q}Tzl, O(CF₂)_{q}Tzl, OCH(Tzl)₂, O(CF₂)_{q}CF(Tzl)₂, O(CF₂)_{q}CR(Tzl)₂, OCF(Tzl)₂, O(CF₂)_{q}P(═O)(OR)(OR1), O(CF₂)_{q}P(═O)(Me)(OR), O(CF₂)_{q}P(═O)(CF₃)(OR), O(CF₂)_{q}P(═O)(Me)(NHR), O(CF₂)_{q}P(═O)(NHR)(OR), O(CF₂)_{q}P(═O)(NHR)(NHR1), O(CFR)_{q}P(═O)(OR)(OR1), O(CFR)_{q}P(═O)(Me)(OR), O(CFR)_{q}P(═O)(CF₃)(OR), O(CFR)_{q}P(═O)(Me)(NHR), O(CFR)_{q}P(═O)(NHR)(OR), O(CFR)_{q}P(═O)(NHR)(NHR1), O(CRR1)_{q}P(═O)(OR)(OR1), O(CRR1)_{q}P(═O)(Me)(OR), O(CRR1)_{q}P(═O)(CF₃)(OR), O(CRR1)_{q}P(═O)(Me)(NHR), O(CRR1)_{q}P(═O)(NHR)(OR), O(CRR1)_{q}P(═O)(Me)(OR), OCH[P(═O)(OR)(OR1)]₂, OCH[P(═O)(Me)(OR)]₂, OCH[P(═O)(Me)(OR)]₂, OCH[P(═O)(CF₃)(NHR)]₂, OCH[P(═O)(NHR)(OR)]₂, OCF[P(═O)(OR)(OR1)]₂, OCF[P(═O)(Me)(OR)]₂, OCF[P(═O)(CF₃)(NHR)]₂, OCF[P(═O)(NHR)(OR)]₂, O(CRR1)_{q}(CF₂)_{q}P(═O)(OR)(OR1), O(CRR1)_{q}(CF₂)_{q}P(═O)(Me)(OR), O(CRR1)_{q}(CF₂)_{q}P(═O)(CF₃)(OR), O(CRR1)_{q}(CF₂)_{q}P(═O)(Me)(NHR), O(CRR1)_{q}(CF₂)_{q}P(═O)(NHR)(OR), ON═CH—C(═O)OR, ON═C[C(═O)OR]CRR1C(═O)OR,

Other A₁-A₆ moieties contain the following heteroaryl, squarate, and related derivatives, including: 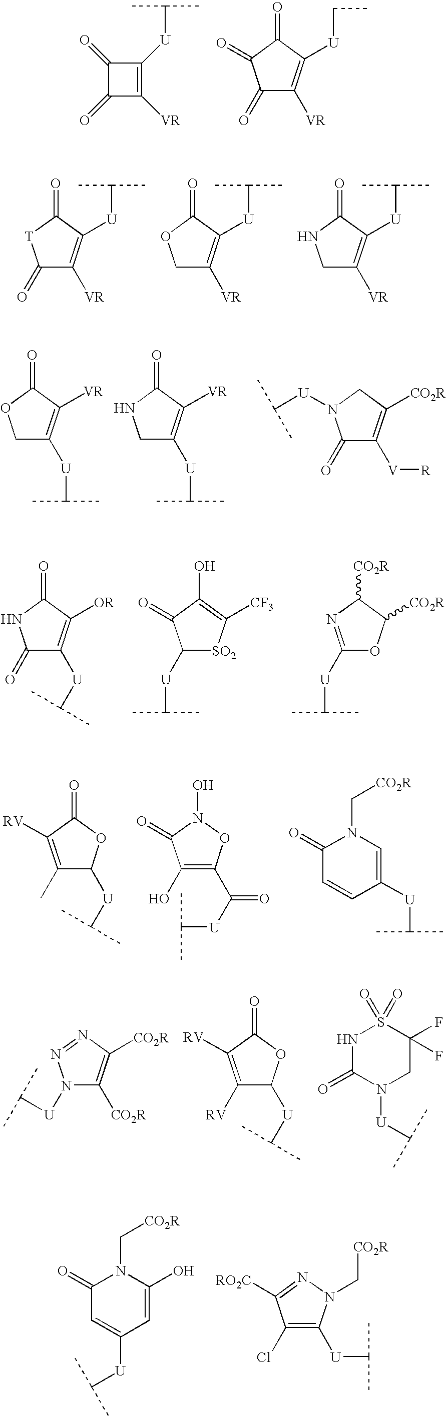 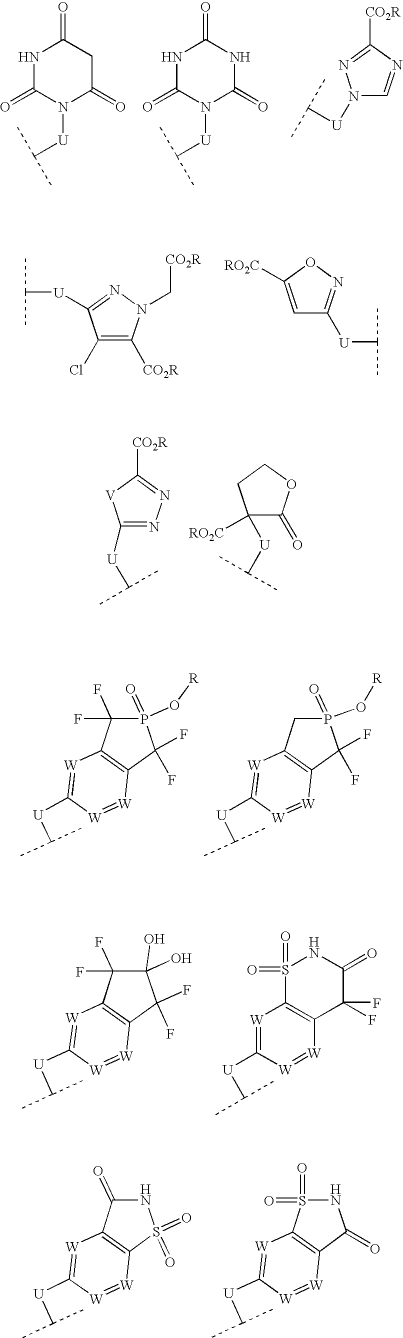
wherein T=O, NR1, CR; U and V are chosen from direct link, (CRR1)_{q}, O, S, NR1; W=CR, N; R and R1 are as defined above.

G₃ is selected from the group consisting of:
(1) alkyl of 1 to about 12 carbon atoms which is optionally unsubstituted or substituted with 1 to 3 substituents independently selected from the group consisting of Y₁, Y₂, and Y₃ as previously defined;
(2) alkyl of 1 to about 3 carbon atoms atoms which is optionally substituted with cycloalkyl of about 3 to about 8 carbon atoms which is optionally substituted with 1 to 3 substituents independently selected from the group consisting of Y₁, Y₂, and Y₃;
(3) cycloalkyl of 3 to about 15 carbon atoms, which is unsubstituted or mono-, di-, or tri-substituted on the ring with 1 to 3 substituents selected from the group consisting of Y₁, Y₂, and Y₃;
(4) alkenyl of 2 to about 6 carbon atoms and which is optionally unsubstituted or mono-, di- or tri-substituted with 1 to 3 substituents selected from the group consisting of Y₁, Y₂, and Y₃;
(5) cycloalkenyl of 4 to about 8 carbon atoms and which is optionally unsubstituted or mono-, di- or tri-substituted with 1 to 3 substituents selected from the group consisting of Y₁, Y₂, and Y₃;
(6) alkyl of 1 to about 3 carbon atoms atoms which is optionally substituted with cycloalkenyl of 4 to about 8 carbon atoms and which is optionally unsubstituted or mono-, di- or tri-substituted with 1 to 3 substituents selected from the group consisting of Y₁, Y₂, and Y₃;
(7) alkynyl of 2 to about 6 carbon atoms which is optionally unsubstituted or mono-, di- or tri-substituted with 1 to 3 substituents selected from the group consisting of Y₁, Y₂, and Y₃;
(8) alkynyl of 2 to about 6 carbon atoms which is optionally substituted with cycloalkyl of about 3 to about 8 carbon atoms, which is optionally substituted with 1 to 3 substituents independently selected from the group consisting of Y₁, Y₂, and Y₃;
(9) aryl of about 6 to about 14 carbon atoms which is optionally unsubstituted or mono-, di- or tri-substituted with 1 to 3 substituents selected from the group consisting of Y₁, Y₂, and Y₃;
(10) alkyl of 1 to about 3 carbon atoms atoms which is optionally substituted with aryl of 6 to about 14 carbon atoms, which is optionally unsubstituted or mono-, di- or tri-substituted with 1 to 3 substituents selected from the group consisting of Y₁, Y₂, and Y₃;
(11) alkenyl of 2 to about 6 carbon atoms which is optionally substituted with aryl of 6 to about 14 carbon atoms, which is optionally unsubstituted or mono-, di- or tri-substituted with 1 to 3 substituents selected from the group consisting of Y₁, Y₂, and Y₃;
(12) alkynyl of 2 to about 6 carbon atoms which is optionally substituted with aryl of 6 to about 14 carbon atoms, which is optionally unsubstituted or mono-, di- or tri-substituted with 1 to 3 substituents selected from the group consisting of Y₁, Y₂, and Y₃;
(13) heteroaryl of about 5 to about 14 ring atoms with the ring atoms selected from carbon and heteroatoms, wherein the heteroatoms are selected from oxygen, nitrogen, and sulfur, and which is optionally unsubstituted or mono-, di- or tri-substituted with 1 to 3 substituents selected from the group consisting of Y₁, Y₂, and Y₃;
(14) alkyl of 1 to about 3 carbon atoms atoms which is optionally substituted with heteroaryl of about 5 to about 14 ring atoms with the ring atoms selected from carbon and heteroatoms, wherein the heteroatoms are selected from oxygen, nitrogen, and sulfur, and which is which is optionally unsubstituted or mono-, di- or tri-substituted with 1 to 3 substituents selected from the group consisting of Y₁, Y₂, and Y₃;
(15) alkenyl of 2 to about 6 carbon atoms which is optionally substituted with heteroaryl of about 5 to about 14 ring atoms with the ring atoms selected from carbon and heteroatoms, wherein the heteroatoms are selected from oxygen, nitrogen, and sulfur, and which is which is optionally unsubstituted or mono-, di- or tri-substituted with 1 to 3 substituents selected from the group consisting of Y₁, Y₂, and Y₃;
(16) alkynyl of 2 to about 6 carbon atoms which is optionally substituted with heteroaryl of about 5 to about 14 ring atoms with the ring atoms selected from carbon and heteroatoms, wherein the heteroatoms are selected from oxygen, nitrogen, and sulfur, and which is which is optionally unsubstituted or mono-, di- or tri-substituted with 1 to 3 substituents selected from the group consisting of Y₁, Y₂, and Y₃;
(17) heterocyclo of 4 to about 10 ring atoms with the ring atoms selected from carbon and heteroatoms, wherein the heteroatoms are selected from the group consisting of oxygen, nitrogen, and S(O)ₘ, wherein m i is 0, 1 or 2, which is unsubstituted or mono-, di-, or tri-substituted on the ring with 1 to 3 substituents selected from the group consisting of Y₁, Y₂, and Y₃;
(18) alkyl of 1 to about 3 carbon atoms atoms which is optionally substituted with heterocyclo of 4 to about 10 ring atoms with the heteroring atoms selected from carbon and heteroatoms, wherein the heteroatoms are selected from the group consisting of oxygen, nitrogen, and S(O)ₘ, wherein m i is 0, 1 or 2, which is unsubstituted or mono-, di-, or tri-substituted on the ring with 1 to 3 substituents selected from the group consisting of Y₁, Y₂, and Y₃;
(19) alkenyl of 2 to about 6 carbon atoms which is optionally substituted with heterocyclo of 4 to about 10 ring atoms with the heteroring atoms selected from carbon and heteroatoms, wherein the heteroatoms are selected from the group consisting of oxygen, nitrogen, and S(O)ₘ, wherein m i is 0, 1 or 2, which is unsubstituted or mono-, di-, or tri-substituted on the ring with 1 to 3 substituents selected from the group consisting of Y₁, Y₂, and Y₃;
(20) alkynyl of 2 to about 6 carbon atoms which is optionally substituted with heterocyclo of 4 to about 10 ring atoms with the heteroring atoms selected from carbon and heteroatoms, wherein the heteroatoms are selected from the group consisting of oxygen, nitrogen, and S(O)ₘ, wherein m i is 0, 1 or 2, which is unsubstituted or mono-, di-, or tri-substituted on the ring with 1 to 3 substituents selected from the group consisting of Y₁, Y₂, and Y₃;
(21) biaryl and heterobiaryl of about 10 to 20 atoms featuring two (hetero)aromatic ring systems linked through a single, double, or triple bond, with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃;
   Wherein Y₁, Y₂, and Y₃ are as previously defined.
   Pro-Drug Compounds

Alternatively, the compounds provided herein can be further modified to act as prodrugs. It is a well-known phenomenon in drug discovery that compounds such as enzyme inhibitors can display potency and selectivity in in vitro assays, yet not readily manifest the same activity in vivo. This lack of “bioavailability” may be due to a number of factors, such as poor absorption in the gut, first-pass metabolism in the liver, and poor uptake in the cells. Although the factors determining bioavailability are not completely understood, there are many techniques known by those skilled in the art to modify compounds, which are potent and selective in biochemical assays but show low or no activity in vivo, into drugs that are biologically and therapeutically active.

It is considered to be within the scope of the present disclosure to modify any of the compounds provided herein (termed the ‘original compound’) by attaching chemical groups that will improve the bioavailability of the original compound. Examples of said modifications include changing of one or more carboxy groups to esters (for instance methyl esters, ethyl esters, acetoxymethyl esters or other acyloxy-methyl esters). Compounds provided herein so modified by attaching chemical groups are termed ‘modified compounds.’

Other examples of modified compounds are compounds that have been cyclized at specific positions (‘cyclic compounds’) which upon uptake in cells or mammals become hydrolyzed at the same specific position(s) in the molecule to yield the compounds provided herein, the original compounds, which are then said to be ‘non-cyclic’. For the avoidance of doubt, it is understood that the latter original compounds in most cases will contain other cyclic or heterocyclic structures that will not be hydrolyzed after uptake in cells or mammals.

Generally, said modified compounds will not show a behavior in biochemical assays similar to that of the original compound, i.e., the corresponding compounds provided herein without the attached chemical groups or said modifications. Said modified compounds may even be inactive in biochemical assays. However, after uptake in cells or mammals these attached chemical groups of the modified compounds may in turn be removed spontaneously or by endogenous enzymes or enzyme systems to yield compounds provided herein, original compounds. ‘Uptake’ is defined as any process that will lead to a substantial concentration of the compound inside cells or in mammals. After uptake in cells or mammals and after removal of said attached chemical group or hydrolysis of said cyclic compound, the compounds may have the same structure as the original compounds and thereby regain their activity and hence become active in cells and/or in vivo after uptake.

A number of techniques well known to those skilled in the art may be used to verify that the attached chemical groups have been removed or that the cyclic compound has been hydrolyzed after uptake in cells or mammals. One example of such techniques is as follows: A mammalian cell line, which can be obtained from the American Type Culture Collection (ATCC) or other similar governmental or commercial sources, is incubated with a modified compound. After incubation under appropriate conditions, the cells are washed, lysed and the lysate is isolated. A number of different procedures, well known to those skilled in the art, may in turn be used to extract and purify the modified compound (or a metabolite thereof) (the ‘purified compound’) from the lysate. The modified compound may or may not retain the attached chemical group or the cyclic compound may or may not have been hydrolyzed. Similarly, a number of different procedures may be used to structurally and chemically characterize the purified compound. Since the purified compound has been isolated from said cell lysate and hence has been taken up by said cell line, a comparison of the structurally and chemically characterized compound with that of the original compound (i.e. without the attached chemical group or other modification) will provide information on whether the attached chemical group as been removed in the cell or if the cyclic compound has been hydrolyzed.

As a further analysis, the purified compound may be subjected to enzyme kinetic analysis as described in detail in the present description. If the kinetic profile is similar to that of the original compound without the attached chemical group, but different from the modified compound, this result confirms that the chemical group has been removed or the cyclic compounds has been hydrolyzed. Similar techniques may be used to analyze compounds provided herein in whole animals and mammals.

One form of prodrug is acetoxymethyl esters of the compounds provided herein, which may be prepared by the following general procedure (C. Schultz et al., J. Biol. Chem. 1993, 268:6316-6322):

A carboxylic acid (1 eq) is suspended in dry acetonitrile (2 mL/0.1 mmol). Diisopropyl amine (3.0 eq) is added followed by bromomethyl acetate (1.5 eq). The mixture is stirred under nitrogen overnight at room temperature. Acetonitrile is removed under reduced pressure to yield an oil, which is diluted in ethylacetate and washed with water (3×). The organic layer is dried over anhydrous magnesium sulfate. Filtration, followed by solvent removal under reduced pressure, affords a crude oil. The product is purified by column chromatography on silica gel, using an appropriate solvent system.

Other prodrugs of the compounds provided herein are prodrugs of difluoromethylphosphonic acids and have the formulae

ArCF₂P(O)(OH)(OCH(H/Me)OC(═O)OiPr, ArCF₂P(O)[(OCH(H/Me)OC(═O)OiPr]₂, ArCF₂P(O)(OH)(OCH(H/Me)OC(═O)tBu, or ArCF2P(O)[(OCH(H/Me)OC(═O)tBu]₂. Other prodrugs of the compounds provided herein have the formulae ROCH₂CHR′CH₂O—P(O)(OH)CF₂Ar or (ROCH₂CHR′CH₂O)₂—P(O)CF₂Ar, where R is C₁₄₋₂₀-n-alkyl and R′ is H, OH or OMe. Further prodrugs of the compounds provided herein are prodrugs as described in EP 0 350 287; EP 0 674 646; U.S. Pat. No. 6,599,887; U.S. Pat. No. 6,448,392; U.S. Pat. No. 6,752,981; U.S. Pat. No. 6,312,662; U.S. 2002/0173490; Friis et al. Eur. J. Pharm. Sci. 4:49-59 (1996); Erion et al. J. Am. Chem. Soc. 126:5154-5163 (2004); WO 03/095665; Krise et al. Adv. Drug. Deliv. Rev. 19:287-310 (1996); and Ettmayer et al. J. Med. Chem. 47:2393-2404 (2004). The disclosures of these patents and publications are incorporated by reference herein in their entirety.

Examples of these prodrugs are shown in the table below.

| Structure | Example | Chemical Name |
|---|---|---|
| 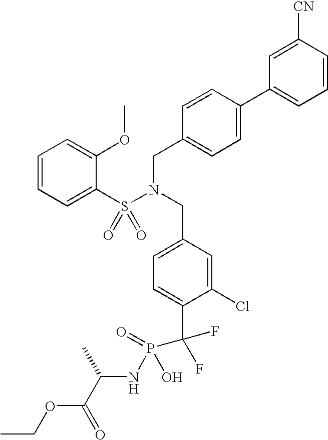 | 219 | 2-{[(2-Chloro-4-{[(3′-cyano-biphenyl-4- ylmethyl)-(2-methoxy-benzenesulfonyl)- amino]-methyl}-phenyl)-difluoro-methyl]- hydroxy-phosphinoylamino}-propionic acid ethyl ester |
| 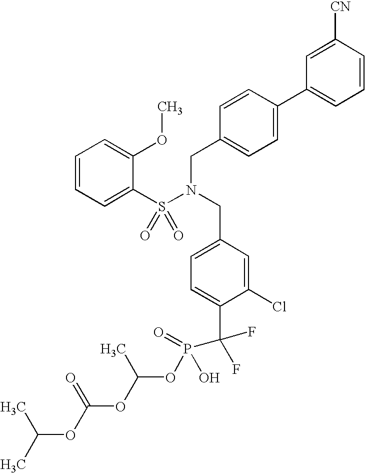 | 220 | [(2-Chloro-4-{[(3′-cyano-biphenyl-4- ylmethyl)-(2-methoxy-benzenesulfonyl)- amino]-methyl}-phenyl)-difluoro-methyl]- phosphonic acid mono-(1- isopropoxycarbonyloxy-ethyl) ester |
| 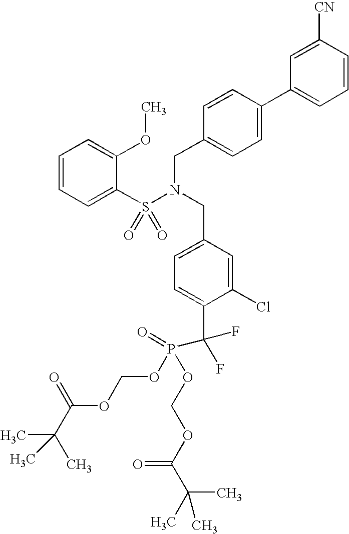 | 226 | 2,2-Dimethyl-propionic acid [(2-chloro-4-{[(3′- cyano-biphenyl-4-ylmethyl)-(2-methoxy- benzenesulfonyl)-amino]-methyl}-phenyl)- difluoro-methyl]-(2,2-dimethyl- propionyloxymethoxy)-phosphinoyloxymethyl ester |
| 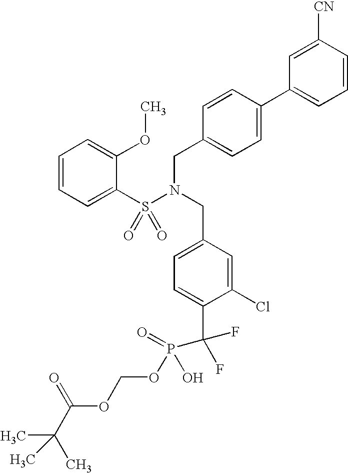 | 227 | 2,2-Dimethyl-propionic acid [(2-chloro-4-{[(3′- cyano-biphenyl-4-ylmethyl)-(2-methoxy- benzenesulfonyl)-amino]-methyl}-phenyl)- difluoro-methyl]-hydroxy- phosphinoyloxymethyl ester |

For example, compound 218 provided herein has been shown to enhance insulin-induced accumulation of pIR (phosphorylated insulin receptor) in FAO cells (about 80% of 300 nM insulin pIR signal when tested at 10 nM insulin and 10 μM compound). The in vitro activity of this compound is about 2500-24999 nM, indicating the improved activity of the prodrug in a cell-based assay. Similar results were also seen with compounds 226 and 227 provided herein.
C. Procedures for the Synthesis of Compounds and Intermediates 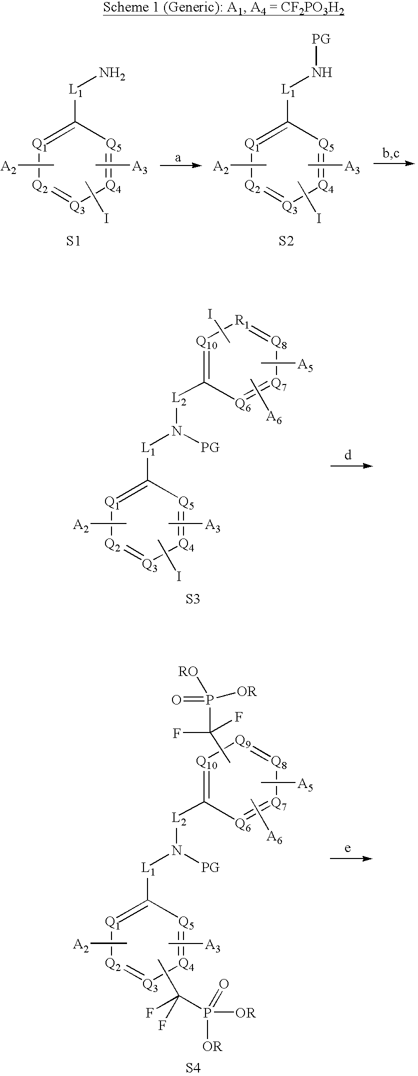 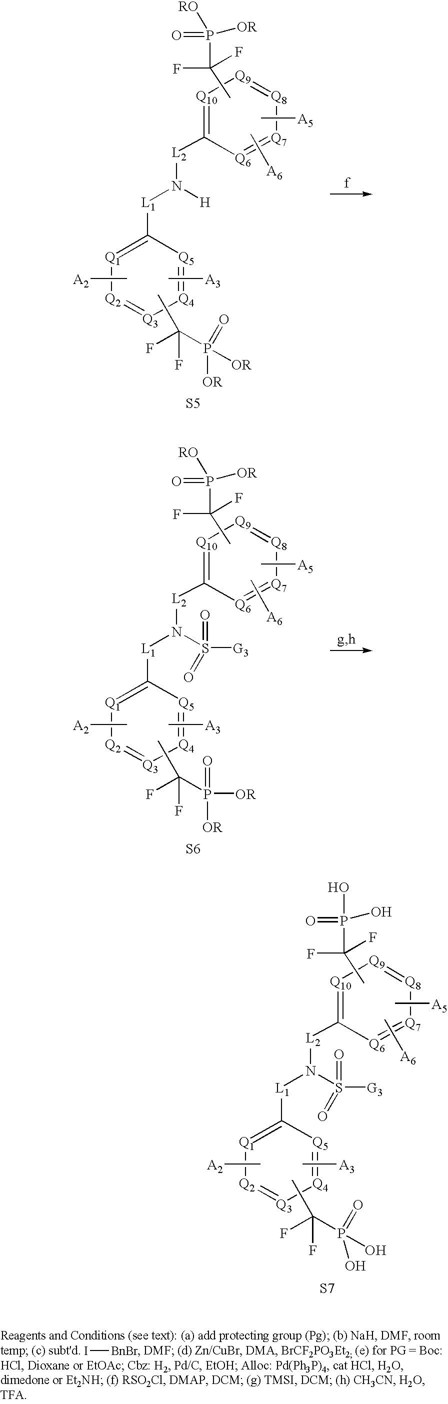
Scheme 1 through Scheme 14 depict generic synthetic routes to compounds provided herein. All generic schemes feature a key that provides a range of reaction conditions, reagents, solvents, catalysts and conditions that would be useful for preparing depicted intermediates and final target molecules to those skilled in the art of organic synthesis. More details concerning the preparation of these targets are provided in the following section. For comprehensive reviews and numerous relevant references to the generic synthetic pathways and reaction conditions presented herein below, the following authoritative works are cited: R. C. Larock, Comprehensive Organic Transformations, 2^{nd} ed., Wiley-VCH, New York, N.Y., 1999; Comprehensive Organic Chemistry, ed. D. H. R. Barton and W. D. Ollis, Pergamon Press, Oxford, 1979; Comprehensive Organic Synthesis, ed. B. M. Trost and I. Fleming, Pergamon Press, Oxford, 1991. For relevant comprehensive reviews and references on protection/deprotection strategies used herein, see: Protective Groups in Organic Synthesis, 3rd ed., ed. T. W. Greene and P. G. M. Wuts, John Wiley and Sons, New York, N.Y., 1999; cf. Ch. 5 (carboxyl), Ch. 3 (phenols), Ch. 7 (amino groups), Ch. 9 (phosphate).

Scheme 1 describes a generic protocol to N,N-disubstituted sulfonamide inhibitors S7 where two difluoromethylphosphonic acid moieties are present. Synthesis of generic target S7 commences with iodo-substituted amine derivative S1, which is protected with a suitable protecting group (PG) to afford intermediate S2. In turn, S2 is treated with a base like sodium hydride, potassium hydride, lithium bis(trimethylsilyl)amide, sodium bis(trimethylsilyl)amide, potassium bis(trimethylsilyl)amide, potassium tert-butoxide, sodium ethoxide, sodium methoxide, sodium hydroxide, potassium hydroxide, sodium carbonate or potassium carbonate in suitably compatible solvents such as DCM, DCE, DMF, DMAC, DMSO, THF, ethanol, methanol, isopropanol, tert-butanol, diethyl ether, dioxane, toluene, xylene, acetonitrile, water or mixtures thereof and is alkylated with a substituted iodo(hetero)arylalkyl halide or similar alkylating agent to provide intermediate S3. The alkylation reaction can be performed in the temperature range of about −10° C. to 100° C. Simultaneous displacement of both iodo-groups of S3 is effected by treatment with a suitable dialkyl α,α-dibromo-α-fluoromethyl phosphonate in the presence of metallic reagents such as Zn and CuBr in a dipolar aprotic solvent such as DMAC at around −20° C. to about 70° C. and affords the bisphosphonate S4. Deprotection of intermediate S4 gives the amine derivative S5 as summarized in the following cases. In the case where PG=t-Boc, intermediate S4 is treated with a hydrogen chloride or hydrogen bromide source, usually in anhydrous media at around −20° C. to about 30° C. In the case where PG=Cbz and analogous substituted benzylic carbamates, intermediate S4 is hydrogenated at ambient room temperature at around 15-70 PSI (PSI=pounds per square inch pressure) of hydrogen pressure in a suitable solvent like ethanol, methanol, THF, ethyl acetate, acetic acid, or mixtures thereof in the presence of a catalyst such as palladium on carbon, palladium hydroxide, and related palladium derivatives. Alternate hydrogenolysis conditions include palladium on carbon catalysts with hydrogen transfer reagents selected from formic acid and formate salts, cyclohexene, and sodium or potassium hypophosphite. Suitable solvents include those listed above.

In the case where PG=Alloc (allyloxycarbonyl), intermediate S4 is treated with a catalytic amount of an organopalladium reagent such as (Ph₃P)₄Pd in the presence of allyl acceptors such as diethyl amine, piperidine, morpholine and dimedone. Addition of catalytic amounts of hydrochloric acid or water may facilitate the deprotection process. Intermediate S5 is next elaborated to sulfonamide S6 by coupling with the appropriate sulfonylating reagent, typically a (hetero)aryl-, (hetero)arylalkyl-, (hetero)arylalkenyl-, (cyclo)alkyl- or (cyclo)alkenyl-sulfonyl halide, anhydride or (optionally quaternized) imidazolide at about −20° to about 30° C. in the presence of a hydrogen chloride scavenger, typically comprised of a tertiary amine like Et₃N, DIPEA, NMM, NMP, DBU, DBN, DABCO, pyridine, collidine, lutidine, or inorganic bases including sodium carbonate, potassium carbonate, sodium hydrogen carbonate, potassium hydrogen carbonate in an inert solvent such as DCM, DCE, DMF, DMAC, THF, diethyl ether, dioxane, ethyl acetate, toluene, acetonitrile or mixtures thereof to afford. Optionally, catalytic amounts of hypernucleophilic acylating agents such as DMAP may be added. Finally, selective hydrolysis of the phosphonate ester moiety is achieved in two stages by initial treatment of S6 with trimethylsilyl iodide at about −20° to 40° C. in a suitable inert solvent like DCM or DCE, followed by hydrolysis of the tetra(trimethylsilyl)phosphonate intermediate in situ with trifluoroacetic acid in a mixture of acetonitrile and water at about 0° to 40° C. This two-stage hydrolysis protocol delivers the target bisphosphonate S7.

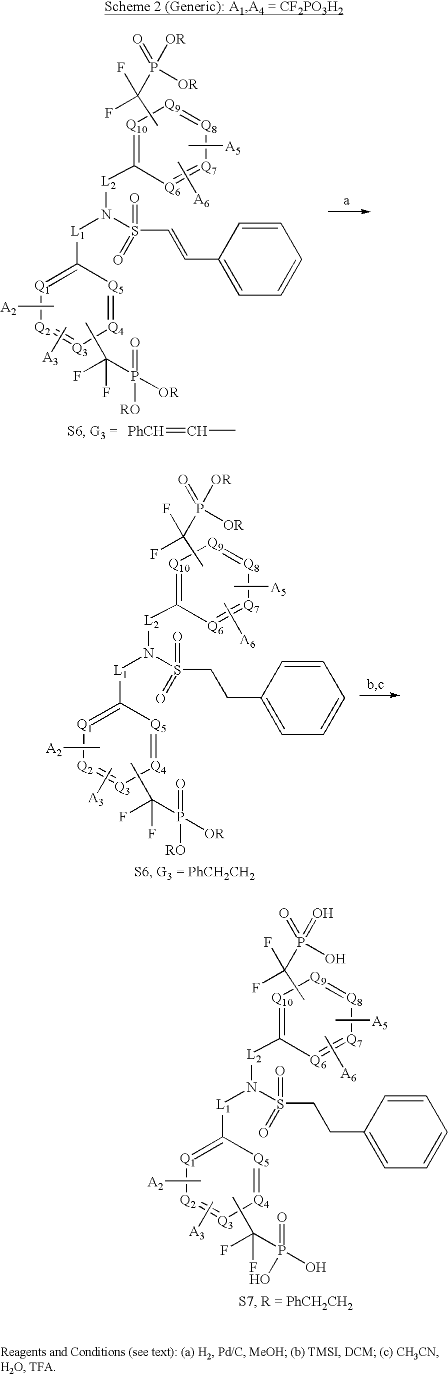

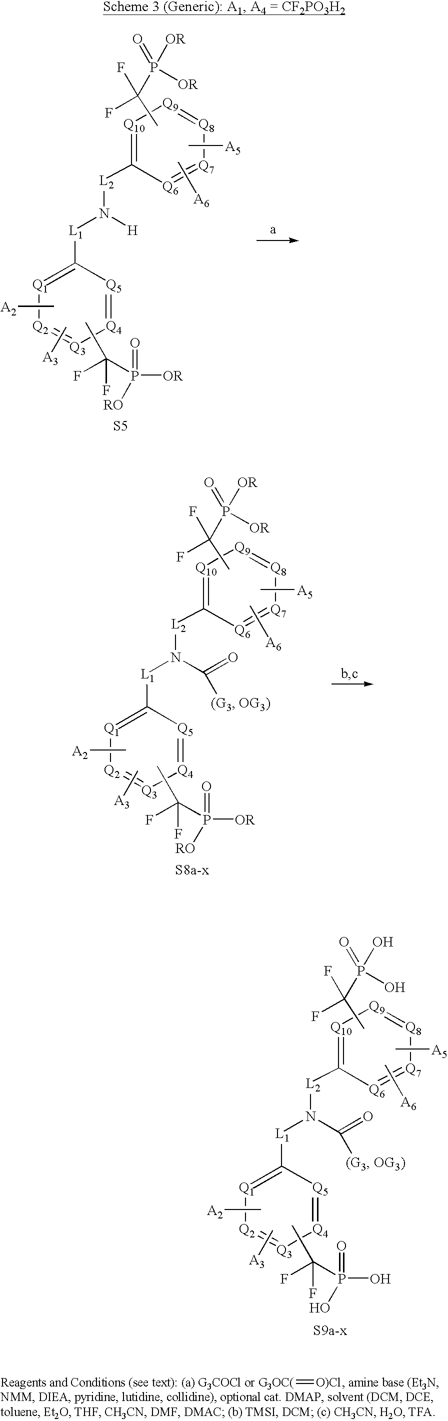

Scheme 2 describes a generic protocol to N,N-disubstituted sulfonamido bis-difluoromethyl-phosphonate inhibitors S7 where R=PhEt that are obtained by selective hydrogenation of the corresponding styryl precursors S6 (R=Ph-CH═CH—). Hence, hydrogenation of intermediate S6 (R=Ph-CH═CH—) at around 15-70 PSI in a suitable inert solvent like ethanol, methanol, THF, ethyl acetate, acetic acid, or mixtures thereof affords S6 (R=PhCH₂CH₂). As described above in Scheme 1, selective hydrolysis of the phosphonate ester moiety is achieved in two stages by initial treatment of S6 with trimethylsilyl iodide at about −20° to 40° C. in a suitable inert solvent like DCM or DCE, followed by hydrolysis of the tetra-(trimethylsilyl)phosphonate intermediate in situ with trifluoroacetic acid in a mixture of acetonitrile and water at about 0° to 40° C. This two-stage hydrolysis protocol delivers the target bisphosphonate S7.

Scheme 3 describes a generic protocol to N,N-disubstituted carboxamide and carbamate inhibitors S9A where two difluoromethylphosphonic acid moieties are present in the inhibitor. Synthesis of target S9A commences by reaction of intermediate S5 with the appropriate acylating or carbamoylating reagents to provide intermediate S8A. In the case of acylamide intermediate S8A, the amine S5 is reacted with reagents selected from a (hetero)aroyl-, (hetero)arylalkanoyl-, (hetero)arylalkenoyl-, (cyclo)alkanoyl- or (cyclo)alkenoyl halide, anhydride or (optionally quaternized) imidazolide at about −20° C. to about 30° C. in the presence of a hydrogen chloride scavenger, typically comprised of a tertiary amine like Et₃N, DIPEA, NMM, NMP, DBU, DBN, DABCO, pyridine, collidine, lutidine, or inorganic bases including sodium carbonate, potassium carbonate, sodium hydrogen carbonate, potassium hydrogen carbonate in an inert solvent such as DCM, DCE, DMF, DMAC, THF, diethyl ether, dioxane, ethyl acetate, toluene, acetonitrile or mixtures thereof to afford. Optionally, catalytic amounts of hypernucleophilic acylating agents such as DMAP may be added.

For preparation of carbamate intermediate S8A, the amine S5 is typically reacted with e.g. a (hetero)aryl, (hetero)arylalkyl-, (hetero)arylalkenyl-, (cyclo)alkyl- or (cyclo)alkenyl-chloroformate or dicarbonate derivative. Optionally, S5 can be reacted with 2-([(hetero)aryloxy-, (hetero)arylalkoxy-, (hetero)arylalkenyloxy-, alkoxy- or alkenyloxy-]carbonyloximino)-2-phenylacetonitrile derivatives to furnish the corresponding carbamate intermediates S8A. Carbamate intermediates S8A formed this way are generally produced using similar reaction conditions (solvents, bases, temperature ranges) described above for the acylamides. Finally, these intermediate carboxamide or carbamate intermediates are selectively hydrolyzed to the corresponding bis-phosphonic acids S9A in two stages by initial treatment of S8A with trimethylsilyl iodide at about −20° to 40° C. in a suitable inert solvent like DCM or DCE, followed by hydrolysis of the tetra-(trimethylsilyl)phosphonate intermediate in situ with trifluoroacetic acid in a mixture of acetonitrile and water at about 0° to 40° C. This two-stage hydrolysis protocol delivers the target bisphosphonate S9A.

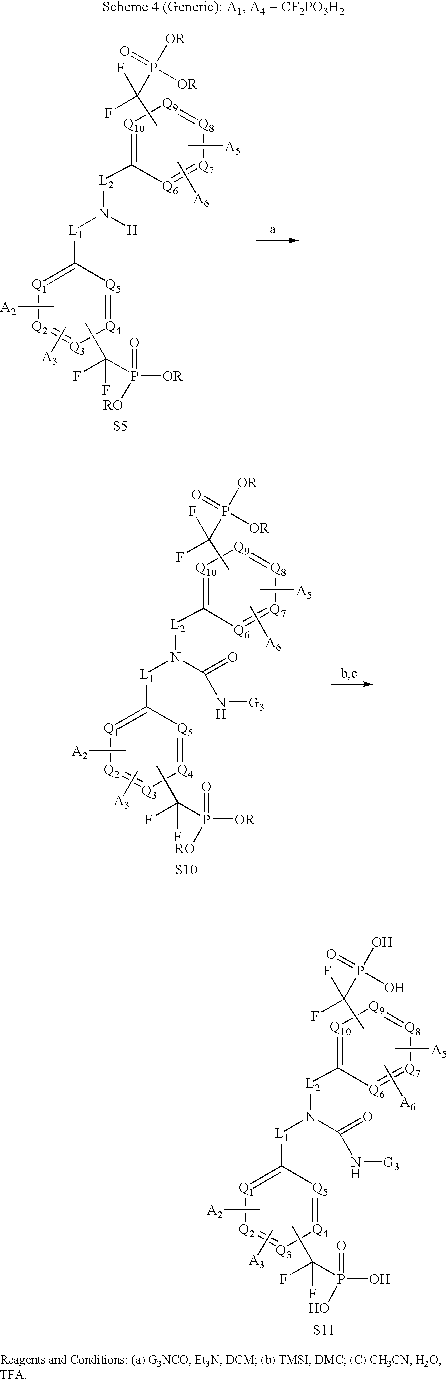

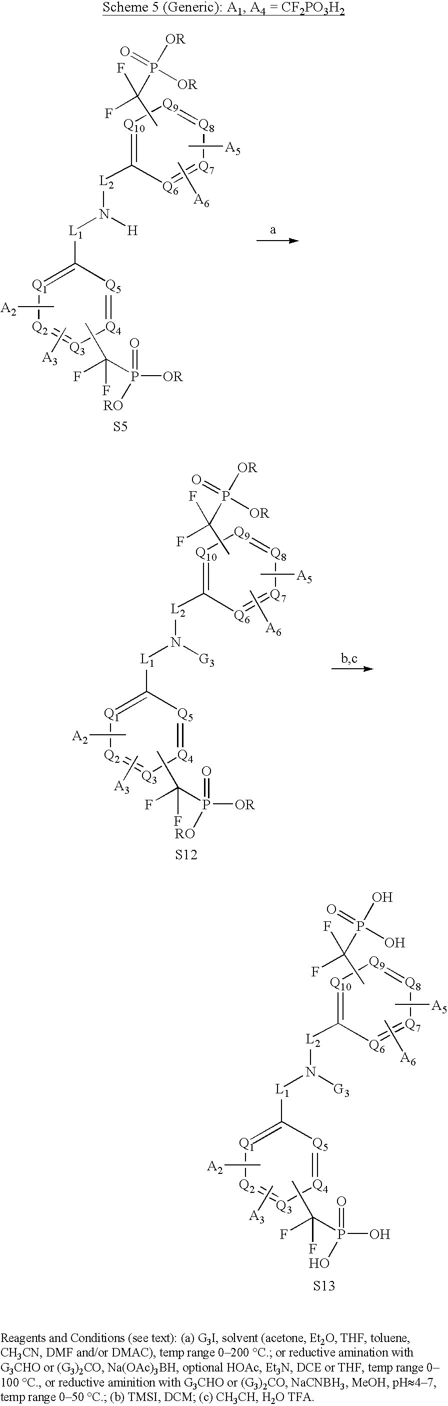

Scheme 4 describes a generic protocol to N,N-disubstituted ureas S11 where two difluoromethyl-phosphonic acid moieties are present in the inhibitor. Synthesis of target S11 commences by reaction of intermediate S5 with the appropriate reagents selected from (hetero)aryl-, (hetero)arylalkyl-, (hetero)arylalkenyl-, (cyclo)alkyl- or (cyclo)alkenyl isocyanates at about −20° C. to about 80° C., optionally in the presence of a catalytic to stoichiometric amount of a tertiary amine like Et₃N, DIPEA, NMM, NMP, DBU, DBN or DABCO in an inert solvent such as DCM, DCE, DMF, DMAC, THF, diethyl ether, dioxane, ethyl acetate, toluene, acetonitrile or mixtures thereof to afford intermediate S10. Optionally, amine S5 can be coupled with CDI (1,1′-carbonyl-diimidazole), p-nitrophenyl chloroformate and like reagents in the presence of a base/inert solvent combination (as listed above) to generate the requisite activated intermediates, which are treated in situ with a primary or secondary amine to provide similar intermediates S10. Hydrolysis of ester derivatives S10 to target bis-phosphonate employs the two-step Me₃SiI/TFA, H₂O protocol as detailed above and provides the urea inhibitors S11.

Scheme 5 describes a generic protocol to N,N,N-trisubstituted amine derivatives S13 where two difluoromethylphosphonic acid moieties are present in the inhibitor. Synthesis of target S13 proceeds via intermediate S12 and commences by alkylation or (hetero)arylation of amine S5 with (hetero)aryl-, (hetero)arylalkyl-, (hetero)arylalkenyl-, (cyclo)alkyl- or (cyclo)alkenyl-halides, mesylates, triflates, or tosylates, optionally in the presence of a base such as a tertiary amine like Et₃N, DIPEA, NMM, NMP, DBU, DBN, DABCO, pyridine, collidine, lutidine, or inorganic bases including sodium carbonate, potassium carbonate, sodium hydrogen carbonate, potassium hydrogen carbonate in an inert solvent such as acetone, 2-butanone, DCM, DCE, DMF, DMAC, THF, diethyl ether, dioxane, ethyl acetate, toluene, acetonitrile or mixtures thereof. In reaction of S5 with (hetero)aromatic substrates to afford S12, optional catalytic through stoichiometric amounts of palladium, nickel, copper, magnesium and/or zinc reagents may be added to the reaction mixture. Alternatively, S5 may be treated with a diverse array of (hetero)aryl-, (hetero)arylalkyl-, (hetero)arylalkenyl-, (cyclo)alkyl- or (cyclo)alkenyl-aldehydes or ketones under reductive amination conditions to afford S12. Representative reductive amination conditions (Organic Reactions, 59, Ch. 1, Wiley, New York, 2002 and references therein) include catalytic hydrogenation at 15-75 PSI with a suitable catalyst such as palladium on carbon, palladium oxide, platinum oxide and Raney nickel. Suitable solvents include ethanol, methanol, THF, ethyl acetate, acetic acid, or mixtures thereof. Reductive amination is also conveniently carried out by using with hydride reagents including borane and borane derivatives (borane-amine, borane-methyl sulfide complexes) typically in an inert solvents like THF, diethyl ether, and/or DCM at about −25° to about 50° C.; sodium cyanoborohydride in solvents including ethanol, methanol, isopropanol, tert-butanol, water or mixtures thereof at about −25° to about 50° C.; and sodium triacetoxyborohydride in solvents such as THF, DCE, acetic acid or mixtures thereof at about −25° to about 90° C. Hydrolysis of ester intermediate S12 employs the two-step Me₃SiI/TFA, H₂O protocol as detailed above and delivers target bis-phosphonate S13.

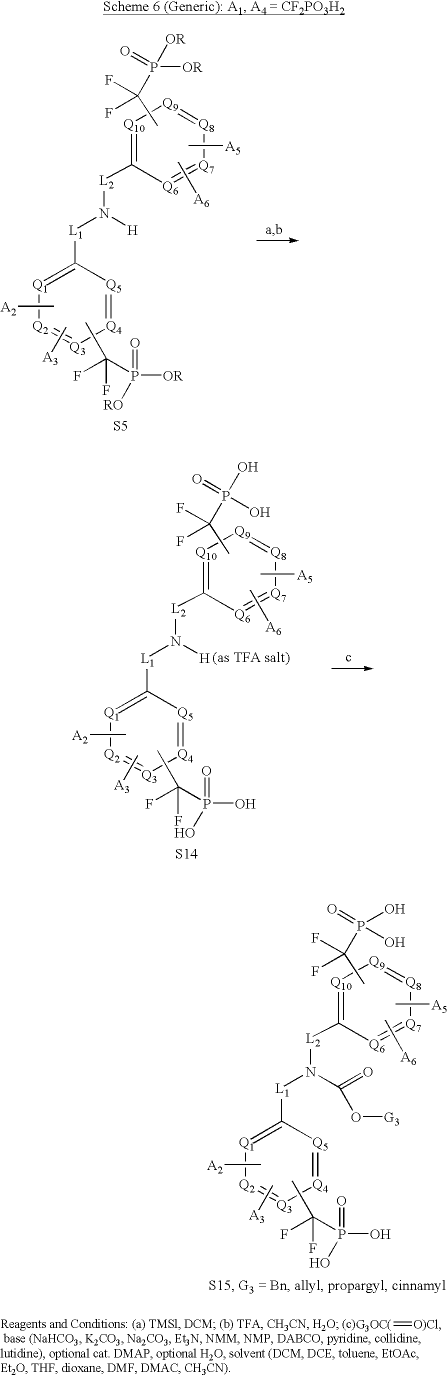

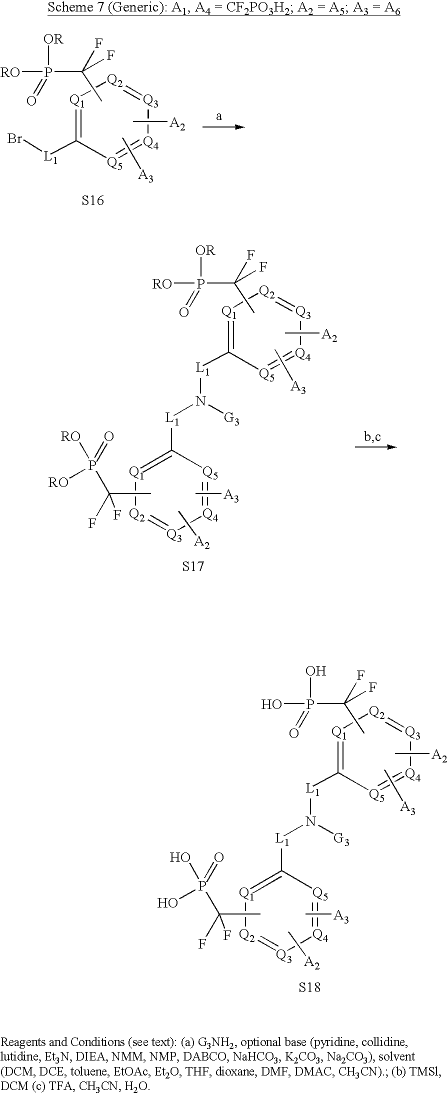

Scheme 6 describes a generic protocol to certain N,N-disubstituted carbamate inhibitors S15 where two difluoromethylphosphonic acid moieties are present in the inhibitor. In contrast to the generic carbamates S9A described above in Scheme 3, inhibitors S15 contain an R group that is introduced at a very late stage of the synthesis due to inherent reactivity or lability issues. This method may be used for inhibitors S15 where R=(substituted)-benzyl-, (substituted)-heteroarylmethyl-, (substituted)-cyclopropyl-, (substituted)-cycloalkenyl-, (substituted)-allyl-, (substituted)-propargyl-, (substituted)-cinnamyl and similar residues.

Intermediate amino ester S5 is hydrolyzed to the corresponding bisphosphonic acid S14 in two stages by initial treatment of S5 with trimethylsilyl iodide at about −20° to 40° C. in a suitable inert solvent like DCM or DCE, followed by hydrolysis of the tetra-(trimethylsilyl)-phosphonate intermediate in situ with trifluoroacetic acid in a mixture of acetonitrile and water at about 0° to 40° C. This two-stage hydrolysis protocol delivers the intermediate bisphosphonic acid S14. Reaction of S14 with a (hetero)aryl-, (hetero)arylalkyl-, (hetero)arylalkenyl-, (cyclo)alkyl- or (cyclo)alkenyl-chloroformate or dicarbonate derivatives affords inhibitors S15. Optionally, S14 can be reacted with 2-([(hetero)arylmethoxy-, (hetero)arylalkenyloxy-, (hetero)arylalkynyloxy-, (cyclo)propoxy-, (cyclo)alkenyloxy- or alkenyloxy-]carbonyloximino)-2-phenylacetonitrile derivatives to furnish the corresponding carbamate inhibitors S15. Carbamates S15 formed this way are generally produced using similar reaction conditions (solvents, bases, temperature ranges) described above for the inhibitors described in Scheme 3.

Scheme 7 describes a generic protocol to N,N,N-trisubstituted amine derivatives S13 where an N-aryl or N-arylalkyl moiety and two difluoromethylphosphonic acid moieties are present in the inhibitor. This method is particularly useful for preparing N-aryl derivatives of structure S18. Synthesis of target inhibitors S18 proceeds via intermediate S17, which is prepared by the procedures outlined below. The dialkylphosphonate alkylating agent S16 is reacted with a primary (hetero)aryl-amine, (hetero)arylalkylamine, (hetero)arylalkenylamine, (hetero)arylalkynylamine, (cyclo)-alkylamine or (cyclo)alkenylamine derivative either neat or in an inert solvent chosen from DCM, DCE, DMF, DMAC, THF, diethyl ether, dioxane, toluene, xylene, acetonitrile or mixtures thereof, optionally in the presence of a base such as a tertiary amine like Et₃N, DIPEA, NMM, NMP, DBU, DBN, DABCO, tertiary amine base derivatives attached to resin supports, aromatic bases including pyridine, collidine, lutidine, or inorganic bases including sodium carbonate, potassium carbonate, sodium hydrogen carbonate and potassium hydrogen carbonate to afford S17. These alkylation reactions may be performed in the temperature range of about −25° C. to 150° C. Application of the two-stage hydrolysis protocol described above (Me₃SiI/TFA, H₂O) provides the target inhibitors S18.

Scheme 8 describes a generic protocol to N,N-disubstituted sulfonamide inhibitors S23A a-x where two tethered benzoic acid moieties are present. Synthesis of generic target S23A a-x commences with substituted amino benzoate ester derivative S19, which is coupled with a substituted aryl sulfonyl halide to afford intermediate S20. This sulfonylation reaction is performed in a solvent chosen from DCM, DCE, DMF, DMAC, THF, diethyl ether, dioxane, toluene, xylene, acetonitrile or mixtures thereof, optionally in the presence of a base such as a tertiary amine like Et₃N, DIPEA, NMM, NMP, DBU, DBN, DABCO, tertiary amine base derivatives attached to resin supports, aromatic bases including pyridine, collidine, lutidine, or inorganic bases including sodium carbonate, potassium carbonate, sodium hydrogen carbonate and potassium hydrogen carbonate. Treatment of sulfonamide intermediate S20 with a base such as sodium hydride, potassium hydride, potassium tert-butoxide, sodium ethoxide, sodium methoxide, sodium hydroxide, potassium hydroxide, sodium carbonate, potassium carbonate in suitable, compatible solvents such as DCM, DCE, DMF, DMAC, DMSO, THF, ethanol, methanol, isopropanol, tert-butanol, diethyl ether, dioxane, toluene, xylene, acetonitrile, water or mixtures thereof is followed by addition of an alkylating reagent of formula S21 and provides N,N-disubstituted sulfonamide S22A. The alkylation reaction to form intermediate S22A can be performed in the temperature range of about −40° C. to 150° C.

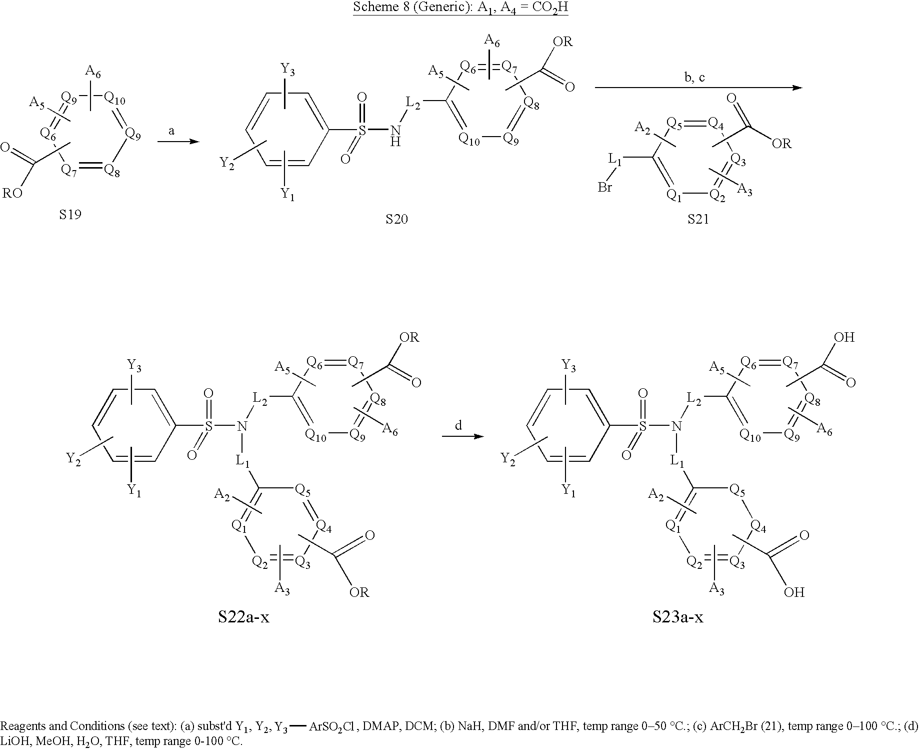

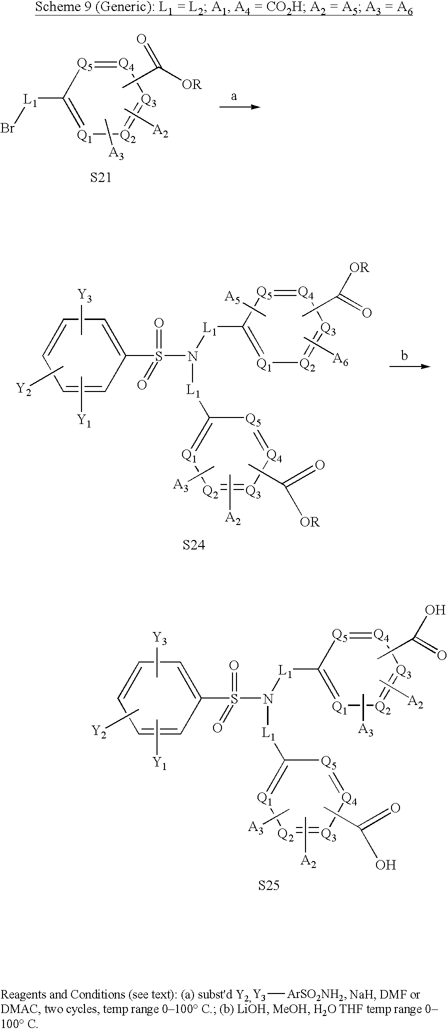

In cases where methyl, ethyl and related primary esters are used as carboxylate protecting groups, hydrolysis of bis-benzoate ester intermediate S22A to the final inhibitors S23A is in one embodiment accomplished by treatment of S22A with a base such as lithium hydroxide, lithium hydroperoxide (generated from LiOH and hydrogen peroxide in situ), sodium hydroxide, potassium hydroxide or tetrabutylammonium hydroxide in a solvent such as methanol, ethanol, isopropanol, tert-butanol, THF, dioxane, acetonitrile, water and mixtures thereof. The hydrolysis reaction can be performed in the temperature range of about −10° C. to 100° C. Ester hydrolysis can also be affected by use of appropriate enzymes including any of the commercially available lipases and esterases, typically in an aqueous solvent system at around 0° C. to 50° C. Alternate hydrolysis methods include treatment of S22A with trimethylsilyl iodide in solvents including DCM, CHCl₃, CCl₄ or DCE at about 0° C. to 100° C., followed by proteolysis of the resultant trimethyl silyl ester intermediate; treatment of S22A with sodium chloride, calcium chloride and related alkaline earth halides in DMSO at about 80° C. to 150° C.; reaction of S22A with a alkali metal thioalkoxide (e.g. sodium thiomethoxide, lithium thioethoxide, lithium thiopropoxide) in a suitable solvent such as DMF, DMAC or DMSO at about 25° C. to 150° C.; cleavage with lithium iodide in pyridine, lutidine or collidine at about 80° C. to 170° C. and cleavage with potassium-, sodium- or lithium trimethylsilanoate in DCM, THF or toluene at about 20° C. to 120° C., followed by acidification of the resultant salt.

In cases where the ester R group=t-butyl, intermediate S22A is treated with a hydrogen chloride or hydrogen bromide source, usually in anhydrous media at around −20° to about 30° C. In cases where the ester R group=(substituted)-benzyl or (substituted)-heteroarylmethyl, intermediate S22A is hydrogenated at ambient room temperature at around 15-70 PSI hydrogen pressure in a suitable inert solvent like ethanol, methanol, THF, ethyl acetate, acetic acid, or mixtures thereof in the presence of a catalyst such as palladium on carbon, palladium hydroxide, and related palladium derivatives. Alternate ester hydrogenolysis conditions include palladium on carbon catalysts with hydrogen transfer reagents selected from formic acid and formate salts, cyclohexene, sodium- or potassium hypophosphite. Suitable solvents include those listed above. Suitable temperature range is about 0° C. to 100° C.

In cases where the ester R group=(substituted)allyl or (substituted)phenylpropenyl (cinnamyl), intermediate S22A is treated with a catalytic amount of an organopalladium reagent such as (Ph₃P)₄Pd in the presence of appropriate allylic group acceptors such as diethyl amine, piperidine, morpholine and dimedone. Suitable solvents include DCM, DCE, THF, DMSO, acetonitrile, water, and mixtures thereof. Addition of catalytic amounts of hydrochloric acid or water may facilitate this deprotection process.

Scheme 9 describes a generic protocol to N,N-disubstituted sulfonamide inhibitors S25 where two symmetrical tethered benzoic acid moieties are present, specifically Q₁-Q₅ are equivalent in both (hetero)aryl ring moieties. This method differs from Scheme 8 since a primary substituted (hetero)arylsulfonamide is dialkylated with the bromide S21, providing a symmetrical intermediate S24. Thus, a Y₁,Y₂,Y₃-substitiuted (hetero)arylsulfonamide is treated with a base such as sodium hydride, potassium hydride, lithium bis(trimethylsilyl)amide, sodium bis(trimethylsilyl)amide, potassium bis(trimethylsilyl)amide, potassium tert-butoxide, sodium ethoxide, sodium methoxide, sodium hydroxide, potassium hydroxide, sodium carbonate or potassium carbonate in suitably compatible solvents such as DCM, DCE, DMF, DMAC, DMSO, THF, ethanol, methanol, isopropanol, tert-butanol, diethyl ether, dioxane, toluene, xylene, acetonitrile, water or mixtures thereof and is alkylated with a substituted (hetero)arylalkyl bromide S21 or similar alkylating agent to provide intermediate S24. The alkylation reaction can be performed in the temperature range of about −10° C. to 100° C. Hydrolysis of ester S23A to inhibitors S25 is achieved by several orthogonal deprotection methods that are detailed above in Scheme 8.

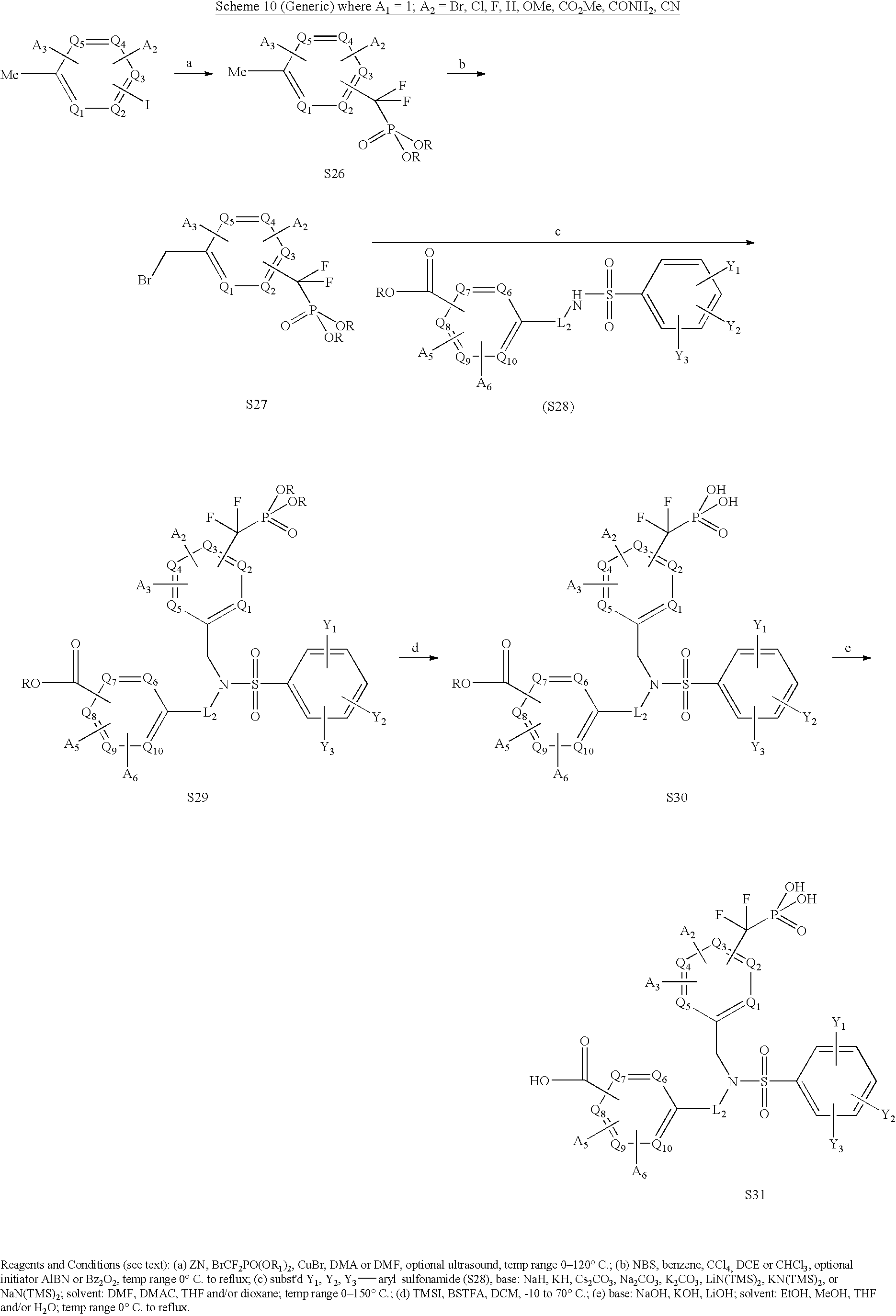

Scheme 10 describes a generic protocol to certain N,N-disubstituted sulfonamide inhibitors S31, which feature one (hetero)arylmethyldifluoromethylphosphonic acid moiety and one tethered (hetero)arylcarboxylate moiety. The intermediate difluoromethylphosphonate ester S26 is obtained from the requisite iodotoluene via displacement of the iodo-group with a suitable dialkyl α,α-dibromo-α-fluoromethyl phosphonate in the presence of metallic reagents such as Zn and CuBr in a dipolar aprotic solvent such as DMF or DMAC at around −20° C. to about 70° C. Bromination of S26 proceeds under free radical-generating conditions utilizing N-bromosuccinimide, 1,3-dibromo-5,5-dimethylhydantoin and similar reagents and provides the bromoalkyl intermediate S27. The bromination reaction is conducted in suitable inert solvents including CCl₄, CHCl₃, DCE, and benzene and is catalyzed by the addition of radical initiators including benzoyl peroxide, 2,2′-azobisisobutyronitrile (AIBN) and related AIBN derivatives, optionally in the presence of a incandescent or ultraviolet light source. Temperature range for the bromination reaction is around 20° C. to 85° C. Sulfonamide S28 (prepared in a fashion analogous to intermediate S20 as detailed in Scheme 8) is treated with a base such as sodium hydride, potassium hydride, lithium bis(trimethyl-silyl)amide, sodium bis(trimethylsilyl)amide, potassium bis(trimethylsilyl)amide, potassium tert-butoxide, sodium ethoxide, sodium methoxide, sodium hydroxide, potassium hydroxide, sodium carbonate or potassium carbonate in suitably compatible solvents such as DCM, DCE, DMF, DMAC, DMSO, THF, ethanol, methanol, isopropanol, tert-butanol, diethyl ether, dioxane, toluene, xylene, acetonitrile, water or mixtures thereof and is then alkylated with the bromo intermediate S27 or similar alkylating agent to provide S29. Temperature range for the alkylation reaction is about −20° C. to 100° C. Selective cleavage of the dialkylphosphonate ester moiety is achieved by limited exposure of S29 to the two-stage hydrolysis protocol described above (Me₃SiI/TFA, H₂O) and provides inhibitors S30. Conditions for the hydrolysis of the carboxylate ester moiety in S30 depend upon the nature of the R protecting group. Appropriate hydrolysis conditions for converting S30 to S31 are chosen as detailed above in Scheme 8.

Scheme 11 describes a generic protocol to certain N,N-disubstituted sulfonamide inhibitors S37, which feature one (hetero)arylmethyldifluoromethylphosphonic acid moiety and one tethered substituted (hetero)aryl moiety. The requisite Y₁,Y₂,Y₃-substituted arylsulfonyl halide is treated with the amine S32, optionally in the presence of a base such as a tertiary amine like Et₃N, DIPEA, NMM, NMP, DBU, DBN, DABCO, tertiary amine base derivatives attached to resin supports, aromatic bases including pyridine, collidine, lutidine, or inorganic bases including sodium carbonate, potassium carbonate, sodium hydrogen carbonate and potassium hydrogen carbonate to afford sulfonamide S34. The sulfonylation reaction is performed in a solvent chosen from DCM, DCE, DMF, DMAC, THF, diethyl ether, dioxane, toluene, xylene, acetonitrile or mixtures thereof. Temperature range for the coupling reaction is about −20° C. to 100° C. Sulfonamide S34 is treated with a base such as sodium hydride, potassium hydride, lithium bis(trimethylsilyl)amide, sodium bis(trimethylsilyl)amide, potassium bis(trimethylsilyl)amide, potassium tert-butoxide, sodium ethoxide, sodium methoxide, sodium hydroxide, potassium hydroxide, sodium carbonate or potassium carbonate in suitably compatible solvents such as DCM, DCE, DMF, DMAC, DMSO, THF, ethanol, methanol, isopropanol, tert-butanol, diethyl ether, dioxane, toluene, xylene, acetonitrile, water or mixtures thereof and is then alkylated with the bromo intermediate S35 or similar alkylating agent to provide S36. Temperature range for the alkylation reaction is about −20° C. to 100° C. Hydrolysis of S36 via the two-stage hydrolysis protocol described above (Me₃SiI/TFA, H₂O) affords the inhibitor S37.

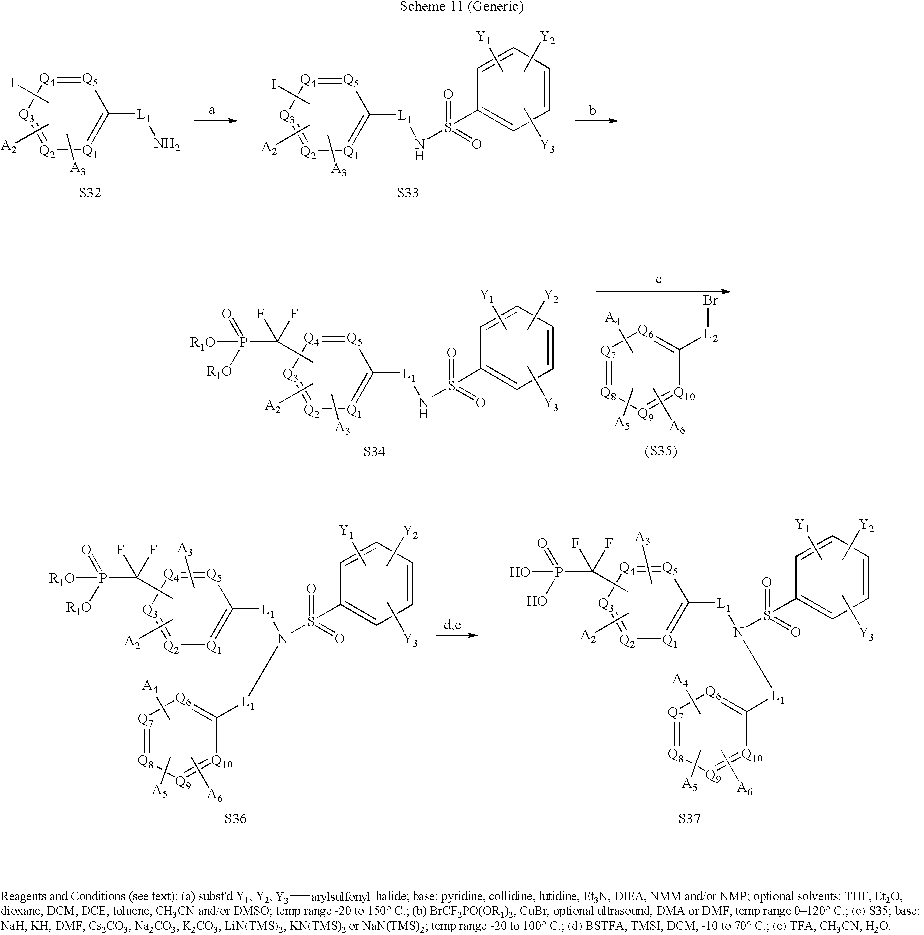

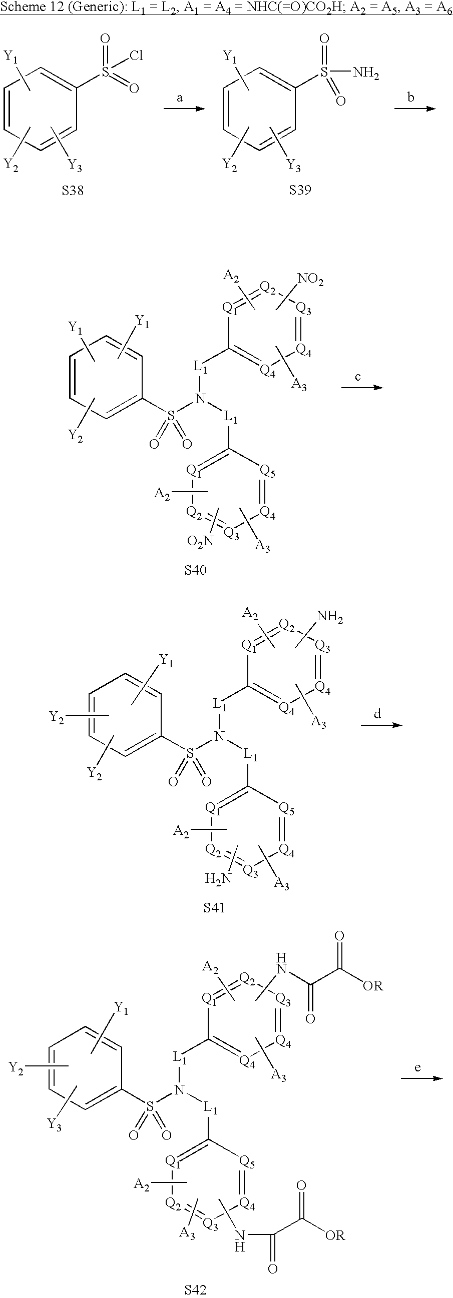

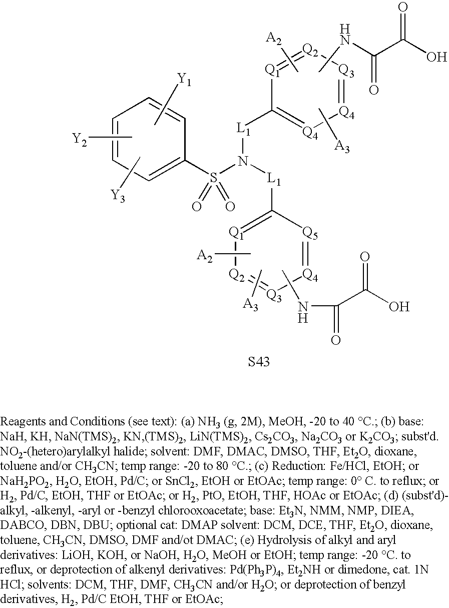

Scheme 12 describes a generic protocol to certain N,N-disubstituted sulfonamide inhibitors S43, which feature symmetrical, tethered bis-(hetero)aryloxamic acid moieties. The requisite Y₁,Y₂,Y₃-substituted arylsulfonamide intermediate S39 is prepared by treatment of arylsulfonyl halide 38 with gaseous ammonia or ammonium hydroxide. The amination reaction is performed neat or in a solvent chosen from DCM, DCE, DMF, DMAC, THF, diethyl ether, dioxane, toluene, xylene, acetonitrile or mixtures thereof. Temperature range for the amination reaction is about −20° C. to 50° C. Sulfonamide S39 is then treated with a base such as sodium hydride, potassium hydride, lithium bis(trimethylsilyl)amide, sodium bis(trimethylsilyl)amide, potassium bis(trimethylsilyl)amide, potassium tert-butoxide, sodium ethoxide, sodium methoxide, sodium hydroxide, potassium hydroxide, sodium carbonate or potassium carbonate in suitably compatible solvents such as DCM, DCE, DMF, DMAC, DMSO, THF, ethanol, methanol, isopropanol, tert-butanol, diethyl ether, dioxane, toluene, xylene, acetonitrile, water or mixtures thereof and alkylated with a substituted nitro-(hetero)arylalkyl halide or similar alkylating agent to provide the symmetrical N,N-dialkylated intermediate S40. Reduction of bis-nitro intermediate S40 to bis-(hetero)arylamine S41 proceeds under the following general reaction conditions: catalytic hydrogenation at 15-75 PSI with a suitable catalyst such as palladium on carbon, palladium oxide, platinum oxide and Raney nickel. Suitable solvents for catalytic hydrogenation include ethanol, methanol, THF, ethyl acetate, acetic acid, water, or mixtures thereof; hydrogen transfer conditions, including palladium on carbon or nickel catalysts with hydrogen transfer reagents selected from hydrazine, formic acid and formate salts, cyclohexene, and sodium or potassium hypophosphite. Suitable solvents include those listed above under catalytic hydrogenation; sodium and lithium borohydride reagents, including complex borohydride derivatives optionally in the presence of transition metal salts, typically in solvents like methanol, ethanol, isopropanol, tert-butanol, THF, diethyl ether, DCE and/or DCM at about −25° to about 85° C.; dissolving metal reductions including zinc or iron in aqueous acidic media at about 0° to 100° C.; and tin (II) chloride in ethanol or ethyl acetate at about 0° C. to 40° C. Bis-arylamine S41 is treated with a (substituted)-alkyl-, -alkenyl, -aryl or -benzyl halooxoacetate, optionally in the presence of a base selected from the tertiary amines Et₃N, DIPEA, NMM, NMP, DBU, DBN, DABCO, tertiary amine base derivatives attached to resin supports, aromatic bases including pyridine, collidine, lutidine, or inorganic bases including sodium carbonate, potassium carbonate, sodium hydrogen carbonate and potassium hydrogen carbonate to afford oxamate ester S42. The reaction is performed in a solvent chosen from DCM, DCE, DMF, DMAC, THF, diethyl ether, dioxane, toluene, xylene, acetonitrile or mixtures thereof. Temperature range for the oxamation reaction is about −20° C. to 100° C. Conditions for the hydrolysis of the oxamate ester moiety in S42 to the inhibitor S43 depend upon the nature of the R protecting group. Appropriate hydrolysis conditions for converting S42 to S43 are thus chosen as detailed above in Scheme 8.

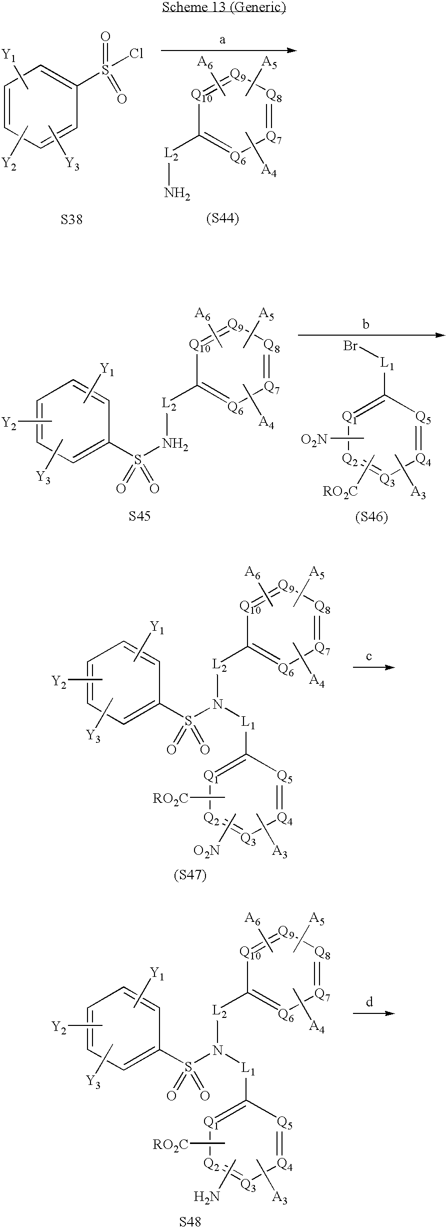

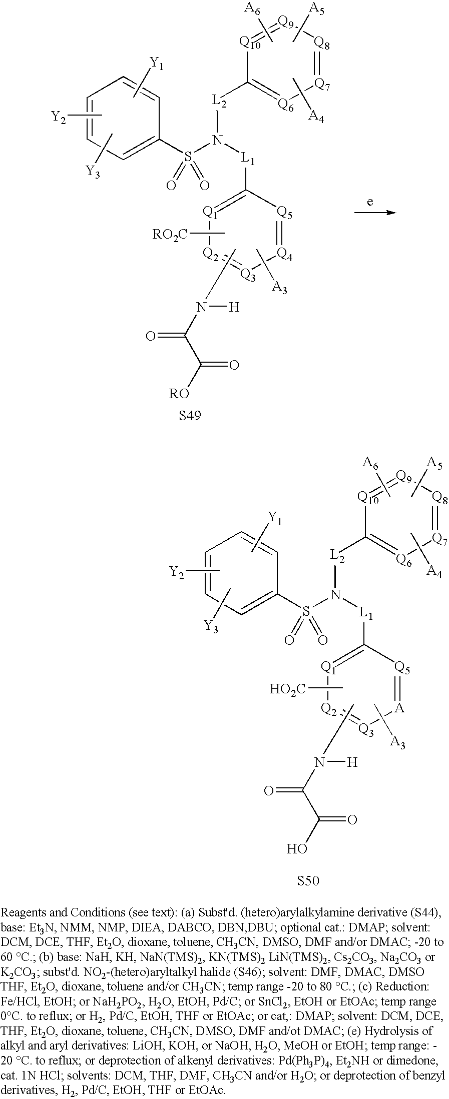

Scheme 13 describes a generic protocol to certain N,N-disubstituted sulfonamide inhibitors S50, which feature one (hetero)arylmethyloxamic acid moiety and one tethered substituted (hetero)aryl moiety. The requisite Y₁,Y₂,Y₃-substituted arylsulfonyl halide S38 is treated with the amine S44, optionally in the presence of a base such as a tertiary amine like Et₃N, DIPEA, NMM, NMP, DBU, DBN, DABCO, tertiary amine base derivatives attached to resin supports, aromatic bases including pyridine, collidine, lutidine, or inorganic bases including sodium carbonate, potassium carbonate, sodium hydrogen carbonate and potassium hydrogen carbonate to afford sulfonamide S45. The sulfonylation reaction is performed in a solvent chosen from DCM, DCE, DMF, DMAC, THF, diethyl ether, dioxane, toluene, xylene, acetonitrile or mixtures thereof. Temperature range for the coupling reaction is about −20° C. to 100° C. Sulfonamide S45 is treated with a base such as sodium hydride, potassium hydride, lithium bis(trimethylsilyl)amide, sodium bis(trimethylsilyl)amide, potassium bis(trimethylsilyl)amide, potassium tert-butoxide, sodium ethoxide, sodium methoxide, sodium hydroxide, potassium hydroxide, sodium carbonate or potassium carbonate in suitably compatible solvents such as DCM, DCE, DMF, DMAC, DMSO, THF, ethanol, methanol, isopropanol, tert-butanol, diethyl ether, dioxane, toluene, xylene, acetonitrile, water or mixtures thereof, and is then alkylated with the nitro(hetero)arylalkyl bromide S46 or similar alkylating agent to provide S47. Temperature range for the alkylation reaction is about −20° C. to 100° C. Reduction of the nitro intermediate S47 to (hetero)arylamine S48 is carried out using methods analogous to those described in Scheme 12 above for the conversion of S40 to S41. (Hetero)arylamine S48 is treated with a (substituted)-alkyl-, -alkenyl, -aryl or -benzyl halooxoacetate to provide advanced intermediate S49 employing conditions analogous to those described in Scheme 12 above for the conversion of S41 to S42. Conditions for the hydrolysis of the oxamate ester moiety in S49 to the inhibitor S50 depend upon the nature of the R protecting group. Appropriate hydrolysis conditions for converting S49 to S50 are thus chosen as detailed above in Scheme 8.

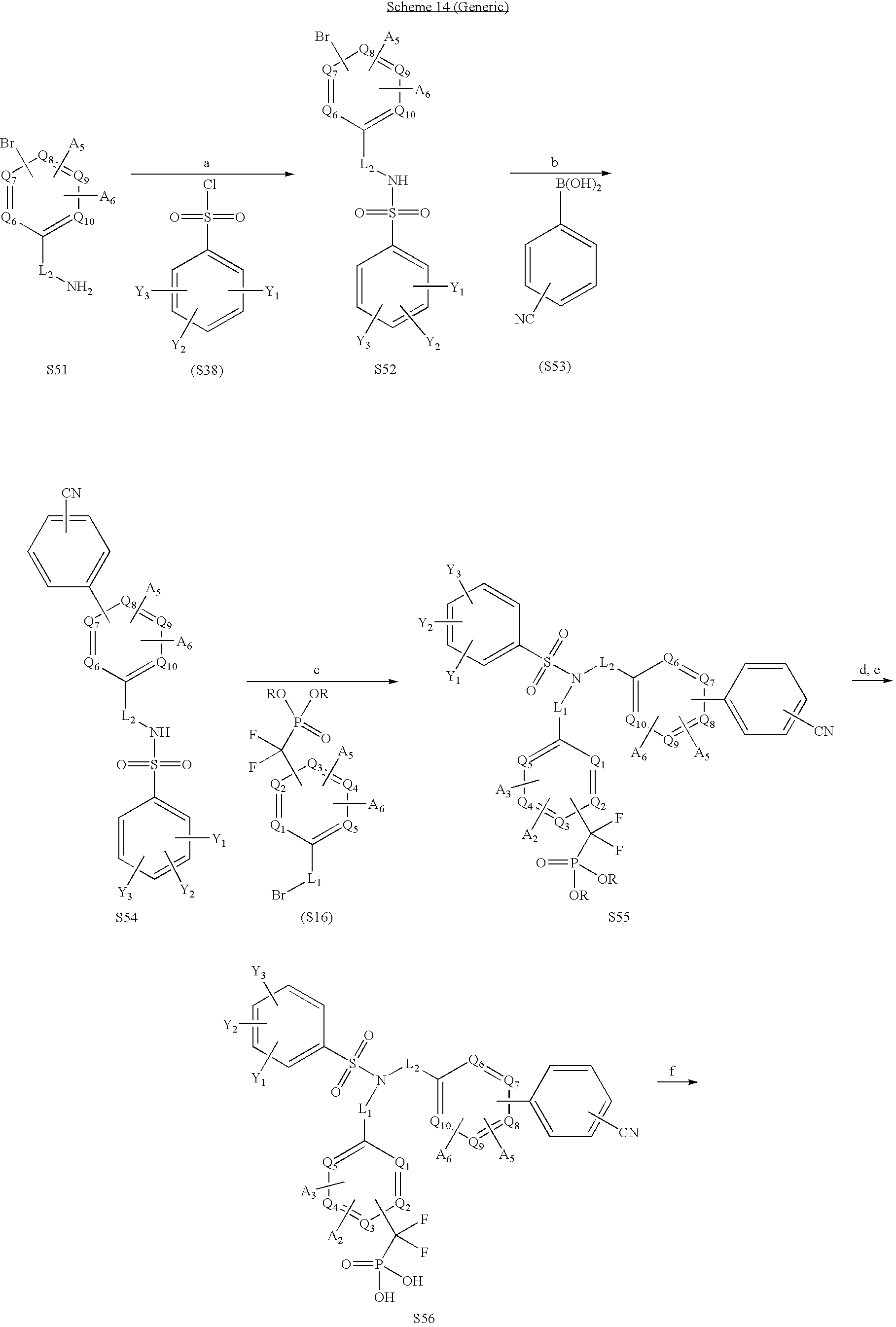

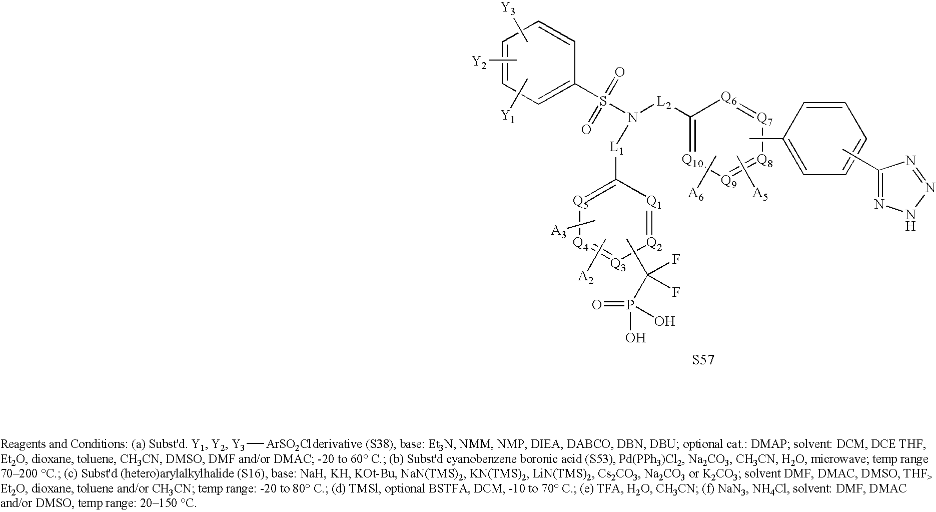

Scheme 14 describes a generic protocol to certain N,N-disubstituted sulfonamide inhibitors S57, which feature one (hetero)aryldifluoromethylphosphonic acid moiety and one tethered substituted (hetero)biaryl moiety. The requisite Y₁,Y₂,Y₃-substituted arylsulfonyl halide S38 is treated with the bromo-substituted amine S51, optionally in the presence of a base such as a tertiary amine like Et₃N, DIPEA, NMM, NMP, DBU, DBN, DABCO, tertiary amine base derivatives attached to resin supports, aromatic bases including pyridine, collidine, lutidine, or inorganic bases including sodium carbonate, potassium carbonate, sodium hydrogen carbonate and potassium hydrogen carbonate to afford sulfonamide S52. The sulfonylation reaction is performed in a solvent chosen from DCM, DCE, DMF, DMAC, THF, diethyl ether, dioxane, toluene, xylene, acetonitrile or mixtures thereof. Temperature range for the reaction is about −20° C. to 100° C. Intermediate S52 is coupled with e.g. a cyano-substituted phenylboronic acid derivative S53 under Suzuki reaction conditions to deliver the (hetero)biaryl intermediate S54. Typical Suzuki coupling reactions employ an organopalladium catalyst such as Pd(Ph₃P)₂Cl₂ in the presence of an alkali metal base like sodium carbonate in polar solvents including acetonitrile, water and mixtures thereof. Use of microwave irradiation significantly accelerates the reaction rate. Temperature range for the microwave reaction is about 70° C. to 200° C. Intermediate S54 is treated with a base such as sodium hydride, potassium hydride, lithium bis(trimethylsilyl)amide, sodium bis(trimethylsilyl)amide, potassium bis(trimethylsilyl)amide, potassium tert-butoxide, sodium ethoxide, sodium methoxide, sodium hydroxide, potassium hydroxide, sodium carbonate or potassium carbonate in suitably compatible solvents such as DCM, DCE, DMF, DMAC, DMSO, THF, ethanol, methanol, isopropanol, tert-butanol, diethyl ether, dioxane, toluene, xylene, acetonitrile, water or mixtures thereof, and alkylated with the (hetero)arylalkyl bromide S27 (see Scheme 14 for preparation of S27) or similar alkylating agent to afford the unsymmetrical N,N-disubstituted sulfonamide S55. Temperature range for the alkylation reaction is about −20° C. to 100° C. Selective hydrolysis of S55 via the two-stage hydrolysis protocol described above (Me₃SiI/TFA, H₂O) affords the inhibitor S56. Reaction of S56 with sodium azide or trimethylsilyl azide in the presence of ammonium chloride in solvents chosen from DMF, DMAC, DMSO or mixtures thereof using a temperature range of 20°-150° C. affords the tetrazole inhibitor S57.

Detailed procedures for the preparation of selected compounds are described in the experimental section of this application.

D. Pharmaceutical Compositions

The pharmaceutical compositions provided herein contain therapeutically effective amounts of one or more of the compounds provided herein that are useful in the prevention, treatment, or amelioration of one or more of the symptoms of diseases or disorders associated with tyrosine phosphatase activity, including PTP-1B activity, or in which tyrosine phosphatase activity is implicated, in a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with tyrosine phosphatase activity or in which tyrosine phosphatase activity is implicated.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein (see, e.g., EXAMPLES 48 and 49) and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with tyrosine phosphatase activity or in which tyrosine phosphatase activity is implicated, as described herein.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re.28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

2. Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

7. Articles of Manufacture

The compounds or pharmaceutically acceptable derivatives may be packaged as articles of manufacture containing packaging material, a compound or pharmaceutically acceptable derivative thereof provided herein, which is effective for modulating the activity of tyrosine phosphatase, or for treatment, prevention or amelioration of one or more symptoms of tyrosine phosphatase, including PTP-1B, mediated diseases or disorders, or diseases or disorders in which tyrosine phosphatase, including PTP-1B, activity, is implicated, within the packaging material, and a label that indicates that the compound or composition, or pharmaceutically acceptable derivative thereof, is used for modulating the activity of tyrosine phosphatase, including PTP-1B, or for treatment, prevention or amelioration of one or more symptoms of tyrosine phosphatase, including PTP-1B, mediated diseases or disorders, or diseases or disorders in which tyrosine phosphatase, including PTP-1B, is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which tyrosine phosphatase, including PTP-1B, is implicated as a mediator or contributor to the symptoms or cause.

E. Methods for the Prophylaxis and Treatment of Disease

The compounds provided herein modulate or inhibit tyrosine phosphatases, including PTP-1B, and thus improve insulin sensitivity, among other benefits. The compounds therefore will find use in preventing or treating Type 1 and Type 2 diabetes [and associated complications such as hypertension, ischemic diseases of the large and small blood vessels, blindness, circulatory problems, kidney failure and atherosclerosis], syndrome X, metabolic syndrome, improving glucose tolerance, improving insulin sensitivity when there is insulin resistance, improving leptin sensitivity where there is leptin resistance, lowering body weight, and preventing or treating obesity. In addition, the compounds will be useful in preventing or treating cancer, neurodegenerative diseases, and the like.

The compounds provided herein modulate or inhibit tyrosine phosphatases, including PTP-1B, and thus improve insulin sensitivity, among other benefits. The compounds therefore will find use in preventing or treating Type 1 and Type 2 diabetes, improving glucose tolerance, improving insulin sensitivity when there is insulin resistance, lowering body weight, and preventing or treating obesity. In addition, the compounds will be useful in preventing or treating cancer, neurodegenerative diseases, and the like.

The present compounds may also be administered in combination with one or more further pharmacologically active substances e.g., selected from antiobesity agents, antidiabetics, antihypertensive agents, agents for the treatment and/or prevention of complications resulting from or associated with diabetes and agents for the treatment and/or prevention of complications and disorders resulting from or associated with obesity.

In another embodiment, the present compounds may be administered in combination with one or more antiobesity agents or appetite regulating agents. Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, B3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator activated receptor) modulators, RXR (retinoid X receptor) modulators or TR B agonists.

In one embodiment the antiobesity agent is leptin. In other embodiments, the antiobesity agent is dexamphetamine or amphetamine, fenfluramine or dexfenfluramine, sibutramine, orlistat, mazindol or phentermine.

Suitable antidiabetics comprise insulin, GLP-1 (glucagons-like peptide-1) derivatives such as those disclosed in WO 98/08871, which is incorporated herein by reference, as well as orally active hypoglycemic agents. The orally active hypoglycemic agents may comprise sulphonylureas, biguanides, meglitinides, oxadiazolidinediones, thizolidinediones, glucosidase inhibitors, glucagons antagonists such as those disclosed in WO 99/01423, GLP-1 agonists, potassium channel openers such as those disclosed in WO 98/26265 and WO 99/03861, insulin sensitizers, DPP-IV (dipeptidyl peptidase-IV) inhibitors, inhibitors of hepatic enzymes involved in stimulation of gluconeogensis and/or glycogenolysis, glucose uptake modulators, compounds modifying the lipid metabolism such as antihyperlipidemic agents and antilipedimic agents as HMG CoA inhibitors (statins), compounds lowering food intake, PPAR and RXR agonists and agents acting on the ATP-dependent potassium channel of the B-cells.

In one embodiment the present compounds are administered in combination with insulin. In further embodiments, the present compounds are administered in combination with a sulphonylurea e.g., tolbutamide, glibenclamide, glipizide or glicazide, a biguanide e.g. metformin, a meglitinide e.g., repaglinide, a thizolidinedione e.g., troglitazone, ciglitazone, pioglitazone, rosiglitazone or compounds disclosed in WO 97/41097 such as 5-[[4-[3-Methyl-4-oxo-3,4-dihydro-2-quinazolinyl]methoxy]phenyl-methyl]thiazolidine-2,4-dione or a pharmaceutically acceptable salt thereof, in one embodiment the potassium salt.

Furthermore, the present compounds may be administered in combination with the insulin sensitizers disclosed in WO 99/19313 such as (−)3-[4-[2-Phenoxazin-10-yl)ethoxy]phenyl]-2-ethoxypropanoic acid or a pharmaceutically acceptable salts thereof, in one embodiment the arginine salt.

In further embodiments, the present compounds are administered in combination with an a-glucosidase inhibitor e.g. miglitol or acarbose, an agent acting on the ATP-dependent potassium channel of the B-cells e.g. tolbutamide, glibenclamide, glipizide, glicazide or repaglinide, nateglinide, an antihyperlipidemic agent or antilipidemic agent e.g., cholestyramine, colestipol, clofibrate, gemfibrozil, lovastatin, pravastatin, simvastatin, probucol or dextrothyroxine,

In still further embodiments, the present compounds are administered in combination with more than one of the above-mentioned compounds e.g., in combination with a sulphonylurea and metformin, a sulphonylurea and acarbose, repaglinide and metformin, insulin and a sulphonylurea, insulin and metformin, insulin, insulin and lovastatin, etc.

Furthermore, the present compounds may be administered in combination with one or more antihypertensive agents. Examples of antihypertensive agents are B-blockers such as alprenolol, atenolol, timolot, pindolol, propranolol and metoprolol, ACE (angiotensin converting enzyme) inhibitors such as benazepril, captopril, analapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the compounds provided herein with one or more of the above-mentioned compounds and optionally one or more further pharmacologically active substances are considered to be within the scope of the present disclosure.

The therapeutically effective amounts of the present compounds will be a function of many variables, including the affinity of the inhibitor for the tyrosine phosphatase, any residual activity exhibited by competitive antagonists, the route of administration, the clinical condition of the patient, and whether the inhibitor is to be used for the prophylaxis or for the treatment of acute episodes.

In practicing the methods provided herein, the therapeutic preparation will be administered to a patient in need of treatment at a therapeutically effective dosage level. The lowest effective dosage levels can be determined experimentally by initiating treatment at higher dosage levels and reducing the dosage level until relief from reaction is no longer obtained. Generally, therapeutic dosage levels will range from about 0.01-100 μg/kg of host body weight.

As discussed above, the present compounds can also administered in conjunction with other agents used in or proposed for the treatment of individual conditions as appropriate. However, when employed together with the present compounds, these agents may be employed in lesser dosages than when used alone.

Where combinations are contemplated, it is not intended that the present disclosure be limited by the particular nature of the combination. The present disclosure contemplates combinations as simple mixtures as well as chemical hybrids. One example of the latter is where the present compound is covalently linked to a pharmaceutical compound, or where two or more compounds are joined. For example, covalent binding of the distinct chemical moieties can be accomplished by any one of many commercially available cross-linking compounds.

In view of the therapeutic urgency attendant acute episodes, the present compounds may be intravenously infused or introduced immediately upon the development of symptoms. However, prophylaxis is suitably accomplished by intramuscular or subcutaneous administration. In this regard, the compositions are prepared as injectables, either as liquid solutions or suspensions; solid forms suitable for solution in, or suspension in, liquid prior to injection may also be prepared. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual hosts.

It is not intended that the present disclosure be limited by the particular nature of the therapeutic preparation. For example, such compositions can be provided together with physiologically tolerable liquid, gel or solid carriers, diluents, adjuvants and excipients. Such compositions are typically prepared as sprays (e.g. intranasal aerosols) for topical use. However, they may also be prepared either as liquid solutions or suspensions, or in solid forms including respirable and nonrespirable dry powders. Oral formulations (e.g. for gastrointestinal administration) usually include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained release formulations, or powders, and typically contain 1%-95% of active ingredient, in one embodiment 2%-70%.

The compounds provided herein are often mixed with diluents or excipients that are physiologically tolerable and compatible. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH buffering agents.

Additional formulations which are suitable for other modes of administration, such as topical administration, include salves, tinctures, creams, lotions, and, in some cases, suppositories. For salves and creams, traditional binders, carriers and excipients may include, for example, polyalkylene glycols or triglycerides.

F. Determination of Activity

The compounds provided herein are evaluated for biological activity as inhibitors of PTP-1B using, for example, a pNPP assay can be used to screen compounds for tyrosine phosphatase inhibitory activity as described in the Examples.

Compounds which demonstrate inhibitory activity against tyrosine phosphatases can have application in the treatment of various diseases. For example, compounds which demonstrate modulatory or inhibitory activity against PTP-1B can find use in the treatment of diabetes. Compounds which demonstrate such activity against CD45 can find use in the treatment of autoimmune diseases, inflammation, transplantation rejection reactions, and other diseases including arthritis, systemic lupus, Crohn's disease, inflammatory bowel disease, and other autoimmune disorders known to those skilled in the art. Compounds which demonstrate such activity against TC-PTP can find use in the treatment of cancer, typically as antiangiogenic agents.

In the case of compounds which demonstrate modulatory or inhibitory activity against PTP-1B, one can test the compounds for blood glucose lowering effects in diabetic obese female ob/ob mice as follows: The mice will be of similar age and body weights and randomized into groups of ten mice. They have free access to food and water during the experiment.

The compounds are administered by either gavage, subcutaneous, intravenous or intraperitoneal injections. Examples of typical dose ranges for such evaluations are 0.1, 0.3, 1.0, 3.0, 10, 30, 100 mg per kg body weight. The blood glucose levels are measured twice before administration of the compounds provided herein. After administration of the compound, the blood glucose levels are measured at the following time points: 1, 2, 4, 6, and 8 hours. A positive response is defined either as (i) a more than 25 percent reduction in blood glucose levels in the group receiving the compound provided herein compared to the group receiving the vehicle at any time point or (ii) statistically significant (i.e., p<0.05) reduction in the area under the blood glucose curve during the whole period (i.e. 8 hrs) in the group treated with the compounds provided herein compared to controls. Compounds that show positive response can be used as development candidates for treatment of human diseases such as diabetes and obesity.

The following detailed examples are provided for illustration and are not to be considered as limiting unless so specified

TABLE 1
Activity of Selected Compounds

| Compound | Example | | Activity |
|---|---|---|---|
| Number | Number | Chemical Name | Class* |
| 5 | 2 | {[4-({(2-Chloro-benzenesulfonyl)-[4- | B |
| | | (difluoro-phosphono-methyl)-benzyl]- | |
| | | amino}-methyl)-phenyl]-difluoro-methyl}- | |
| | | phosphonic acid | |
| 9 | 3 | [(4-{[[4-(Difluoro-phosphono-methyl)- | C |
| | | benzyl]-(2-phenyl-ethanesulfonyl)-amino]-methyl}- | |
| | | phenyl)-difluoro-methyl]-phosphonic | |
| | | acid | |
| 11 | 4 | {[4-({Benzoyl-[4-(difluoro-phosphono- | D |
| | | methyl)-benzyl]-amino}-methyl)-phenyl]- | |
| | | difluoro-methyl}-phosphonic acid | |
| 13 | 5 | [(4-{3-Benzyl-1-[4-(difluoro-phosphono- | E |
| | | methyl)-benzyl]-ureidomethyl}-phenyl)- | |
| | | difluoro-methyl]-phosphonic acid | |
| 15 | 6 | {[4-({[4-(Difluoro-phosphono-methyl)- | E |
| | | benzyl]-methyl-amino}-methyl)-phenyl]- | |
| | | difluoro-methyl}-phosphonic acid | |
| 17 | 7 | {[4-({Benzyloxycarbonyl-[4-(difluoro- | D |
| | | phosphono-methyl)-benzyl]-amino}- | |
| | | methyl)-phenyl]-difluoro-methyl}- | |
| | | phosphonic acid | |
| 20 | 8 | {[4-({Biphenyl-4-ylmethyl-[4-(difluoro- | D |
| | | phosphono-methyl)-benzyl]-amino}- | |
| | | methyl)-phenyl]-difluoro-methyl}- | |
| | | phosphonic acid | |
| 30 | 9 | 4-({Benzenesulfonyl-[3-bromo-4-(difluoro- | C |
| | | phosphono-methyl)-benzyl]-amino}- | |
| | | methyl)-benzoic acid methyl ester | |
| 31 | 10 | 4-({Benzenesulfonyl-[3-bromo-4-(difluoro- | B |
| | | phosphono-methyl)-benzyl]-amino}- | |
| | | methyl)-benzoic acid | |
| 34 | 11 | [(4-{[Benzenesulfonyl-(3,4-dichloro- | C |
| | | phenyl)-amino]-methyl}-2-bromo-phenyl)- | |
| | | difluoro-methyl]-phosphonic acid | |
| 38 | 12 | {[4-({Benzenesulfonyl-[4-(difluoro- | A |
| | | phosphono-methyl)-benzyl]-amino}- | |
| | | methyl)-2-bromo-phenyl]-difluoro-methyl}- | |
| | | phosphonic acid | |
| 42 | 13 | [(2-Bromo-4-{[[4-(difluoro-phosphono- | A |
| | | methyl)-benzyl]-(naphthalene-2-sulfonyl)- | |
| | | amino]-methyl}-phenyl)-difluoro-methyl]- | |
| | | phosphonic acid | |
| 46 | 14 | [(2-Chloro-4-{[(3′-cyano-biphenyl-4- | B |
| | | ylmethyl)-(2-methoxy-benzenesulfonyl)- | |
| | | amino]-methyl}-phenyl)-difluoro-methyl]- | |
| | | phosphonic acid | |
| 47 | 15 | {[2-Chloro-4-({(2-methoxy- | A |
| | | benzenesulfonyl)-[3′-(2H-tetrazol-5-yl)- | |
| | | biphenyl-4-ylmethyl]-amino}-methyl)- | |
| | | phenyl]-difluoro-methyl}-phosphonic acid | |
| 56 | 17 | 5-{[(3,4-Dichloro-benzyl)-(2-methoxy- | E |
| | | benzenesulfonyl)-amino]-methyl}-2- | |
| | | (oxalyl-amino)-benzoic acid | |
| 57 | | N-[4-({(2-Methoxy-benzenesulfonyl)-[4- | E |
| | | (oxalyl-amino)-benzyl]-amino}-methyl)- | |
| | | phenyl]-oxalamic acid | |
| 58 | | 4-{3-[2-Cyano-2-(naphthalen-2- | E |
| | | ylcarbamoyl)-vinyl]-2,5-dimethyl-pyrrol-1- | |
| | | yl}-benzoic acid | |
| 59 | | {[4-({Acetyl-[4-(difluoro-phosphono- | E |
| | | methyl)-benzyl]-amino}-methyl)-phenyl]- | |
| | | difluoro-methyl}-phosphonic acid | |
| 60 | | {[4-({Benzenesulfonyl-[4-(difluoro- | C |
| | | phosphono-methyl)-benzyl]-amino}- | |
| | | methyl)-phenyl]-difluoro-methyl}- | |
| | | phosphonic acid | |
| 61 | | {[4-({[4-(Difluoro-phosphono-methyl)- | D |
| | | benzyl]-isobutoxycarbonyl-amino}- | |
| | | methyl)-phenyl]-difluoro-methyl}- | |
| | | phosphonic acid | |
| 62 | | {[4-({Biphenyl-4-yl-[4-(difluoro- | D |
| | | phosphono-methyl)-benzyl]-amino}- | |
| | | methyl)-phenyl]-difluoro-methyl}- | |
| | | phosphonic acid | |
| 63 | | {[4-({Cyclopentyl-[4-(difluoro-phosphono- | E |
| | | methyl)-benzyl]-amino}-methyl)-phenyl]- | |
| | | difluoro-methyl}-phosphonic acid | |
| 64 | | {[4-({[4-(Difluoro-phosphono-methyl)- | D |
| | | benzyl]-phenyl-amino}-methyl)-phenyl]- | |
| | | difluoro-methyl}-phosphonic acid | |
| 65 | | {[4-({Benzyl-[4-(difluoro-phosphono- | E |
| | | methyl)-benzyl]-amino}-methyl)-phenyl]- | |
| | | difluoro-methyl}-phosphonic acid | |
| 66 | | {[4-({(Biphenyl-4-sulfonyl)-[4-(difluoro- | C |
| | | phosphono-methyl)-benzyl]-amino}- | |
| | | methyl)-phenyl]-difluoro-methyl}- | |
| | | phosphonic acid | |
| 67 | | [(4-{[[4-(Difluoro-phosphono-methyl)- | D |
| | | benzyl]-(naphthalene-1-sulfonyl)-amino]- | |
| | | methyl}-phenyl)-difluoro-methyl]- | |
| | | phosphonic acid | |
| 68 | | [(4-{[Benzenesulfonyl-(3,4-dichloro- | C |
| | | benzyl)-amino]-methyl}-2-bromo-phenyl)- | |
| | | difluoro-methyl]-phosphonic acid | |
| 69 | | {[4-({[4-(Difluoro-phosphono-methyl)- | D |
| | | benzyl]-phenylmethanesulfonyl-amino}- | |
| | | methyl)-phenyl]-difluoro-methy}- | |
| | | phosphonic acid | |
| 70 | | [(4-{[[4-(Difluoro-phosphono-methyl)- | C |
| | | benzyl]-(4-phenoxy-benzenesulfonyl)- | |
| | | amino]-methyl}-phenyl)-difluoro-methyl]- | |
| | | phosphonic acid | |
| 71 | | [(4-{[[4-(Difluoro-phosphono-methyl)- | C |
| | | benzyl]-(naphthalene-2-sulfonyl)-amino]- | |
| | | methyl}-phenyl)-difluoro-methyl]- | |
| | | phosphonic acid | |
| 72 | | [(4-{[[4-(Difluoro-phosphono-methyl)- | C |
| | | benzyl]-(3-phenoxy-benzenesulfonyl)- | |
| | | amino]-methyl}-phenyl)-difluoro-methyl]- | |
| | | phosphonic acid | |
| 73 | | {[4-({(4-Chloro-benzenesulfonyl)-[4- | C |
| | | (difluoro-phosphono-methyl)-benzyl]- | |
| | | amino}-methyl)-phenyl]-difluoro-methyl}- | |
| | | phosphonic acid | |
| 74 | | {[4-({(Biphenyl-3-sulfonyl)-[4-(difluoro- | C |
| | | phosphono-methyl)-benzyl]-amino}- | |
| | | methyl)-phenyl]-difluoro-methyl}- | |
| | | phosphonic acid | |
| 75 | | [(4-{[[4-(Difluoro-phosphono-methyl)- | C |
| | | benzyl]-(2-phenyl-ethenesulfonyl)-amino]- | |
| | | methyl}-phenyl)-difluoro-methyl]- | |
| | | phosphonic acid | |
| 76 | | {[4-({(3-Chloro-benzenesulfonyl)-[4- | C |
| | | (difluoro-phosphono-methyl)-benzyl]- | |
| | | amino}-methyl)-phenyl]-difluoro-methyl}- | |
| | | phosphonic acid | |
| 77 | | {[4-({(3,4-Dichloro-benzenesulfonyl)-[4- | C |
| | | (difluoro-phosphono-methyl)-benzyl]- | |
| | | amino}-methyl)-phenyl]-difluoro-methyl}- | |
| | | phosphonic acid | |
| 78 | | 4-([Benzenesulfonyl-(4-carboxybenzyl)- | E |
| | | amino]-methyl)-benzoic acid | |
| 79 | | 2-bromo-4-([Benzenesulfonyl-(4- | E |
| | | carboxybenzyl)-amino]-methyl)-benzoic | |
| | | acid | |
| 80 | | 4-([Benzenesulfonyl-(2-bromo-4- | E |
| | | carboxybenzyl)-amino]-methyl)-benzoic | |
| | | acid | |
| 81 | | 2-Chloro-N-(3,4-dichloro-benzyl)-N-(4- | E |
| | | nitro-benzyl)-benzenesulfonamide | |
| 82 | | (2-Bromo-4-{[(2-chloro-benzenesulfonyl)- | B |
| | | (3,4-dichloro-benzyl)-amino]-methyl}- | |
| | | phenyl)-difluoro-methyl]-phosphonic acid | |
| 83 | | [(4-{[[4-(Difluoro-phosphono-methyl)- | C |
| | | benzyl]-(2-fluoro-benzenesulfonyl)-amino]- | |
| | | methyl}-phenyl)-difluoro-methyl]- | |
| | | phosphonic acid | |
| 84 | | {[4-({(2-Bromo-benzenesulfonyl)-[4- | C |
| | | (difluoro-phosphono-methyl)-benzyl]- | |
| | | amino}-methyl)-phenyl]-difluoro-methyl}- | |
| | | phosphonic acid | |
| 85 | | [(4-{[[4-(Difluoro-phosphono-methyl)- | C |
| | | benzyl]-(2-methoxy-benzenesulfonyl)- | |
| | | amino]-methyl}-phenyl)-difluoro-methyl]- | |
| | | phosphonic acid | |
| 86 | | [(4-{[[4-(Difluoro-phosphono-methyl)- | C |
| | | benzyl]-(3-methoxy-benzenesulfonyl)- | |
| | | amino]-methyl}-phenyl)-difluoro-methyl]- | |
| | | phosphonic acid | |
| 87 | | 2-Chloro-N-(3,4-dichloro-benzyl)-N-(4- | E |
| | | trifluoromethanesulfonylamino-benzyl)- | |
| | | benzenesulfonamide | |
| 88 | | 1-(Thiophene-2-sulfonyl)-piperidine-4- | E |
| | | carboxylic acid (2-ethylamino-5- | |
| | | trifluoromethanesulfonyl-phenyl)-amide | |
| 89 | | [(4-{[[4-(Difluoro-phosphono-methyl)- | C |
| | | benzyl]-(4-methoxy-benzenesulfonyl)- | |
| | | amino]-methyl}-phenyl)-difluoro-methyl]- | |
| | | phosphonic acid | |
| 90 | | [(4-{[[4-(Difluoro-phosphono-methyl)- | C |
| | | benzyl]-(2-trifluoromethyl- | |
| | | benzenesulfonyl)-amino]-methyl}-phenyl)- | |
| | | difluoro-methyl]-phosphonic acid | |
| 91 | | {[4-({(2,3-Dichloro-benzenesulfonyl)-[4- | D |
| | | (difluoro-phosphono-methyl)-benzyl]- | |
| | | amino}-methyl)-phenyl]-difluoro-methyl}- | |
| | | phosphonic acid | |
| 92 | | 2-{Bis-[4-(difluoro-phosphono-methyl)- | D |
| | | benzyl]-sulfamoyl}-benzoic acid methyl | |
| | | ester | |
| 93 | | {[4-({(Biphenyl-2-sulfonyl)-[4-(difluoro- | C |
| | | phosphono-methyl)-benzyl]-amino}- | |
| | | methyl)-phenyl]-difluoro-methyl}- | |
| | | phosphonic acid | |
| 94 | | 2-Bromo-4-{[(2-chloro-benzenesulfonyl)- | E |
| | | (3,4-dichloro-benzyl)-amino]-methyl}- | |
| | | benzoic acid | |
| 95 | | N-(3-Bromo-4- | E |
| | | trifluoromethanesulfonylaminocarbonyl- | E |
| | | benzyl)-2-chloro-N-(3,4-dichloro-benzyl)- | |
| | | benzenesulfonamide | |
| 96 | | 2-{Bis-[4-(difluoro-phosphono-methyl)- | |
| | | benzyl]-sulfamoyl}-benzoic acid | |
| 97 | | [(2-Bromo-4-{[(3,4-dichloro-benzyl)-(2- | C |
| | | cyanobenzenesulfonyl)-amino]-methyl}- | |
| | | phenyl)-difluoro-methyl]-phosphonic acid | |
| 98 | | [(2-Bromo-4-{[(3,4-dichloro-benzyl)-(2- | C |
| | | trifluoromethoxy-benzenesulfonyl)-amino]- | |
| | | methyl}-phenyl)-difluoro-methyl]- | |
| | | phosphonic acid | |
| 99 | | [(2-Bromo-4-{[[3-bromo-4-(difluoro- | A |
| | | phosphono-methyl)-benzyl]-(naphthalene-2- | |
| | | sulfonyl)-amino]-methyl}-phenyl)-difluoro- | |
| | | methyl]-phosphonic acid | |
| 100 | | [(2-Bromo-4-{[(3,4-dichloro-benzyl)- | C |
| | | (pyridine-2-sulfonyl)-amino]-methyl}- | |
| | | phenyl)-difluoro-methyl]-phosphonic acid | |
| 101 | | [(2-Bromo-4-{[(3,4-dichloro-benzyl)-(2- | B |
| | | methoxy-benzenesulfonyl)-amino]-methyl}- | |
| | | phenyl)-difluoro-methyl]-phosphonic acid | |
| 102 | | [(2-Bromo-4-{[(2-chloro-benzenesulfonyl)- | B |
| | | (4-methanesulfonyl-benzyl)-amino]- | |
| | | methyl}-phenyl)-difluoro-methyl]- | |
| | | phosphonic acid | |
| 103 | | [(2-Bromo-4-{[(4-cyano-benzyl)-(2- | B |
| | | chlorobenzenesulfonyl)-amino]-methyl}- | |
| | | phenyl)-difluoro-methyl]-phosphonic acid | |
| 104 | | 4-{[[3-Bromo-4-(difluoro-phosphono- | B |
| | | methyl)-benzyl]-(2-chloro- | |
| | | benzenesulfonyl)-amino]-methyl}-benzoic | |
| | | acid methyl ester | |
| 105 | | [(2-Bromo-4-{[(4-cyano-benzyl)-(2- | B |
| | | methoxybenzenesulfonyl)-amino]-methyl}- | |
| | | phenyl)-difluoro-methyl]-phosphonic acid | |
| 106 | | N-(2-Ethylamino-5- | E |
| | | trifluoromethanesulfonyl-phenyl)-4-methyl- | |
| | | 3-(piperidine-1-sulfonyl)-benzamide | |
| 107 | | 4-{[[3-Bromo-4-(difluoro-phosphono- | A |
| | | methyl)-benzyl]-(2-chloro- | |
| | | benzenesulfonyl)-amino]-methyl}-benzoic | |
| | | acid | |
| 108 | | [(2-Bromo-4-{[(4-methanesulfonyl-benzyl)- | C |
| | | (2-methoxy-benzenesulfonyl)-amino]- | |
| | | methyl}-phenyl)-difluoro-methyl]- | |
| | | phosphonic acid | |
| 109 | | [(2-Bromo-4-{[(3,4-dichloro-benzyl)-(4- | C |
| | | phenoxy-benzenesulfonyl)-amino]-methyl}- | |
| | | phenyl)-difluoro-methyl]-phosphonic acid | |
| 110 | | 2-Methoxy-N,N-bis-[4-(2H-tetrazol-5-yl)- | E |
| | | benzyl]-benzenesulfonamide | |
| 111 | | (4-{[(2,4-Dimethoxy-benzenesulfonyl)-(4- | E |
| | | methanesulfonyl-benzyl)-amino]-methyl}- | |
| | | phenyl)-oxo-acetic acid | |
| 112 | | N-(4-[bis(N-trifluoro- | E |
| | | methanesulfonyl)amino]benzyl)-N-(4- | |
| | | cyanobenzyl)-(2- | |
| | | methoxy)benzenesulfonamide | |
| 113 | | (4-{[(2,5-Dimethoxy-benzenesulfonyl)-(4- | E |
| | | methanesulfonyl-benzyl)-amino]-methyl}- | |
| | | phenyl)-oxo-acetic acid | |
| 114 | | [(2-Bromo-4-{[(4-methanesulfonyl-phenyl)- | B |
| | | (2-methoxy-benzenesulfonyl)-amino]- | |
| | | methyl}-phenyl)-difluoro-methyl]- | |
| | | phosphonic acid | |
| 115 | | N-(4-Methanesulfonyl-benzyl)-2-methoxy- | E |
| | | N-[4-(1H-tetrazol-5-yl)-benzyl]- | |
| | | benzenesulfonamide | |
| 116 | | N-(4-[hydroxycarbonyl]-benzyl)-2- | E |
| | | methoxy-N-[4-(1H-tetrazol-5-yl)-benzyl]- | |
| | | benzenesulfonamide | |
| 117 | | N-(4-(N-trifluoro-methanesulfonyl- | E |
| | | amino]benzyl)-N-(4-cyanobenzyl)-(2- | |
| | | methoxy)benzenesulfonamide | |
| 118 | | N-(4-(N-oxalylamino]benzyl)-N-(4- | E |
| | | cyanobenzyl)-(2- | |
| | | methoxy)benzenesulfonamide | |
| 119 | | N-[4-(1,2-Dihydroxy-ethyl)-benzyl]-N-(4- | E |
| | | methanesulfonyl-benzyl)-2,5-dimethoxy- | |
| | | benzenesulfonamide | |
| 120 | | 3-{2-Bromo-4-[N-(4-cyanobenzyl)-N-(2- | E |
| | | methoxy-benzenesulfonyl)aminomethyl]- | |
| | | phenyl}-acrylic acid | |
| 121 | | 3-{2-Bromo-4-[N-(4-cyanobenzyl)-N-(2- | E |
| | | methoxy-benzenesulfonyl)aminomethyl]- | |
| | | phenoxy}-acetic acid | |
| 122 | | 3-{4-[[3-Bromo-4-(difluoro-phosphono- | B |
| | | methyl)-benzyl]-(3,4-dichloro-benzyl)- | |
| | | sulfamoyl]-phenyl}-propionic acid | |
| 123 | | 1,2-dihydroxy-3-{2-Bromo-4-[N-(4- | E |
| | | cyanobenzyl)-N-(2-methoxy- | |
| | | benzenesulfonyl)aminomethyl]-phenyl}- | |
| | | propionic acid | |
| 124 | | 3-{4-[N-(4-cyanobenzyl)-N-(2-methoxy- | E |
| | | benzenesulfonyl)aminomethyl]-phenyl}- | |
| | | acrylic acid | |
| 125 | | (4-{[(4-Methanesulfonyl-benzyl)-(2- | E |
| | | methoxy-benzenesulfonyl)-amino]-methyl}- | |
| | | phenyl)-oxo-acetic acid | |
| 126 | | Hydroxyimino-(4-{[(4-methanesulfonyl- | E |
| | | benzyl)-(2-methoxy-benzenesulfonyl)- | |
| | | amino]-methyl}-phenyl)-acetic acid | |
| 127 | | Hydroxy-(4-{[(4-methanesulfonyl-benzyl)- | E |
| | | (2-methoxy-benzenesulfonyl)-amino]- | |
| | | methyl}-phenyl)-acetic acid | |
| 128 | | N-(4-Methanesulfonyl-benzyl)-2-methoxy- | E |
| | | N-[4-(2H-tetrazol-5-ylmethoxy)-benzyl]- | |
| | | benzenesulfonamide | |
| 129 | | N-(4-(N-oxalylamino]benzyl)-N-(4- | E |
| | | cyanobenzyl)-(2- | |
| | | methoxy)benzenesulfonamide | |
| 130 | | (4-{[(4-Cyano-benzyl)-(2-methoxy- | E |
| | | benzenesulfonyl)-amino]-methyl}-phenyl)- | |
| | | difluoro-acetic acid | |
| 131 | | {[4-({[4-(Difluoro-phosphono-methyl)- | D |
| | | benzyl]-methanesulfonyl-amino}-methyl)- | |
| | | phenyl]-difluoro-methyl}-phosphonic acid | |
| 132 | | 3-(4-{[[4-(2-Carboxy-vinyl)-benzyl]-(2- | E |
| | | methoxy-benzenesulfonyl)-amino]-methyl}- | |
| | | phenyl)-acrylic acid | |
| 133 | | 5-[N-(2-Methoxy-benzenesulfonyl)-N- | E |
| | | (4cyanobenzyl)-amino)-methyl]-2-(oxalyl- | |
| | | amino)-benzoic acid | |
| 134 | | 2-Carboxymethoxy-5-{[(4- | E |
| | | methanesulfonyl-benzyl)-(2-methoxy- | |
| | | benzenesulfonyl)-amino]-methyl}-benzoic | |
| | | acid | |
| 135 | | N-(4-Cyano-benzyl)-N-[4-(2-hydroxy-3,4- | E |
| | | dioxo-cyclobut-1-enylamino)-benzyl]-2- | |
| | | methoxy-benzenesulfonamide | |
| 136 | | N-Carboxymethyl-N-(4-{[(4- | E |
| | | methanesulfonyl-benzyl)-(2-methoxy- | |
| | | benzenesulfonyl)-amino]-methyl}-phenyl)- | |
| | | oxalamic acid | |
| 137 | | [(2-Chloro-4-{[(4-methanesulfonyl-benzyl)- | C |
| | | (2-methoxy-benzenesulfonyl)-amino]- | |
| | | methyl}-phenyl)-difluoro-methyl]- | |
| | | phosphonic acid | |
| 138 | | 2-Methoxy-N,N-bis-[4-(1H-tetrazol-5- | E |
| | | ylmethoxy)-benzyl]-benzenesulfonamide | |
| 139 | | 5-{[(4-Cyano-benzyl)-(2-methoxy- | E |
| | | benzenesulfonyl)-amino]-methyl}-2- | |
| | | trifluoromethanesulfonylamino-benzoic | |
| | | acid | |
| 140 | | 2-(4-{[(4-Cyano-benzyl)-(2-methoxy- | E |
| | | benzenesulfonyl)-amino]-methyl}-benzyl)- | |
| | | malonic acid | |
| 141 | | N-[4-({(2-Methoxy-benzenesulfonyl)-[4- | E |
| | | (methyl-oxalyl-amino)-benzyl]-amino}- | |
| | | methyl)-phenyl]-N-methyl-oxalamic acid | |
| 142 | | [(2-Chloro-4-{[(3′-methanesulfonyl- | C |
| | | biphenyl-3-ylmethyl)-(2-methoxy- | |
| | | benzenesulfonyl)-amino]-methyl}-phenyl)- | |
| | | difluoro-methyl]-phosphonic acid | |
| 143 | | [(2-Chloro-4-{[(4′-methanesulfonyl- | C |
| | | biphenyl-3-ylmethyl)-(2-methoxy- | |
| | | benzenesulfonyl)-amino]-methyl}-phenyl)- | |
| | | difluoro-methyl]-phosphonic acid | |
| 144 | | [(2-Chloro-4-{[(4-cyano-benzyl)-(3′- | C |
| | | methanesulfonyl-biphenyl-4-sulfonyl)- | |
| | | amino]-methyl}-phenyl)-difluoro-methyl]- | |
| | | phosphonic acid | |
| 145 | | 4′-{[[3-Chloro-4-(difluoro-phosphono- | C |
| | | methyl)-benzyl]-(2-methoxy- | |
| | | benzenesulfonyl)-amino]-methyl}-biphenyl- | |
| | | 3-carboxylic acid methyl ester | |
| 146 | | [(2-Chloro-4-{[(3,4-dichloro-benzyl)-(2- | C |
| | | methoxy-benzenesulfonyl)-amino]-methyl}- | |
| | | phenyl)-difluoro-methyl]-phosphonic acid | |
| 147 | | [(2-Chloro-4-{[(4′-methanesulfonyl- | B |
| | | biphenyl-4-ylmethyl)-(2-methoxy- | |
| | | benzenesulfonyl)-amino]-methyl}-phenyl)- | |
| | | difluoro-methyl]-phosphonic acid | |
| 148 | | [(2-Chloro-4-{[(3′-methanesulfonyl- | B |
| | | biphenyl-4-ylmethyl)-(2-methoxy- | |
| | | benzenesulfonyl)-amino]-methyl}-phenyl)- | |
| | | difluoro-methyl]-phosphonic acid | |
| 149 | | 4′-{[[3-Chloro-4-(difluoro-phosphono- | A |
| | | methyl)-benzyl]-(2-methoxy- | |
| | | benzenesulfonyl)-amino]-methyl}-biphenyl- | |
| | | 3-carboxylic acid | |
| 150 | | 2,2-Dimethyl-propionic acid [(2-chloro-4- | C |
| | | {[(3,4-dichloro-benzyl)-(2-methoxy- | |
| | | benzenesulfonyl)-amino]-methyl}-phenyl)- | |
| | | difluoro-methyl]-hydroxy- | |
| | | phosphinoyloxymethyl ester | |
| 151 | | [(2-Chloro-4-{[(4′-methanesulfonyl-2- | C |
| | | methyl-biphenyl-4-ylmethyl)-(2-methoxy- | |
| | | benzenesulfonyl)-amino]-methyl}-phenyl)- | |
| | | difluoro-methyl]-phosphonic acid | |
| 152 | | [(2-Chloro-4-{[(4′-cyanomethyl-biphenyl-4- | B |
| | | ylmethyl)-(2-methoxy-benzenesulfonyl)- | |
| | | amino]-methyl}-phenyl)-difluoro-methyl]- | |
| | | phosphonic acid | |
| 153 | | {[2-Chloro-4-({(2-methoxy- | B |
| | | benzenesulfonyl)-[4′-(2H-tetrazol-5- | |
| | | ylmethyl)-biphenyl-4-ylmethyl]-amino}- | |
| | | methyl)-phenyl]-difluoro-methyl}- | |
| | | phosphonic acid | |
| 154 | | [(2-Chloro-4-{[(3′-cyano-biphenyl-4- | C |
| | | ylmethyl)-(naphthalene-2-sulfonyl)-amino]- | |
| | | methyl}-phenyl)-difluoro-methyl]- | |
| | | phosphonic acid | |
| 155 | | [(2-Chloro-4-{[(4′-cyano-biphenyl-4- | B |
| | | ylmethyl)-(2-methoxy-benzenesulfonyl)- | |
| | | amino]-methyl}-phenyl)-difluoro-methyl]- | |
| | | phosphonic acid | |
| 156 | | 4′-{[[3-Chloro-4-(difluoro-phosphono- | A |
| | | methyl)-benzyl]-(2-methoxy- | |
| | | benzenesulfonyl)-amino]-methyl}-4- | |
| | | hydroxy-biphenyl-3-carboxylic acid | |
| 157 | | [(2-Chloro-4-{[(4′-cyano-biphenyl-4- | B |
| | | ylmethyl)-(2-methoxy-benzenesulfonyl)- | |
| | | amino]-methyl}-phenyl)-difluoro-methyl]- | |
| | | phosphonic acid | |
| 158 | | {[2-Chloro-4-({(naphthalene-2-sulfonyl)- | A |
| | | [3′-(2H-tetrazol-5-yl)-biphenyl-4-ylmethyl]- | |
| | | amino}-methyl)-phenyl]-difluoro-methyl}- | |
| | | phosphonic acid | |
| 159 | | {[2-Chloro-4-({(2-methoxy- | A |
| | | benzenesulfonyl)-[4′-(2H-tetrazol-5-yl)- | |
| | | biphenyl-4-ylmethyl]-amino}-methyl)- | |
| | | phenyl]-difluoro-methyl}-phosphonic acid | |
| 160 | | 4′-{[[3-Chloro-4-(difluoro-phosphono- | C |
| | | methyl)-benzyl]-(2-methoxy- | |
| | | benzenesulfonyl)-amino]-methyl}-4- | |
| | | hydroxy-5-propyl-biphenyl-3-carboxylic | |
| | | acid methyl ester | |
| 161 | | [(4-{[(3′-Carbamoyl-biphenyl-4-ylmethyl)- | B |
| | | (2-methoxy-benzenesulfonyl)-amino]- | |
| | | methyl}-2-chloro-phenyl)-difluoro-methyl]- | |
| | | phosphonic acid | |
| 162 | | [(2-Chloro-4-{[(2-chloro-4-fluoro- | B |
| | | benzenesulfonyl)-(3′-cyano-biphenyl-4- | |
| | | ylmethyl)-amino]-methyl}-phenyl)- | |
| | | difluoro-methyl]-phosphonic acid | |
| 163 | | [(2-Chloro-4-{[(2-chloro-benzenesulfonyl)- | B |
| | | (3′-cyano-biphenyl-4-ylmethyl)-amino]- | |
| | | methyl}-phenyl)-difluoro-methyl]- | |
| | | phosphonic acid | |
| 164 | | [(2-Chloro-4-{[(2-chloro-4-fluoro- | A |
| | | benzenesulfonyl)-(3′-cyano-biphenyl-4- | |
| | | ylmethyl)-amino]-methyl}-phenyl)- | |
| | | difluoro-methyl]-phosphonic acid | |
| 165 | | [(2-Chloro-4-{[(3′-cyano-biphenyl-4- | D |
| | | ylmethyl)-(2-methoxy-benzenesulfonyl)- | |
| | | amino]-methyl}-phenyl)-difluoro-methyl]- | |
| | | phosphonic acid monomethyl ester | |
| 166 | | [(2-Chloro-4-{[(2-methoxy- | D |
| | | benzenesulfonyl)-(1-phenyl-ethyl)-amino]- | |
| | | methyl}-phenyl)-difluoro-methyl]- | |
| | | phosphonic acid | |
| 167 | | [(2-Chloro-4-{[(2-methoxy- | C |
| | | benzenesulfonyl)-(2-methyl-benzyl)- | |
| | | amino]-methyl}-phenyl)-difluoro-methyl]- | |
| | | phosphonic acid | |
| 168 | | [(4-{[Biphenyl-2-ylmethyl-(2-methoxy- | B |
| | | benzenesulfonyl)-amino]-methyl}-2-chloro- | |
| | | phenyl)-difluoro-methyl]-phosphonic acid | |
| 169 | | {[2-Chloro-4-({(2-chloro-4-fluoro- | A |
| | | benzenesulfonyl)-[3′-(2H-tetrazol-5-yl)- | |
| | | biphenyl-4-ylmethyl]-amino}-methyl)- | |
| | | phenyl]-difluoro-methyl}-phosphonic acid | |
| 170 | | [(4-{[Benzyl-(2-methoxy-benzenesulfonyl)- | C |
| | | amino]-methyl}-2-chloro-phenyl)-difluoro- | |
| | | methyl]-phosphonic acid | |
| 171 | | [(2-Chloro-4-{[(1,2-diphenyl-ethyl)-(2- | D |
| | | methoxy-benzenesulfonyl)-amino]-methyl}- | |
| | | phenyl)-difluoro-methyl]-phosphonic acid | |
| 172 | | [(4-{[(4-Benzyloxy-benzyl)-(2-methoxy- | C |
| | | benzenesulfonyl)-amino]-methyl}-2-chloro- | |
| | | phenyl)-difluoro-methyl]-phosphonic acid | |
| 173 | | [(2-Chloro-4-{[(2-methoxy- | B |
| | | benzenesulfonyl)-(2-phenoxy-benzyl)- | |
| | | amino]-methyl}-phenyl)-difluoro-methyl]- | |
| | | phosphonic acid | |
| 174 | | 4′-{[[3-Chloro-4-(difluoro-phosphono- | B |
| | | methyl)-benzyl]-(2-methoxy- | |
| | | benzenesulfonyl)-amino]-methyl}-3-fluoro- | |
| | | biphenyl-4-carboxylic acid methyl ester | |
| 175 | | 4-Chloro-4′-{[[3-chloro-4-(difluoro- | C |
| | | phosphono-methyl)-benzyl]-(2-methoxy- | |
| | | benzenesulfonyl)-amino]-methyl}-biphenyl- | |
| | | 3-carboxylic acid ethyl ester | |
| 176 | | 4-Chloro-4′-{[[3-chloro-4-(difluoro- | A |
| | | phosphono-methyl)-benzyl]-(2-methoxy- | |
| | | benzenesulfonyl)-amino]-methyl}-biphenyl- | |
| | | 3-carboxylic acid | |
| 177 | | 4′-{[[3-Chloro-4-(difluoro-phosphono- | A |
| | | methyl)-benzyl]-(2-methoxy- | |
| | | benzenesulfonyl)-amino]-methyl}-3-fluoro- | |
| | | biphenyl-4-carboxylic acid | |
| 178 | | [(4-{[(2-Bromo-benzyl)-(2-methoxy- | B |
| | | benzenesulfonyl)-amino]-methyl}-2-chloro- | |
| | | phenyl)-difluoro-methyl]-phosphonic acid | |
| 179 | | [(2-Chloro-4-{[(2-methoxy- | B |
| | | benzenesulfonyl)-(2-pyridin-3-yl-benzyl)- | |
| | | amino]-methyl}-phenyl)-difluoro-methyl]- | |
| | | phosphonic acid | |
| 180 | | [(2-Chloro-4-{[(2-methoxy- | B |
| | | benzenesulfonyl)-(2-pyridin-4-yl-benzyl)- | |
| | | amino]-methyl}-phenyl)-difluoro-methyl]- | |
| | | phosphonic acid | |
| 181 | | [(2-Chloro-4-{[(4′-chloro-3′- | A |
| | | hydroxycarbamoyl-biphenyl-4-ylmethyl)- | |
| | | (2-methoxy-benzenesulfonyl)-amino]- | |
| | | methyl}-phenyl)-difluoro-methyl]- | |
| | | phosphonic acid | |
| 182 | | 4′-{2-[[3-Chloro-4-(difluoro-phosphono- | C |
| | | methyl)-benzyl]-(2-methoxy- | |
| | | benzenesulfonyl)-amino]-ethyl}-biphenyl- | |
| | | 3-carboxylic acid methyl ester | |
| 183 | | [(2-Chloro-4-{[(2-methoxy- | A |
| | | benzenesulfonyl)-(3′-methoxy-biphenyl-2- | |
| | | ylmethyl)-amino]-methyl}-phenyl)- | |
| | | difluoro-methyl]-phosphonic acid | |
| 184 | | [(2-Chloro-4-{[(3′-hydroxy-biphenyl-2- | B |
| | | ylmethyl)-(2-methoxy-benzenesulfonyl)- | |
| | | amino]-methyl}-phenyl)-difluoro-methyl]- | |
| | | phosphonic acid | |
| 185 | | 3′-{2-[[3-Chloro-4-(difluoro-phosphono- | C |
| | | methyl)-benzyl]-(2-methoxy- | |
| | | benzenesulfonyl)-amino]-ethyl}-biphenyl- | |
| | | 3-carboxylic acid methyl ester | |
| 186 | | [(4-{[(3′-Carbamoyl-biphenyl-2-ylmethyl)- | B |
| | | (2-methoxy-benzenesulfonyl)-amino]- | |
| | | methyl}-2-chloro-phenyl)-difluoro-methyl]- | |
| | | phosphonic acid | |
| 187 | | [(4-{[(3′-Carbamoyl-biphenyl-2-ylmethyl)- | A |
| | | (2-methoxy-benzenesulfonyl)-amino]- | |
| | | methyl}-2-chloro-phenyl)-difluoro-methyl]- | |
| | | phosphonic acid | |
| 188 | | 4′-{2-[[3-Chloro-4-(difluoro-phosphono- | C |
| | | methyl)-benzyl]-(2-methoxy- | |
| | | benzenesulfonyl)-amino]-ethyl}-biphenyl- | |
| | | 3-carboxylic acid | |
| 189 | | 3′-{2-[[3-Chloro-4-(difluoro-phosphono- | C |
| | | methyl)-benzyl]-(2-methoxy- | |
| | | benzenesulfonyl)-amino]-ethyl}-biphenyl- | |
| | | 3-carboxylic acid | |
| 190 | 40 | {[4-(difluorophosphonomethyl)-3- | A |
| | | chlorophenyl]methyl}{[2-(4- | |
| | | methoxyphenyl)phenyl]methyl}[(2- | |
| | | methoxyphenyl)sulfonyl]amine | |
| 191 | 26 | Methyl 2-(3-{4-[({[4- | A |
| | | (difluorophosphonomethyl)-3- | |
| | | chlorophenyl]methyl}[(2- | |
| | | methoxyphenyl)sulfonyl]amino)methyl]phenyl} | |
| | | phenyl)acetate | |
| 192 | 41 | [(4-{[Benzyl-(2-carbamoyl- | C |
| | | benzenesulfonyl)-amino]-methyl}-2-chloro- | |
| | | phenyl)-difluoromethyl]-phosphonic acid | |
| 193 | 27 | 2-(3-{4-[({[4-(difluorophosphonomethyl)-3- | A |
| | | chlorophenyl]methyl}[(2- | |
| | | methoxyphenyl)sulfonyl]amino)methyl]phenyl} | |
| | | phenyl)acetic acid | |
| 194 | 28 | Methyl 3-{4-[({[4- | C |
| | | (difluorophosphonomethyl)-3- | |
| | | chlorophenyl]methyl}[(2- | |
| | | methoxyphenyl)sulfonyl]amino)methyl]phenyl}- | |
| | | 5-bromobenzoate | |
| 195 | 29 | 3-{4-[({[4-(difluorophosphonomethyl)-3- | A |
| | | chlorophenyl]methyl}[(2- | |
| | | methoxyphenyl)sulfonyl]amino)methyl]phenyl}- | |
| | | 5-bromobenzoic acid | |
| 196 | 39 | {[4-(difluorophosphonomethyl)-3- | B |
| | | chlorophenyl]methyl}{[2-(3- | |
| | | ethylphenyl)phenyl]methyl}[(2- | |
| | | methoxyphenyl)sulfonyl]amine | |
| 197 | 30 | 6-{4-[({[4-(difluorophosphonomethyl)-3- | B |
| | | chlorophenyl]methyl}[(2- | |
| | | methoxyphenyl)sulfonyl]amino)methyl]phenyl}- | |
| | | 3-hydroisobenzofuran-1-one | |
| 198 | 38 | {[4-(difluorophosphonomethyl)-3- | A |
| | | chlorophenyl]methyl}[(2- | |
| | | methoxyphenyl)sulfonyl]{[2-(4- | |
| | | methylphenyl)phenyl]methyl}amine | |
| 199 | 37 | 4-{2-[({[4-(difluorophosphonomethyl)-3- | A |
| | | chlorophenyl]methyl}[(2- | |
| | | methoxyphenyl)sulfonyl]amino)methyl]phenyl} | |
| | | benzamide | |
| 200 | 31 | 5-{4-[({[4-(difluorophosphonomethyl)-3- | B |
| | | chlorophenyl]methyl}[(2- | |
| | | methoxyphenyl)sulfonyl]amino)methyl]phenyl}- | |
| | | 2-(hydroxymethyl)benzoic | |
| | | acid,trisodium salt | |
| 201 | 18 | (Adamantanylmethyl){[4- | C |
| | | (difluorophosphonomethyl)-3- | |
| | | chlorophenyl]methyl}[(2- | |
| | | methoxyphenyl)sulfonyl]amine | |
| 202 | 19 | {[4-(difluorophosphonomethyl)-3- | B |
| | | chlorophenyl]methyl}(cycloheptylmethyl) | |
| | | [(2-methoxyphenyl)sulfonyl]amine | |
| 203 | 20 | {[4-(difluorophosphonomethyl)-3- | C |
| | | chlorophenyl]methyl}(cyclohexylmethyl) | |
| | | [(2-methoxyphenyl)sulfonyl]amine | |
| 204 | 36 | {[4-(difluorophosphonomethyl)-3- | A |
| | | chlorophenyl]methyl}[(2-(2,3- | |
| | | dihydrobenzo[b]furan-5- | |
| | | yl)phenyl)methyl][(2- | |
| | | methoxyphenyl)sulfonyl]amine | |
| 205 | 32 | Methyl 5-{4-[({[4- | B |
| | | (difluorophosphonomethyl)-3- | |
| | | chlorophenyl]methyl}[(2- | |
| | | methoxyphenyl)sulfonyl]amino)methyl]phenyl}- | |
| | | 2-methoxybenzoate | |
| 206 | 35 | {[4-(difluorophosphonomethyl)-3- | A |
| | | chlorophenyl]methyl}[(2 | |
| | | methoxyphenyl)sulfonyl]{[2-(3- | |
| | | methylthiophenyl)phenyl]methyl}amine | |
| 207 | 33 | 5-{4-[({[4-(difluorophosphonomethyl)-3- | A |
| | | chlorophenyl]methyl}[(2- | |
| | | methoxyphenyl)sulfonyl]amino)methyl]phenyl}- | |
| | | 2-methoxybenzoic acid | |
| 208 | 21 | [(2-Chloro-4-{[(2-methoxy- | C |
| | | benzenesulfonyl)-(2-piperidin-1-yl-benzyl)- | |
| | | amino]-methyl}-phenyl)-difluoro-methyl]- | |
| | | phosphonic acid | |
| 209 | 22 | {[4-(difluorophosphonomethyl)-3- | D |
| | | chlorophenyl]methyl}[(2- | |
| | | methoxyphenyl)sulfonyl](3- | |
| | | piperidylmethyl)amine | |
| 210 | 34 | 5-{4-[({[4- | C |
| | | (difluorophosphonomethyl)phenyl]methyl} | |
| | | [(2- | |
| | | methoxyphenyl)sulfonyl]amino)methyl]phenyl}- | |
| | | 2-hydroxybenzoic acid | |
| 211 | 23 | {[4-(difluorophosphonomethyl)-3- | C |
| | | chlorophenyl]methyl}[(2- | |
| | | methoxyphenyl)sulfonyl]{[2- | |
| | | (methylethoxy)phenyl]methyl}amine | |
| 212 | 24 | {[4-(difluorophosphonomethyl)-3- | C |
| | | chlorophenyl]methyl}[(2- | |
| | | ethoxyphenyl)methyl][(2- | |
| | | methoxyphenyl)sulfonyl]amine | |
| 213 | 25 | {[4-(difluorophosphonomethyl)-3- | D |
| | | chlorophenyl]methyl}[(2- | |
| | | methoxyphenyl)sulfonyl](2- | |
| | | piperidylmethyl)amine | |
| 214 | | [(4-{[Biphenyl-2-ylmethyl-(3-carbamoyl- | C |
| | | benzenesulfonyl)-amino]-methyl}-2-chloro- | |
| | | phenyl)-difluoro-methyl]-phosphonic acid | |
| 215 | | {[2-Chloro-4-({(2-methoxy- | C |
| | | benzenesulfonyl)-[2-(4-methyl-pentyloxy)- | |
| | | benzyl]-amino}-methyl)-phenyl]-difluoro- | |
| | | methyl}-phosphonic acid | |
| 216 | | [(4-{[Biphenyl-2-ylmethyl-(3-cyano- | C |
| | | benzenesulfonyl)-amino]-methyl}-2-chloro- | |
| | | phenyl)-difluoro-methyl]-phosphonic acid | |
| 217 | | [(2-Chloro-4-{[(3′-ethoxy-biphenyl-2- | A |
| | | ylmethyl)-(2-methoxy-benzenesulfonyl)- | |
| | | amino]-methyl}-phenyl)-difluoro-methyl]- | |
| | | phosphonic acid | |
| 218 | | [(2-Chloro-4-{[(3′-cyano-biphenyl-4- | D |
| | | ylmethyl)-(2-methoxy-benzenesulfonyl)- | |
| | | amino]-methyl}-phenyl)-difluoro-methyl]- | |
| | | phosphonic acid mono-(1- | |
| | | isopropoxycarbonyloxy-ethyl) ester | |
| 219 | 42 | [S]-N-[(2-Chloro-4-{[(3′-cyano-biphenyl-4- | C |
| | | ylmethyl)-(2-methoxy-benzenesulfonyl)- | |
| | | amino]-methyl}-phenyl)-difluoro-methyl]- | |
| | | phosphonyl-(ethyl alaninate) | |
| 221 | | [(2-Chloro-4-{[(3′-ethoxy-biphenyl-2- | A |
| | | ylmethyl)-(2-methoxy-benzenesulfonyl)- | |
| | | amino]-methyl}-phenyl)-difluoro-methyl]- | |
| | | phosphonic acid | |
| 222 | | 4′-{[(4-Carboxymethoxy-3-chloro-benzyl)- | E |
| | | (2-methoxy-benzenesulfonyl)-amino]- | |
| | | methyl}-4-hydroxy-biphenyl-3-carboxylic | |
| | | acid | |
| 223 | | 3-[(4-{[{4-[(2-Carboxy-propyl)-oxalyl- | E |
| | | amino]-benzyl}-(2-methoxy- | |
| | | benzenesulfonyl)-amino]-methyl}-phenyl)- | |
| | | oxalyl-amino]-2-methyl-propionic acid | |
| 224 | | 5-{[Biphenyl-2-ylmethyl-(2-methoxy- | E |
| | | benzenesulfonyl)-amino]-methyl}-2- | |
| | | carboxymethoxy-benzoic acid | |
| 225 | | 3-[(4-{[(2-Methoxy-benzenesulfonyl)- | E |
| | | naphthalen-1-ylmethyl-amino]-methyl}- | |
| | | phenyl)-oxalyl-amino]-butyric acid | |
| 226 | 46 | 2,2-Dimethyl-propionic acid [(2-chloro-4- | E |
| | | {[(3′-cyano-biphenyl-4-ylmethyl)-(2- | |
| | | methoxy-benzenesulfonyl)-amino]-methyl}- | |
| | | phenyl)-difluoro-methyl]-(2,2-dimethyl- | |
| | | propionyloxymethoxy)- | |
| | | phosphinoyloxymethyl ester | |
| 227 | | 2,2-Dimethyl-propionic acid [(2-chloro-4- | E |
| | | {[(3′-cyano-biphenyl-4-ylmethyl)-(2- | |
| | | methoxy-benzenesulfonyl)-amino]-methyl}- | |
| | | phenyl)-difluoro-methyl]-hydroxy- | |
| | | phosphinoyloxymethyl ester | |
| 228 | | 2,2-Dimethyl-propionic acid [(2-bromo-4- | D |
| | | {[(4-methanesulfonyl-phenyl)-(2-methoxy- | |
| | | benzenesulfonyl)-amino]-methyl}-phenyl)- | |
| | | difluoro-methyl]-hydroxy- | |
| | | phosphinoyloxymethyl ester | |
| 229 | | 2,2-Dimethyl-propionic acid [(2-bromo-4- | E |
| | | {[(4-methanesulfonyl-phenyl)-(2-methoxy- | |
| | | benzenesulfonyl)-amino]-methyl}-phenyl)- | |
| | | difluoro-methyl]-(2,2-dimethyl- | |
| | | propionyloxymethoxy)- | |
| | | phosphinoyloxymethyl ester | |
| 230 | | [(4-{[Biphenyl-2-ylmethyl-(4-iodo- | B |
| | | pyridine-3-sulfonyl)-amino]-methyl}-2- | |
| | | chloro-phenyl)-difluoro-methyl]- | |
| | | phosphonic acid | |
| 231 | | 4-{[[3-Chloro-4-(difluoro-phosphono- | C |
| | | methyl)-benzyl]-(2-methoxy- | |
| | | benzenesulfonyl)-amino]-methyl}-benzoic | |
| | | acid methyl ester | |
| 232 | | {[2-Chloro-4-({[3-chloro-4-(difluoro- | C |
| | | phosphono-methyl)-benzyl]-indan-1-yl- | |
| | | amino}-methyl)-phenyl]-difluoro-methyl}- | |
| | | phosphonic acid | |
| 233 | | 4-{[[3-Chloro-4-(difluoro-phosphono- | B |
| | | methyl)-benzyl]-(2-methoxy- | |
| | | benzenesulfonyl)-amino]-methyl}-benzoic | |
| | | acid | |
| 234 | | 4-({Benzenesulfonyl-[3-chloro-4-(difluoro- | C |
| | | phosphono-methyl)-benzyl]-amino}- | |
| | | methyl)-benzoic acid methyl ester | |
| 235 | | 4-({Benzenesulfonyl-[3-chloro-4-(difluoro- | C |
| | | phosphono-methyl)-benzyl]-amino}- | |
| | | methyl)-benzoic acid | |

IC50 ranges:
A=<99 nM
B=100-249 nM
C=250-499 nM
D=2500-24999 nM

E=>25000 nM

TABLE 2
Mass Spectral Data for Selected Compounds

| Compound | Example | | | | |
|---|---|---|---|---|---|
| Number | Number | Chemical Name | [M + H]⁺ | [M + NH₄]⁺ | [M − H]⁻ |
| 46 | 14 | [(2-Chloro-4-{[(3′-cyano- | | 650, 652 | 631, |
| | | biphenyl-4-ylmethyl)-(2- | | | 633 |
| | | methoxy-benzenesulfonyl)- | | | |
| | | amino]-methyl}-phenyl)- | | | |
| | | difluoro-methyl]- | | | |
| | | phosphonic acid | | | |
| 47 | 15 | {[2-Chloro-4-({(2- | 676, 678 | | 674, |
| | | methoxy-benzenesulfonyl)- | | | 676 |
| | | [3′-(2H-tetrazol-5-yl)- | | | |
| | | biphenyl-4-ylmethyl]- | | | |
| | | amino}-methyl)-phenyl]- | | | |
| | | difluoro-methyl}- | | | |
| | | phosphonic acid | | | |
| 56 | 17 | 5-{[(3,4-Dichloro-benzyl)- | | 581, 586 | 565, 567 |
| | | (2-methoxy- | | | |
| | | benzenesulfonyl)-amino]- | | | |
| | | methyl}-2-(oxalyl-amino)- | | | |
| | | benzoic acid | | | |
| 82 | | (2-Bromo-4-{[(2-chloro- | 648, 650 | | 646, |
| | | benzenesulfonyl)-(3,4- | | | 648, |
| | | dichloro-benzyl)-amino]- | | | 650 |
| | | methyl}-phenyl)-difluoro- | | | |
| | | methyl]-phosphonic acid | | | |
| 87 | | 2-Chloro-N-(3,4-dichloro- | | 604, 606, | 585, |
| | | benzyl)-N-(4- | | 608 | 587, |
| | | trifluoromethanesulfonylamino- | | | 589 |
| | | benzyl)- | | | |
| | | benzenesulfonamide | | | |
| 97 | | [(2-Bromo-4-{[(3,4- | | | 637, |
| | | dichloro-benzyl)-(2- | | | 639, |
| | | cyanobenzenesulfonyl)- | | | 641 |
| | | amino]-methyl}-phenyl)- | | | |
| | | difluoro-methyl]- | | | |
| | | phosphonic acid | | | |
| 98 | | [(2-Bromo-4-{[(3,4- | | | 696, |
| | | dichloro-benzyl)-(2- | | | 698, |
| | | trifluoromethoxy- | | | 700 |
| | | benzenesulfonyl)-amino]- | | | |
| | | methyl}-phenyl)-difluoro- | | | |
| | | methyl]-phosphonic acid | | | |
| 100 | | [(2-Bromo-4-{[(3,4- | 615, | | 613, |
| | | dichloro-benzyl)-(pyridine- | 617, 619 | | 615, |
| | | 2-sulfonyl)-amino]- | | | 617 |
| | | methyl}-phenyl)-difluoro- | | | |
| | | methyl]-phosphonic acid | | | |
| 101 | | [(2-Bromo-4-{[(3,4- | | 661, 663, | 642, |
| | | dichloro-benzyl)-(2- | | 665 | 644, |
| | | methoxy-benzenesulfonyl)- | | | 646 |
| | | amino]-methyl}-phenyl)- | | | |
| | | difluoro-methyl]- | | | |
| | | phosphonic acid | | | |
| 102 | | [(2-Bromo-4-{[(2-chloro- | 659, 657 | | 657, 655 |
| | | benzenesulfonyl)-(4- | | | |
| | | methanesulfonyl-benzyl)- | | | |
| | | amino]-methyl}-phenyl)- | | | |
| | | difluoro-methyl]- | | | |
| | | phosphonic acid | | | |
| 103 | | [(2-Bromo-4-{[(4-cyano- | | 622, 624, | 603, |
| | | benzyl)-(2- | | 626 | 605, |
| | | chlorobenzenesulfonyl)- | | | 607 |
| | | amino]-methyl}-phenyl)- | | | |
| | | difluoro-methyl]- | | | |
| | | phosphonic acid | | | |
| 104 | | 4-{[[3-Bromo-4-(difluoro- | 638, | | |
| | | phosphono-methyl)- | 640, 642 | | |
| | | benzyl]-(2-chloro- | | | |
| | | benzenesulfonyl)-amino]- | | | |
| | | methyl}-benzoic acid | | | |
| | | methyl ester | | | |
| 105 | | [(2-Bromo-4-{[(4-cyano- | | 618, 620 | |
| | | benzyl)-(2- | | | |
| | | methoxybenzenesulfonyl)- | | | |
| | | amino]-methyl}-phenyl)- | | | |
| | | difluoro-methyl]- | | | |
| | | phosphonic acid | | | |
| 107 | | 4-{[[3-Bromo-4-(difluoro- | 624, 626 | | 622, |
| | | phosphono-methyl)- | | | 625 |
| | | benzyl]-(2-chloro- | | | |
| | | benzenesulfonyl)-amino]- | | | |
| | | methyl}-benzoic acid | | | |
| 108 | | [(2-Bromo-4-{[(4- | 654, 656 | | 652, |
| | | methanesulfonyl-benzyl)- | | | 654 |
| | | (2-methoxy- | | | |
| | | benzenesulfonyl)-amino]- | | | |
| | | methyl}-phenyl)-difluoro- | | | |
| | | methyl]-phosphonic acid | | | |
| 110 | | 2-Methoxy-N,N-bis-[4- | | 520 | |
| | | (2H-tetrazol-5-yl)-benzyl]- | | | |
| | | benzenesulfonamide | | | |
| 111 | | (4-{[(2,4-Dimethoxy- | | 565 | 546 |
| | | benzenesulfonyl)-(4- | | | |
| | | methanesulfonyl-benzyl)- | | | |
| | | amino]-methyl}-phenyl)- | | | |
| | | oxo-acetic acid | | | |
| 112 | | N-(4-[bis (N-trifluoro- | 672 | | |
| | | methanesulfonyl)amino]benzyl)- | | | |
| | | N-(4-cyanobenzyl)- | | | |
| | | (2- | | | |
| | | methoxy)benzenesulfonamide | | | |
| 113 | | (4-{[(2,5-Dimethoxy- | | 565 | 546 |
| | | benzenesulfonyl)-(4- | | | |
| | | methanesulfonyl-benzyl)- | | | |
| | | amino]-methyl}-phenyl)- | | | |
| | | oxo-acetic acid | | | |
| 114 | | [(2-Bromo-4-{[(4- | 639, 641 | 656, 658 | 638, |
| | | methanesulfonyl-phenyl)- | | | 640 |
| | | (2-methoxy- | | | |
| | | benzenesulfonyl)-amino]- | | | |
| | | methyl}-phenyl)-difluoro- | | | |
| | | methyl]-phosphonic acid | | | |
| 115 | | N-(4-Methanesulfonyl- | 514 | | 512 |
| | | benzyl)-2-methoxy-N-[4- | | | |
| | | (1H-tetrazol-5-yl)-benzyl]- | | | |
| | | benzenesulfonamide | | | |
| 116 | | N-(4-[hydroxycarbonyl]- | 480 | | 478 |
| | | benzyl)-2-methoxy-N-[4- | | | |
| | | (1H-tetrazol-5-yl)-benzyl]- | | | |
| | | benzenesulfonamide | | | |
| 117 | | N-(4-(N-trifluoro- | | 557 | 538 |
| | | methanesulfonyl- | | | |
| | | amino]benzyl)-N-(4- | | | |
| | | cyanobenzyl)-(2- | | | |
| | | methoxy)benzenesulfonamide | | | |
| 118 | | N-(4-(N- | | 497 | 478 |
| | | oxalylamino]benzyl)-N-(4- | | | |
| | | cyanobenzyl)-(2- | | | |
| | | methoxy)benzenesulfonamide | | | |
| 119 | | N-[4-(1,2-Dihydroxy- | | 553 | |
| | | ethyl)-benzyl]-N-(4- | | | |
| | | methanesulfonyl-benzyl)- | | | |
| | | 2,5-dimethoxy- | | | |
| | | benzenesulfonamide | | | |
| 120 | | 3-{2-Bromo-4-[N-(4- | | 558, 560 | 539, |
| | | cyanobenzyl)-N-(2- | | | 541 |
| | | methoxy- | | | |
| | | benzenesulfonyl)aminomethyl]- | | | |
| | | phenyl}-acrylic acid | | | |
| 121 | | 3-{2-Bromo-4-[N-(4- | | 518, 520 | 499, |
| | | cyanobenzyl)-N-(2- | | | 501 |
| | | methoxy- | | | |
| | | benzenesulfonyl)aminomethyl]- | | | |
| | | phenoxy}-acetic acid | | | |
| 123 | | 1,2-dihydroxy-3-{2- | | 592, 594 | 573, |
| | | Bromo-4-[N-(4- | | | 575 |
| | | cyanobenzyl)-N-(2- | | | |
| | | methoxy- | | | |
| | | benzenesulfonyl)aminomethyl]- | | | |
| | | phenyl}-propionic | | | |
| | | acid | | | |
| 124 | | 3-{4-[N-(4-cyanobenzyl)- | | | 461 |
| | | N-(2-methoxy- | | | |
| | | benzenesulfonyl)aminomethyl]- | | | |
| | | phenyl}-acrylic acid | | | |
| 125 | | (4-{[(4-Methanesulfonyl- | | 535 | 516 |
| | | benzyl)-(2-methoxy- | | | |
| | | benzenesulfonyl)-amino]- | | | |
| | | methyl}-phenyl)-oxo- | | | |
| | | acetic acid | | | |
| 126 | | Hydroxyimino-(4-{[(4- | 533 | | 531 |
| | | methanesulfonyl-benzyl)- | | | |
| | | (2-methoxy- | | | |
| | | benzenesulfonyl)-amino]- | | | |
| | | methyl}-phenyl)-acetic | | | |
| | | acid | | | |
| 127 | | Hydroxy-(4-{[(4- | | 537 | |
| | | methanesulfonyl-benzyl)- | | | |
| | | (2-methoxy- | | | |
| | | benzenesulfonyl)-amino]- | | | |
| | | methyl}-phenyl)-acetic | | | |
| | | acid | | | |
| 128 | | N-(4-Methanesulfonyl- | 544, 545 | | 542 |
| | | benzyl)-2-methoxy-N-[4- | | | |
| | | (2H-tetrazol-5-ylmethoxy)- | | | |
| | | benzyl]- | | | |
| | | benzenesulfonamide | | | |
| 129 | | N-(4-(N- | 487 | 504 | |
| | | oxalylamino]benzyl)-N-(4- | | | |
| | | cyanobenzyl)-(2- | | | |
| | | methoxy)benzenesulfonamide | | | |
| 130 | | (4-{[(4-Cyano-benzyl)-(2- | 508 | | 506 |
| | | methoxy-benzenesulfonyl)- | | | |
| | | amino]-methyl}-phenyl)- | | | |
| | | difluoro-acetic acid | | | |
| 132 | | 3-(4-{[[4-(2-Carboxy- | | | 506 |
| | | vinyl)-benzyl]-(2-methoxy- | | | |
| | | benzenesulfonyl)-amino]- | | | |
| | | methyl}-phenyl)-acrylic | | | |
| | | acid | | | |
| 133 | | 5-[N-(2-Methoxy- | | 541 | 522 |
| | | benzenesulfonyl)-N- | | | |
| | | (4cyanobenzyl)-amino)- | | | |
| | | methyl]-2-(oxalyl-amino)- | | | |
| | | benzoic acid | | | |
| 134 | | 2-Carboxymethoxy-5-{[(4- | | 581, 582 | 562 |
| | | methanesulfonyl-benzyl)- | | | |
| | | (2-methoxy- | | | |
| | | benzenesulfonyl)-amino]- | | | |
| | | methyl}-benzoic acid | | | |
| 135 | | N-(4-Cyano-benzyl)-N-[4- | 504 | 521 | 502 |
| | | (2-hydroxy-3,4-dioxo- | | | |
| | | cyclobut-1-enylamino)- | | | |
| | | benzyl]-2-methoxy- | | | |
| | | benzenesulfonamide | | | |
| 136 | | N-Carboxymethyl-N-(4- | 591 | 589 | |
| | | {[(4-methanesulfonyl- | | | |
| | | benzyl)-(2-methoxy- | | | |
| | | benzenesulfonyl)-amino]- | | | |
| | | methyl}-phenyl)-oxalamic | | | |
| | | acid | | | |
| 137 | | [(2-Chloro-4-{[(4- | 610, 612 | | 608 |
| | | methanesulfonyl-benzyl)- | | | |
| | | (2-methoxy- | | | |
| | | benzenesulfonyl)-amino]- | | | |
| | | methyl}-phenyl)-difluoro- | | | |
| | | methyl]-phosphonic acid | | | |
| 138 | | 2-Methoxy-N,N-bis-[4- | 564 | | 562 |
| | | (1H-tetrazol-5-ylmethoxy)- | | | |
| | | benzyl]- | | | |
| | | benzenesulfonamide | | | |
| 139 | | 5-{[(4-Cyano-benzyl)-(2- | | 601 | 582 |
| | | methoxy-benzenesulfonyl)- | | | |
| | | amino]-methyl}-2- | | | |
| | | trifluoromethanesulfonylamino- | | | |
| | | benzoic acid | | | |
| 140 | | 2-(4-{[(4-Cyano-benzyl)- | 509 | 526 | 507 |
| | | (2-methoxy- | | | |
| | | benzenesulfonyl)-amino]- | | | |
| | | methyl}-benzyl)-malonic | | | |
| | | acid | | | |
| 141 | | N-[4-({(2-Methoxy- | 570 | 587 | 568 |
| | | benzenesulfonyl)-[4- | | | |
| | | (methyl-oxalyl-amino)- | | | |
| | | benzyl]-amino}-methyl)- | | | |
| | | phenyl]-N-methyl- | | | |
| | | oxalamic acid | | | |
| 142 | | [(2-Chloro-4-{[(3′- | | 703 | 684 |
| | | methanesulfonyl-biphenyl- | | | |
| | | 3-ylmethyl)-(2-methoxy- | | | |
| | | benzenesulfonyl)-amino]- | | | |
| | | methyl}-phenyl)-difluoro- | | | |
| | | methyl]-phosphonic acid | | | |
| 143 | | [(2-Chloro-4-{[(4′- | 686 | 703 | 684 |
| | | methanesulfonyl-biphenyl- | | | |
| | | 3-ylmethyl)-(2-methoxy- | | | |
| | | benzenesulfonyl)-amino]- | | | |
| | | methyl}-phenyl)-difluoro- | | | |
| | | methyl]-phosphonic acid | | | |
| 144 | | [(2-Chloro-4-{[(4-cyano- | 681, 683 | 698, 700 | 679, |
| | | benzyl)-(3′- | | | 681 |
| | | methanesulfonyl-biphenyl- | | | |
| | | 4-sulfonyl)-amino]- | | | |
| | | methyl}-phenyl)-difluoro- | | | |
| | | methyl]-phosphonic acid | | | |
| 145 | | 4′-{[[3-Chloro-4-(difluoro- | 666, 668 | 683, 685 | 664, |
| | | phosphono-methyl)- | | | 666 |
| | | benzyl]-(2-methoxy- | | | |
| | | benzenesulfonyl)-amino]- | | | |
| | | methyl}-biphenyl-3- | | | |
| | | carboxylic acid methyl | | | |
| | | ester | | | |
| 146 | | [(2-Chloro-4-{[(3,4- | | 619, 617 | 598, 600 |
| | | dichloro-benzyl)-(2- | | | |
| | | methoxy-benzenesulfonyl)- | | | |
| | | amino]-methyl}-phenyl)- | | | |
| | | difluoro-methyl]- | | | |
| | | phosphonic acid | | | |
| 147 | | [(2-Chloro-4-{[(4′- | 686, 688 | 703, 705 | 684, |
| | | methanesulfonyl-biphenyl- | | | 686 |
| | | 4-ylmethyl)-(2-methoxy- | | | |
| | | benzenesulfonyl)-amino]- | | | |
| | | methyl}-phenyl)-difluoro- | | | |
| | | methyl]-phosphonic acid | | | |
| 148 | | [(2-Chloro-4-{[(3′- | 686, 688 | 703, 705 | 684, |
| | | methanesulfonyl-biphenyl- | | | 686 |
| | | 4-ylmethyl)-(2-methoxy- | | | |
| | | benzenesulfonyl)-amino]- | | | |
| | | methyl}-phenyl)-difluoro- | | | |
| | | methyl]-phosphonic acid | | | |
| 149 | | 4′-{[[3-Chloro-4-(difluoro- | 652, 654 | 669, 671 | 650, |
| | | phosphono-methyl)- | | | 652 |
| | | benzyl]-(2-methoxy- | | | |
| | | benzenesulfonyl)-amino]- | | | |
| | | methyl}-biphenyl-3- | | | |
| | | carboxylic acid | | | |
| 150 | | 2,2-Dimethyl-propionic | | 731, 733 | |
| | | acid [(2-chloro-4-{[(3,4- | | | |
| | | dichloro-benzyl)-(2- | | | |
| | | methoxy-benzenesulfonyl)- | | | |
| | | amino]-methyl}-phenyl)- | | | |
| | | difluoro-methyl]-hydroxy- | | | |
| | | phosphinoyloxymethyl | | | |
| | | ester | | | |
| 151 | | [(2-Chloro-4-{[(4′- | | 717, 719 | 698, |
| | | methanesulfonyl-2-methyl- | | | 700 |
| | | biphenyl-4-ylmethyl)-(2- | | | |
| | | methoxy-benzenesulfonyl)- | | | |
| | | amino]-methyl}-phenyl)- | | | |
| | | difluoro-methyl]- | | | |
| | | phosphonic acid | | | |
| 152 | | [(2-Chloro-4-{[(4′- | 647, 649 | 664, 666 | |
| | | cyanomethyl-biphenyl-4- | | | |
| | | ylmethyl)-(2-methoxy- | | | |
| | | benzenesulfonyl)-amino]- | | | |
| | | methyl}-phenyl)-difluoro- | | | |
| | | methyl]-phosphonic acid | | | |
| 153 | | {[2-Chloro-4-({(2- | 690, 692 | | 688, |
| | | methoxy-benzenesulfonyl)- | | | 690 |
| | | [4′-(2H-tetrazol-5- | | | |
| | | ylmethyl)-biphenyl-4- | | | |
| | | ylmethyl]-amino}-methyl)- | | | |
| | | phenyl]-difluoro-methyl}- | | | |
| | | phosphonic acid | | | |
| 154 | | [(2-Chloro-4-{[(3′-cyano- | | 670, 672 | 651, |
| | | biphenyl-4-ylmethyl)- | | | 653 |
| | | (naphthalene-2-sulfonyl)- | | | |
| | | amino]-methyl}-phenyl)- | | | |
| | | difluoro-methyl]- | | | |
| | | phosphonic acid | | | |
| 155 | | [(2-Chloro-4-{[(4′-cyano- | | 650, 652 | 631, |
| | | biphenyl-4-ylmethyl)-(2- | | | 633 |
| | | methoxy-benzenesulfonyl)- | | | |
| | | amino]-methyl}-phenyl)- | | | |
| | | difluoro-methyl]- | | | |
| | | phosphonic acid | | | |
| 156 | | 4′-{[[3-Chloro-4-(difluoro- | | 685, 687 | 666, |
| | | phosphono-methyl)- | | | 668 |
| | | benzyl]-(2-methoxy- | | | |
| | | benzenesulfonyl)-amino]- | | | |
| | | methyl}-4-hydroxy- | | | |
| | | biphenyl-3-carboxylic acid | | | |
| 157 | | [(2-Chloro-4-{[(4′-cyano- | | | 710, |
| | | biphenyl-4-ylmethyl)-(2- | | | 712 |
| | | methoxy-benzenesulfonyl)- | | | |
| | | amino]-methyl}-phenyl)- | | | |
| | | difluoro-methyl]- | | | |
| | | phosphonic acid | | | |
| 158 | | {[2-Chloro-4- | 696, 698 | | |
| | | ({(naphthalene-2-sulfonyl)- | | | |
| | | [3′-(2H-tetrazol-5-yl)- | | | |
| | | biphenyl-4-ylmethyl]- | | | |
| | | amino}-methyl)-phenyl]- | | | |
| | | difluoro-methyl}- | | | |
| | | phosphonic acid | | | |
| 159 | | {[2-Chloro-4-({(2- | 676, 678 | | |
| | | methoxy-benzenesulfonyl)- | | | |
| | | [4′-(2H-tetrazol-5-yl)- | | | |
| | | biphenyl-4-ylmethyl]- | | | |
| | | amino}-methyl)-phenyl]- | | | |
| | | difluoro-methyl}- | | | |
| | | phosphonic acid | | | |
| 160 | | 4′-{[[3-Chloro-4-(difluoro- | 724, 726 | 741, 743 | 722, |
| | | phosphono-methyl)- | | | 724 |
| | | benzyl]-(2-methoxy- | | | |
| | | benzenesulfonyl)-amino]- | | | |
| | | methyl}-4-hydroxy-5- | | | |
| | | propyl-biphenyl-3- | | | |
| | | carboxylic acid methyl | | | |
| | | ester | | | |
| 161 | | [(4-{[(3′-Carbamoyl- | 651, 653 | | 649, |
| | | biphenyl-4-ylmethyl)-(2- | | | 651 |
| | | methoxy-benzenesulfonyl)- | | | |
| | | amino]-methyl}-2-chloro- | | | |
| | | phenyl)-difluoro-methyl]- | | | |
| | | phosphonic acid | | | |
| 162 | | [(2-Chloro-4-{[(2-chloro- | | 672, 674 | 653, 655 |
| | | 4-fluoro-benzenesulfonyl)- | | | |
| | | (3′-cyano-biphenyl-4- | | | |
| | | ylmethyl)-amino]-methyl}- | | | |
| | | phenyl)-difluoro-methyl]- | | | |
| | | phosphonic acid | | | |
| 163 | | [(2-Chloro-4-{[(2-chloro- | | | 635 |
| | | benzenesulfonyl)-(3′- | | | |
| | | cyano-biphenyl-4- | | | |
| | | ylmethyl)-amino]-methyl}- | | | |
| | | phenyl)-difluoro-methyl]- | | | |
| | | phosphonic acid | | | |
| 164 | | [(2-Chloro-4-{[(2-chloro- | 710, 712 | 727, 729 | 708, |
| | | 4-fluoro-benzenesulfonyl)- | | | 710 |
| | | (3′-cyano-biphenyl-4- | | | |
| | | ylmethyl)-amino]-methyl}- | | | |
| | | phenyl)-difluoro-methyl]- | | | |
| | | phosphonic acid | | | |
| 165 | | [(2-Chloro-4-{[(3′-cyano- | | 664, 666 | 645, |
| | | biphenyl-4-ylmethyl)-(2- | | | 647 |
| | | methoxy-benzenesulfonyl)- | | | |
| | | amino]-methyl}-phenyl)- | | | |
| | | difluoro-methyl]- | | | |
| | | phosphonic acid | | | |
| | | monomethyl ester | | | |
| 166 | | [(2-Chloro-4-{[(2- | | 563, 565 | 544, |
| | | methoxy-benzenesulfonyl)- | | | 546 |
| | | (1-phenyl-ethyl)-amino]- | | | |
| | | methyl}-phenyl)-difluoro- | | | |
| | | methyl]-phosphonic acid | | | |
| 167 | | [(2-Chloro-4-{[(2- | | 563, 565 | 544, |
| | | methoxy-benzenesulfonyl)- | | | 546 |
| | | (2-methyl-benzyl)-amino]- | | | |
| | | methyl}-phenyl)-difluoro- | | | |
| | | methyl]-phosphonic acid | | | |
| 168 | | [(4-{[Biphenyl-2-ylmethyl- | | 625, 627 | 606, |
| | | (2-methoxy- | | | 608 |
| | | benzenesulfonyl)-amino]- | | | |
| | | methyl}-2-chloro-phenyl)- | | | |
| | | difluoro-methyl]- | | | |
| | | phosphonic acid | | | |
| 169 | | {[2-Chloro-4-({(2-chloro- | 698 | | 696 |
| | | 4-fluoro-benzenesulfonyl)- | | | |
| | | [3′-(2H-tetrazol-5-yl)- | | | |
| | | biphenyl-4-ylmethyl]- | | | |
| | | amino}-methyl)-phenyl]- | | | |
| | | difluoro-methyl}- | | | |
| | | phosphonic acid | | | |
| 170 | | [(4-{[Benzyl-(2-methoxy- | | 549, 551 | 530, |
| | | benzenesulfonyl)-amino]- | | | 532 |
| | | methyl}-2-chloro-phenyl)- | | | |
| | | difluoro-methyl]- | | | |
| | | phosphonic acid | | | |
| 171 | | [(2-Chloro-4-{[(1,2- | | 639, 641 | 620, |
| | | diphenyl-ethyl)-(2- | | | 622 |
| | | methoxy-benzenesulfonyl)- | | | |
| | | amino]-methyl}-phenyl)- | | | |
| | | difluoro-methyl]- | | | |
| | | phosphonic acid | | | |
| 172 | | [(4-{[(4-Benzyloxy- | | 655, 657 | 636, 638 |
| | | benzyl)-(2-methoxy- | | | |
| | | benzenesulfonyl)-amino]- | | | |
| | | methyl}-2-chloro-phenyl)- | | | |
| | | difluoro-methyl]- | | | |
| | | phosphonic acid | | | |
| 173 | | [(2-Chloro-4-{[(2- | | 641, 643 | 622, 624 |
| | | methoxy-benzenesulfonyl)- | | | |
| | | (2-phenoxy-benzyl)- | | | |
| | | amino]-methyl}-phenyl)- | | | |
| | | difluoro-methyl]- | | | |
| | | phosphonic acid | | | |
| 174 | | 4′-{[[3-Chloro-4-(difluoro- | 684, 686 | 701, 703 | 682, |
| | | phosphono-methyl)- | | | 684 |
| | | benzyl]-(2-methoxy- | | | |
| | | benzenesulfonyl)-amino]- | | | |
| | | methyl}-3-fluoro-biphenyl- | | | |
| | | 4-carboxylic acid methyl | | | |
| | | ester | | | |
| 175 | | 4-Chloro-4′-{[[3-chloro-4- | 714, | 731, 733 | 712, |
| | | (difluoro-phosphono- | 716, 718 | 735 | 714, |
| | | methyl)-benzyl]-(2- | | | 716 |
| | | methoxy-benzenesulfonyl)- | | | |
| | | amino]-methyl}-biphenyl- | | | |
| | | 3-carboxylic acid ethyl | | | |
| | | ester | | | |
| 176 | | 4-Chloro-4′-{[[3-chloro-4- | 686, | 703, 705, | 684, |
| | | (difluoro-phosphono- | 688, 670 | 707 | 686, |
| | | methyl)-benzyl]-(2- | | | 688 |
| | | methoxy-benzenesulfonyl)- | | | |
| | | amino]-methyl}-biphenyl- | | | |
| | | 3-carboxylic acid | | | |
| 177 | | 4′-{[[3-Chloro-4-(difluoro- | | 687, 689 | 668, |
| | | phosphono-methyl)- | | | 670 |
| | | benzyl]-(2-methoxy- | | | |
| | | benzenesulfonyl)-amino]- | | | |
| | | methyl}-3-fluoro-biphenyl- | | | |
| | | 4-carboxylic acid | | | |
| 178 | | [(4-{[(2-Bromo-benzyl)-(2- | | 627, 629 | 608, |
| | | methoxy-benzenesulfonyl)- | | | 610 |
| | | amino]-methyl}-2-chloro- | | | |
| | | phenyl)-difluoro-methyl]- | | | |
| | | phosphonic acid | | | |
| 179 | | [(2-Chloro-4-{[(2- | 609, 61 | | 607, |
| | | methoxy-benzenesulfonyl)- | | | 609 |
| | | (2-pyridin-3-yl-benzyl)- | | | |
| | | amino]-methyl}-phenyl)- | | | |
| | | difluoro-methyl]- | | | |
| | | phosphonic acid | | | |
| 180 | | [(2-Chloro-4-{[(2- | 609, 611 | | 607, |
| | | methoxy-benzenesulfonyl)- | | | 609 |
| | | (2-pyridin-4-yl-benzyl)- | | | |
| | | amino]-methyl}-phenyl)- | | | |
| | | difluoro-methyl]- | | | |
| | | phosphonic acid | | | |
| 181 | | [(2-Chloro-4-{[(4′-chloro- | 701, | | 699, |
| | | 3′-hydroxycarbamoyl- | 703, 705 | | 701, |
| | | biphenyl-4-ylmethyl)-(2- | | | 703 |
| | | methoxy-benzenesulfonyl)- | | | |
| | | amino]-methyl}-phenyl)- | | | |
| | | difluoro-methyl]- | | | |
| | | phosphonic acid | | | |
| 182 | | 4′-{2-[[3-Chloro-4- | 680, 682 | 697, 699 | 678, |
| | | (difluoro-phosphono- | | | 680 |
| | | methyl)-benzyl]-(2- | | | |
| | | methoxy-benzenesulfonyl)- | | | |
| | | amino]-ethyl}-biphenyl-3- | | | |
| | | carboxylic acid methyl | | | |
| | | ester | | | |
| 183 | | [(2-Chloro-4-{[(2- | | 655, 657 | 636, |
| | | methoxy-benzenesulfonyl)- | | | 638 |
| | | (3′-methoxy-biphenyl-2- | | | |
| | | ylmethyl)-amino]-methyl}- | | | |
| | | phenyl)-difluoro-methyl]- | | | |
| | | phosphonic acid | | | |
| 184 | | [(2-Chloro-4-{[(3′- | | 641, 643 | 622, |
| | | hydroxy-biphenyl-2- | | | 624 |
| | | ylmethyl)-(2-methoxy- | | | |
| | | benzenesulfonyl)-amino]- | | | |
| | | methyl}-phenyl)-difluoro- | | | |
| | | methyl]-phosphonic acid | | | |
| 185 | | 3′-{2-[[3-Chloro-4- | | 697, 699 | 678, |
| | | (difluoro-phosphono- | | | 680 |
| | | methyl)-benzyl]-(2- | | | |
| | | methoxy-benzenesulfonyl)- | | | |
| | | amino]-ethyl}-biphenyl-3- | | | |
| | | carboxylic acid methyl | | | |
| | | ester | | | |
| 186 | | [(4-{[(3′-Carbamoyl- | 651, 653 | | 649, |
| | | biphenyl-2-ylmethyl)-(2- | | | 651 |
| | | methoxy-benzenesulfonyl)- | | | |
| | | amino]-methyl}-2-chloro- | | | |
| | | phenyl)-difluoro-methyl]- | | | |
| | | phosphonic acid | | | |
| 187 | | [(4-{[(3′-Carbamoyl- | | 639, 641 | 620, |
| | | biphenyl-2-ylmethyl)-(2- | | | 622 |
| | | methoxy-benzenesulfonyl)- | | | |
| | | amino]-methyl}-2-chloro- | | | |
| | | phenyl)-difluoro-methyl]- | | | |
| | | phosphonic acid | | | |
| 188 | | 4′-{2-[[3-Chloro-4- | | 683, 685 | 664, |
| | | (difluoro-phosphono- | | | 666 |
| | | methyl)-benzyl]-(2- | | | |
| | | methoxy-benzenesulfonyl)- | | | |
| | | amino]-ethyl}-biphenyl-3- | | | |
| | | carboxylic acid | | | |
| 189 | | 3′-{2-[[3-Chloro-4- | | 683, 685 | 664, |
| | | (difluoro-phosphono- | | | 666 |
| | | methyl)-benzyl]-(2- | | | |
| | | methoxy-benzenesulfonyl)- | | | |
| | | amino]-ethyl}-biphenyl-3- | | | |
| | | carboxylic acid | | | |
| 214 | | [(4-{[Biphenyl-2-ylmethyl- | 621, 623 | | 619, 621 |
| | | (3-carbamoyl- | | | |
| | | benzenesulfonyl)-amino]- | | | |
| | | methyl}-2-chloro-phenyl)- | | | |
| | | difluoro-methyl]- | | | |
| | | phosphonic acid | | | |
| 215 | | {[2-Chloro-4-({(2- | | 649, 651 | 630, 632 |
| | | methoxy-benzenesulfonyl)- | | | |
| | | [2(4-methyl-pentyloxy)- | | | |
| | | benzyl]-amino}-methyl)- | | | |
| | | phenyl]-difluoro-methyl}- | | | |
| | | phosphonic acid | | | |
| 216 | | [(4-{[Biphenyl-2-ylmethyl- | | 619, 621 | 601, 603 |
| | | (3-cyano- | | | |
| | | benzenesulfonyl)-amino]- | | | |
| | | methyl}-2-chloro-phenyl)- | | | |
| | | difluoro-methyl]- | | | |
| | | phosphonic acid | | | |
| 217 | | 3-[(4-{[(2-Methoxy- | 618 | | 616 |
| | | benzenesulfonyl)-(2- | | | |
| | | pyridin-4-yl-benzyl)- | | | |
| | | amino]-methyl}-phenyl)- | | | |
| | | oxalyl-amino]-2-methyl- | | | |
| | | propionic acid | | | |
| 218 | | [(2-Chloro-4-{[(3′-cyano- | | 780, 782 | 761, |
| | | biphenyl-4-ylmethyl)-(2- | | | 763 |
| | | methoxy-benzenesulfonyl)- | | | |
| | | amino]-methyl}-phenyl)- | | | |
| | | difluoro-methyl]- | | | |
| | | phosphonic acid mono-(1- | | | |
| | | isopropoxycarbonyloxy- | | | |
| | | ethyl) ester | | | |
| 219 | 42 | [S]-N-[(2-Chloro-4-{[(3′- | | 749, 751 | 730, |
| | | cyano-biphenyl-4- | | | 732 |
| | | ylmethyl)-(2-methoxy- | | | |
| | | benzenesulfonyl)-amino]- | | | |
| | | methyl}-phenyl)-difluoro- | | | |
| | | methyl]-phosphonyl-(ethyl | | | |
| | | alaninate) | | | |
| 221 | | [(2-Chloro-4-{[(3′-ethoxy- | | 669, 671 | 650, 652 |
| | | biphenyl-2-ylmethyl)-(2- | | | |
| | | methoxy-benzenesulfonyl)- | | | |
| | | amino]-methyl}-phenyl)- | | | |
| | | difluoro-methyl]- | | | |
| | | phosphonic acid | | | |
| 222 | | 4′-{[(4-Carboxymethoxy- | | 629, 631 | 610, |
| | | 3-chloro-benzyl)-(2- | | | 612 |
| | | methoxy-benzenesulfonyl)- | | | |
| | | amino]-methyl}-4- | | | |
| | | hydroxy-biphenyl-3- | | | |
| | | carboxylic acid | | | |
| 223 | | 3-[(4-{[{4-[(2-Carboxy- | 714 | 731 | 712 |
| | | propyl)-oxalyl-amino]- | | | |
| | | benzyl}-(2-methoxy- | | | |
| | | benzenesulfonyl)-amino]- | | | |
| | | methyl}-phenyl)-oxalyl- | | | |
| | | amino]-2-methyl-propionic | | | |
| | | acid | | | |
| 224 | | 5-{[Biphenyl-2-ylmethyl- | 562 | 579 | 560 |
| | | (2-methoxy- | | | |
| | | benzenesulfonyl)-amino]- | | | |
| | | methyl}-2- | | | |
| | | carboxymethoxy-benzoic | | | |
| | | acid | | | |
| 225 | | 3-[(4-{[(2-Methoxy- | | 648 | 629 |
| | | benzenesulfonyl)- | | | |
| | | naphthalen-1-ylmethyl- | | | |
| | | amino]-methyl}-phenyl)- | | | |
| | | oxalyl-amino]-butyric acid | | | |
| 226 | 46 | 2,2-Dimethyl-propionic | | 878, 880 | |
| | | acid [(2-chloro-4-{[(3′- | | | |
| | | cyano-biphenyl-4- | | | |
| | | ylmethyl)-(2-methoxy- | | | |
| | | benzenesulfonyl)-amino]- | | | |
| | | methyl}-phenyl)-difluoro- | | | |
| | | methyl]-(2,2-dimethyl- | | | |
| | | propionyloxymethoxy)- | | | |
| | | phosphinoyloxymethyl | | | |
| | | ester | | | |
| 227 | | 2,2-Dimethyl-propionic | | 764, 766 | 745, |
| | | acid [(2-chloro-4-{[(3′- | | | 747 |
| | | cyano-biphenyl-4- | | | |
| | | ylmethyl)-(2-methoxy- | | | |
| | | benzenesulfonyl)-amino]- | | | |
| | | methyl}-phenyl)-difluoro- | | | |
| | | methyl]-hydroxy- | | | |
| | | phosphinoyloxymethyl | | | |
| | | ester | | | |
| 228 | | i2,2-Dimethyl-propionic | | 771, 773 | 752, 754 |
| | | acid [(2-bromo-4-{[(4- | | | |
| | | methanesulfonyl-phenyl)- | | | |
| | | (2-methoxy- | | | |
| | | benzenesulfonyl)-amino]- | | | |
| | | methyl}-phenyl)-difluoro- | | | |
| | | methyl]-hydroxy- | | | |
| | | phosphinoyloxymethyl | | | |
| | | ester | | | |
| 229 | | 2,2-Dimethyl-propionic | | 885, 887 | |
| | | acid [(2-bromo-4-{[(4- | | | |
| | | methanesulfonyl-phenyl)- | | | |
| | | (2-methoxy- | | | |
| | | benzenesulfonyl)-amino]- | | | |
| | | methyl}-phenyl)-difluoro- | | | |
| | | methyl]-(2,2-dimethyl- | | | |
| | | propionyloxymethoxy)- | | | |
| | | phosphinoyloxymethyl | | | |
| | | ester | | | |
| 230 | | [(4-{[Biphenyl-2-ylmethyl- | 705 | | 703 |
| | | (4-iodo-pyridine-3- | | | |
| | | sulfonyl)-amino]-methyl}- | | | |
| | | 2-chloro-phenyl)-difluoro- | | | |
| | | methyl]-phosphonic acid | | | |
| 231 | | 4-{[[3-Chloro-4-(difluoro- | 590, 592 | | 588, |
| | | phosphono-methyl)- | | | 590 |
| | | benzyl]-(2-methoxy- | | | |
| | | benzenesulfonyl)-amino]- | | | |
| | | methyl}-benzoic acid | | | |
| | | methyl ester | | | |
| 232 | | {[2-Chloro-4-({[3-chloro- | | | 640, |
| | | 4-(difluoro-phosphono- | | | 642 |
| | | methyl)-benzyl]-indan-1- | | | |
| | | yl-amino}-methyl)- | | | |
| | | phenyl]-difluoro-methyl}- | | | |
| | | phosphonic acid | | | |
| 233 | | 4-{[[3-Chloro-4-(difluoro- | 576, 578 | 593, 595 | 574, |
| | | phosphono-methyl)- | | | 576 |
| | | benzyl]-(2-methoxy- | | | |
| | | benzenesulfonyl)-amino]- | | | |
| | | methyl}-benzoic acid | | | |
| 234 | | 4-({Benzenesulfonyl-[3- | 560, 562 | | 558, 559 |
| | | chloro-4-(difluoro- | | | |
| | | phosphono-methyl)- | | | |
| | | benzyl]-amino}-methyl)- | | | |
| | | benzoic acid methyl ester | | | |
| 235 | | 4-({Benzenesulfonyl-[3- | 546, 548 | 563, 565 | 544, 546 |
| | | chloro-4-(difluoro- | | | |
| | | phosphono-methyl)- | | | |
| | | benzyl]-amino}-methyl)- | | | |
| | | benzoic acid | | | |

### EXPERIMENTAL

In the experimental disclosure which follows, all weights are given in grams (g), milligrams (mg), micrograms (μg), nanograms (ng), or picograms (pg), all amounts are given in moles (mol), millimoles (mmol), micromoles (μmol), nanomoles (nmol), picomoles (pmol), or femtomoles (fmol), all concentrations are given as percent by volume (%), proportion by volume (v:v), molar (M), millimolar (mM), micromolar (μM), nanomolar (nM), picomolar (pM), femtomolar (fM), or normal (N), all volumes are given in liters (L), milliliters (mL), or microliters (μL), and linear measurements are given in millimeters (mm), micrometers (μm), or nanometers (nm) and mp is melting point, unless otherwise indicated.

### Example 1

### {[4-({4-[(Diethoxy-phosphoryl)-difluoro-methyl]-benzylamino}-methyl)-phenyl]-difluoro-methyl}-phosphonic acid diethyl ester HCl salt

Bis-(4-iodo-benzyl)-carbamic acid tert-butyl ester (2):

Step A: A solution of 4-iodo-benzylamine (18.64 g, 80 mmol), di-tert-butyl dicarbonate (19.21 g, 88 mmol), and NaOH (4.80 g, 120 mmol) in dioxane/H₂O (1:1, 250 mL) was stirred at room temp for 18 hours. The solvent was removed and the residue was dissolved in EtOAc (200 mL). The organic layer was washed with H₂O (50 mL), HCl (1N, 2×50 mL), NaHCO₃ (10%, 50 mL) and H₂O (50 mL). The organic solution was dried over anhydrous Na₂SO₄. The solvent was evaporated to give (4-iodobenzyl)carbamic acid tert-butyl ester (1, 26.50 g, 99%).

Step B: A solution of (4-iodobenzyl)carbamic acid tert-butyl ester (1, 26.50 g, 79 mmol) in DMF (100 mL) was stirred with NaH (60%, 3.80 g, 95 mmol) at room temp under Ar for 2 hours. A solution of 1-bromomethyl-4-iodo-benzene (28.33 g, 95 mmol) in DMF (10 mL) was added and stirred for 20 hours. The solution was concentrated on a rotary evaporator. The residue was dissolved in EtOAc. The organic layer was washed with H₂O. The solvent was removed and the residue was purified by column chromatography on silica gel, eluting with hexanes/EtOAc (9:1) to provide Bis-(4-iodo-benzyl)-carbamic acid tert-butyl ester as a white solid (2, 32.05 g, 73%).

¹H NMR (300 MHz, CDCl₃) δ 7.64 (d, J=8.1 Hz, 4H), 6.93 (bs, 4H), 4.33 (bs, 2H), 4.26 (bs, 2H), 1.47 (s, 9H).

Step C: A solution of (bromodifluoromethyl)phosphonic acid diethyl ester (21.36 g, 80 mmol) in N,N-dimethylacetamide (25 mL) was added dropwise into a suspension of activated Zn (5.23 g, 80 mmol) under Ar. The reaction was initialized by heating and kept below 50° C. After the mixture was stirred for 3 hours, CuBr (11.48 g, 80 mmol) was added and stirred for 1 hour. A solution of bis-(4-iodobenzyl)carbamic acid tert-butyl ester (2, 10.98 g, 20 mmol) in N,N-dimethylacetamide (25 mL) was added slowly. The resulting suspension was stirred at room temp for 18 hours. Water (50 mL) was added and the mixture was filtered through a bed of Celite. The filtrate was diluted with EtOAc (250 mL) and the organic layer was washed with H₂O (50 mL), NaHCO₃ (5%, 50 mL) and H₂O (50 mL). The solvent was removed and the residue was purified by column chromatography on silica gel, eluting with hexanes/EtOAc (1:1) to provide ({4-[(tert-Butoxycarbonyl-{4-[(diethoxy-phosphoryl)-difluoro-methyl]-benzyl}-amino)-methyl]-phenyl}-difluoro-methyl)phosphonic acid diethyl ester as a colorless oil (3, 6.50 g, 48%).

¹H NMR (300 MHz, CDCl₃) δ 7.59 (d, J=8.4 Hz, 4H), 7.29 (bs, 4H), 4.46-4.09 (overlapping, 12H), 1.48 (s, 9H), 1.35 (m, 12H).

Step D: ({4-[(tert-Butoxycarbonyl-{4-[(diethoxy-phosphoryl)-difluoro-methyl]-benzyl}-amino)-methyl]-phenyl}-difluoro-methyl)-phosphonic acid diethyl ester (3, 3.00 g, 4.48 mmol) was stirred with HCl (4N) in dioxane (15 mL) at room temp for 5 hours. The solvent and excess HCl was removed to provide the title product as a white solid (4, 2.70 g, 99%).

¹H NMR (300 MHz, CDCl₃) δ 10.55 (bs, 2H), 7.71-7.58 (overlapping, 8H), 4.23 (m, 8H), 3.94 (s, 4H), 1.30 (m, 12H).

### Example 2

### {[4-({(2-Chloro-benzenesulfonyl)-[4-(difluoro-phosphono-methyl)-benzyl]-amino}-methyl)-phenyl]-difluoro-methyl}-phosphonic acid (5)

Step A: A solution of {[4-({4-[(diethoxy-phosphoryl)-difluoro-methyl]-benzylamino}-methyl)-phenyl]-difluoro-methyl}-phosphonic acid diethyl ester HCl salt (4, 122 mg, 0.20 mmol), 2-chloro-benzenesulfonyl chloride (106 mg, 0.50 mmol), and DMAP (367 mg, 3.00 mmol) in CH₂Cl₂ (10 mL) was stirred at room temp for 3 hours. More CH₂Cl₂ (80 mL) was added. The solution was washed with H₂O (20 mL), HCl (1N, 20 mL), NaHCO₃ (saturated, 20 mL) and H₂O (20 mL). The solvent was removed to provide a residue, which was purified by preparative layer chromatography on silica gel (EM-5717-7), developing with hexanes/EtOAc (1:1) to provide ({4-[((2-chloro-benzenesulfonyl)-{4-[(diethoxy-phosphoryl)-difluoro-methyl]-benzyl}-amino)-methyl]-phenyl}-difluoro-methyl)-phosphonic acid diethyl ester (5, 76 mg, 51%).

Step B: A solution of ({4-[((2-chloro-benzenesulfonyl)-{4-[(diethoxy-phosphoryl)-difluoro-methyl]-benzyl}-amino)-methyl]-phenyl}-difluoro-methyl)-phosphonic acid diethyl ester (76 mg, 0.10 mmol) and iodotrimethysilane (0.5 mL) in CH₂Cl₂ (5 mL) was stirred at room temp for 3 hours. The solvent and excess iodotrimethysilane was removed. The residue was treated with CH₃CN (8 mL), TFA (1 mL) and H₂O (2 mL), and stirred for 5 hours at room temp. The solvent was removed to provide a white solid (6, 32 mg, 51%): mp 170-172° C.;

¹H NMR (300 MHz, MeOH-d₄) δ 8.09 (d, J=7.8 Hz, 1H), 7.62-7.46 (overlapping, 7H), 7.20 (d, J=8.1 Hz, 4H), 4.50 (s, 4H); MS m/z 629.8 (M−H)⁻, 314.6 (M−2H)²⁻.

### Example 3

### [(4-{[[4-(Difluoro-phosphono-methyl)-benzyl]-(2-phenyl-ethanesulfonyl)-amino]-methyl}-phenyl)-difluoro-methyl]-phosphonic acid (9)

Step A: A solution of [(4-{[{4-[(diethoxyphosphoryl)difluoromethyl]benzyl}-(2-phenyl-E-ethenesulfonyl)-amino]-methyl}-phenyl)-difluoromethyl]phosphonic acid diethyl ester (7, 215 mg, 0.29 mmol) in methanol (20 mL) was hydrogenated with 10% Pd on activated carbon (100 mg) at room temp for 2 hours. After filtration through a bed of Celite, the filtrate was evaporated to provide [(4-{[{4-[(diethoxy-phosphoryl)-difluoro-methyl]-benzyl}-(2-phenyl-ethanesulfonyl)-amino]-methyl}-phenyl)-difluoro-methyl]-phosphonic acid diethyl ester (8, 155 mg, 72%).

Step B: Intermediate 8 was converted to the title product 9 using the procedure in Example 2, step B: (9, 87 mg, 66%): mp 181-183° C.;

¹H NMR (300 MHz, DMSO-d₆) δ 9.0-11.0 (b, 4H), 8.29 (d, J=7.8 Hz, 4H), 8.20 (d, J=8.4 Hz, 4H), 8.12-8.03 (overlapping, 5H), 5.27 (s, 4H), 4.26 (dd, 2H), 3.81 (dd, 2H); MS m/z 623.9 (M−H)⁻, 311.5 (M−2H)²⁻.

### Example 4

### General procedure for synthesis of acyl and carbamoyl derivatives: {[4-({Benzoyl-[4-(difluoro-phosphono-methyl)-benzyl]-amino}-methyl)-phenyl]-difluoro-methyl}-phosphonic acid (11)

A solution of {[4-({4-[(diethoxy-phosphoryl)-difluoromethyl]-benzylamino}-methyl)-phenyl]-difluoro-methyl}-phosphonic acid diethyl ester HCl salt (4, 61 mg, 0.10 mmol), benzoyl chloride (70 mg, 0.50 mmol), and DMAP (122 mg, 1.00 mmol) in CH₂Cl₂ (5 mL) was stirred at room temp for 2 hours. Additional CH₂Cl₂ (50 mL) was added. The solution was washed with H₂O (20 mL), HCl (1N, 20 mL), NaHCO₃ (saturated, 20 mL) and H₂O (20 mL). The solvent was removed to provide a residue, which was purified by preparative layer chromatography on silica gel (EM-5717-7), developing with hexanes/EtOAc (1:1) to provide ({4-[(benzoyl-{4-[(diethoxy-phosphoryl)-difluoro-methyl]-benzyl}-amino)-methyl]-phenyl}-difluoro-methyl)-phosphonic acid diethyl ester (10, 13 mg, 19%).

A solution of ({4-[(benzoyl-{4-[(diethoxy-phosphoryl)-difluoro-methyl]-benzyl}-amino)-methyl]-phenyl}-difluoro-methyl)-phosphonic acid diethyl ester (10, 13 mg, 0.02 mmol) and iodotrimethysilane (0.3 mL) in CH₂Cl₂ (5 mL) was stirred at room temp for 3 hours. The solvent and excess iodotrimethysilane was removed. The residue was treated with CH₃CN (8 mL), TFA (1 mL) and H₂O (2 mL), and stirred for 5 hours at room temp. The solvent was removed to provide a slightly yellow solid (11, 9 mg, 83%): mp 87-89° C.;

¹H NMR (300 MHz, MeOH-d₄) δ 7.64 (d, J=6.3 Hz, 4H), 7.51-7.44 (overlapping, 5H), 7.37 (d, J=6.9 Hz, 2H), 7.22 (d, J=7.2 Hz, 2H), 4.73 (s, 2H), 4.49 (s, 2H); MS m/z 559.9 (M−H)⁻, 279.6 (M−2H)²⁻.

### Example 5

### [(4-{3-Benzyl-1-[4-(difluorophosphonomethyl)-benzyl]-ureidomethyl}-phenyl)-difluoro-methyl]-phosphonic acid (13)

A solution of {[4-({4-[(diethoxy-phosphoryl)-difluoro-methyl]-benzylamino}-methyl)-phenyl]-difluoro-methyl}-phosphonic acid diethyl ester HCl salt (4, 122 mg, 0.20 mmol), isocyanatomethylbenzene (80 mg, 0.60 mmol), and NEt₃ (0.5 mL) in CH₂Cl₂ (10 mL) was stirred at room temp for 2 hours. More CH₂Cl₂ (80 mL) was added. The solution was washed with H₂O (20 mL), HCl (1N, 20 mL), NaHCO₃ (saturated, 20 mL) and H₂O (20 mL). The solvent was removed to provide a residue, which was purified by preparative layer chromatography on silica gel (EM-5717-7), developing with hexanes/EtOAc (1:1) to provide {[4-(3-benzyl-1-{4-[(diethoxy-phosphoryl)-difluoro-methyl]-benzyl}-ureidomethyl)-phenyl]-difluoro-methyl}-phosphonic acid diethyl ester (12, 55 mg, 39%).

A solution of {[4-(3-benzyl-1-{4-[(diethoxyphosphoryl)-difluoro-methyl]-benzyl}-ureidomethyl)-phenyl]-difluoro-methyl}-phosphonic acid diethyl ester (12, 55 mg, 0.08 mmol) and iodotrimethysilane (0.5 mL) in CH₂Cl₂ (5 mL) was stirred at room temp for 3 hours. The solvent and excess iodotrimethysilane was removed. The residue was treated with CH₃CN (8 mL), TFA (1 mL) and H₂O (2 mL), and stirred for 5 hours at room temp. The solvent was removed to provide a slightly brown solid (13, 32 mg, 65%): mp 124-127° C.;

¹H NMR (300 MHz, DMSO-d₆) δ 7.50 (d, J=6.9 Hz, 4H), 7.32 (d, J=8.4 Hz, 4H), 7.27 (d, J=6.3 Hz, 2H), 7.18 (overlapping, 3H), 4.52 (s, 4H), 4.29 (d, J=4.8 Hz, 2H); MS m/z 590.9 (M+H)⁺, 588.9 (M−H)⁻, 294.1 (M−2H)²⁻.

### Example 6

### {[4-({[4-(Difluoro-phosphono-methyl)-benzyl]-methyl-amino}-methyl)-phenyl]-difluoro-methyl}-phosphonic acid (15)

A solution of {[4-({4-[(diethoxy-phosphoryl)-difluoro-methyl]-benzylamino}-methyl)-phenyl]-difluoro-methyl}-phosphonic acid diethyl ester HCl salt (4, 122 mg, 0.20 mmol), CH₃I (72 mg, 0.50 mmol), K₂CO₃ (316 mg, 2.00 mmol) in acetone (10 mL) was stirred at room temp for 2 hours. The filtrate was concentrated on a rotary evaporator at room temp. The residue was purified by preparative layer chromatography on silica gel (EM-5717-7), developing with hexanes/EtOAc (1:1) to provide ({4-[({4-[(diethoxy-phosphoryl)-difluoro-methyl]-benzyl}-methyl-amino)-methyl]-phenyl}-difluoro-methyl)-phosphonic acid diethyl ester (14, 35 mg, 30%). A solution of ({4-[({4-[(diethoxy-phosphoryl)-difluoro-methyl]-benzyl}-methyl-amino)-methyl]-phenyl}-difluoro-methyl)-phosphonic acid diethyl ester (14, 35 mg, 0.06 mmol) and iodotrimethysilane (0.3 mL) in CH₂Cl₂ (5 mL) was stirred at room temp for 3 hours. The solvent and excess iodotrimethysilane was removed. The residue was treated with CH₃CN (8 mL), TFA (1 mL) and H₂O (2 mL), and stirred for 5 hours at room temp. The solvent was removed to provide a white solid (15, 8 mg, 28%): mp 268-270° C.;

¹H NMR (300 MHz, DMSO-d₆) δ 7.49 (m, 8H), 4.23 (s, 4H), 2.23 (s, 3H); MS m/z 472.0 (M+H)⁺, 470.0 (M−H)⁻, 234.6 (M−2H)²⁻.

### Example 7

### {[4-({Benzyloxycarbonyl-[4-(difluoro-phosphono-methyl)-benzyl]-amino}-methyl)-phenyl]-difluoro-methyl}-phosphonic acid (17)

A solution of {[4-({4-[(diethoxy-phosphoryl)-difluoro-methyl]-benzylamino}-methyl)-phenyl]-difluoro-methyl}-phosphonic acid diethyl ester HCl salt (4, 700 mg, 1.16 mmol) and iodotrimethysilane (1 mL) in CH₂Cl₂ was stirred at room temp for 16 hours. The solvent and excess iodotrimethysilane was removed. The residue was treated with CH₃CN (8 mL), TFA (1 mL) and H₂O (2 mL), and stirred for 5 hours at room temp. The solvent was removed to provide [(4-{[4-(difluoro-phosphono-methyl)-benzylamino]-methyl}-phenyl)-difluoro-methyl]-phosphonic acid (16, 569 mg, 99%). A solution of [(4-{[4-(difluoro-phosphono-methyl)-benzylamino]-methyl}-phenyl)-difluoro-methyl]-phosphonic acid (16, 502 mg, 1.00 mmol), benzyl chloroformate (409 mg, 2.40 mmol) and DMAP (440 mg, 3.60 mmol) in CH₂Cl₂ (10 mL) was stirred at room temp for 18 hours. The solvent was removed by rotary evaporation at room temp. The residue was dissolved in EtOAc and extracted by H₂O. The aqueous layer was acidified using HCl (1N) to pH 1 and extracted by EtOAc (3×30 mL). The organic layer was dried over anhydrous Na₂SO₄. The solvent was removed to provide a white solid (17, 130 mg, 22%): mp 106-108° C.;

¹H NMR (300 MHz, DMSO-d₆) δ 7.46 (m, 5H), 7.32 (m, 8H), 5.16 (s, 2H), 4.51 (s, 4H); MS m/z 589.9 (M−H)⁻, 294.6 (M−2H)²⁻.

### Example 8

### {[4-({Biphenyl-4-ylmethyl-[4-(difluoro-phosphono-methyl)-benzyl]-amino}-methyl)-phenyl]-difluoro-methyl}-phosphonic acid (20)

A solution of [(4-bromomethyl-phenyl)-difluoro-methyl]-phosphonic acid diethyl ester (18, 714 mg, 2.00 mmol), 4-phenylbenzylamine (73 mg, 0.4 mmol), and NEt₃ (40 mg, 0.40 mmol) in CH₂Cl₂ (10 mL) was stirred at room temp for 20 hours. The solvent was removed to provide a residue, which was purified by column chromatography on silica gel, eluting with hexanes/EtOAc (1:1) to provide ({4-[(biphenyl-4-ylmethyl-{4-[(diethoxy-phosphoryl)-difluoro-methyl]-benzyl}-amino)-methyl]-phenyl}-difluoro-methyl)-phosphonic acid diethyl ester (19, 100 mg, 34%). A solution of ({4-[(biphenyl-4-ylmethyl-{4-[(diethoxy-phosphoryl)-difluoro-methyl]-benzyl}-amino)-methyl]-phenyl}-difluoro-methyl)-phosphonic acid diethyl ester (19, 100 mg, 0.14 mmol) and iodotrimethysilane (0.5 mL) in CH₂Cl₂ (5 mL) was stirred at room temp for 3 hours. The solvent and excess iodotrimethysilane was removed. The residue was treated with CH₃CN (8 mL), TFA (1 mL) and H₂O (2 mL), and stirred for 5 hours at room temp. The solvent was removed to provide a slightly brown solid (20, 70 mg, 80%): mp 125-128 C;

¹H NMR (300 MHz, MeOH-d₄) δ 7.71-7.62 (overlapping, 8H), 7.54-7.45 (overlapping, 9H), 4.32 (s, 4H), 4.29 (s, 2H); MS m/z 624.0 (M+H)⁺, 621.9 (M−H)⁻, 310.5 (M−2H)²⁻.

### Example 9

### [(2-Bromo-4-methyl-phenyl)-difluoromethyl]-phosphonic acid diethyl ester (27)

To activated Zn (1.2 g, 19 mmol) in DMA (7 mL) was added bromodifluoromethyldiethyl-phosphonate (5.0 g, 19 mmol) in DMA (7 mL). The resulting mixture was stirred at 45° C. for 3 hours, after which copper(I) bromide (2.7 g, 19 mmol) was added and stirring was continued for 0.5 hours at room temperature. 3-Bromo-4-iodotoluene (2.8 g, 9.4 mmol) was then added and the mixture was sonicated at room temperature for 12 hours. The reaction mixture was partitioned between ether and H₂O, filtered through Celite, and the organic layer was dried over MgSO₄ and concentrated in vacuo to yield 2.1 g (63%) of 27 as a clear, colorless oil;

¹H NMR (300 MHz, CDCl₃) δ 7.51 (d, J=6 Hz, 1H), 7.27 (s, 1H), 7.20 (d, J=7.5 Hz, 1H), 4.27 (m, 4H), 1.36 (t, J=8.1 Hz)

### [(2-Bromo-4-bromomethyl-phenyl)-difluoro-methyl]-phosphonic acid diethyl ester (28)

To [(2-Bromo-4-methyl-phenyl)-difluoromethyl]-phosphonic acid diethyl ester (27, 2.1 g, 5.8 mmol) in benzene (50 mL) was added N-bromosuccinimide (1.2 g, 6.8 mmol) and AIBN (0.050 g). The resulting mixture was stirred for 12 hours at room temperature in front of a 100 W bulb. It was then washed with H₂O, sat. NaHCO₃, and brine, and the organic layer was dried over MgSO4, and concentrated in vacuo. The resulting material was purified via column chromatography (4/1 hexanes/ethyl acetate) to yield 1.7 g (66%) of 28 as a clear, colorless oil;

¹H NMR (300 MHz, CDCl₃) δ 7.71 (s, 1H), 7.61 (d, J=8.1 Hz, 2H), 7.41 (d, J=8.1 Hz, 1H), 4.41 (s, 2H), 4.27 (m, 4H), 1.36 (t, J=8.1 Hz)

### 4-(Benzenesulfonylamino-methyl)-benzoic acid methyl ester (21)

A solution of benzenesulfonyl chloride (1.76 g, 10 mmol), 4-aminomethyl-benzoic acid methyl ester HCl salt (2.02 g, 10 mmol) and Dimethyl-pyridin-4-yl-amine (DMAP, 3.66 g, 30 mmol) in CH₂Cl₂ (20 mL) was stirred at room temp for 16 hours. After dilution with CH₂Cl₂ (100 mL) and the resulting solution was washed with H₂O (50 mL), HCl (1N, 50 mL), NaHCO₃ (10%, 50 mL) and H₂O (50 mL). The organic solvent was dried over anhydrous Na₂SO₄. The solvent was evaporated to provide 21 as a white solid (3.02 g, 99%):

¹H NMR (300 MHz, DMSO-d₆) δ 8.29 (t, J=5.7 Hz, 1H), 7.86 (d, J=8.4 Hz, 2H), 7.80 (d, J=7.2 Hz, 2H), 7.63-7.57 (overlapping, 3H), 7.39 (d, J=7.5 Hz, 2H), 4.08 (d, J=5.4 Hz, 2H), 3.83 (s, 3H).

### 4-[(Benzenesulfonyl-{3-bromo-4-[(diethoxy-phosphoryl)-difluoro-methyl]-benzyl}-amino)-methyl]-benzoic acid methyl ester (29)

To 4-(Benzenesulfonylamino-methyl)-benzoic acid methyl ester (21, 0.19 g, 0.62 mmol) in DMF (5 mL) was added NaH [60% in oil] (0.025 g, 0.62 mmol). After 0.1 hour, [(2-Bromo-4-bromomethyl-phenyl)-difluoro-methyl]-phosphonic acid diethyl ester (28, 0.27 g, 0.62 mmol) was added and the resulting mixture was stirred at room temperature for 1 hour. The reaction was partitioned between Et₂O and H₂O, after which the organic layer was dried over MgSO₄ and concentrated in vacuo. The resulting material was purified via column chromatography (1/1 hexanes/ethyl acetate) to yield 0.34 g (83%) of 29 as a clear, colorless oil;

¹H NMR (300 MHz, CDCl₃) δ 7.89 (t, J=8.9 Hz, 4H), 7.66-7.44 (m, 4H), 7.21-7.05 (m, 4H), 4.39 (s, 2H), 4.30 (s, 2H), 4.21 (m, 4H), 3.91 (s, 3H), 1.34 (t, J=6.6, 6H).

### 4-({Benzenesulfonyl-[3-bromo-4-(difluoro-phosphono-methyl)-benzyl]-amino}-methyl)-benzoic acid methyl ester (30)

To 4-[(Benzenesulfonyl-{3-bromo-4-[(diethoxy-phosphoryl)-difluoro-methyl]-benzyl}-amino)-methyl]-benzoic acid methyl ester (29, 0.34 g, 0.52 mmol) in CH₂Cl₂ (5 mL) was added bistrimethylsilyl-trifluoroacetamide (1.3 g, 5.2 mmol) and iodotrimethylsilane (0.52 g, 2.6 mmol). The resulting mixture was stirred at room temperature for 1.5 hours, after which it was concentrated in vacuo. The resulting material was stirred in CH₃CN (4 mL), H₂O (0.5 mL), and TFA (0.5 mL) for 0.5 hours, after which it was concentrated in vacuo and partitioned between EtOAc and acidic Na₂S₂O₄. The EtOAc layer was dried over MgSO₄ and concentrated in vacuo to yield 0.31 g (99%) of 30 as a white foam;

¹H NMR (300 MHz, DMSO-d₆) δ 7.90 (d, J=7.2 Hz, 2H), 7.80 (d, J=8.1 Hz, 2H), 7.76-7.61 (m, 3H), 7.40 (d, J=8.1 Hz, 1H), 7.28-7.26 (m, 3H), 7.17 (d, J=7.8 Hz, 1H), 4.14 (s, 2H), 4.37 (s, 2H); LCMS m/z 602 [M⁻]

### Example 10

### 4-({Benzenesulfonyl-[3-bromo-4-(difluoro-phosphono-methyl)-benzyl]-amino}-methyl)-benzoic acid (31)

To 4-({Benzenesulfonyl-[3-bromo-4-(difluoro-phosphono-methyl)-benzyl]-amino}-methyl)-benzoic acid methyl ester (30, 0.30 g, 0.49 mmol) in 1/1 MeOH/THF (10 mL) was added 2.5M NaOH (1 mL), and the resulting mixture was stirred for 12 hours at 65° C. The solution was partitioned between 1 N HCl and EtOAc, after which the organic layer was dried over MgSO₄ and concentrated in vacuo to yield 0.11 g (38%) of 31 as a white solid; mp 160-162° C.;

¹H NMR (300 MHz, DMSO-d₆) δ 7.90 (d, J=8.1 Hz, 2H), 7.82 (d, J=10.2 Hz, 2H), 7.78-7.62 (m, 3H), 7.45 (d, J=9 Hz, 1H), 7.25-7.15 (m, 5H); LCMS m/z 588 [M⁻]

### Example 11

### N-(3,4-Dichloro-phenyl)-benzenesulfonamide (32)

To a solution of 3,4-dichloroaniline (0.12 g, 0.71 mmol) in pyridine (5 mL) was added benzenesulfonyl chloride (0.13 g, 0.71 mmol). The resulting mixture was stirred at room temperature for 3 hours. The mixture was partitioned between EtOAc and 2N HCl, after which the organic layer was washed with sat. NaHCO₃ and brine, dried over MgSO₄ and concentrated in vacuo. The resulting material was recrystallized from 2/1 MeOH/H₂O to yield 0.15 g (70%) of 32.

### [(4-{[Benzenesulfonyl-(3,4-dichlorophenyl)-amino]-methyl}-2-bromo-phenyl)-difluoro-methyl]-phosphonic acid diethyl ester (33)

To N-(3,4-Dichlorophenyl)-benzenesulfonamide (32, 0.10 g, 0.34 mmol) in DMF (5 mL) was added NaH [60% in oil] (0.014 g, 0.34 mmol). After 0.1 hour, [(2-Bromo-4-bromomethyl-phenyl)-difluoro-methyl]-phosphonic acid diethyl ester (28, 0.15 g, 0.34 mmol) was added and the resulting mixture was stirred at room temperature for 1 hour. It was then partitioned between Et₂O and H₂O, after which the organic layer was dried over MgSO₄ and concentrated in vacuo. The resulting material was purified via column chromatography with 1/1 hexanes/ethyl acetate to yield 0.17 g (77%) of 33 as a clear, colorless oil.

### [(4-{[Benzenesulfonyl-(3,4-dichlorophenyl)-amino]-methyl}-2-bromo-phenyl)-difluoro-methyl]-phosphonic acid (34)

To [(4-{[Benzenesulfonyl-(3,4-dichlorophenyl)-amino]-methyl}-2-bromo-phenyl)-difluoro-methyl]-phosphonic acid diethyl ester (33, 0.17 g, 0.26 mmol) in CH₂Cl₂ (5 mL) was added bistrimethylsilyl-trifluoroacetamide (0.48 g, 1.9 mmol) and iodotrimethylsilane (0.18 g, 0.9 mmol). The resulting mixture was stirred at room temperature for 1.5 hours, after which it was concentrated in vacuo. The resulting material was stirred in CH₃CN (4 mL), H₂O (0.5 mL), and TFA (0.5 mL), after which it was concentrated in vacuo and partitioned between EtOAc and acidic Na₂S₂O₄. The organic layer was dried over MgSO₄ and concentrated in vacuo to yield 0.10 g (66%) of 34 as a white foam;

¹H NMR (300 MHz, DMSO-d₆) δ 7.77-7.41 (m, 10H), 7.17 (dd, J=8.4,1.2 Hz, 1H), 4.87 (s, 2H); LCMS m/z 598 [M⁻].

### Example 12

### N-(4-Iodo-benzyl)-benzenesulfonamide (35)

To 4-iodobenzylamine (1.0 g, 4.2 mmol) in pyridine (7.0 mL) was added benzenesulfonyl chloride (1.3 g, 7.1 mmol). The resulting mixture was stirred at room temperature for 3 hours, after which it was diluted with ethyl acetate (40 mL) and washed with 2×40 mL 2N HCl, 1×40 mL sat. NaHCO₃ and then 1×40 mL brine. The organic layer was dried over MgSO₄ and the solvent removed by rotary evaporation yielding a brown solid. The crude product was recrystallized in a 2:1 methanol:water mixture affording the desired product (35, 1.3 g, 83%) as white crystals;

¹H NMR (300 MHz, DMSO-d₆) δ 8.21 (t, J=6.2 Hz, 1H), 7.80 (d, J=6.0, 2H), 7.65-7.56 (m, 5H), 7.06 (d, J=8.1 Hz, 2H), 3.95 (d, J=5.4 Hz, 2 Hz).

### {[4-(Benzenesulfonylamino-methyl)-phenyl]-difluoro-methyl}-phosphonic acid diethyl ester (36)

To activated Zn (0.53 g, 8.1 mmol) in DMA (3.0 mL) was added bromodifluoromethyldiethyl-phosphonate (2.2 g, 8.2 mmol) in DMA (4.0 mL). The resulting mixture was stirred at room temperature for 3 hours, after which copper(I) bromide (0.98 g, 6.8 mmol) was added and stirring was continued for 0.5 hours at room temperature. N-(4-Iodo-benzyl)-benzenesulfonamide (35, 1.2 g, 3.2 mmol) in DMA (2.0 mL) was then added and the mixture was sonicated at room temperature for 3 hours and then stirred for an additional 20 hours. The reaction mixture was partitioned between ether and H₂O and filtered through Celite. The aqueous layer was extracted with 3×100 mL ether and the combined organic layers were washed with brine, dried over MgSO₄ and concentrated in vacuo to yield a yellow oil which was purified by column chromatography (4:1 hexanes:ethyl acetate) to afford the desired product (36, 0.47 g, 33%) as a white solid;

¹H NMR (300 MHz, DMSO-d₆) δ 8.28 (t, J=6.2 Hz, 1H), 7.83 (d, J=6.0, 2H), 7.66-7.57 (m, 3H), 7.50-7.41 (m, 4H), 4.17-3.99 (m, 4H), 3.35 (s, 2H), 1.28-1.22 (m, 6H).

### ({4-[(Benzenesulfonyl-{4-[(diethoxy-phosphoryl)-difluoro-methyl]-benzyl}-amino)-methyl]-2-bromo-phenyl}-difluoro-methyl)-phosphonic acid diethyl ester (37)

To {[4-(Benzenesulfonylamino-methyl)-phenyl]-difluoro-methyl}-phosphonic acid diethyl ester (36, 0.42 g, 0.96 mmol) in DMF (8.0 mL) was added NaH (0.027 g, 1.0 mmol). After 20 minutes, [(2-Bromo-4-bromomethyl-phenyl)-difluoro-methyl]-phosphonic acid diethyl ester (28, 0.41 g, 0.93 mmol) was added and the resulting mixture was stirred at room temperature for 1 hour. The reaction was partitioned between Et₂O and H₂O. The aqueous layer was extracted with 3×100 mL ether and the combined organic layers were washed with brine, dried over MgSO₄ and concentrated in vacuo to yield a yellow syrup which was purified by column chromatography (2:1 hexanes:ethyl acetate) to afford the desired product (37, 0.42 g, 56%) as a white solid;

¹H NMR (300 MHz, CDCl₃) δ 7.85 (d, J=8.4 Hz, 2H), 7.65-7.47 (m, 6H), 7.26 (d, J=5.4, 1H), 7.16 (d,J=7.2, 2H), 7.07 (d, J=7.8, 1H), 4.39-4.08 (m, 12H), 1.37-1.24 (m, 12H).

### {[4-({Benzenesulfonyl-[4-(difluoro-phosphono-methyl)-benzyl]-amino}-methyl)-2-bromo-phenyl]-difluoro-methyl}-phosphonic acid (38)

To ({4-[(Benzenesulfonyl-{4-[(diethoxy-phosphoryl)-difluoro-methyl]-benzyl}-amino)-methyl]-2-bromo-phenyl}-difluoro-methyl)-phosphonic acid diethyl ester (37, 0.42 g, 0.53 mmol) in CH₂Cl₂ (8.0 mL) was added bistrimethylsilyltrifluoroacetamide (1.5 g, 5.6 mmol) and iodotrimethylsilane (1.1 g, 5.3 mmol). The resulting mixture was stirred at room temperature for 2.0 hours, after which it was concentrated in vacuo. The resulting material was stirred in CH₃CN (4.0 mL), H₂O (1.0 mL), and TFA (0.5 mL) for 1.0 hours, after which it was concentrated in vacuo and partitioned between EtOAc and acidic 2% Na₂S₂O₄. The EtOAc layer was dried over MgSO₄ and concentrated in vacuo to yield a pale yellow solid. This solid was recrystallized in a 1:1 CH₂Cl₂:ether mixture yielding the product (38, 0.046 g, 13%) as an off-white solid;

¹H NMR (300 MHz, DMSO-d₆) δ 7.96 (d, J=7.5 Hz, 2H), 7.76-7.65 (m, 3H), 7.44 (s, 1H), 7.27-7.19 (m, 5H), 7.08-7.02 (m, 1H), 4.38 (s, 2H), 4.35 (s, 2H); LCMS m/z 676 [M⁻]

### Example 13

### Naphthalene-2-sulfonic acid 4-iodo-benzylamide (39)

To 4-iodo-benzylamine (3.0 g, 13.0 mmol) in pyridine (21.0 mL) was added 2-naphthalene-sulfonyl chloride (4.4 g, 19.3 mmol). The resulting mixture was stirred at room temperature for 3 hours, after which it was diluted with ethyl acetate and washed with 2×100 mL 2N HCl, 1×100 mL sat. NaHCO₃ and then 1×100 mL brine. The organic layer was dried over MgSO₄ and the solvent removed by rotary evaporation yielding an off-white solid. The crude product was recrystallized in a 2:1 methanol:water mixture affording the desired product (39, 5.0 g, 92%) as an off white solid;

¹H NMR (300 MHz, DMSO-d₆) δ 8.42 (s, 1H), 8.31 (t, J=6.3, 1H), 8.16-8.04 (m, 3H), 7.84-7.59 (m, 5H), 7.06 (d, J=8.1 Hz, 2 Hz), 4.00 (d, J=5.1, 2H).

### (Difluoro-{4-[(naphthalene-2-sulfonylamino)-methyl]-phenyl}-methyl)-phosphonic acid diethyl ester (40)

To activated Zn (1.9 g, 29.6 mmol) in DMA (8.0 mL) was added bromodifluoromethyldiethyl-phosphonate (8.0 g, 29.8 mmol) in DMA (14.0 mL). The resulting mixture was stirred at room temperature for 3 hours, after which copper(I) bromide (4.3 g, 29.6 mmol) was added and stirring was continued for 0.5 hours at room temperature. Naphthalene-2-sulfonic acid 4-iodo-benzylamide (39, 5.0 g, 11.8 mmol) in DMA (11.0 mL) was then added and the mixture was sonicated at room temperature for 3 hours and then stirred for an additional 23 hours. The reaction mixture was partitioned between ether and H₂O and filtered through Celite. The aqueous layer was extracted with 3×100 mL ether and the combined organic layers were washed with brine, dried over MgSO₄ and concentrated in vacuo to yield a yellow oil which was purified by column chromatography (4:1 hexanes:ethyl acetate) to afford the desired product (40, 0.88 g, 15%) as a thick yellow syrup;

¹H NMR (300 MHz, DMSO-d₆) δ 8.46 (s, 1H), 8.35 (t, J=6.2, 1H), 8.16-8.11 (m, 2H), 8.04 (d, J=7.8, 1H), 7.84 (dd, J=8.9, 1.4, 1H), 7.74-7.65 (m, 2H), 7.44 (s, 4H), 4.13-3.95 (m, 6H), 1.30-1.18 (m, 6H).

### [(2-Bromo-4-{[{4-[(diethoxy-phosphoryl)-difluoro-methyl]-benzyl}-(naphthalene-2-sulfonyl)-amino]-methyl}-phenyl)-difluoro-methyl]-phosphonic acid diethyl ester (41)

To (Difluoro-{4-[(naphthalene-2-sulfonylamino)-methyl]-phenyl}-methyl)-phosphonic acid diethyl ester (40, 0.51 g, 1.1 mmol) in DMF (8.0 mL) was added NaH (0.039 g, 1.6 mmol). After 20 minutes, [(2-Bromo-4-bromomethyl-phenyl)-difluoro-methyl]-phosphonic acid diethyl ester (28, 0.47 g, 1.1 mmol) was added and the resulting mixture was stirred at room temperature for 2 hours. The reaction was partitioned between Et₂O and H₂O. The aqueous layer was extracted with 3×50 mL ether and the combined organic layers were washed with brine, dried over MgSO₄ and concentrated in vacuo to yield a yellow oil which was purified by column chromatography (2:1 hexanes:ethyl acetate) to afford the desired product (41, 0.35 g, 39%) as a thick pale yellow syrup;

¹H NMR (300 MHz, DMSO-d₆) δ 8.60 (s, 1H), 8.18 (d, J=9.0, 2H), 8.10 (d, J=8.7, 1H), 7.92 (d, J=9.3, 1H), 7.78-7.68 (m, 2H), 7.42-7.26 (m, 7H), 4.49 (s, 2H), 4.46 (s, 2H), 4.13-3.95 (m, 8H), 1.38-1.13 (m, 12H).

### [(2-Bromo-4-{[[4-(difluoro-phosphono-methyl)-benzyl]-(naphthalene-2-sulfonyl)-amino]-methyl}-phenyl)-difluoro-methyl]-phosphonic acid (42)

To [(2-Bromo-4-{[{4-[(diethoxy-phosphoryl)-difluoro-methyl]-benzyl}-(naphthalene-2-sulfonyl)-amino]-methyl}-phenyl)-difluoro-methyl]-phosphonic acid diethyl ester (41, 0.33 g, 0.39 mmol) in CH₂Cl₂ (6.0 mL) was added bistrimethylsilyltrifluoroacetamide (0.97 g, 3.8 mmol) and iodotrimethylsilane (0.93 g, 4.6 mmol). The resulting mixture was stirred at room temperature for 2.0 hours, after which it was concentrated in vacuo. The resulting material was stirred in CH₃CN (4.0 mL), H₂O (1.0 mL), and TFA (0.5 mL) for 1.0 hours, after which it was concentrated in vacuo and partitioned between EtOAc and acidic 2% Na₂S₂O₄. The EtOAc layer was dried over MgSO₄ and concentrated in vacuo to yield a pale yellow solid. This solid was recrystallized in a 1:1 CH₂Cl₂:ether mixture yielding the product (42, 0.020 g, 7%) as a white solid;

¹H NMR (300 MHz, DMSO-d₆) δ 8.63 (s, 1H), 8.20 (d, J=7.8, 2H), 8.10 (d, J=6.9, 1H), 7.95 (d, J=8.7, 1H), 7.78-7.68 (m, 2H), 7.42-7.23 (m, 6H), 7.09 (d, J=9.3, 1H), 4.44 (s, 2H), 4.41 (s, 2H); LCMS m/z 362 [M²⁻].

### Example 14

### {[4-(difluorophosphonomethyl)-3-chlorophenyl]methyl}[(2-methoxyphenyl)sulfonyl]{[4-(3-cyanophenyl)phenyl]methyl}amine (46)

Step A. A mixture of 4-bromobenzylamine (0.990 g, 5.32 mmol), DIEA (0.84 mL, 4.84 mmol) and DMAP (0.010 g, 0.08 mmol) in dry dichloromethane (30 mL) were stirred nitrogen at 0° C. 2-Methoxybenzene sulfonyl chloride (1.00 g, 4.84 mmol) was added and the reaction mixture slowly warmed up to room temperature for overnight. The reaction mixture was concentrated in vacuo. The residue was purified by flash chromatography (ethyl acetate/hexanes, 1:1) to yield 1.707 g (99%) of [(4-bromophenyl)methyl][(2-methoxyphenyl)sulfonyl]amine (43) as a white solid. MS (M+H)⁺ 356, 358; (M−H)⁻ 354, 356.

Step B. In a 10 mL glass tube was placed (3-cyanophenyl)boronic acid (0.0825 g, 0.56 mmol), 43 (0.200 g, 0.56 mmol), bis(triphenylphosphine)palladium (II) chloride (0.020 g, 0.028 mmol), 1M Na₂CO₃ (in water) (1.2 mL), acetonitrile (1.2 mL) and a magnetic stir bar. The vessel was sealed with a septum and placed into the microwace cavity. Microwace irradiation was used, and the reaction mixture was keep at 150° C. for 250 seconds. After the mixture was allowed to cool to room temperature, the reaction vessel was opened and the contents were filter through the Celite. The filtrate was concentrated under vacuum. The crude residue was purified by flash chromatography (ethyl acetate/hexanes, 1:1) to yield 0.173 g (82%) of 3-[4-({[(2-methoxyphenyl)sulfonyl]amino}methyl)phenyl]benzenecarbonitrile (44) as white solid. MS (M+H)⁺ 379; (M−H)⁻ 377.

Step C. To a solution of 44 (0.200 g, 0.53 mmol) in 5 mL of dry DMF was added potassium tert-butoxide (0.53 mL, 0.53 mmol, 1M in tert-butanol) at room temperature under an nitrogen atmosphere. After 5 minute, {[4-(bromomethyl)-2-chlorophenyl]difluoromethyl}diethoxyphosphino-1-one (0.207 g, 0.53 mmol) was injected, and the solution was stirred at room temperature for overnight. The DMF was evaporated (rotavap) under vacuum. The crude residue was purified by flash chromatography (ethyl acetate/hexanes, 100% hexanes to 1:1) to isolate the 3-(4-{[({4-[(diethoxycarbonyl)difluoromethyl]-3-chlorophenyl}methyl)[(2-methoxyphenyl)sulfonyl]amino]methyl}phenyl)benzenecarbonitrile (45) (0.289 g) in 79% yield as colorless oil. MS (M+NH₄)⁺ 706, 708; (M−Et)⁻ 659, 661.

Step D. A solution of 45 (0.269 g, 0.39 mmol) and iodotrimethylsilane (0.89 mL, 6.24 mmol) in dry dichloromethane (7 mL) was stirred at −20° C. for 1 hour. The reaction mixture was warmed up to room temperature to stir for an additional 2 hour. The solvent and excess iodotrimethylsilane was removed by vacuum. The residue was treated with acetonitrile (16 mL), TFA (2 mL) and H₂O (4 mL) and stirred for overnight at room temperature. The reaction was then rotary evaporated to an oil which was dissolved in ethyl acetate and washed with 5% Na₂S₂O₄ (acidified) followed by a wash with saturated NaCl. The organic layer was dried (Na₂SO₄) and concentrated. The crude residue was purified by C-18 flash chromatography (MeOH/H₂O, 100% H₂O to 6:4) to give the 3-{4-[({[4-(difluorophosphonomethyl)-3-chlorophenyl]methyl}[(2-methoxyphenyl)sulfonyl]amino)methyl]phenyl}benzenecarbonitrile (46) (0.201 g) in 81% yield as white solid. MS (M+NH₄)⁺ 650,652; (M−H)⁻ 631, 633.

### Example 15

### {[4-(difluorophosphonomethyl)-3-chlorophenyl]methyl}[(2-methoxyphenyl)sulfonyl]{[4-(3-(2H-1,2,3,4-tetraazol-5-yl)phenyl)phenyl]methyl}amine (47)

A solution of 46 (0.133 g, 0.21 mmol), sodium azide (0.137 g, 2.10 mmol) and ammonium chloride (0.112 g, 2.10 mmol) in DMF (4 mL) was stirred at 120° C. for overnight. The solvent was removed by vacuum. The residue was dissolved in ethyl acetate and washed with water, 1N HCl and brine. The organic layer was dried (Na₂SO₄) and concentrated. The crude residue was purified by C-18 flash chromatography (MeOH/H₂O, 100% H₂O to 1:1) to give the {[4-(difluorophosphonomethyl)-3-chlorophenyl]methyl}[(2-methoxyphenyl)sulfonyl]{[4-(3-(2H-1,2,3,4-tetraazol-5-yl)phenyl)phenyl]methyl}amine (0.104 g) in 73% yield as white solid. MS (M+H)⁺ 676, 678; (M−H)⁻ 674, 676.

¹H NMR (600 MHz, d₆-DMSO): δ 12.5 (3H, br s), 8.29 (1H, s), 8.04 (1H, d), 7.86 (1H, d), 7.83 (1H, d), 7.70-7.65 (2H, m), 7.60 (2H, d), 7.48 (1H, d), 7.26 (1H, d), 7.22 (2H, d), 7.18 (1H, d), 7.12-7.10 (2H, m), 4.47 (4H, s), 3.91 (3H, s).

### Example 16

### N-[4-({(2-Methoxy-benzenesulfonyl)-[4-(oxalyl-amino)-benzyl]-amino}-methyl)-phenyl]-oxalamic acid (51)

Step A

A solution of 2-methoxybenzenesulfonyl chloride (2.2 g, 10.6 mmol) in 50 mL of THF was added dropwise with stirring to a chilled (0° C.) methanolic solution of ammonia (50 mL of 2M). The stirred mixture was allowed to warm to room temperature overnight. The reaction mixture was then concentrated in vacuo and the residue taken up in ethyl acetate. The ethyl acetate solution was washed successively with 0.1M HCl, saturated sodium bicarbonate, saturated brine, then dried over anhydrous sodium sulfate and concentrated in vacuo. The resulting solids were recrystallized from hot ethyl acetate/ether to yield 2-methoxybenzenesulfonamide (47) as white crystals (1.48 g, 75%).

¹H NMR (DMSO-d₆, 300 MHz) δ 7.73 (d, J=8.4, 1H), 7.53-7.59 (m, 1H), 7.20 (d, J=9.0, 1H), 7.02-7.08 (m, 3H).

Step B

Potassium carbonate (0.58 g, 4.2 mmol) was added to a solution of 47 (100 mg, 0.53 mmol) and 4-nitrobenzyl bromide (281 mg, 1.3 mmol) in DMF (1 mL). The resulting mixture was stirred for 3 hours at 60° C. The reaction mixture was taken up in ethyl acetate. The ethyl acetate solution was washed successively with 0.1 M HCl, saturated sodium bicarbonate, saturated brine, then dried over anhydrous sodium sulfate and concentrated in vacuo. The resulting solids (0.30 g) were purified by flash chromatography (silica gel, DCM eluent) to yield bis[(4-nitrophenyl)methyl][(2-methoxyphenyl)sulfonyl]amine (48) as a yellow solid (170 mg, 70%).

¹H NMR (CDCl3, 300 MHz) δ 8.08 (d, J=9.0 MHz, 4H), 8.01 (d, J=6.9, 1H), 7.58-7.64 (m, 1H), 7.24 (d, J=9.0, 2H), 7.03-7.12 (m, 2H), 4.52 (s, 4H), 3.91 (s, 3H).

Step C

Iron powder (420 mg, 7.5 mmol), water (0.75 mL), and HCl (14 μL of 12M) were added consecutively to a solution of 48 (160 mg, 0.35 mmol) in ethanol (3 mL). After stirring at 95° C. for 90 minutes, the reaction mixture was filtered hot. Following an ethanol wash, the filtrates were combined and the solvent was removed in vacuo. Isolated bis[(4-aminophenyl)methyl][(2-methoxyphenyl)sulfonyl]amine (49) as a yellow solid (125 mg, 89%). TLC (MeOH/DCM/NH₄₀H; 1:9:0.1) Rf=0.4.

Step D

Ethyl chlorooxoacetate (58 μL, 0.52 mmol) was added to a solution of 49 (83 mg, 0.21 mmol) and triethylamine (87 μL, 0.63 mmol) in 1 mL of THF. After complete reaction as evidenced by TLC, the solvent was removed in vacuo. The resulting solids were taken up in ethyl acetate. The ethyl acetate solution was washed successively with 0.1 M HCl, saturated sodium bicarbonate, saturated brine, then dried over anhydrous sodium sulfate and concentrated in vacuo.

Ethyl [N-(4-{[({4-[(ethoxycarbonyl)carbonylamino]phenyl}methyl)[(2-methoxyphenyl)sulfonyl]amino]methyl}phenyl)carbamoyl]formate (50) was Recovered as a light yellow solid, (125 mg, 94%). TLC (4% MeOH/DCM) Rf=0.39.

Step E

Added Lithium hydroxide (0.8 mL of 1M) to a solution of 50 in 0.8 mL of 1:3 water/THF. The reaction was followed by LCMS. Upon completion, the reaction mixture was concentrated in vacuo and purified by column chromatography (C-18 silica, product eluted with 60% aqueous methanol). N-[4-({(2-Methoxy-benzenesulfonyl)-[4-(oxalyl-amino)-benzyl]-amino}-methyl)-phenyl]-oxalamic acid (51) was recovered as a light yellow solid (68 mg, 60%). mp 187-189 (d). MS (M−H)−: 540 expected, 540 observed; (M−2H)−: 269.5 expected, 269.5 observed.

¹H NMR (DMSO-d₆, 300 MHz) δ 14.2 (br s, 2H), 10.7 (s, 2H), 7.81 (d, J=6.9, 1H), 7.57-7.61 (m, 5H), 7.18 (d, J=8.7, 1H), 7.04-7.10 (m, 1H), 6.95 (d, J=7.5, 4H), 4.24 (s, 4H), 3.75 (s, 3H).

### Example 17

### 5-({[(3,4-dichlorophenyl)methyl][(2-methoxyphenyl)sulfonyl]amino}methyl)-2-(carboxycarbonylamino)benzoic acid (57)

Step A

(3,4-dichlorophenyl)methylamine (0.74 g, 4.2 mmol) in 20 mL of methylene chloride was stirred at 0° C. DMAP (0.046 g, 0.38 mmol), DIEA (0.49 g, 3.8 mmol), and (chloro(2-methoxyphenyl)sulfonyl chloride were added respectively. The reaction mixture was stirred at room temperature for overnight. The methylene chloride was evaporated (rotavap) under vacuum. The crude residue was purified by flash chromatography (ethyl acetate/hexanes, 1:2) to give [(3,4-dichlorophenyl)methyl][(2-methoxyphenyl)sulfonyl]amine (52) as a white solid (1.05 g) in 72% yield. MS (M+H)⁺ 345, 347.

Step B

52 (2.0 g, 10.2 mmol) in 100 mL benzene was stirred at room temperature. NBS (1.99 g, 11.2 mmol) and AIBN (0.084 g, 0.51 mmol) were added respectively. The reaction mixture was refluxed overnight. The benzene was evaporated (rotavap) under vacuum. The crude material was extracted with ethyl acetate and washed with 1M NaOH. The organic layer was washed with brine, dried (Na₂SO₄), concentrated, and dried under vacuum to give methyl 5-(bromomethyl)-2-nitrobenzoate (53) as a brown oil (2.4 g) in 85% yield. MS (M+NH4)⁺ 290,292.

Step C

Under dry conditions 53 (0.5 g, 1.44 mmol) in 10 mL DMF was stirred at room temperature. NaH (0.058 g, 1.44 mmol) was added and stirred for 30 min. methyl 5-(bromomethyl)-2-nitrobenzoate (0.946 g, 1.59 mmol) was added to the mixture. The reaction was stirred overnight. The DMF was evaporated (rotavap) under vacuum. The crude residue was purified by flash chromatography (ethyl acetate/hexanes, 1:1) to give methyl-5-({[(3,4-dichlorophenyl)methyl][(2-methoxyphenyl)sulfonyl]amino}methyl)-2-nitrobenzoate (54) as a brown liquid (0.7 g) in 90% yield. MS (M+H)⁺ 539.

Step D

54 (0.69 g, 1.28 mmol), iron (1.4 g, 25.6 mmol), water (5 mL), HCl (0.1 mL), and ethanol (20 mL) were refluxed for 4 hours. The crude residue was purified by flash chromatography (ethyl acetate/hexanes, 1:2) to yield methyl 2-amino-5-({[(3,4-dichlorophenyl)methyl][(2-methoxyphenyl)sulfonyl]amino}methyl)benzoate (55) as a yellow liquid (22%). (M+H)⁺ 508, 510.

Step E

55 (0.136 g, 0.26 mmol) in 20 mL methylene chloride was stirred at room temperature. DMAP (0.049 g, 0.4 mmol) and methyl (chlorocarbonyl)formate (0.049 g, 0.4 mmol) were added to the reaction mixture. The reaction was stirred for overnight, diluted with methylene chloride and washed with brine, NaCl (aq), 1N HCl, sat. NaHCO₃, and brine. The organic layer was dried (Na₂SO₄), concentrated, and dried under vacuum to give methyl{N-[4-({[(3,4-dichlorophenyl)methyl][(2-methoxyphenyl)sulfonyl]amino}methyl)-2-(methoxycarbonyl)phenyl]carbamoyl}formate (56) as a yellow liquid (0.098 g) in 63% yield. (M+H)⁺ 594,596.

Step F

56 (0.095 g, 0.16 mmol), and 0.25M LiOH in methanol (15 mL) in THF (1.5 mL) was stirred for 4 hours. The crude residue was purified by reverse phase C18 (methanol/water 60:40) to yield 20% of 5-{[(3,4-Dichloro-benzyl)-(2-methoxy-benzenesulfonyl)-amino]-methyl}-2-(oxalyl-amino)-benzoic acid (57) as a white solid. R_{f}=0.41 (butanol/acidic acid/water 4:2:1). MS (M+NH₄)⁺ 583, 585.

### Example 18

### (Adamantanylmethyl){[4-(difluorophosphonomethyl)-3-chlorophenyl]methyl}[(2-methoxyphenyl)sulfonyl]amine

(Adamantanylmethyl)[(2-methoxyphenyl)sulfonyl]amine: Adamantanyl methyl)[(2-methoxyphenyl)sulfonyl]amine was prepared by treating 2-methoxybenzene sulfonyl chloride (0.41 g, 2 mmols) with Adamantanylmethyl amine (0.33 g, 2 mmols) in Dichloromethane (10 ml) in presence of Diisopropylethyl amine (0.696 ml, 4 mmols). Yield: 0.65 g (97%). MH⁺: 336.

{[4-({(Adamantanylmethyl)[(2-methoxyphenyl)sulfonyl]amino}methyl)-2-chlorophenyl]difluoromethyl}diethoxyphosphino-1-one: {[4-({(Adamantanylmethyl)[(2-methoxyphenyl)sulfonyl]amino}methyl)-2-chlorophenyl]difluoromethyl}diethoxy phosphino-1-one was prepared by treating (Adamantanylmethyl)[(2-methoxyphenyl)sulfonyl]amine (0.335 g, 1 mmol) with 95% sodium hydride (28 mg, 1.1 mmol) and [(4-(Bromomomethyl-2-chloro-phenyl)-difluoro-methyl-phosphonic acid diethyl ether in DMF. Yield: 0.42 g (65%). MH⁺: 646 and 648.

(Adamantanylmethyl){[4-(difluorophosphonomethyl)-3-chlorophenyl]methyl}[(2-methoxyphenyl)sulfonyl]amine: (Adamantanylmethyl){[4-(difluorophosphonomethyl)-3-chlorophenyl]methyl}[(2-methoxyphenyl)sulfonyl]amine was prepared by treating {[4-({(Adamantanylmethyl)[(2-methoxyphenyl)sulfonyl]amino}methyl)-2-chlorophenyl]difluoromethyl}diethoxyphosphino-1-one (0.39 g 0.6 mmols) with Trimethyl silylidodide (0.82 ml, 6 mmol) in dichloromethane (10 ml). Yield: 0.3 g (84%). MH⁺: 590 and 592. ¹H NMR: (600 MHz, DMSO-d₆) 12.50δ (2H, b), 7.776 (1H, d); 7.62δ (1H, d); 7.26δ (2H, s); 7.23δ (1H, d); 7.07δ (1H, t); 4.55δ (2H, s); 3.90δ (3H, s), 2.95δ (2H, s); 1.90δ (3H, s); 1.59δ (3H, d); 1.47δ (3H, d), 1.39δ (6H, s).

### Example 19

### {[4-(difluorophosphonomethyl)-3-chlorophenyl]methyl}(cycloheptylmethyl)[(2-methoxyphenyl)sulfonyl]amine

(cycloheptylmethyl)[(2-methoxyphenyl)sulfonyl]amine: (cycloheptyl methyl)[(2-methoxyphenyl)sulfonyl]amine was prepared by treating cycloheptylmethyl amine (0.25 g, 2 mmols) with 2-methoxybenzenesulfonylchloride (0.41 g, 2 mmols) in dichloromethane (10 ml) in presence of diisopropylethylamine (0.7 ml 4 mmols). Yield: 0.57 g (93%). MH⁺: 298.

{[2-chloro-4-({(cycloheptylmethyl)[(2-methoxyphenyl)sulfonyl]amino}methyl)phenyl]difluoromethyl}diethoxyphosphino-1-one: {[2-chloro-4-({(cycloheptylmethyl)[(2-methoxyphenyl)sulfonyl]amino}methyl)phenyl]difluoromethyl}diethoxyphosphino-1-one was prepared by treating (cycloheptylmethyl)[(2-methoxyphenyl)sulfonyl]amine (0.45 g, 1.5 mmols) with 95% sodiumhydride (42 mg, 1.65 mmols) in DMF and then stirring with [(4-(Bromomomethyl-2-chloro-phenyl)-difluoro-methyl-phosphonic acid diethyl ether for overnight. Yield: 0.67 g, 73%). MH⁺: 608 and 610.

{[4-(difluorophosphonomethyl)-3-chlorophenyl]methyl}(cycloheptylmethyl)[(2-methoxyphenyl)sulfonyl]amine: {[4-(difluorophosphono methyl)-3-chlorophenyl]methyl}(cycloheptylmethyl)[(2-methoxyphenyl)sulfonyl]amine was prepared by treating {[2-chloro-4-({(cycloheptylmethyl)[(2-methoxyphenyl)sulfonyl]amino}methyl)phenyl]difluoromethyl}diethoxyphosphino-1-one (0.6 g 1 mmol) with trimethylsilyliodide (1.36 ml 10 mmols)in dichloromethane (8 ml). Yield: 0.47 g (84%). MH⁺: 552 and 554. ¹H NMR: (600 MHz, DMSO-d₆) 7.81δ (1H, d), 7.65δ (1H, d); 7.56δ (1H, d); 7.40δ (1H, s); 7.35δ (1H, d); 7.26δ (1H, d); 7.10δ (1H, s); 4.48δ (2H, s), 3.91δ (3H, s); 2.90δ (2H, s); 1.48δ (5H, m); 1.34δ (4H, s), 1.05δ (2H, s); 0.90δ (2H, d).

### Example 20

### {[4-(difluorophosphonomethyl)-3-chlorophenyl]methyl}(cyclohexylmethyl)[(2-methoxyphenyl)sulfonyl]amine

(cyclohexylmethyl)[(2-methoxyphenyl)sulfonyl]amine: (cyclohexyl methyl)[(2-methoxyphenyl)sulfonyl]amine was prepared by treating cyclohexylmethyl amine (0.23 g, 2 mmols) with 2-methoxybenzenesulfonylchloride (0.41 g, 2 mmols) in dichloromethane (10 ml) in presence of diisopropylethylamine (0.7 ml 4 mmols). Yield: 0.53 g (94%). MH⁺: 284.

{[2-chloro-4-({(cyclohexylmethyl)[(2-methoxyphenyl)sulfonyl]amino}methyl)phenyl]difluoromethyl}diethoxyphosphino-1-one: {[2-chloro-4-({(cyclohexyl methyl)[(2-methoxyphenyl)sulfonyl]amino}methyl)phenyl]difluoromethyl}diethoxy phosphino-1-one was prepared by treating (cyclohexylmethyl)[(2-methoxyphenyl)sulfonyl]amine (0.43 g, 1.5 mmols) with 95% sodiumhydride (42 mg, 1.65 mmols) in DMF and then stirring with [(4-(Bromomomethyl-2-chloro-phenyl)-difluoro-methyl-phosphonic acid diethyl ether for overnight. Yield: 0.53 g, 60%). MH⁺: 594 and 596.

{[4-(difluorophosphonomethyl)-3-chlorophenyl]methyl}(cyclohexylmethyl)[(2-methoxyphenyl)sulfonyl]amine: {[4-(difluorophosphono methyl)-3-chlorophenyl]methyl}(cyclohexylmethyl)[(2-methoxyphenyl)sulfonyl]amine was prepared by treating {[2-chloro-4-({(cyclohexylmethyl)[(2-methoxyphenyl)sulfonyl]amino}methyl)phenyl]difluoromethyl}diethoxyphosphino-1-one (0.6 g 1 mmol) with trimethylsilyliodide (1.36 ml 10 mmols)in dichloromethane (8 ml). Yield: 0.43 g (80%). MH⁺: 524 and 526. ¹H NMR: (600 MHz, DMSO-d₆) 7.81δ (1H, d), 7.65δ (1H, d); 7.55δ (1H, d); 7.33δ (2H, m); 7.25δ (1H, d); 7.10δ (1H, d); 4.48δ (2H, s), 3.90δ (3H, s); 2.96δ (2H, s); 1.51δ (5H, m); 0.97δ (3H, m); 1.29δ (1H, m); 0.67δ (2H, d).

### Example 21

### {[4-(difluorophosphonomethyl)-3-chlorophenyl]methyl}[(2-methoxyphenyl)sulfonyl][(2-piperidylphenyl)methyl]amine

[(2-methoxyphenyl)sulfonyl][(2-piperidylphenyl)methyl]amine: [(2-methoxyphenyl)sulfonyl][(2-piperidylphenyl)methyl]amine was prepared by treating 2-piperdinobenzylamine (0.48 g 2.0 mmols) with 2-methoxybenzensulfonylchloride (0.41 g, 2 mmols) in dichloromethane (10 ml), in presence of diisopropylethylamine (0.87 ml 5 mmols). Yield: 0.63 g (88%). MH⁺: 361.

{Difluoro[4-({[(2-methoxyphenyl)sulfonyl][(2-piperidylphenyl)methyl]amino}methyl)phenyl]methyl}diethoxyphosphino-1-one: {difluoro[4-({[(2-methoxyphenyl)sulfonyl][(2-piperidylphenyl)methyl]amino}methyl)phenyl]methyl}diethoxyphosphino-1-one was prepared by treating [(2-methoxyphenyl)sulfonyl][(2-piperidylphenyl)methyl]amine (0.36 g, 1 mmol) with 95% sodiumhydride (30 mg, 1.1 mmols) in DMF (5 ml) and stirring with [(4-(Bromomomethyl-2-chloro-phenyl)-difluoro-methyl-phosphonic acid diethyl ether (0.39 g 1 mmol) for overnight. Yield: 0.52 g (77%). MH⁺: 671 and 673.

{[4-(Difluorophosphonomethyl)-3-chlorophenyl]methyl}[(2-methoxyphenyl)sulfonyl][(2-piperidylphenyl)methyl]amine: {[4-(difluorophosphono methyl)-3-chlorophenyl]methyl}[(2-methoxyphenyl)sulfonyl][(2-piperidylphenyl)methyl]amine was prepared by treating {Difluoro[4-({[(2-methoxy phenyl)sulfonyl][(2-piperidylphenyl)methyl]amino}methyl)phenyl]methyl}diethoxy phosphino-1-one (0.47 g, 0.7 mmol) with trimethylsilyliodide (0.95 ml 7 mmols) in dichloromethane (10 ml). Yield: 0.25 g (58%). MH⁺: 615 and 617. ¹H NMR: (600 MHz, DMSO-d₆) 7.87δ (1H, d), 7.66δ (1H, d); 7.37δ (1H, d); 7.24δ (1H, m); 7.23δ (2H, m); 7.12δ (1H, s); 7.01δ (2H, d), 6.93δ (1H, d); 6.91δ (1H, s); 4.50δ (2H, s); 4.24δ (2H, d); 3.85δ (3H, s); 2.61δ (3H, s); 1.47δ (6H, m).

### Example 22

### {[4-(difluorophosphonomethyl)-3-chlorophenyl]methyl}[(2-methoxyphenyl)sulfonyl](3-piperidylmethyl)amine tert-butyl 3-({[(2-methoxyphenyl)sulfonyl]amino}methyl)piperidinecarboxylate: tert-butyl 3-({[(2-methoxyphenyl)sulfonyl]amino}methyl)piperidinecarboxylate was prepared by treating 3-(aminomethyl)-1-N-Boc-piperdine (0.43 g, 2 mmols) with 2-methoxybenzenesulfonylchloride (0.41 g, 2 mmols) in dichloromethane in presence of diisopropylethylamine (0.87 ml, 5 mmols). Yield: 0.63 g (82%). MH⁺: 385.

tert-butyl 3-{[({4-[(diethoxycarbonyl)difluoromethyl]phenyl}methyl)[(2-methoxyphenyl)sulfonyl]amino]methyl}piperidinecarboxylate: tert-butyl 3-{[({4-[(diethoxycarbonyl)difluoromethyl]phenyl}methyl)[(2-methoxyphenyl)sulfonyl]amino]methyl}piperidinecarboxylate was prepared by treating tert-butyl 3-({[(2-methoxy phenyl)sulfonyl]amino}methyl)piperidinecarboxylate (0.43 g, 1.1 mmols) with 95% sodiumhydride (31 mg, 1.2 mmols) in DMF and then treated with [(4-(Bromomomethyl-2-chloro-phenyl)-difluoro-methyl-phosphonic acid diethyl ether (0.44 g 1.1 mmol) for overnight. Yield: 0.60 g (86%). MH⁺: 695 and 697.

{[4-(difluorophosphonomethyl)-3-chlorophenyl]methyl}[(2-methoxyphenyl)sulfonyl](3-piperidylmethyl)amine: {[4-(difluorophosphonomethyl)-3-chlorophenyl]methyl}[(2-methoxyphenyl)sulfonyl](3-piperidylmethyl)amine was prepared by treating tert-butyl 3-{[({4-[(diethoxycarbonyl)difluoromethyl]phenyl}methyl)[(2-methoxyphenyl)sulfonyl]amino]methyl}piperidinecarboxylate (0.56 g, 0.8 mmols) with trimethylsilyliodide (1.09 ml 8 mmols) in dichloromethane (10 ml). Yield: 0.3 g (69%). MH⁺: 539 and 541. ¹H NMR: (600 MHz, DMSO-d₆) 9.48δ (1H, b), 8.53δ (1H, b); 7.84δ (1H, d); 7.68δ (1H, m); 7.52δ (1H, d); 7.41δ (1H, s); 7.37δ (1H, d), 7.31δ (1H, d); 7.13δ (1H, s); 4.35δ (1H, d); 4.257δ (1H, d); 3.95δ (3H, s); 3.09δ (1H, d); 3.94δ (2H, s); 2.57δ (1H, s), 2.04δ (1H, d). 1.57δ (3H, s); 1.11δ (1H, b); 0.90δ (1H, d).

### Example 23

### {[4-(difluorophosphonomethyl)-3-chlorophenyl]methyl}[(2-methoxyphenyl)sulfonyl]{[2-(methylethoxy)phenyl]methyl}amine

[(2-methoxyphenyl)sulfonyl]{[2-(methylethoxy)phenyl]methyl}amine: [(2-methoxyphenyl)sulfonyl]{[2-(methylethoxy)phenyl]methyl}amine was prepared by treating 1-(2-Isopropoxyphenyl)methylamine (0.33 g, 2 mmols) with 2-methoxybenzene sulfonylchloride (0.41 g, 2 mmols) in dichloromethane (10 ml) in presence of disiopropyl ethylamine (0.87 ml 5 mmols). Yield: 0.58 (87%). MH⁺: 336.

{[4-(difluorophosphonomethyl)-3-chlorophenyl]methyl}[(2-methoxyphenyl)sulfonyl]{[2-(methylethoxy)phenyl]methyl}amine: {[4-(difluorophosphono methyl)-3-chlorophenyl]methyl}[(2-methoxyphenyl)sulfonyl]{[2-(methylethoxy)phenyl]methyl}amine was prepared by treating [(2-methoxyphenyl)sulfonyl]{[2-(methylethoxy)phenyl]methyl}amine (0.34 g, 1 mmols) with 95% sodiumhydride (30 mg, 1.1 mmols) in DMF and then treating with [(4-(Bromomomethyl-2-chloro-phenyl)-difluoro-methyl-phosphonic acid diethyl ether (0.39 g 1 mmol) for overnight. Yield: 0.48 g (75%). MH⁺: 646 and 648.

{[4-(difluorophosphonomethyl)-3-chlorophenyl]methyl}[(2-methoxyphenyl)sulfonyl]{[2-(methylethoxy)phenyl]methyl}amine: {[4-(difluoro phosphonomethyl)-3-chlorophenyl]methyl}[(2-methoxyphenyl)sulfonyl]{[2-(methyl ethoxy)phenyl]methyl}amine was prepared by treating {[4-(difluorophosphonomethyl)-3-chlorophenyl]methyl}[(2-methoxyphenyl)sulfonyl]{[2-(methylethoxy)phenyl]methyl}amine (0.45 g, 0.6 mmols) with trimethylsyliliodide (0.82 ml 6 mmols) in dichloromethane (10 ml). Yield: 0.16 g (44%). MH⁺: 590 and 592. ¹H NMR: (600 MHz, DMSO-d₆) 7.79δ (1H, d), 7.61δ (1H, d); 7.44δ (1H, d); 7.20δ (1H, m); 7.15δ (3H, m); 6.98δ (1H, d); 6.88δ (1H, d), 6.74δ (1H, d); 4.50δ (1H, m); 4.45δ (2H, s); 4.397δ (2H, s); 3.84δ (3H, s); 1.16δ (6H, s).

### Example 24

### {[4-(difluorophosphonomethyl)-3-chlorophenyl]methyl}[(2-ethoxyphenyl)methyl][(2-methoxyphenyl)sulfonyl]amine

[(2-ethoxyphenyl)methyl][(2-methoxyphenyl)sulfonyl]amine: was prepared by treating 2-ethoxybenzylamine (0.3 g, 2 mmols) with 2-methoxybenzene sulfonylchloride (0.41 g, 2 mmols) with disiopropylethylamine (0.87 ml, 5 mmols) in dichloromethane (10 ml). Yield: 0.63 g (82%). MH⁺: 323.

diethoxy{[4-({[(2-ethoxyphenyl)methyl][(2-methoxyphenyl)sulfonyl]amino}methyl)phenyl]difluoromethyl}phosphino-1-one: was prepared by treating [(2-ethoxyphenyl)methyl][(2-methoxyphenyl)sulfonyl]amine (0.32 g, 1 mmols) with 95% sodiumhydride (30 mg, 1.1 mools) in dry DMF (5 ml) and then stirring with [(4-(Bromomo methyl-2-chloro-phenyl)-difluoro-methyl-phosphonic acid diethyl ether (0.39 g 1 mmol) for overnight. Yield: 0.42 g (67%). MH⁺: 632 and 634.

{[4-(difluorophosphonomethyl)-3-chlorophenyl]methyl}[(2-ethoxyphenyl)methyl][(2-methoxyphenyl)sulfonyl]amine: {[4-(difluorophosphonomethyl)-3-chlorophenyl]methyl}[(2-ethoxyphenyl)methyl][(2-methoxyphenyl)sulfonyl]amine was prepared by treating diethoxy{[4-({[(2-ethoxy phenyl)methyl][(2-methoxyphenyl)sulfonyl]amino}methyl)phenyl]difluoromethyl}phosphino-1-one (0.63 g, 0.6 mmols) with trimethylsyliliodide (0.82 ml, 6 mmols) in dichloromethane. Yield: 0.12 g (35%). MH⁺: 576 and 578. ¹H NMR: (600 MHz, DMSO-d₆) 7.76δ (1H, d), 7.61δ (1H, d); 7.44δ (1H, d); 7.19δ (2H, m); 7.14δ (2H, m); 7.05δ (1H, d); 6.99δ (1H, d), 6.80δ (1H, d); 6.76δ (1H, d); 4.62δ (2H, m); 4.34δ (2H, s); 3.85δ (3H, s); 1.23δ (3H, s).

### Example 25

### {[4-(difluorophosphonomethyl)-3-chlorophenyl]methyl}[(2-methoxyphenyl)sulfonyl](2-piperidylmethyl)amine

tert-butyl 2-({[(2-methoxyphenyl)sulfonyl]amino}methyl)piperidinecarboxylate: tert-butyl 2-({[(2-methoxyphenyl)sulfonyl]amino}methyl)piperidinecarboxylate was prepared by treating tert-butyl 2-(aminomethyl)piperidinecarboxylate (0.43 g, 2 mmols) with 2-methoxybenzenesulfonylchloride (0.41 g, 2 mmols) in dichloromethane (10 ml) in presence of diisopropylethylamine (0.70 ml 4 mmols). Yield: 0.73 g (95%). MH⁺: 385.

tert-butyl 2-{[({4-[(diethoxycarbonyl)difluoromethyl]phenyl}methyl)[(2-methoxy phenyl)sulfonyl]amino]methyl}piperidinecarboxylate: tert-butyl 2-{[({4-[(diethoxycarbonyl)difluoromethyl]phenyl}methyl)[(2-methoxyphenyl)sulfonyl]amino]methyl}piperidinecarboxylate was prepared by treating tert-butyl 2-({[(2-methoxy phenyl)sulfonyl]amino}methyl)piperidinecarboxylate (0.42 g, 1.25 mmols) with 95% sodiumhydride in dry DMF (5 ml) and then stirring with [(4-(Bromomo methyl-2-chloro-phenyl)-difluoro-methyl-phosphonic acid diethyl ether (0.49 g 1.25 mmol) for overnight. Yield: 0.48 g (55%). MH⁺: 695 and 697.

{[4-(difluorophosphonomethyl)-3-chlorophenyl]methyl}[(2-methoxyphenyl)sulfonyl](2-piperidylmethyl)amine: {[4-(difluorophosphonomethyl)-3-chlorophenyl]methyl}[(2-methoxyphenyl)sulfonyl](2-piperidylmethyl)amine was prepared by treating tert-butyl 2-{[({4-[(diethoxycarbonyl)difluoromethyl]phenyl}methyl)[(2-methoxy phenyl)sulfonyl]amino]methyl}piperidinecarboxylate (0.35 g, 0.5 mmols) with trimethylsyliliodide (0.34 ml, 5 mmols) in dichloromethane (10 ml). Yield: 0.12 g (43%). MH⁺: 539 and 541. ¹H NMR: (600 MHz, DMSO-d₆) 7.81δ (1H, b), 7.63δ (2H, m); 7.54δ (1H, d); 7.41δ (1H, s); 7.24δ (1H, d); 7.08δ (1H, d); 4.31δ (1H, d), 4.08δ (2H, s); 3.90δ (3H, s); 3.70δ (2H, d); 3.18δ (2H, d); 1.67δ (1H, m); 1.46δ (3H, m); 1.20δ (1H, m); 1.05δ (1H, m).

### Example 26

### Methyl 2-(3-{4-[({[4-(difluorophosphonomethyl)-3-chlorophenyl]methyl}[(2-methoxyphenyl)sulfonyl]amino)methyl]phenyl}phenyl)acetate

Step A. A mixture of (4-aminomethylphenyl)boronic acid, hydrochloride (10.00 g, 53.3 mmol), DIEA (37.16 mL, 213.3 mmol) and DMAP (0.040 g, 0.32 mmol) in dry dichloromethane (200 mL) were stirred nitrogen at 0° C. 2-Methoxybenzene sulfonyl chloride (10.02 g, 48.5 mmol) was added and the reaction mixture warmed up to room temperature for overnight. The reaction was then rotary evaporated to an oil which was dissolved in ethyl acetate and washed with 3% HCl followed by a wash with saturated NaCl. The organic layer was dried (Na₂SO₄) and concentrated. The crude residue solid was washed by ether to yield 11.653 g (74%) of (4-({[(2-methoxyphenyl)sulfonyl]amino}methyl)phenyl)boronic acid as light yellow solid. MS (M+H)⁺ 322.

Step B. In a 10 mL glass tube was placed (4-({[(2-methoxyphenyl)sulfonyl]amino}methyl)phenyl)boronic acid (0.289 g, 0.90 mmol), methyl 2-(3-bromophenyl)acetate (0.206 g, 0.90 mmol), bis(triphenylphosphine)palladium (II) chloride (0.031 g, 0.045 mmol), Et₃N (0.38 mL, 2.70 mmol), ethanol (3.8 mL) and a magnetic stir bar. The vessel was sealed with a septum and placed into the microwace cavity. Microwace irradiation was used, and the reaction mixture was keep at 150° C. for 250 seconds. After the mixture was allowed to cool to room temperature, the reaction vessel was opened and the contents were filter through the Celite. The filtrate was concentrated under vacuum. The crude residue was purified by flash chromatography (ethyl acetate/hexanes, 100% hexanes to 1:1) to yield 0.260 g (68%) of methyl 2-{3-[4-({[(2-methoxyphenyl)sulfonyl]amino}methyl)phenyl]phenyl}acetate as colorless oil. MS (M+NH₄)⁺ 443; (M−H)⁻ 424.

Step C. To a solution of methyl 2-{3-[4-({[(2-methoxyphenyl)sulfonyl]amino}methyl)phenyl]phenyl}acetate (0.223 g, 0.52 mmol) in 5 mL of dry DMF was added potassium tert-butoxide (0.52 mL g, 0.52 mmol, 1M in tert-butanol) at room temperature under an nitrogen atmosphere. After 5 minute, {[4-(bromomethyl)-2-chlorophenyl]difluoromethyl}diethoxyphosphino-1-one (0.205 g, 0.52 mmol) was injected, and the solution was stirred at room temperature for overnight. The DMF was evaporated (rotavap) under vacuum. The crude residue was purified by flash chromatography (ethyl acetate/hexanes, 100% hexanes to 1:1) to isolate the methyl 2-[3-(4-{[({4-[(diethoxycarbonyl)difluoromethyl]-3-chlorophenyl}methyl)[(2-methoxyphenyl)sulfonyl]amino]methyl}phenyl)phenyl]acetate (0.273 g) in 71% yield as colorless oil. MS (M+NH₄)⁺ 753, 755; (M−Et)⁻ 706,708.

Step D. A solution of methyl 2-[3-(4-{[({4-[(diethoxycarbonyl)difluoromethyl]-3-chlorophenyl}methyl)[(2-methoxyphenyl)sulfonyl]amino]methyl}phenyl)phenyl]acetate (0.273 g, 0.37 mmol) and BSTFA (1.18 mL, 4.45 mmol) in dry dichloromethane (3.2 mL) was stirred at room temperature for 1 hour. The reaction mixture was cool to −20° C., then added iodotrimethylsilane (0.89 mL, 6.24 mmol). The reaction mixture was slowly warmed up to room temperature to stir for an additional 2 hour. The solvent and excess iodotrimethylsilane was removed by vacuum. The residue was treated with acetonitrile (16 mL), TFA (2 mL) and H₂O (4 mL) and stirred for overnight at room temperature. The reaction was then rotary evaporated to an oil which was dissolved in ethyl acetate and washed with 5% Na₂S₂O₄ (acidified) followed by a wash with saturated NaCl. The organic layer was dried (Na₂SO₄) and concentrated. The crude residue was purified by C-18 flash chromatography (MeOH/H₂O, 100% H₂O to 6:4) to give the methyl 2-(3-{4-[({[4-(difluorophosphonomethyl)-3-chlorophenyl]methyl}[(2-methoxyphenyl)sulfonyl]amino)methyl]phenyl}phenyl)acetate (0.032 g) in 13% yield as white solid and 0.161 g of acid and ester mixture. MS (M+NH₄)⁺ 697, 699; (M−H)⁻ 678, 680.

### Example 27

### 2-(3-{4-[({[4-(difluorophosphonomethyl)-3-chlorophenyl]methyl}[(2-methoxyphenyl)sulfonyl]amino)methyl]phenyl}phenyl)acetic acid

The methyl 2-(3-{4-[({[4-(difluorophosphonomethyl)-3-chlorophenyl]methyl}[(2-methoxyphenyl)sulfonyl]amino)methyl]phenyl}phenyl)acetate (0.161 g, 0.23 mmol) was dissolved in 20 mL of 0.25 M LiOH solution (in MeOH/H₂O, 75/25). The mixture was stirred overnight at room temperature and concentrated in vacuo. The residue added water, then added 1 N HCl aq. solution until pH<7. The crude residue was purified by C-18 flash chromatography (MeCN/H₂O, 100% H₂O to 6:4) to give the 2-(3-{4-[({[4-(difluorophosphonomethyl)-3-chlorophenyl]methyl}[(2-methoxyphenyl)sulfonyl]amino)methyl]phenyl}phenyl)acetic acid (0.130 g) in 85% yield as white solid. MS (M+NH₄)⁺ 683, 685; (M−H)⁻ 664, 666.

### Example 28

### Methyl 3-{4-[({[4-(difluorophosphonomethyl)-3-chlorophenyl]methyl}[(2-methoxyphenyl)sulfonyl]amino)methyl]phenyl}-5-bromobenzoate

Step A. In a 10 mL glass tube was placed (4-({[(2-methoxyphenyl)sulfonyl]amino}methyl)phenyl)boronic acid (0.289 g, 0.90 mmol), methyl 5-bromo-3-iodobenzoate (0.307 g, 0.90 mmol), bis(triphenylphosphine)palladium (II) chloride (0.031 g, 0.045 mmol), Et₃N (0.38 mL, 2.70 mmol), ethanol (3.8 mL) and a magnetic stir bar. The vessel was sealed with a septum and placed into the microwace cavity. Microwace irradiation was used, and the reaction mixture was keep at 140° C. for 250 seconds. After the mixture was allowed to cool to room temperature, the reaction vessel was opened and the contents were filter through the Celite. The filtrate was concentrated under vacuum. The crude residue was purified by flash chromatography (ethyl acetate/hexanes, 100% hexanes to 1:1) to yield 0.276 g (63%) of methyl 5-bromo-3-[4-({[(2-methoxyphenyl)sulfonyl]amino}methyl)phenyl]benzoate as yellow oil. MS (M−H)⁻ 488,490.

Step B. To a solution of methyl 5-bromo-3-[4-({[(2-methoxyphenyl)sulfonyl]amino}methyl)phenyl]benzoate (0.224 g, 0.46 mmol) in 5 mL of dry DMF was added potassium tert-butoxide (0.46 mL g, 0.46 mmol, 1M in tert-butanol) at room temperature under an nitrogen atmosphere. After 5 minute, {[4-(bromomethyl)-2-chlorophenyl]difluoromethyl}diethoxyphosphino-1-one (0.179 g, 0.46 mmol) was injected, and the solution was stirred at room temperature for overnight. The DMF was evaporated (rotavap) under vacuum. The crude residue was purified by flash chromatography (ethyl acetate/hexanes, 100% hexanes to 1:1) to isolate the methyl 3-(4-{[({4-[(diethoxycarbonyl)difluoromethyl]-3-chlorophenyl}methyl)[(2-methoxyphenyl)sulfonyl]amino]methyl}phenyl)-5-bromobenzoate (0.314 g) in 85% yield as colorless oil. MS (M+NH₄)⁺ 817, 819; (M−Et)⁻ 770,772.

Step C. A solution of methyl 3-(4-{[({4-[(diethoxycarbonyl)difluoromethyl]-3-chlorophenyl}methyl)[(2-methoxyphenyl)sulfonyl]amino]methyl}phenyl)-5-bromobenzoate (0.310 g, 0.39 mmol) and BSTFA (1.23 mL, 4.64 mmol) in dry dichloromethane (3.4 mL) was stirred at room temperature for 1 hour. The reaction mixture was cool to −20° C., then added iodotrimethylsilane (0.44 mL, 3.10 mmol). The reaction mixture was slowly warmed up to room temperature to stir for an additional 2 hour. The solvent and excess iodotrimethylsilane was removed by vacuum. The residue was treated with acetonitrile (8 mL), TFA (1 mL) and H₂O (2 mL) and stirred for overnight at room temperature. The reaction was then rotary evaporated to an oil which was dissolved in ethyl acetate and washed with 5% Na₂S₂O₄ (acidified) followed by a wash with saturated NaCl. The organic layer was dried (Na₂SO₄) and concentrated. The crude residue was purified by C-18 flash chromatography (MeOH/H₂O, 100% H₂O to 7:3) to give the methyl 3-{4-[({[4-(difluorophosphonomethyl)-3-chlorophenyl]methyl}[(2-methoxyphenyl)sulfonyl]amino)methyl]phenyl}-5-bromobenzoate (0.271 g) in 93% yield as white solid. MS (M+NH₄)⁺ 761,763; (M−H)⁻ 742, 744.

### Example 29

### 3-{4-[({[4-(difluorophosphonomethyl)-3-chlorophenyl]methyl}[(2-methoxyphenyl)sulfonyl]amino)methyl]phenyl}-5-bromobenzoic acid

The methyl 3-{4-[({[4-(difluorophosphonomethyl)-3-chlorophenyl]methyl}[(2-methoxyphenyl)sulfonyl]amino)methyl]phenyl}-5-bromobenzoate (0.145 g, 0.19 mmol) was dissolved in 20 mL of 0.25 M LiOH solution (in MeOH/H₂O, 75/25). The mixture was stirred overnight at room temperature and concentrated in vacuo. The residue added water, then added 1 N HCl aq. solution until pH<7. The crude residue was purified by C-18 flash chromatography (MeOH/H₂O, 100% H₂O to 6:4) to give the title compound (0.121 g) in 85% yield as white solid. MS (M+NH₄)⁺ 747, 749; (M−H)⁻ 728, 730.

### Example 30

### 6-{4-[({[4-(difluorophosphonomethyl)-3-chlorophenyl]methyl}[(2-methoxyphenyl)sulfonyl]amino)methyl]phenyl}-3-hydroisobenzofuran-1-one

Step A. In a 10 mL glass tube was placed (4-({[(2-methoxyphenyl)sulfonyl]amino}methyl)phenyl)boronic acid (0.321 g, 1.00 mmol), 5-bromophthalide (0.213 g, 1.00 mmol), bis(triphenylphosphine)palladium (II) chloride (0.035 g, 0.05 mmol), Et₃N (0.42 mL, 3.00 mmol), ethanol (4.2 mL) and a magnetic stir bar. The vessel was sealed with a septum and placed into the microwace cavity. Microwace irradiation was used, and the reaction mixture was keep at 150° C. for 350 seconds. After the mixture was allowed to cool to room temperature, the reaction vessel was opened and the contents were filter through the Celite. The filtrate was concentrated under vacuum. The crude residue was purified by flash chromatography (ethyl acetate/hexanes, 100% hexanes to 1:1) to yield 0.324 g (79%) of 6-[4-({[(2-methoxyphenyl)sulfonyl]amino}methyl)phenyl]-3-hydroisobenzofuran-1-one as yellow solid. MS (M+H)⁺ 410; (M−H)⁻ 408.

Step B. To a solution of 6-[4-({[(2-methoxyphenyl)sulfonyl]amino}methyl)phenyl]-3-hydroisobenzofuran-1-one (0.324 g, 0.79 mmol) in 5 mL of dry DMF was added potassium tert-butoxide (0.79 mL g, 0.79 mmol, 1M in tert-butanol) at room temperature under an nitrogen atmosphere. After 5 minute, {[4-(bromomethyl)-2-chlorophenyl]difluoromethyl}diethoxyphosphino-1-one (0.310 g, 0.79 mmol) was injected, and the solution was stirred at room temperature for overnight. The DMF was evaporated (rotavap) under vacuum. The crude residue was purified by flash chromatography (ethyl acetate/hexanes, 100% hexanes to 2:1) to isolate the ({2-chloro-4-[([(2-methoxyphenyl)sulfonyl]{[4-(3-oxohydroisobenzofuran-5-yl)phenyl]methyl}amino)methyl]phenyl}difluoromethyl)diethoxyphosphino-1-one (0.468 g) in 82% yield as colorless oil. MS (M+NH₄)⁺ 737, 739; (M−Et)⁻ 690, 692.

Step C. A solution of ({2-chloro-4-[([(2-methoxyphenyl)sulfonyl]{[4-(3-oxohydroisobenzofuran-5-yl)phenyl]methyl}amino)methyl]phenyl}difluoromethyl)diethoxyphosphino-1-one (0.460 g, 0.64 mmol) and BSTFA (2.04 mL, 7.66 mmol) in dry dichloromethane (5.6 mL) was stirred at room temperature for 1 hour. The reaction mixture was cool to −20° C., then added iodotrimethylsilane (0.73 mL, 5.11 mmol). The reaction mixture was slowly warmed up to room temperature to stir for an additional 2 hour. The solvent and excess iodotrimethylsilane was removed by vacuum. The residue was treated with acetonitrile (16 mL) and H₂O (4 mL) and stirred for overnight at room temperature. The reaction was then rotary evaporated to an oil, and the crude residue was purified by C-18 flash chromatography (MeOH/H₂O, 100% H₂O to 6:4) to give the title compound (0.337 g) in 79% yield as white solid. MS (M+NH₄)⁺ 681, 683; (M−H)⁻ 662, 664.

### Example 31

### 5-{4-[({[4-(difluorophosphonomethyl)-3-chlorophenyl]methyl}[(2-methoxyphenyl)sulfonyl]amino)methyl]phenyl}-2-(hydroxymethyl)benzoic acid, three sodium salt

The 6-{4-[({[4-(difluorophosphonomethyl)-3-chlorophenyl]methyl}[(2-methoxyphenyl)sulfonyl]amino)methyl]phenyl}-3-hydroisobenzofuran-1-one (0.264 g, 0.40 mmol) was dissolved in 10 mL of MeOH and added 10 mL of 0.5 M NaOH solution (in H₂O). The mixture was stirred overnight at room temperature and concentrated in vacuo. The crude residue was purified by C-18 flash chromatography (MeOH/H₂O, 100% H₂O to 3:7) to give the title compound (0.286 g) in 96% yield as white solid. MS (M+H)⁺ 682, 684; (M−H)⁻ 680, 682.

### Example 32

### Methyl 5-{4-[({[4-(difluorophosphonomethyl)-3-chlorophenyl]methyl}[(2-methoxyphenyl)sulfonyl]amino)methyl]phenyl}-2-methoxybenzoate

Step A. In a 10 mL glass tube was placed (4-({[(2-methoxyphenyl)sulfonyl]amino}methyl)phenyl)boronic acid (0.321 g, 1.00 mmol), methyl 5-bromo-2-methoxybenzoate (0.245 g, 1.00 mmol), bis(triphenylphosphine)palladium (II) chloride (0.035 g, 0.05 mmol), Et₃N (0.42 mL, 3.00 mmol), ethanol (4.2 mL) and a magnetic stir bar. The vessel was sealed with a septum and placed into the microwace cavity. Microwace irradiation was used, and the reaction mixture was keep at 150° C. for 350 seconds. After the mixture was allowed to cool to room temperature, the reaction vessel was opened and the contents were filter through the Celite. The filtrate was concentrated under vacuum. The crude residue was purified by flash chromatography (ethyl acetate/hexanes, 100% hexanes to 1:1) to yield 0.234 g (53%) of methyl 2-methoxy-5-[4-({[(2-methoxyphenyl)sulfonyl]amino}methyl)phenyl]benzoate as light yellow solid. MS (M+H)+ 442; (M−H)⁻ 440.

Step B. To a solution of methyl 2-methoxy-5-[4-({[(2-methoxyphenyl)sulfonyl]amino}methyl)phenyl]benzoate (0.206 g, 0.47 mmol) in 5 mL of dry DMF was added potassium tert-butoxide (0.47 mL g, 0.47 mmol, 1M in tert-butanol) at room temperature under an nitrogen atmosphere. After 5 minute, {[4-(bromomethyl)-2-chlorophenyl]difluoromethyl}diethoxyphosphino-1-one (0.183 g, 0.47 mmol) was injected, and the solution was stirred at room temperature for overnight. The DMF was evaporated (rotavap) under vacuum. The crude residue was purified by flash chromatography (ethyl acetate/hexanes, 100% hexanes to 1:1) to isolate the methyl 5-(4-{[({4-[(diethoxycarbonyl)difluoromethyl]-3-chlorophenyl}methyl)[(2-methoxyphenyl)sulfonyl]amino]methyl}phenyl)-2-methoxybenzoate (0.231 g) in 66% yield as colorless oil. MS (M+NH₄)⁺ 769, 771; (M−Et)⁻ 722,724.

Step C. A solution of methyl 5-(4-{[({4-[(diethoxycarbonyl)difluoromethyl]-3-chlorophenyl}methyl)[(2-methoxyphenyl)sulfonyl]amino]methyl}phenyl)-2-methoxybenzoate (0.370 g, 0.49 mmol) and BSTFA (1.57 mL, 5.90 mmol) in dry dichloromethane (4.3 mL) was stirred at room temperature for 1 hour. The reaction mixture was cool to −20° C., then added iodotrimethylsilane (0.56 mL, 3.94 mmol). The reaction mixture was slowly warmed up to room temperature to stir for an additional 2 hour. The solvent and excess iodotrimethylsilane was removed by vacuum. The residue was treated with acetonitrile (16 mL) and H₂O (4 mL) and stirred for overnight at room temperature. The reaction was then rotary evaporated to an oil which was dissolved in ethyl acetate and washed with 5% Na₂S₂O₄ (acidified) followed by a wash with saturated NaCl. The organic layer was dried (Na₂SO₄) and concentrated. The crude residue was purified by C-18 flash chromatography (MeOH/H₂O, 100% H₂O to 55:45) to give the title compound (0.031 g) in 9% yield as white solid and 0.250 g of acid plus ester mixture. MS (M+NH₄)⁺ 713, 715; (M−H)⁻ 694, 696.

### Example 33

### 5-{4-[({[4-(difluorophosphonomethyl)-3-chlorophenyl]methyl}[(2-methoxyphenyl)sulfonyl]amino)methyl]phenyl}-2-methoxybenzoic acid

The methyl 5-{4-[({[4-(difluorophosphonomethyl)-3-chlorophenyl]methyl}[(2-methoxyphenyl)sulfonyl]amino)methyl]phenyl}-2-methoxybenzoate (0.250 g, 0.37 mmol) was dissolved in 20 mL of 0.25 M LiOH solution (in MeOH/H₂O, 75/25). The mixture was stirred overnight at room temperature and concentrated in vacuo. The residue added water, then added 1 N HCl aq. solution until pH<7. The crude residue was purified by C-18 flash chromatography (MeCN/H₂O, 100% H₂O to 4:6) to give the title compound (0.190 g) in 76% yield as white solid. MS (M−H)⁻ 680, 682.

### Example 34

### 5-{4-[({[4-(difluorophosphonomethyl)-3-chlorophenyl]methyl}[(2-methoxyphenyl)sulfonyl]amino)methyl]phenyl}-2-hydroxybenzoic acid and 5-{4-[({[4-(difluorophosphonomethyl)phenyl]methyl}[(2-methoxyphenyl)sulfonyl]amino)methyl]phenyl}-2-hydroxybenzoic acid

Step A. In a 10 mL glass tube was placed (4-({[(2-methoxyphenyl)sulfonyl]amino}methyl)phenyl)boronic acid (0.321 g, 1.00 mmol), phenylmethyl 5-bromo-2-(phenylmethoxy)benzoate (0.397 g, 1.00 mmol), bis(triphenylphosphine)palladium (II) chloride (0.035 g, 0.05 mmol), Et₃N (0.35 mL, 2.5 mmol), ethanol (4.0 mL) and a magnetic stir bar. The vessel was sealed with a septum and placed into the microwace cavity. Microwace irradiation was used, and the reaction mixture was keep at 150° C. for 350 seconds. After the mixture was allowed to cool to room temperature, the reaction vessel was opened and the contents were filter through the Celite. The filtrate was concentrated under vacuum. The crude residue was purified by flash chromatography (ethyl acetate/hexanes, 100% hexanes to 1:1) to yield 0.302 g (51%) of phenylmethyl 5-[4-({[(2-methoxyphenyl)sulfonyl]amino}methyl)phenyl]-2-(phenylmethoxy)benzoate as light yellow solid. MS (M−H)⁻ 592.

Step B. To a solution of phenylmethyl 5-[4-({[(2-methoxyphenyl)sulfonyl]amino}methyl)phenyl]-2-(phenylmethoxy)benzoate (11.19 g, 18.85 mmol) in 60 mL of dry DMF was added potassium tert-butoxide (18.85 mL g, 18.85 mmol, 1M in tert-butanol) at room temperature under an nitrogen atmosphere. After 5 minute, {[4-(bromomethyl)-2-chlorophenyl]difluoromethyl}diethoxyphosphino-1-one (7.38 g, 18.85 mmol) was injected, and the solution was stirred at room temperature for overnight. The DMF was evaporated (rotavap) under vacuum. The crude residue was purified by flash chromatography (ethyl acetate/hexanes, 100% hexanes to 1:1) to isolate the phenylmethyl 5-(4-{[({4-[(diethoxycarbonyl)difluoromethyl]-3-chlorophenyl}methyl)[(2-methoxyphenyl)sulfonyl]amino]methyl}phenyl)-2-(phenylmethoxy)benzoate (14.65 g) in 86% yield as yellow oil. MS (M+NH₄)⁺ 921, 923.

Step C. A solution of phenylmethyl 5-(4-{[({4-[(diethoxycarbonyl)difluoromethyl]-3-chlorophenyl}methyl)[(2-methoxyphenyl)sulfonyl]amino]methyl}phenyl)-2-(phenylmethoxy)benzoate (14.65 g, 16.20 mmol) in mixture of methanol (100 mL) and ethyl acetate (100 mL) was stirred in the presence of 10% palladium on carbon (3.00 g)under an atmosphere of hydrogen at room temperature for 5 hour. The solution was filtered through Celite, and the filtrate was evaporated under a vacuum. The crude product was direct use for next step without any purification.

Step D. A solution of 5-(4-{[({4-[(diethoxycarbonyl)difluoromethyl]-3-chlorophenyl}methyl)[(2-methoxyphenyl)sulfonyl]amino]methyl}phenyl)-2-hydroxybenzoic acid (10.00 g, 13.81 mmol) and BSTFA (44.02 mL, 165.72 mmol) in dry dichloromethane (120 mL) was stirred at room temperature for 1 hour. The reaction mixture was cool to −20° C., then added iodotrimethylsilane (15.72 mL, 110.48 mmol). The reaction mixture was slowly warmed up to room temperature to stir for an additional 2 hour. The solvent and excess iodotrimethylsilane was removed by vacuum. The residue was treated with acetonitrile (48 mL), TFA (6 mL) and H₂O (12 mL) and stirred for overnight at room temperature. The reaction was then rotary evaporated to an oil which was dissolved in ethyl acetate and washed with 5% Na₂S₂O₄ (acidified) followed by a wash with saturated NaCl. The organic layer was dried (Na₂SO₄) and concentrated. The crude residue was purified by C-18 flash chromatography (MeOH/H₂O, 100% H₂O to 5/5) to give the 5-{4-[({[4-(difluorophosphonomethyl)phenyl]methyl}[(2-methoxyphenyl)sulfonyl]amino)methyl]phenyl}-2-hydroxybenzoic acid (0.065 g) in 0.7% yield as white solid; LC (RT: 1.42 min.); MS (M+NH₄)⁺ 651; (M−H)⁻ 632, and 5-{4-[({[4-(difluorophosphonomethyl)-3-chlorophenyl]methyl}[(2-methoxyphenyl)sulfonyl]amino)methyl]phenyl}-2-hydroxybenzoic acid (6.53 g) in 71% yield as white solid. LC (RT: 1.51 min.); MS (M+NH₄)⁺ 741, 743; (M−H)⁻ 722, 724.

### Example 35

### {[4-(difluorophosphonomethyl)-3-chlorophenyl]methyl}[(2 methoxyphenyl)sulfonyl]{[2-(3-methylthiophenyl)phenyl]methyl}amine

Using the procedure from example 46, step D, ({2-chloro-4-[([(2-methoxyphenyl)sulfonyl]{[2-(3-methylthiophenyl)phenyl]methyl}amino)methyl]phenyl}difluoromethyl)diethoxyphosphino-1-one (0.600 g, 0.84 mmol) and iodotrimethylsilane (0.96 mL, 6.76 mmol) in dry dichloromethane (5 mL) The crude residue was purified by C-18 flash chromatography (MeOH/H₂O, 100% H₂O to 6:4) to give {[4-(difluorophosphonomethyl)-3-chlorophenyl]methyl}[(2-methoxyphenyl)sulfonyl]{[2-(3-methylthiophenyl)phenyl]methyl}amine (0.60 g) in 11% yield as yellow solid. MS (M+NH₄)⁺ 670, 672; (M−H)⁻ 651, 653.

Using the procedure from example 46, step C, [(2-methoxyphenyl)sulfonyl]{[2-(3methylthiophenyl)phenyl]methyl}amine (0.660 g, 1.65 mmol) in 8 mL of dry DMF, potassium tert-butoxide (1.65 mL, 1.65 mmol, 1M in tert-butanol), and {[4-(bromomethyl)-2-chlorophenyl]difluoromethyl}diethoxyphosphino-1-one (0.860 g, 1.65 mmol) were reacted. The crude residue was purified by flash chromatography (ethyl acetate/hexanes, 1:1) to isolate ({2-chloro-4-[([(2-methoxyphenyl)sulfonyl]{[2-(3 methylthiophenyl)phenyl]methyl}amino)methyl]phenyl}difluoromethyl)diethoxyphosphino-1-one (0.845 g) in 72% yield as colorless oil. MS (M+NH₄)⁺ 727, 729.

Using the procedure from example 46, step B, (3-methylthiophenyl)boronic acid (0.252 g, 1.50 mmol), [(2-bromophenyl)methyl][(2-methoxyphenyl)sulfonyl]amine (0.534 g, 1.50 mmol), bis(triphenylphosphine)palladium (II) chloride (0.07 g, 0.1 mmol), 1M Na₂CO₃ (in water) (5 mL), acetonitrile (5 mL) and a magnetic stir bar. The crude residue was purified by flash chromatography (ethyl acetate/hexanes, 1:2) to yield 0.670 g of [(2-methoxyphenyl)sulfonyl]{[2-(3methylthiophenyl)phenyl]methyl}amine as a yellow oil. MS (M−H)⁻ 398.

### Example 36

### {[4-(difluorophosphonomethyl)-3-chlorophenyl]methyl}[(2-(2,3-dihydrobenzo[b]furan-5-yl)phenyl)methyl][(2-methoxyphenyl)sulfonyl]amine

Using the procedure from example 46, step D, {[2-chloro-4-({[(2-(2,3-dihydrobenzo[b]furan-5-yl)phenyl)methyl][(2-methoxyphenyl)sulfonyl]amino}methyl)phenyl]difluoromethyl}diethoxyphosphino-1-one (0.527 g, 0.75 mmol) and iodotrimethylsilane (0.849 mL, 5.97 mmol) in dry dichloromethane (5 mL) The crude residue was purified by C-18 flash chromatography (MeOH/H₂O, 100% H₂O to 1:1) to give {[4-(difluorophosphonomethyl)-3-chlorophenyl]methyl}[(2-(2,3-dihydrobenzo[b]furan-5-yl)phenyl)methyl][(2-methoxyphenyl)sulfonyl]amine (0.01 g) in 2% yield as yellow solid. MS (M+NH₄)⁺ 666; (M−H)⁻ 647,649.

Using the procedure from example 46, step C, [(2-(2,3-dihydrobenzo[b]furan-5-yl)phenyl)methyl][(2-methoxyphenyl)sulfonyl]amine (0.428 g, 1.08 mmol) in 8 mL of dry DMF, potassium tert-butoxide (1.08 mL, 1.08 mmol, 1M in tert-butanol), and {[4-(bromomethyl)-2-chlorophenyl]difluoromethyl}diethoxyphosphino-1-one (0.565 g, 1.08 mmol) were reacted. The crude residue was purified by flash chromatography (ethyl acetate/hexanes, 1:2) to isolate {[2-chloro-4-({[(2-(2,3-dihydrobenzo[b]furan-5-yl)phenyl)methyl][(2-methoxyphenyl)sulfonyl]amino}methyl)phenyl]difluoromethyl}diethoxyphosphino-1-one (0.559 g) in 73% yield as a yellow oil. MS (M+H)⁺ 707.

Using the procedure from example 46, step B, 2,3-dihydro-1-benzofuran-5-ylboronic acid (0.246 g, 1.50 mmol), [(2-bromophenyl)methyl][(2-methoxyphenyl)sulfonyl]amine (0.534 g, 1.50 mmol), bis(triphenylphosphine)palladium (II) chloride (0.07 g, 0.1 mmol), 1M Na₂CO₃ (in water) (5 mL), acetonitrile (5 mL) and a magnetic stir bar. The crude residue was purified by flash chromatography (ethyl acetate/hexanes, 1:2) to yield 0.447 g (75%) of [(2-(2,3-dihydrobenzo[b]furan-5-yl)phenyl)methyl][(2-methoxyphenyl)sulfonyl]amine as a white solid. MS (M−H)⁻ 394.

### Example 37

### 4-{2-[({[4-(difluorophosphonomethyl)-3-chlorophenyl]methyl}[(2-methoxyphenyl)sulfonyl]amino)methyl]phenyl}benzamide

Using the procedure from example 46, step D, 4-(2-{[({4-[(diethoxycarbonyl)difluoromethyl]-3-chlorophenyl}methyl)[(2-methoxyphenyl)sulfonyl]amino]methyl}phenyl)benzamide (0.369 g, 0.52 mmol) and iodotrimethylsilane (0.592 mL, 4.16 mmol) in dry dichloromethane (5 mL) The crude residue was purified by stirring in water and washing with a small amount of methanol and ethyl acetate to give 4-{2-[({[4-(difluorophosphonomethyl)-3-chlorophenyl]methyl}[(2-methoxyphenyl)sulfonyl]amino)methyl]phenyl}benzamide (0.250 g) in 74% yield as white solid. MS (M+H)⁺ 651; (M−H)⁻ 649.

Using the procedure from example 46, step C, 4-[2-({[(2-methoxyphenyl)sulfonyl]amino}methyl)phenyl]benzamide (0.396 g, 1.00 mmol) in 5 mL of dry DMF, potassium tert-butoxide (1.00 mL, 1.00 mmol, 1M in tert-butanol), and {[4-(bromomethyl)-2-chlorophenyl]difluoromethyl}diethoxyphosphino-1-one (0.391 g, 1.00 mmol) were reacted. The crude residue was purified by flash chromatography (dichloromethane/methanol, 100:4), then washed with (dichloromethane/hexanes, 1:1) to isolate 4-(2-{[({4-[(diethoxycarbonyl)difluoromethyl]-3-chlorophenyl}methyl)[(2-methoxyphenyl)sulfonyl]amino]methyl}phenyl)benzamide (0.400 g) in 57% yield as white solid. MS (M+H)⁺ 708.

Using the procedure from example 46, step B, 4-aminocarbonylphenylboronic acid (0.330 g, 2.00 mmol), [(2-bromophenyl)methyl][(2-methoxyphenyl)sulfonyl]amine (0.712 g, 2.00 mmol), bis(triphenylphosphine)palladium (II) chloride (0.07 g, 0.1 mmol), 1M Na₂CO₃ (in water) (5 mL), acetonitrile (5 mL) and a magnetic stir bar. The crude residue was purified by washing with dichloromethane to yield 0.628 g (79%) of 4-[2-({[(2-methoxyphenyl)sulfonyl]amino}methyl)phenyl]benzamide as a white solid. MS (M−H)⁻ 395.

### Example 38

### {[4-(difluorophosphonomethyl)-3-chlorophenyl]methyl}[(2-methoxyphenyl)sulfonyl]{[2-(4-methylphenyl)phenyl]methyl}amine

Using the procedure from example 46, step D, ({2-chloro-4-[([(2-methoxyphenyl)sulfonyl]{[2-(4-methylphenyl)phenyl]methyl}amino)methyl]phenyl}difluoromethyl)diethoxyphosphino-1-one (0.475 g, 0.7 mmol) and iodotrimethylsilane (0.797 mL, 5.60 mmol) in dry dichloromethane (5 mL) The crude residue was purified by C-18 flash chromatography (MeOH/H₂O, 100% H₂O to 6:4) to give {[4-(difluorophosphonomethyl)-3-chlorophenyl]methyl}[(2-methoxyphenyl)sulfonyl]{[2-(4-methylphenyl)phenyl]methyl}amine (0.290 g) in 67% yield as white solid. MS (M+NH₄)⁺ 639,640; (M−H)⁻ 620,622.

Using the procedure from example 46, step C, [(2-methoxyphenyl)sulfonyl]{[2-(4-methylphenyl)phenyl]methyl}amine (0.367 g, 1.00 mmol) in 5 mL of dry DMF, potassium tert-butoxide (1.00 mL, 1.00 mmol, 1M in tert-butanol), and {[4-(bromomethyl)-2-chlorophenyl]difluoromethyl}diethoxyphosphino-1-one (0.391 g, 1.00 mmol) were reacted. The crude residue was purified by flash chromatography (ethyl acetate/hexanes, 1:2) to isolate ({2-chloro-4-[([(2-methoxyphenyl)sulfonyl]{[2-(4-methylphenyl)phenyl]methyl}amino)methyl]phenyl}difluoromethyl)diethoxyphosphino-1-one (0.479 g) in 71% yield as yellow oil. MS (M+NH₄)⁺ 694.

Using the procedure from example 46, step B, 4-tolylboronic acid (0.272 g, 2.00 mmol), [(2-bromophenyl)methyl][(2-methoxyphenyl)sulfonyl]amine (0.534 g, 1.50 mmol), bis(triphenylphosphine)palladium (II) chloride (0.07 g, 0.1 mmol), 1M Na₂CO₃ (in water) (5 mL), acetonitrile (5 mL) and a magnetic stir bar. The crude residue was purified by flash chromatography (ethyl acetate/hexanes, 1:2) to yield 0.675 g (92%) of, [(2-methoxyphenyl)sulfonyl]{[2-(4-methylphenyl)phenyl]methyl}amine as a brown solid. MS (M+H)⁺ 368.

### Example 39

### {[4-(difluorophosphonomethyl)-3-chlorophenyl]methyl}{[2-(3-ethylphenyl)phenyl]methyl}[(2-methoxyphenyl)sulfonyl]amine

Using the procedure from example 46, step D, ({2-chloro-4-[({[2-(3-ethylphenyl)phenyl]methyl}[(2-methoxyphenyl)sulfonyl]amino)methyl]phenyl}difluoromethyl)diethoxyphosphino-1-one (0.326 g, 0.47 mmol) and iodotrimethylsilane (0.536 mL, 3.76 mmol) in dry dichloromethane (5 mL) The crude residue was purified by C-18 flash chromatography (MeOH/H₂O, 100% H₂O to 6:4) to give {[4-(difluorophosphonomethyl)-3-chlorophenyl]methyl}{[2-(3-ethylphenyl)phenyl]methyl}[(2-methoxyphenyl)sulfonyl]amine (0.216 g) in 72% yield as white solid. MS (M+NH₄)⁺ 653; 655(M−H)⁻ 634,636.

Using the procedure from example 46, step C, {[2-(3-ethylphenyl)phenyl]methyl}[(2-methoxyphenyl)sulfonyl]amine (0.719 g, 1.88 mmol) in 8 mL of dry DMF, potassium tert-butoxide (1.88 mL, 1.88 mmol, 1M in tert-butanol), and {[4-(bromomethyl)-2-chlorophenyl]difluoromethyl}diethoxyphosphino-1-one (0.738 g, 1.88 mmol) were reacted. The crude residue was purified by flash chromatography (ethyl acetate/hexanes, 1:2) to isolate ({2-chloro-4-[({[2-(3-ethylphenyl)phenyl]methyl}[(2-methoxyphenyl)sulfonyl]amino)methyl]phenyl}difluoromethyl)diethoxyphosphino-1-one (0.921 g) in 70% yield as yellow oil. MS (M+H)⁺ 693.

Using the procedure from example 46, step B, 3-ethylphenylboronic acid (0.300 g, 2.00 mmol), [(2-bromophenyl)methyl][(2-methoxyphenyl)sulfonyl]amine (0.712 g, 2.00 mmol), bis(triphenylphosphine)palladium (II) chloride (0.07 g, 0.1 mmol), 1M Na₂CO₃ (in water) (5 mL), acetonitrile (5 mL) and a magnetic stir bar. The crude residue was purified by flash chromatography (ethyl acetate/hexanes, 1:4) to yield 0.759 g (99%) of {[2-(3-ethylphenyl)phenyl]methyl}[(2-methoxyphenyl)sulfonyl]amine as a yellow oil. MS (M−H)⁻ 380.

A solution of 1-bromo-3-ethylbenzene (2.80 g, 15 mmol) and tetrahydrofuran (40 mL) were stirred at −78° C. under nitrogen. N-butyllithium (10 mL, 16 mmol) was added and stirred for 10 mins. Triisopropyl borate in THF (6.92 mL, 30 mmol) was added and allowed to come to room temperature. 1M HCl (50 mL) was added and extract with ether (100 mL). Ether was dried over Na₂SO₄, filtered, and dried. The crude residue was recystallized in water, and washed in hexanes to yield 0.591 g (26%) of 3-ethylphenylboronic acid. MS (M+ACN)⁺ 195.

### Example 40

### {[4-(difluorophosphonomethyl)-3-chlorophenyl]methyl}{[2-(4-methoxyphenyl)phenyl]methyl}[(2-methoxyphenyl)sulfonyl]amine

Using the procedure from example 46, step D, ({2-chloro-4-[({[2-(4-methoxyphenyl)phenyl]methyl}[(2-methoxyphenyl)sulfonyl]amino)methyl]phenyl}difluoromethyl)diethoxyphosphino-1-one (0.340 g, 0.489 mmol), bis(trimethylsilyl)trifluoroacetamide (1.6 mL, 5.87 mmol) and iodotrimethylsilane (0.782 mL, 3.91 mmol) in dry dichloromethane (4 mL) The crude residue was purified by C-18 flash chromatography (MeOH/H₂O, 100% H₂O to 6:4) to give {[4-(difluorophosphonomethyl)-3-chlorophenyl]methyl}{[2-(4-methoxyphenyl)phenyl]methyl}[(2-methoxyphenyl)sulfonyl]amine (0.27 g) in 87% yield as white solid. MS (M+NH₄)⁺ 655, 657; (M−H)⁻ 636.

Using the procedure from example 46, step C {[2-(4-methoxyphenyl)phenyl]methyl}[(2-methoxyphenyl)sulfonyl]amine (0.272 g, 0.71 mmol) in 5 mL of dry DMF, potassium tert-butoxide (0.8 mL, 0.8 mmol, 1M in tert-butanol), and {[4-(bromomethyl)-2-chlorophenyl]difluoromethyl}diethoxyphosphino-1-one (0.277 g, 0.71 mmol) were reacted. The crude residue was purified by flash chromatography (ethyl acetate/hexanes, 1:1) to isolate ({2-chloro-4-[({[2-(4-methoxyphenyl)phenyl]methyl}[(2-methoxyphenyl)sulfonyl]amino)methyl]phenyl}difluoromethyl)diethoxyphosphino-1-one (0.350 g) in 71% yield as yellow oil. MS (M+H)⁺ 694.

Using the procedure from example 46, step B, 4-methoxyphenylboronic acid (0.304 g, 2.00 mmol), [(2-bromophenyl)methyl][(2-methoxyphenyl)sulfonyl]amine (0.712 g, 2.00 mmol), bis(triphenylphosphine)palladium (II) chloride (0.07 g, 0.1 mmol), 1M Na₂CO₃ (in water) (5 mL), acetonitrile (5 mL) and a magnetic stir bar. The crude residue was purified by flash chromatography (ethyl acetate/hexanes, 1:2) to yield 0.305 g of {[2-(4-methoxyphenyl)phenyl]methyl}[(2-methoxyphenyl)sulfonyl]amine as a yellow oil. MS (M−H)⁻ 382.

### Example 41

### [(4-{[Benzyl-(2-carbamoyl-benzenesulfonyl)-amino]-methyl}-2-chloro-phenyl)-difluoromethyl]-phosphonic acid

[(2-Chloro-4-methyl-phenyl)-difluoro-methyl]-phosphonic acid diethyl ester

To a stirred suspension of activated Zn dust (25.3 g, 0.39 mol) in dry DMA (150 mL) was slowly added a solution of diethyl (bromodifluoromethyl)phosphonate (103.6 g, 0.39 mmol) in DMA (75 mL) in a 1-L flask. During the addition, an exothermic reaction occurred. The addition was controlled so that the internal temperature was maintained at 50-60° C. After the addition was completed, the solution was stirred at room temperature for an additional 3 hours, and then CuBr (55.7 g, 0.39 mol) was added in one portion. The mixture was stirred at the same temperature for 30 minutes to give the organocopper reagent. Methyl 3-chloro-4-iodobenzoate (49 g, 0.194 mol) in DMA (65 mL) was added dropwise at room temperature (exothermic reaction). After being stirred >18 hours at room temperature, the mixture was portioned between water and ether. The biphasic mixture was passed through Celite and extracted with ether. The extract was washed with brine and dried over MgSO₄. The ether was removed by rotary evaporation, and the residue purified by silica chromatography (1:9 ethyl acetate/hexanes Yield 38 g, 62%, of clear yellow oil. MS (M+H)⁺ 313.

[(4-Bromomethyl-2-chloro-phenyl)-difluoro-methyl]-phosphonic acid diethyl ester

N-Bromosuccinimide (23 g, 0.13 mol) and AIBN (1 g, 6 mmol) were added to a solution of [(2-Chloro-4-methyl-phenyl)-difluoro-methyl]-phosphonic acid diethyl ester (38.3 g, 0.122 mol) in 1 liter of benzene. The mixture was warmed to reflux, and stirred at reflux overnight. The mixture was cooled, solids removed by filtration, and volatiles removed under vacuum. The residue was purified by silica column chromatography. Recovered 46 g of yellow oil which contained 62% by weight of product. (Impurities included dibromo compound and starting material). Used without further purification. MS (M+H)⁺ 392.

2-Benzyl-1,1-dioxo-1,2-dihydro-1lambda*6*-benzo[d]isothiazol-3-one

Sodium hydride (440 mg of 60% in mineral oil, 11 mmol) was added to a stirred solution of saccharin (1.83 g, 10 mmol) in DMF. Benzyl bromide (2.0 g, 11.7 mmol) was added dropwise to the stirred mixture. Upon completion of reaction, the mixture was taken up in ethyl acetate and washed with 0.1 M HCl, sat'd sodium bicarbonate, and sat'd brine. The solvent was removed under vacuum, and the residue purified by column chromatography, yielding white solids (2.57 g, 94%) MS (M+H)⁺ 274.

2-Benzylsulfamoyl-benzamide

2-Benzyl-1,1-dioxo-1,2-dihydro-1lambda*6*-benzo[d]isothiazol-3-one (273 mg, 1 mmol) was dissolved in 6 mL of 2M ammonia in methanol and heated at 60° C. overnight. The volatiles were removed, and the solid residue was purified on silica (19:1 DCM/methanol). Recovered white solids (219 mg, 75% yield). MS (M+H)⁺ 291.

### [(4-{[Benzyl-(2-carbamoyl-benzenesulfonyl)-amino]-methyl}-2-chloro-phenyl)-difluoro-methyl]-phosphonic acid diethyl ester

[(4-Bromomethyl-2-chloro-phenyl)-difluoro-methyl]-phosphonic acid diethyl ester was added dropwise under nitrogen to a stirred mixture of 2-Benzylsulfamoyl-benzamide (74 mg, 0.25 mmol) and potassium carbonate (69 mg, 0.5 mmol) in DMF (2 mL). Upon completion of reaction as indicated by TLC, the mixture was taken up in ethyl acetate, washed with 0.1M HCl, sat'd sodium bicarbonate, and sat'd brine. After rotary evaporation, the oily solids were recrystallized from ethyl acetate/hexane, yielding white crystals (119 mg, 79% yield). MS (M+H)⁺ 601.

[(4-{[Benzyl-(2-carbamoyl-benzenesulfonyl)-amino]-methyl}-2-chloro-phenyl)-difluoromethyl]-phosphonic acid

A solution of [(4-{[Benzyl-(2-carbamoyl-benzenesulfonyl)-amino]-methyl}-2-chloro-phenyl)-difluoro-methyl]-phosphonic acid diethyl ester (116 mg, 0.193 mmol) in dichloromethane (2 mL) was chilled to −20° C. Trimethylsilyl iodide (0.28 ml, 2.0 mmol) was added dropwise with stirring, and the mixture was allowed to warm to 0° C. over 1 hour. The solvent was removed by rotary evaporation. The resulting oil was dissolved in 5 ml of 4:1 acetonitrile/water, and TFA (0.5 mL) was added. The reaction was reduced to an oil by rotary evaporation, taken up in ethyl acetate, and washed with acidified sodium thiosulfate solution, saturated brine, and dried under vacuum. The residue was purified by RP column chromatography to yield white solid (77 mg, 73% yield). MS (M+H)⁺ 544.

### Example 42

### Ethyl (2S)-2-{[({2-chloro-4-[({[4-(3-cyanophenyl)phenyl]methyl}[(2-methoxyphenyl)sulfonyl]amino)methyl]phenyl}difluoromethyl)(hydroxyphosphoryl)]amino}propanoate

To a solution of 3-{4-[({[4-(difluorophosphonomethyl)-3-chlorophenyl]methyl}[(2-methoxyphenyl)sulfonyl]amino)methyl]phenyl}benzenecarbonitrile (0.150 g, 0.24 mmol) and L-alanine ethyl ester hydrochloride (0.221 g, 1.44 mmol) in 10 mL of t-BuOH was added EDC (0.331 g, 1.73 mmol), followed by 1 N NaOH— (3.65 mL, 3.65 mmol at 0° C. The reaction mixture was refluxed overnight under nitrogen. The reaction mixture was neutralized with 1 N HCl solution and extracted with ethyl acetate. The organic layer was washed with brine, dried (Na₂SO₄), and evaporated. The crude residue was purified by C-18 flash chromatography (MeOH/H₂O, 100% H₂O to 65:35) to give 0.006 g (3%) of title compound as light yellow solid. MS (M+H)⁺ 732, 734; MS (M+NH₄)⁺ 749, 751; (M−H)⁻ 730,732.

LC condition: Column: Capcell-pak 3μ C18 UG120A 0-3.5 min. 100% of A to 100% of B; 3.5-4.0 min. A: 95/5 (H₂O/CH₃CN) contained 5 mM Ammonium formate B: CH₃CN

### Example 43

### [({2-chloro-4-[({[4-(3-cyanophenyl)phenyl]methyl}[(2-methoxyphenyl)sulfonyl]amino)methyl]phenyl}difluoromethyl)(hydroxyphosphoryl)oxy]ethyl(methylethoxy)formate, sodium salt

A mixture of 3-{4-[({[4-(difluorophosphonomethyl)-3-chlorophenyl]methyl}[(2-methoxyphenyl)sulfonyl]amino)methyl]phenyl}benzenecarbonitrile (0.788 g, 1.24 mmol), 1-chloroethyl isopropyl carbonate (2.074 g, 12.45 mmol) and Cs₂CO₃ (0.811 g, 2.49 mmol) in CH₃CN (20 mL) was refluxed for 18 h. The reaction mixture was partitioned between saturated NH₄Cl and ethyl acetate. The organic phase was separated, dried over Na₂SO₄ and evaporated under reduced pressure. The crude residue was dissolved in 15 mL of saturated NaHCO₃ and 15 mL of MeOH. The mixture was stirred overnight at room temperature and purified by C-18 flash chromatography (MeOH/H₂O, 100% H₂O to 50:50) to give 0.495 g (51%) of title compound as white solid. MS (M+NH₄)⁺ 780, 782; (M−H)⁻ 761,763.

LC condition: Column: Capcell-pak 3μ C18 UG120A 0-3.5 min. 100% of A to 100% of B; 3.5-4.0 min. A: 95/5 (H₂O/CH₃CN) contained 5 mM Ammonium formate B: CH₃CN

### Example 44

### [[(2,2-dimethylpropanoyloxy)methoxy]({2-chloro-4-[({[4-(3-cyanophenyl)phenyl]methyl}[(2-methoxyphenyl)sulfonyl]amino)methyl]phenyl}difluoromethyl)carbonyloxy]methyl 2,2-dimethylpropanoate

### and

### [({2-chloro-4-[({[4-(3-cyanophenyl)phenyl]methyl}[(2-methoxyphenyl)sulfonyl]amino)methyl]phenyl}difluoromethyl)(hydroxyphosphoryl)oxy]methyl 2,2-dimethylpropanoate, sodium salt

A mixture of 3-{4-[({[4-(difluorophosphonomethyl)-3-chlorophenyl]methyl}[(2-methoxyphenyl)sulfonyl]amino)methyl]phenyl}benzenecarbonitrile (0.100 g, 0.16 mmol), chloromethyl pivalate (0.238 g, 1.58 mmol) and Cs₂CO₃ (0.103 g, 0.32 mmol) in CH₃CN (5 mL) was refluxed for 5 h. The reaction mixture was partitioned between saturated NH₄Cl and ethyl acetate. The organic phase was separated, dried over Na₂SO₄ and evaporated under reduced pressure. The crude residue was suspended in 10 mL of saturated NaHCO₃ and purified by C-18 flash chromatography (CH₃CN/H₂O, 100% H₂O to 80:20) to give 0.035 g (25%) of [[(2,2-dimethylpropanoyloxy)methoxy]({2-chloro-4-[({[4-(3-cyanophenyl)phenyl]methyl}[(2-methoxyphenyl)sulfonyl]amino)methyl]phenyl}difluoromethyl)carbonyloxy]methyl 2,2-dimethylpropanoate as white solid. LC: RT, 3.4 min; MS (M+NH₄)⁺ 878, 880; (M−CH₂OC(═O)Me₃)⁻ 745, 747. And 0.065 g (53%) of [({2-chloro-4-[({[4-(3-cyanophenyl)phenyl]methyl}[(2-methoxyphenyl)sulfonyl]amino)methyl]phenyl}difluoromethyl)(hydroxyphosphoryl)oxy]methyl 2,2-dimethylpropanoate, sodium salt as white solid. LC: RT, 2.4 min; MS (M+NH₄)⁺ 764, 766; (M−H)⁻ 745, 747.

LC condition: Column: Capcell-pak 3μ C18 UG120A 0-3.5 min. 100% of A to 100% of B; 3.5-4.0 min. A: 95/5 (H₂O/CH₃CN) contained 5 mM Ammonium formate B: CH₃CN

### Example 45

### [({2-chloro-4-[({[4-(3-cyanophenyl)phenyl]methyl}[(2-methoxyphenyl)sulfonyl]amino)methyl]phenyl}difluoromethyl)(hydroxyphosphoryl)oxy]ethyl(methylethoxy)formate, sodium salt

A mixture of 3-{4-[({[4-(difluorophosphonomethyl)-3-chlorophenyl]methyl}[(2-methoxyphenyl)sulfonyl]amino)methyl]phenyl}benzenecarbonitrile (0.788 g, 1.24 mmol), 1-chloroethyl isopropyl carbonate (2.074 g, 12.45 mmol) and Cs₂CO₃ (0.811 g, 2.49 mmol) in CH₃CN (20 mL) was refluxed for 18 h. The reaction mixture was partitioned between saturated NH₄Cl and ethyl acetate. The organic phase was separated, dried over Na₂SO₄ and evaporated under reduced pressure. The crude residue was dissolved in 15 mL of saturated NaHCO₃ and 15 mL of MeOH. The mixture was stirred overnight at room temperature and purified by C-18 flash chromatography (MeOH/H₂O, 100% H₂O to 50:50) to give 0.495 g (51%) of title compound as white solid. MS (M+NH₄)⁺ 780, 782; (M−H)⁻ 761,763.

### Example 46

### [[(2,2-dimethylpropanoyloxy)methoxy]({2-chloro-4-[({[4-(3-cyanophenyl)phenyl]methyl}[(2-methoxyphenyl)sulfonyl]amino)methyl]phenyl}difluoromethyl)carbonyloxy]methyl 2,2-dimethylpropanoate

### Example 47

### [({2-chloro-4-[({[4-(3-cyanophenyl)phenyl]methyl}[(2-methoxyphenyl)sulfonyl]amino)methyl]phenyl}difluoromethyl)(hydroxyphosphoryl)oxy]methyl 2,2-dimethylpropanoate, sodium salt

A mixture of 3-{4-[({[4-(difluorophosphonomethyl)-3-chlorophenyl]methyl}[(2-methoxyphenyl)sulfonyl]amino)methyl]phenyl}benzenecarbonitrile (0.100 g, 0.16 mmol), chloromethyl pivalate (0.238 g, 1.58 mmol) and Cs₂CO₃ (0.103 g, 0.32 mmol) in CH₃CN (5 mL) was refluxed for 5 h. The reaction mixture was partitioned between saturated NH₄Cl and ethyl acetate. The organic phase was separated, dried over Na₂SO₄ and evaporated under reduced pressure. The crude residue was suspension in 10 mL of saturated NaHCO₃ and purified by C-18 flash chromatography (CH₃CN/H₂O, 100% H₂O to 80:20) to give 0.035 g (25%) of [[(2,2-dimethylpropanoyloxy)methoxy]({2-chloro-4-[({[4-(3-cyanophenyl)phenyl]methyl}[(2-methoxyphenyl)sulfonyl]amino)methyl]phenyl}difluoromethyl)carbonyloxy]methyl 2,2-dimethylpropanoate as white solid. LC: RT, 3.4 min; MS (M+NH₄)⁺ 878, 880; (M−CH₂OC(═O)Me₃)⁻ 745, 747. And 0.065 g (53%) of [({2-chloro-4-[({[4-(3-cyanophenyl)phenyl]methyl}[(2-methoxyphenyl)sulfonyl]amino)methyl]phenyl}difluoromethyl)(hydroxyphosphoryl)oxy]methyl 2,2-dimethylpropanoate, sodium salt as white solid. LC: RT, 2.4 min; MS (M+NH₄)⁺ 764, 766; (M−H)⁻ 745, 747.

### Example 48

A 5× stock of pNPP (p-nitrophenol phosphate) substrate is prepared as 50 mM pNPP in assay buffer. Various tyrosine phosphatase solutions can be prepared as follows:
PTP-1B (purified, 1 mg/mL) as a 1:250 dilution (to a final concentration of 4:g/mL);
TC-PTP (NEB, 1000 units in 100:L) as a 1:50 dilution (to a final concentration of 2U/10:L (4:g/mL));
CD45 (Calbiochem, 20:g, 400 units in 100:L) as a 1:50 dilution (to a final concentration of 0.8U/10:L (4:g/mL));
LAR (NEB, 1000 units in 200:L) as a 1:75 dilution (to a final concentration of 0.7U/10:L (4:g/mL)); and
PTP-β (UBI, #14-350, 10,000 units, 40:g/571:L) as a 1:17.5 dilution (to a final concentration of 10U/10:L (4:g/mL));

The compound to be tested is prepared as 1:16.7 and 1:50 dilutions from stock in a total volume of 100:M DMSO to give final concentrations of 626 and 200:M. The reaction mixtures are prepared in a 96-well microtiter plate (on ice) as 55:L assay buffer, 5:L of the diluted compound (to a final concentration of 31.3 and 10:M), 20:L of the pNPP substrate solution (to a final concentration of 10 mM) and 20:L PTPase in assay buffer. The reactants are mixed well, the plate placed in a water bath at 30° C. and incubated for 10 minutes. The reaction is then terminated by adding 100:L of 2M K₂CO₃ per well, and the absorbance is measured at 405 nm by conventional means.

Unless otherwise indicated, this assay was used to determine activity for the selected compounds whose activity is provided in Table 1.

### Example 49

Cell Based Assays

Antibodies and Chemicals. The antibody against phosphorylated insulin receptor (pIR) and the ELISA kit for detection of pIR were from Biosource (Camarillo, Calif.). Rabbit anti-IR/IGF-IR [pYpY1162/1163] phosphospecific antibody recognizes both the insulin receptor (IR) and the insulin-like growth factor-1 receptor (IGF-1R) phosphorylated at the active site tyrosine residues, 1162 and 1163 (1135 and 1136 for IGF-1R) (pIR/pIGF-1R). The Insulin Receptor [pYpY1162/1163] ELISA kit specifically recognizes IR phosphorylated at tyrosine residues 1162 and 1163 (and does not recognize phosphorylated IGF-1R). HRP-conjugated secondary antibodies were from Cell Signaling Technology (Beverly, Mass.). The ECL detection system was from Amersham (Buckinghamshire, UK), and human insulin was from Invitrogen (Carlsbad, Calif.).

Tissue Culture. FAO rat hepatoma cells were obtained from ECACC (#89042701) and maintained at 37° C. in a 5% CO₂ environment in Dulbecco's modified Eagle's medium with high glucose (DMEM-high glucose) (4500 mg/liter) supplemented with 10% FBS and 50 units/ml penicillin, 100 μg/ml streptomycin and 0.292 mg/ml L-glutamine. For assays, cells were seeded in 24-well plates at a density of 2×10⁵ cells/well and maintained until they reached confluency (about 3 days).

IR phosphorylation assays. Cells in 24-well plates were serum starved overnight in DMEM-low glucose (1000 mg/liter) without serum. Just before use, the starvation medium was discarded and replaced with 0.5 ml of DMEM without serum. Cells were treated for 1 hour with indicated concentrations of compounds, followed by stimulation with or without insulin for 15-30 minutes. For Western analysis, the reaction was stopped by discarding the medium and adding 80 μl of boiling SDS sample lysis buffer [62.5 mM Tris-HCl (pH 6.8), 50 mM DTT, 2% w/v SDS, 10% glycerol, 50 mM NaF, 1 mM Na₃VO₄, 2 mM pNPP, 20 mM β-glycerol phosphate and 0.1% w/v bromophenol blue]. 20 μl of the lysates were loaded onto 4-20% Tris-Glycine grandient gels (Invitrogen, Carlsbad, Calif.) and the proteins resolved by SDS-PAGE and transferred to nitrocellulose membranes. The membranes were probed for detection of pIR/pIGF-1Rand total PTP-1B using the ECL chemiluminescence detection system. The pIR/pIGF-1R signals were scanned (HP scanjet 3570c) and quantified (Scion Image). For ELISA analysis, the medium was discarded and the plates placed onto a dry ice/ethanol bath for 3 minutes to stop the reaction, then placed on ice. The cells were then lysed and processed according to the ELISA instruction kit manuals for detection of pIR and pAkt (Biosource, Camarillo, Calif.)

What is claimed is:

1. A compound that has the formula I: 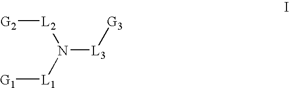
or a pharmaceutically acceptable derivative thereof, wherein:
L₁, L₂ and L₃ are independently selected from:
N—C single bond (i.e. G₁, G₂, or G₃ are directly bonded to N by a single bond), alkylene, alkenylene, alkynylene, cycloalkylene, oxocycloalkylene, amidocycloalkylene, heterocyclylene, heteroarylene, C═O, sulfonyl, alkylsulfonyl, alkenylsulfonyl, alkynylsulfonyl, amido, carboxamido, alkylamido, alkylcarboxamido, and alkoxyoxo; and
where each of the above substituents are unsubstituted or substituted with one or more substituents, in one embodiment, one, two, or three substituents, each independently selected from D¹, where D¹ is H, alkyl, alkenyl, alkynyl, aryl, cycloalkyl, heterocyclyl, heteroaryl, amido, cyano, cyanoalkenyl, amidoalkyl, amidoalkenyl, and oxo;
G₁, G₂, and G₃ are independently selected from:
(i) alkyl, alkenyl, alkynyl, aryl, alkaryl, arylalkyl, alkarylalkyl, alkenylaryl, alkylsulfonyl, alkenylsulfonyl, alkynylsulfonyl, amido, alkylamino, alkylaminoaryl, arylamino, aminoalkyl, aminoaryl, alkoxy, alkoxyaryl, aryloxy, alkylamido, alkylcarboxamido, arylcarboxamido, alkoxyoxo, biaryl, alkoxyoxoaryl, amidocycloalkyl, carboxyalkylaryl, carboxyaryl, carboxyamidoaryl, carboxamido, cyanoalkyl, cyanoalkenyl, cyanobiaryl, cycloalkyl, cycloalkyloxo, cycloalkylaminoaryl, haloalkyl, haloalkylaryl, haloaryl, heterocyclyl, heteroaryl, hydroxyalkylaryl, and sulfonyl; and
(ii) G₁ and G₂ can be linked together to form a cycloalkyl, oxocycloalkyl, cycloalkyloxo, amidocycloalkyl, cycloalkylamido, alkenylaryl, amidoalkenylaryl, and the following groups, 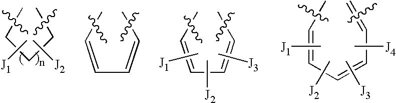
where J₁, J₂, J₃, and J₄ are selected from H, alkyl, alkenyl, alkynyl, aryl, cycloalkyl, heterocyclyl, heteroaryl, amido, cyano, cyanoalkenyl, amidoalkyl, amidoalkenyl, oxo, and CH═C(CN)C(O)NH; and
where each of the above substituents are unsubstituted or substituted with one or more substituents, in one embodiment, one, two, or three substituents, each independently selected from M¹, where M¹ is H, alkyl, alkenyl, alkynyl, aryl, arylalkyl, alkoxy, alkoxyoxo, alkylthia, amino, amido, arylamino, aryloxy, alkylamino, alkylsulfonyl, alkylcarboxyalkylphosphonato, arylcarboxamido, carboxy, carboxyoxo, carboxyalkyl, carboxyalkyloxa, carboxyalkenyl, carboxyamido, carboxyhydroxyalkyl, cycloalkyl, amido, cyano, cyanoalkenyl, cyanoaryl, amidoalkyl, amidoalkenyl, halo, haloalkyl, haloalkylsulfonyl, heterocyclyl, heteroaryl, heteroarylalkyl, heteroarylalkoxy, hydroxy, hydroxyalkyl, hydroxyamino, hydroxyimino, heteroarylalkyloxa, nitro, phosphonato, phosphonatoalkyl, and phosphonatohaloalkyl.

2. The compound of claim 1, wherein:
L₁, L₂ and L₃ are independently selected from:
N—C single bond (i.e. G1, G2, or G3 are directly bonded to N by a single bond), (CRR1)ₘ, CF₂, CF₂CF₂, C(═O), C(═O)C(═O), C(═O)(CRR1)ₘ, (CRR1)ₘC(═O)(CRR1)ₘ, C(═O)O(CRR1)ₘ, (CRR1)ₘC(═O)O, N(R), —C(═O)N(R)N(R1), N(R)SO₂N(R1), C(═O)N(R), N(R)C(═O)N(R1), O, OC(═O)N(R), P(═O)(OR), P(═O)(NR), P(═S)(OR), P(═S)(NR), SO₂, S(═O)ₙ(CRR1)ₘ, (CRR1)ₘS(═O)ₙ(CRR1)ₘ, where m=0-6 and n=0-2, S(═O)(═NR), S(═NR)(═NR1), SO₂NR, wherein R and R1 are independently selected from hydrogen, C1-C6 alkyl which is optionally substituted with 1 to 3 substituents selected from the group consisting of Y₁, Y₂, and Y₃, —OC(R2R3)OC(═O)R4, —OC(R2R3)OC(═O)OR4, where R2, R3 and R4 are independently selected from H, C₁-C₇ alkyl, R2, R3 and R4 can be combined to form a 5-7-membered ring, C2-C6 alkenyl which is optionally substituted with 1 to 3 substituents selected from the group consisting of Y₁, Y₂, and Y₃, C2-C6 alkynyl which is optionally substituted with 1 to 3 substituents selected from the group consisting of Y₁, Y₂, and Y₃, C3-C8 cycloalkyl which is optionally substituted with 1 to 3 substituents selected from the group consisting of Y₁, Y₂, and Y₃, C6-C14 aryl which is optionally substituted with 1 to 3 substituents selected from the group consisting of Y₁, Y₂, and Y₃, C10-C20 linked biaryl and heterobiaryl, which is optionally substituted with 1 to 3 substituents selected from the group consisting of Y₁, Y₂, and Y₃, C7-C16 aralkyl, which is optionally substituted with 1 to 3 substituents selected from the group consisting of Y₁, Y₂, and Y₃,
C5-C14 monocyclic-heteroaryl and bicyclic-heteroaryl, which is optionally substituted with 1 to 3 substituents selected from the group consisting of Y₁, Y₂, and Y₃, and C5-C14 heteroaralkyl which is optionally substituted on the alkyl chain and on the ring with 1 to 3 substituents selected from the group consisting of Y₁, Y₂, and Y₃, R and R1 can be joined together to form an alicyclic or heterocyclic ring;
R and R1 are independently and optionally substituted with 1 to 3 substituents Y₁, Y₂, and Y₃.

3. The compound of claim 1, wherein:
L₁, L₂ and L₃ are independently selected from:
N—C single bond (i.e. G₁, G₂, or G₃ are directly bonded to N by a single bond), alkylene, alkenylene, alkynylene, cycloalkylene, oxocycloalkylene, amidocycloalkylene, heterocyclylene, heteroarylene, C═O, sulfonyl, alkylsulfonyl, alkenylsulfonyl, alkynylsulfonyl, amido, carboxamido, alkylamido, alkylcarboxamido, and alkoxyoxo; where each of the above substituents are unsubstituted or substituted with one or more substituents, in one embodiment, one, two, or three substituents, each independently selected from D¹, where D¹ is H, alkyl, alkenyl, alkynyl, cycloalkyl, heterocyclyl, heteroaryl, amido, cyano, cyanoalkenyl, amidoalkyl, amidoalkenyl, and oxo.

4. The compound of claim 1, wherein:
L₁, L₂ and L₃ are independently selected from:
N—C single bond, C1-C5 alkylene, C1-C5 alkenylene, C1-C5 alkynylene, C3-C15 cycloalkylene, C3-C15 amidocycloalkylene, C3-C15 heterocyclylene, C3-C15 heteroarylene, oxo, sulfonyl, alkylsulfonyl, alkenylsulfonyl, alkynylsulfonyl, amido, carboxamido, alkylamido, alkylcarboxamido, and alkoxyoxo; where each of the above substituents are unsubstituted or substituted with one or more substituents, in one embodiment, one, two, or three substituents, each independently selected from D¹, where D¹ is H, alkyl, alkenyl, alkynyl, cycloalkyl, heterocyclyl, heteroaryl, amido, cyano, cyanoalkenyl, amidoalkyl, amidoalkenyl, and oxo.

5. The compound of claim 1, wherein:
L₁, L₂ and L₃ are independently selected from:
N—C single bond, C1-C5 alkylene, C1-C5 alkenylene, C1-C5 alkynylene, C3-C15 cycloalkylene, C3-C15 amidocycloalkylene, C3-C15 heterocyclylene, C3-C15 heteroarylene, oxo, sulfonyl, alkylsulfonyl, alkenylsulfonyl, alkynylsulfonyl, amido, carboxamido, alkylamido, alkylcarboxamido, and alkoxyoxo.

6. The compound of claim 1, wherein:
L₁, L₂ and L₃ are independently selected from:
N—C single bond, —CH₂, —C(═O), —CH₂CH₂, —SO₂, —S(═O)₂CH₂, —S(═O)₂CH₂CH₂, —C(═O)NHCH₂, —C(═O)OCH₂, and —S(═O)₂CH═CH,

7. The compound of claim 1, wherein:
L₁, L₂ or L₃ is independently N—C single bond.

8. The compound of claim 1, wherein:
L₁, L₂ or L₃ is independently —CH₂.

9. The compound of claim 1, wherein:
L₁, L₂ or L₃ is independently —CH₂CH₂.

10. The compound of claim 1, wherein:
L₁, L₂ or L₃ is independently —C(═O)NHCH₂.

11. The compound of claim 1, wherein:
L₁, L₂ or L₃ is independently —C(═O).

12. The compound of claim 1, wherein:
L₁, L₂ or L₃ is independently —C(═O)OCH₂.

13. The compound of claim 1, wherein:
L₁, L₂ or L₃ is independently —SO₂.

14. The compound of claim 1, wherein:
L₁, L₂ or L₃ is independently —S(═O)₂CH₂.

15. The compound of claim 1, wherein:
L₁, L₂ or L₃ is independently —S(═O)₂CH₂CH₂.

16. The compound of claim 1, wherein:
L₁, L₂ or L₃ is independently —S(═O)₂CH═CH.

17. The compound of claim 1, wherein:
G₁, G₂ and G₃ are independently selected from:
H, C1-6 alkyl and which is optionally substituted with 1 to 3 substituents selected from the group consisting of Y₁, Y₂, and Y₃, C2-C6 alkenyl which is optionally substituted with 1 to 3 substituents selected from the group consisting of Y₁, Y₂, and Y₃, C2-C6 alkynyl which is optionally substituted with 1 to 3 substituents selected from the group consisting of Y₁, Y₂, and Y₃; C3-C8 cycloalkyl which is optionally substituted with 1 to 3 substituents selected from the group consisting of Y₁, Y₂, and Y₃; C6-C14 aryl which is optionally substituted with 1 to 3 substituents selected from the group consisting of Y₁, Y₂, and Y₃; C7-C16 aralkyl which is optionally substituted with 1 to 3 substituents selected from the group consisting of Y₁, Y₂, and Y₃; C5-C14 heteroaryl, which is optionally substituted with 1 to 3 substituents selected from the group consisting of Y₁, Y₂, and Y₃, and C5-C14 heteroaralkyl, which is optionally substituted on the ring and the alkyl chain with 1 to 3 substituents selected from the group consisting of Y₁, Y₂, and Y₃; and
Y₁, Y₂, and Y₃ are selected from R, (CRR1)ₙOR, OH, (CRR1)ₙNRR1, C(═NR)NRR1, C(═NOR)NRR1, halogen (F, Cl, Br, I), cyano, nitro, CF₃, CF₂CF₃, CH₂CF₃, CH(CF₃)₂, C(OH)(CF₃)₂, OCHCl₂, OCF₃, OCF₂H, OCF₂CF₃, OCH₂CF₃, (CRR1)ₙOC(═O)NRR1, (CRR1)ₙNHC(═O)C(═O)OR, (CRR1)ₙNHC(═O)NRSO₂(Me, CF₃), (CRR1)ₙNHSO₂(Me, CF₃), (CRR1)ₙNHSO₂NRR1, NHSO₂NRC(═O)(Me, CF₃), (CRR1)ₙNHC(═O)R, (CRR1)ₙNHC(═O)NRR1, C(═O)OH, (CRR1)ₙC(═O)OH, C(═O)OR, C(═O)O(CRR1)OC(═O)R, C(═O)O(CRR1)OC(═O)OR, C(═O)R, —(CRR1)ₙC(═O)R, (CF₂)ₙC(═O)R, (CFR)ₙC(═O)R, tetrazolyl (Tzl), (CRR1)ₙTzl, (CF₂)ₙTzl, (CFR)ₙTzl, (CRR1)ₙC(═O)OR, (CRR1)ₙC(═O)NH₂, (CRR1)ₙC(═O)NRR1, (CRR1)ₙC(═O)C(═O)OR, (CRR1)ₙCH(OR)C(═O)OR, (CF₂)ₙC(═O)OH, (CF₂)ₙC(═O)OR, (CF₂)ₙC(═O)NH₂, (CF₂)ₙC(═O)NRR1, (CRR1)ₙC(═O)C(═O)OR, (CRR1)ₙCH(OR)C(═O)OR, C(R)═C(R1), C(═O)OR, C(R)═C(R1)-Tzl, (CRR1)ₙP(═O)(OH)₂, (CRR1)ₙP(═O)(OR)(OR1), P(═O)(OR)[(OCRR1)OC(═O)R], P(═O)(OR)[(OCRR1)OC(═O)OR], P(═O)[(OCRR1)OC(═O)R)][(OCRR1)OC(═O)R], P(═O)[(OCRR1)OC(═O)OR)][(OCRR1)OC(═O)OR], (CRR1)ₙP(═O)(Me)(OR), (CRR1)ₙP(═O)(CF₃)(OR), (CF₂)ₙP(═O)(OR)(OR1), (CF₂)ₙP(═O)(Me)(OR), (CF₂)ₙP(═O)(CF₃)(OR), (CFR)_{q}P(═O)(OR)(OR1), CR═CR—P(═O)(OR)(OR1), CR═CR—P(═O)(Me)(OR), CC—P(═O)(OR)(OR1), (C═O)P(═O)(OR)(OR1), (C═O)P(═O)(Me)(OR), (C═O)P(═O)(CF₃)(OR), (CROR1)ₙP(═O)(OR)(OR1), (CROR1)ₙP(═O)(Me)(OR), (CROR1)ₙP(═O)(CF₃)(OR), O(CRR1)ₙC(═O)OR, O(CF₂)ₙC(═O)OR, OCH[C(═O)OR]₂, O(CRR1)ₙCH[C(═O)OR]₂, OCF[C(═O)OR]₂, O(CRR1)ₙC(═O)C(═O)OR, O(CF₂)ₙC(═O)C(═O)OR, O(CRR1)ₙTzl, O(CF₂)ₙTzl, OCH(Tzl)₂, O(CF₂)ₙP(═O)(OR)(OR1), O(CF₂)ₙP(═O)(Me)(OR), O(CF₂)ₙP(═O)(CF₃)(OR), O(CFR)ₙP(═O)(OR)(OR1), O(CFR)ₙP(═O)(Me)(OR), O(CFR)ₙP(═O)(CF₃)(OR), (CRR1)ₙP(═O)(OR)(OR1), O(CRR1)ₙP(═O)(Me)(OR), O(CRR1)ₙP(═O)(CF₃)(OR), OCF[P(═O)(Me)(OR)]₂, SO₃H, —(CRR1)ₙSO₃H, S(O)ₙR, SCF₃, SCHF₂, SO₂CF₃, SO₂Ph, (CRR1)ₙS(O)ₙR, (CRR1)ₙS(O)₂CF₃, (CRR1)ₙSO₂NRR1, (CRR1)ₙSO₂NRC(═O)(Me, CF₃), (CF₂)ₙSO₃H, (CFR)ₙSO₃H, (CF₂)ₙSO₂NRR1, wherein n=0-2, and R and R1 are as defined above; Y₁, Y₂ and/or Y₃ may also be selected together to be (CRR1)₂₋₆ and substituted variants thereof, —O[C(R2)(R3)]ᵣO— or —O[C(R2)(R3)]ᵣ₊₁—, wherein r is an integer from 1 to 4 and R2 and R3 are independently selected from the group consisting of hydrogen, C1-C12 alkyl, C6-14 aryl, C5-C14 heteroaryl, C7-C15 aralkyl, and C5-C14 heteroarylalkyl.

18. The compound of claim 1, wherein:
G₁, G₂, and G₃ are independently selected from: alkyl, alkenyl, alkynyl, aryl, alkaryl, arylalkyl, alkarylalkyl, alkenylaryl, alkylsulfonyl, alkenylsulfonyl, alkynylsulfonyl, amido, alkylamino, alkylaminoaryl, arylamino, aminoalkyl, aminoaryl, alkoxy, alkoxyaryl, aryloxy, alkylamido, alkylcarboxamido, arylcarboxamido, alkoxyoxo, biaryl, alkoxyoxoaryl, amidocycloalkyl, carboxyalkylaryl, carboxyaryl, carboxyamidoaryl, carboxamido, cyanoalkyl, cyanoalkenyl, cyanobiaryl, cycloalkyl, cycloalkyloxo, cycloalkylaminoaryl, haloalkyl, haloalkylaryl, haloaryl, heterocyclyl, heteroaryl, hydroxyalkylaryl, and sulfonyl;
where each of the above substituents are unsubstituted or substituted with one or more substituents, in one embodiment, one, two, or three substituents, each independently selected from M¹, where M¹ is H, alkyl, alkenyl, alkynyl, aryl, arylalkyl, alkoxy, alkoxyoxo, alkylthia, amino, amido, arylamino, aryloxy, alkylamino, alkylsulfonyl, alkylcarboxyalkylphosphonato, arylcarboxamido, carboxy, carboxyoxo, carboxyalkyl, carboxyalkyloxa, carboxyalkenyl, carboxyamido, carboxyhydroxyalkyl, cycloalkyl, amido, cyano, cyanoalkenyl, cyanoaryl, amidoalkyl, amidoalkenyl, halo, haloalkyl, haloalkylsulfonyl, heterocyclyl, heteroaryl, heteroarylalkyl, heteroarylalkoxy, hydroxy, hydroxyalkyl, hydroxyamino, hydroxyimino, heteroarylalkyloxa, nitro, phosphonato, phosphonatoalkyl, and phosphonatohaloalkyl.

19. The compound of claim 1, wherein:
G₁ and G₂ can be linked together to form a cycloalkyl, oxocycloalkyl, cycloalkyloxo, amidocycloalkyl, cycloalkylamido, alkenylaryl, amidoalkenylaryl, and the following groups, 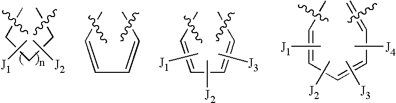
where J₁, J₂, J₃, and J₄ are selected from H, alkyl, alkenyl, alkynyl, cycloalkyl, heterocyclyl, heteroaryl, amido, cyano, cyanoalkenyl, amidoalkyl, amidoalkenyl, oxo, and CH═C(CN)C(O)N.

20. The compound of claim 1, wherein:
G₁ and G₂ can be linked together to form the following groups,

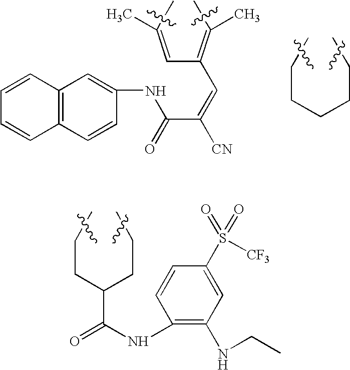

21. The compound of claim 1, wherein:
G₁ and G₂ can be linked together to form

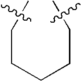

22. The compound of claim 1, wherein:
G₁ and G₂ can be linked together to form.

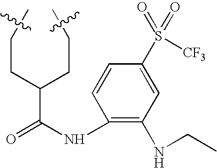

23. The compound of claim 1, wherein:
G₁ and G₂ can be linked together to form

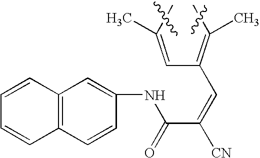

24. The compound of claim 1, wherein:
G₁, G₂, and G₃ are independently selected from, —CH(E¹)₂, —CH═CH(E¹), —CH(E¹)CH₂(E¹), —CH═C(E¹)C(O)NHE¹, 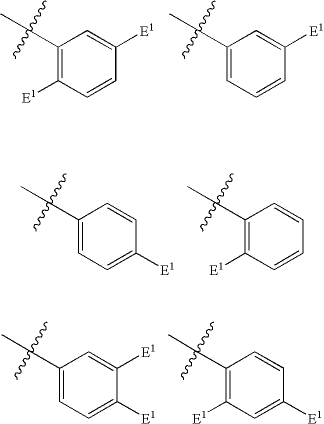
where E¹ at each instance is independently selected from —CN, —OCH₃, —COOH, Cl, F, Br, CH₃,

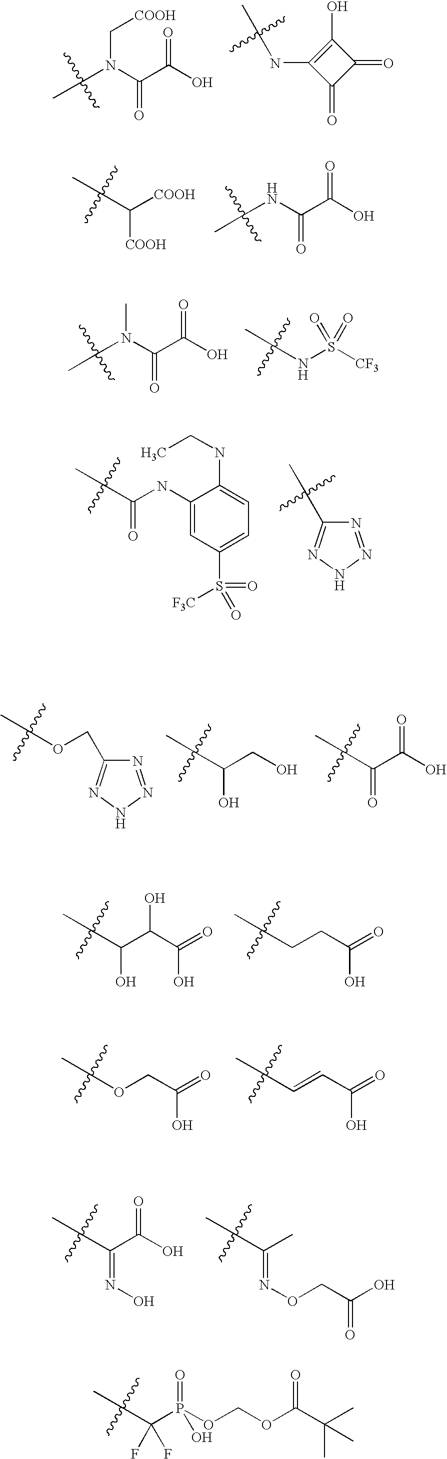

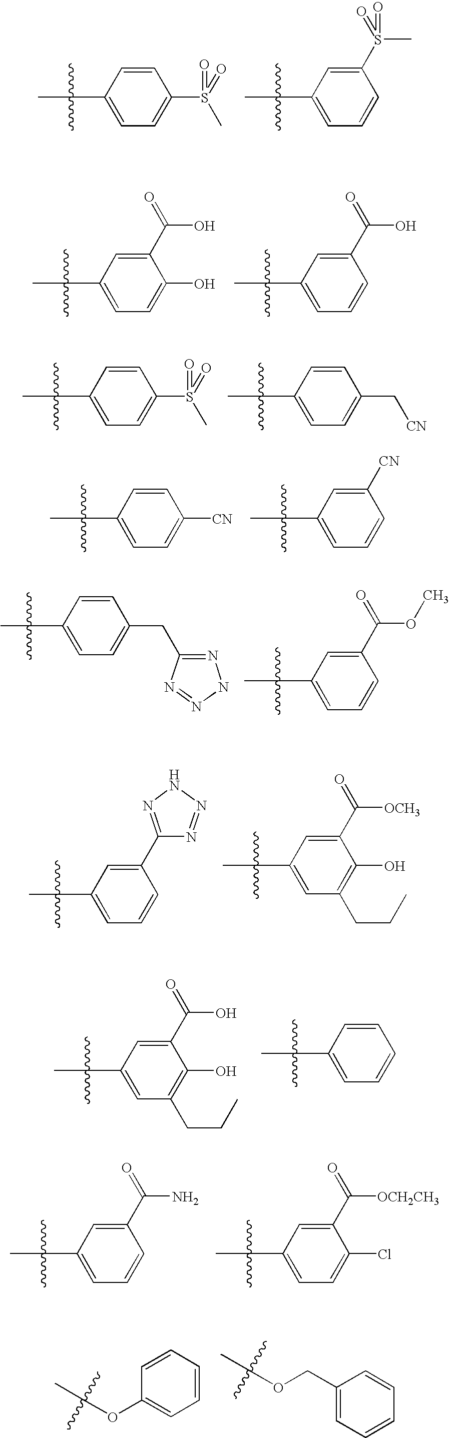

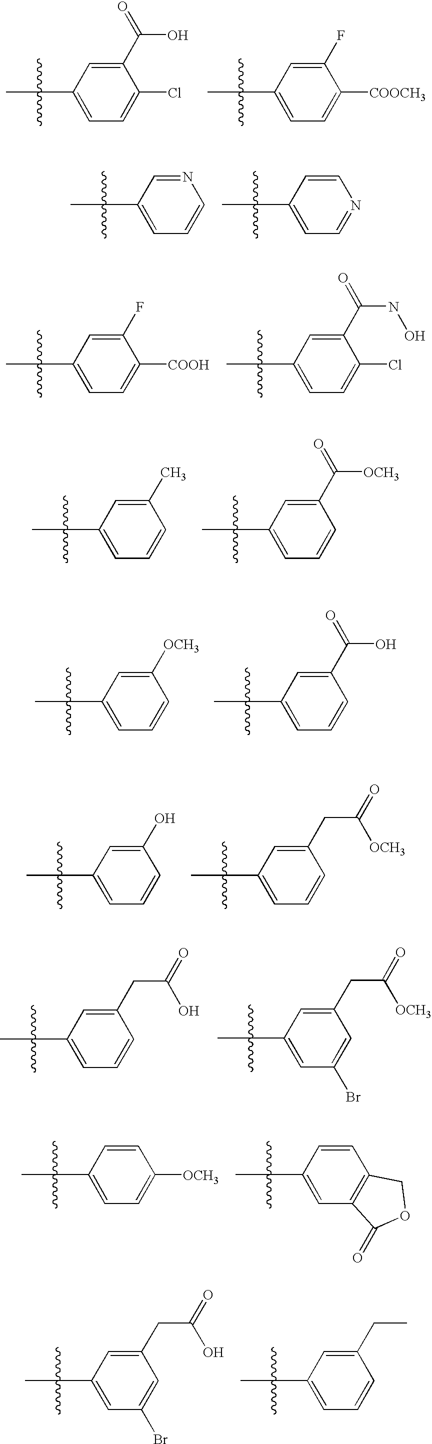

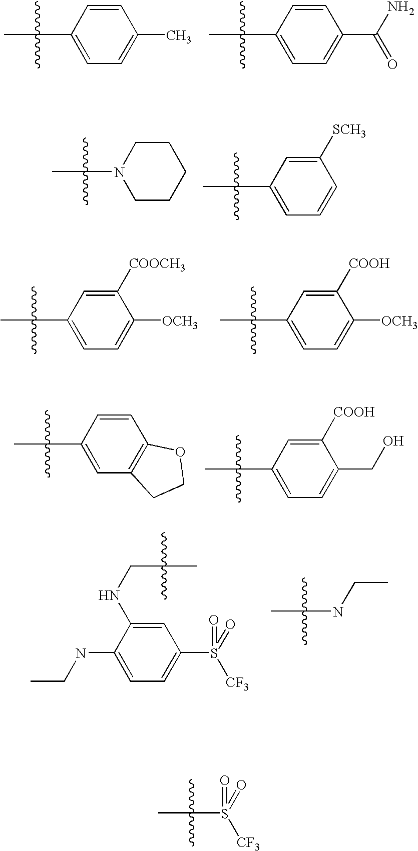

25. The compound of claim 1, wherein:
G₁, G₂, and G₃ are independently selected from, CH₃, C(═O)CH₃, CH(CH₃)CH₃,

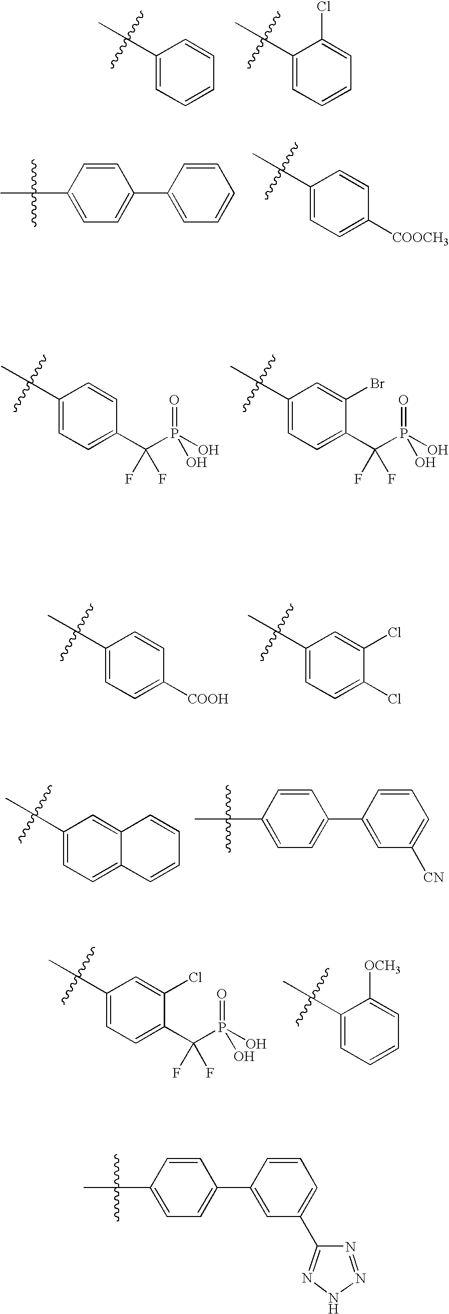

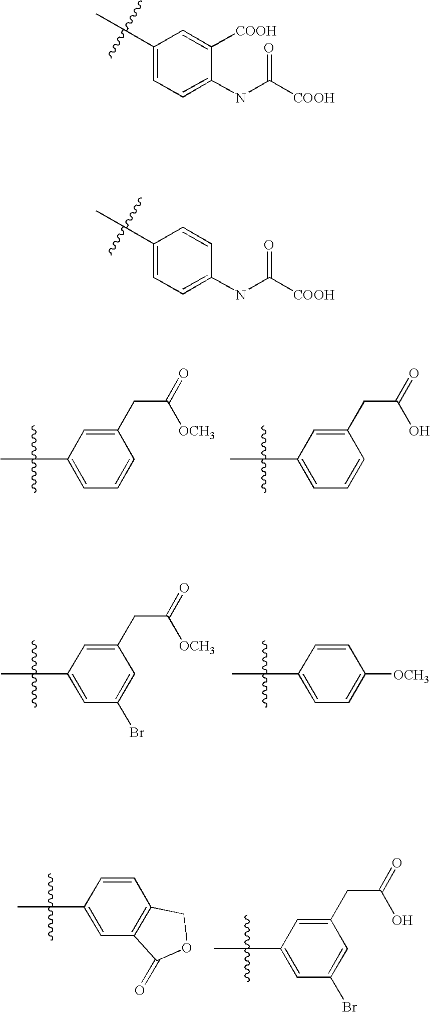

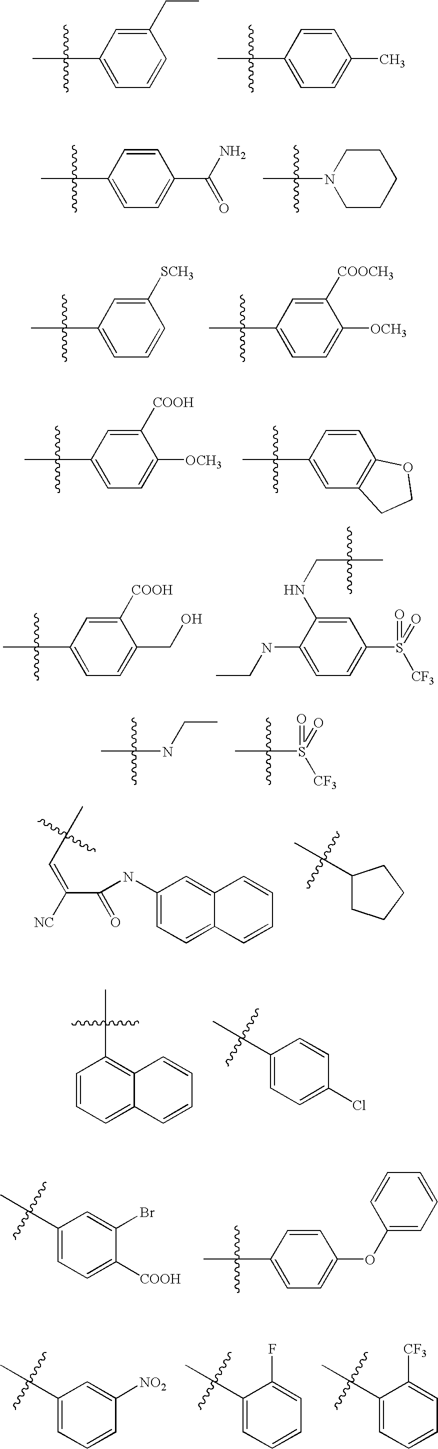

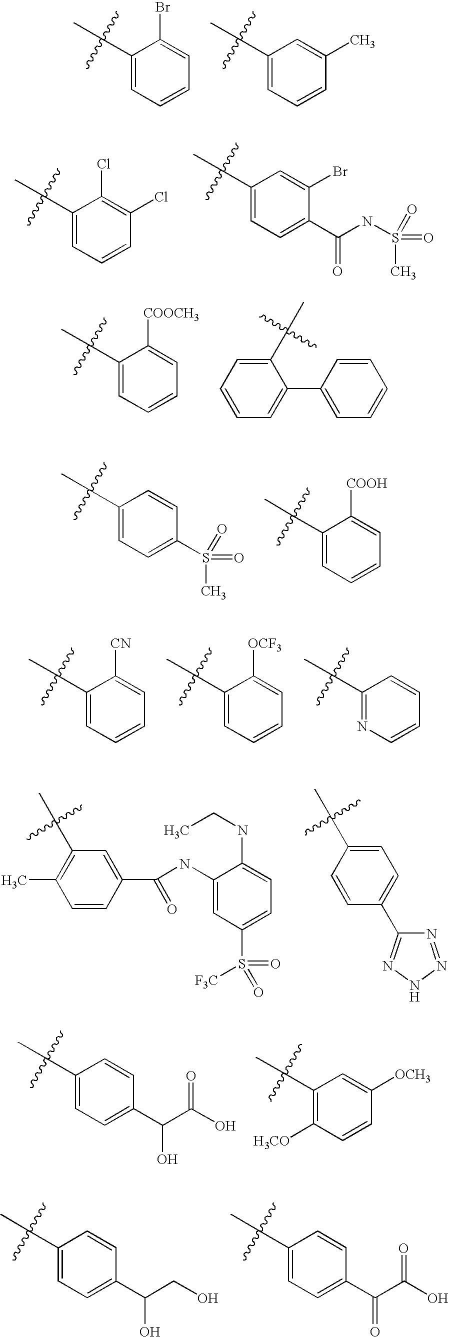

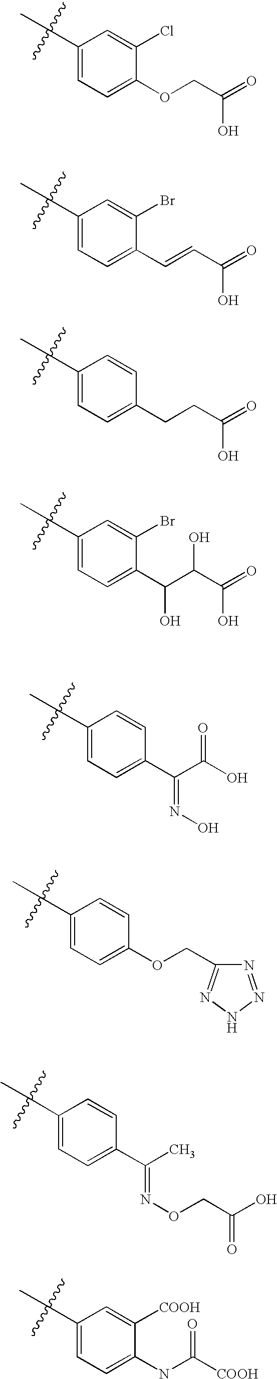

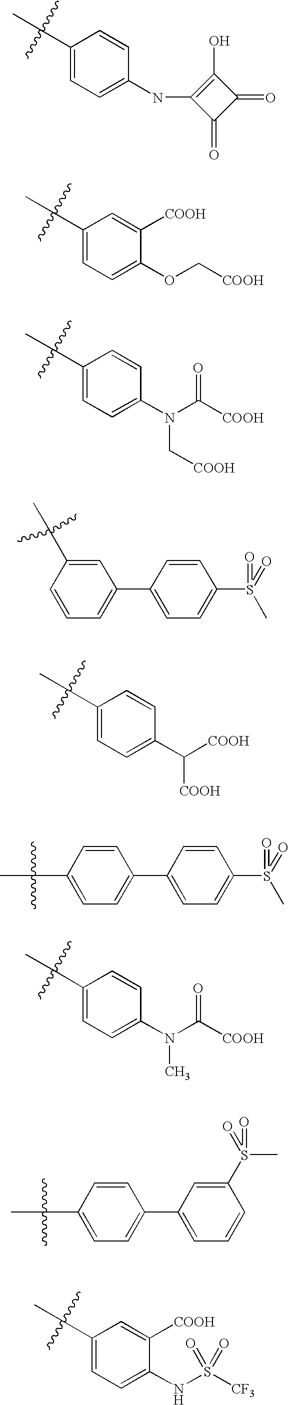

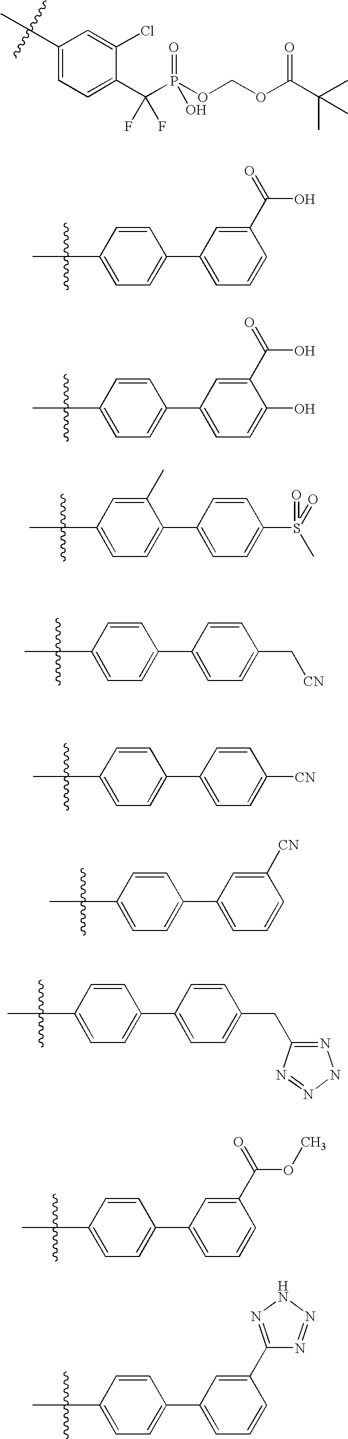

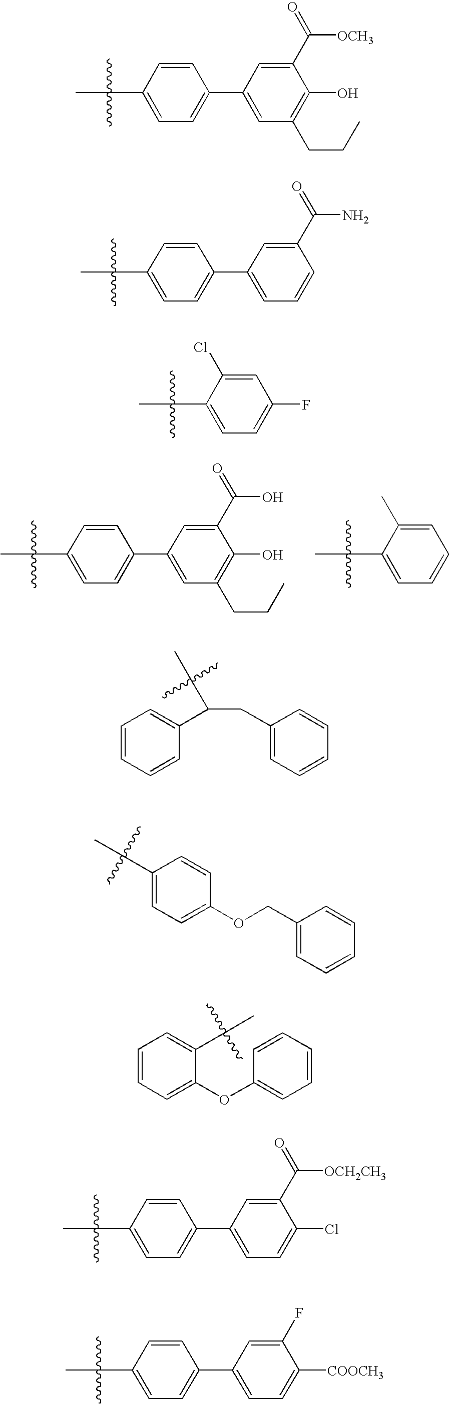

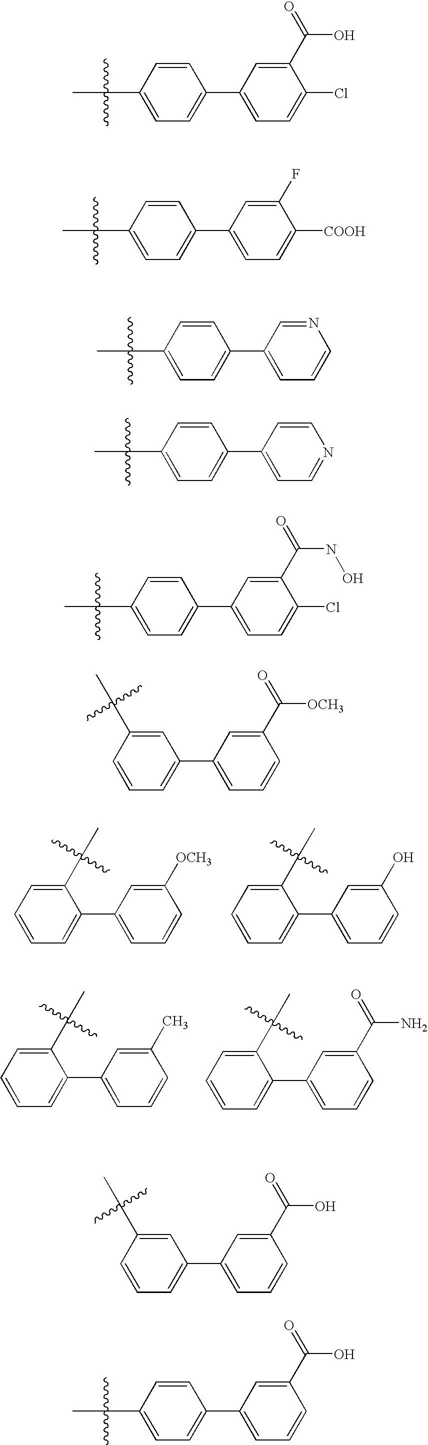

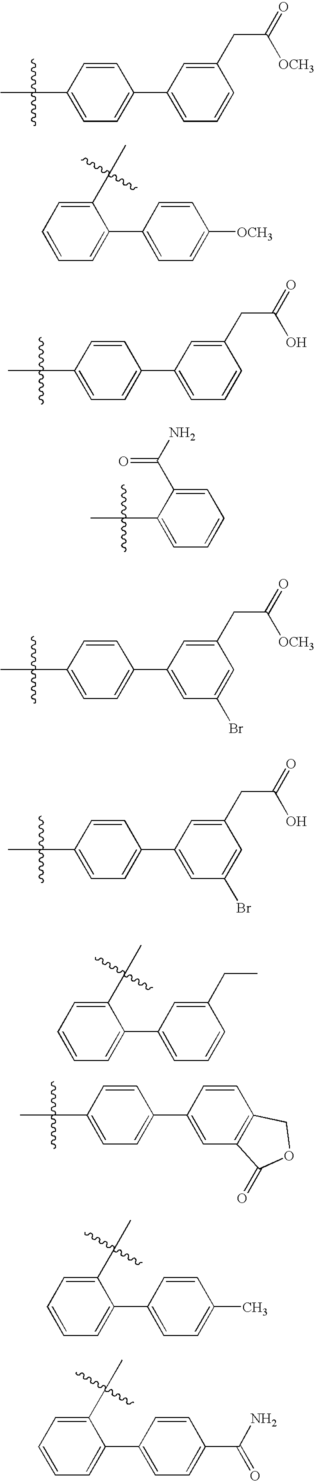

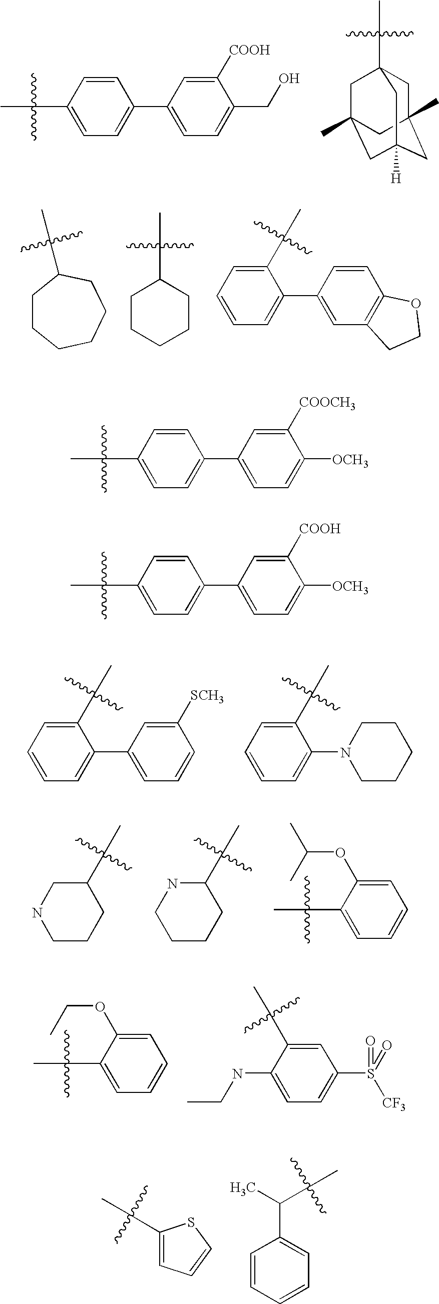

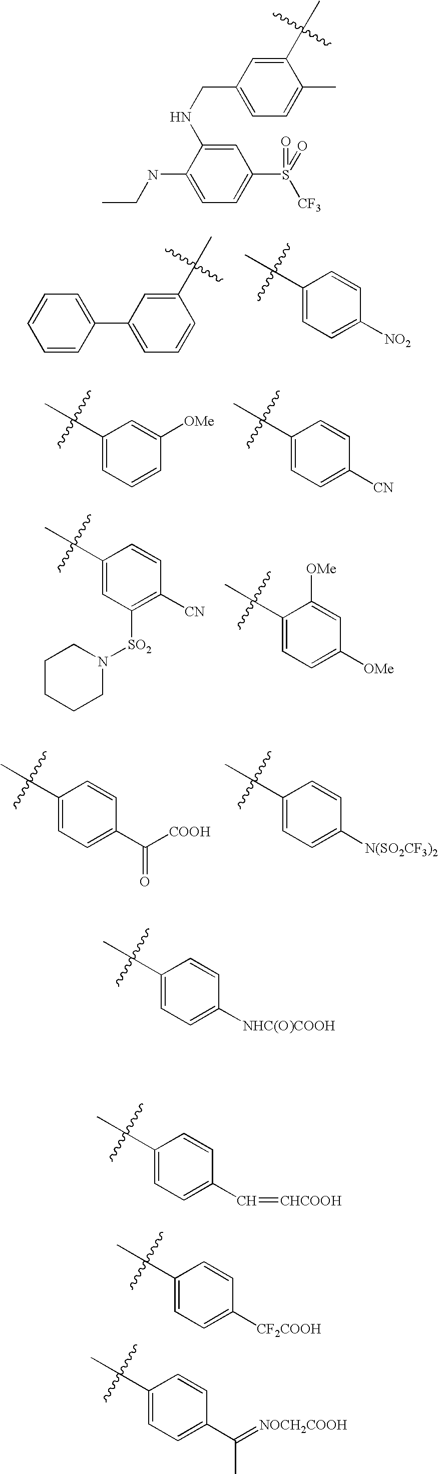

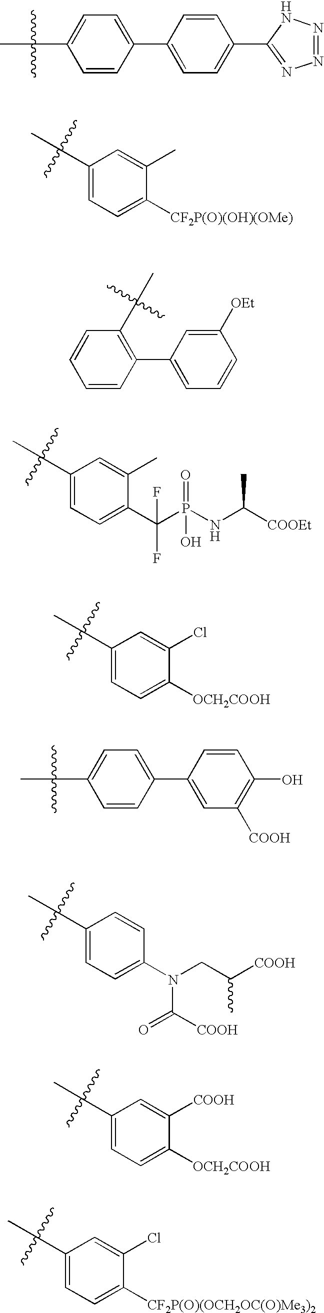

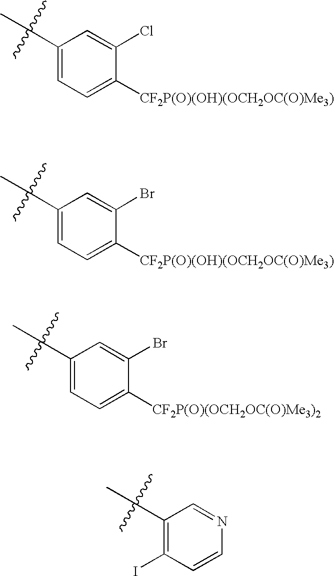

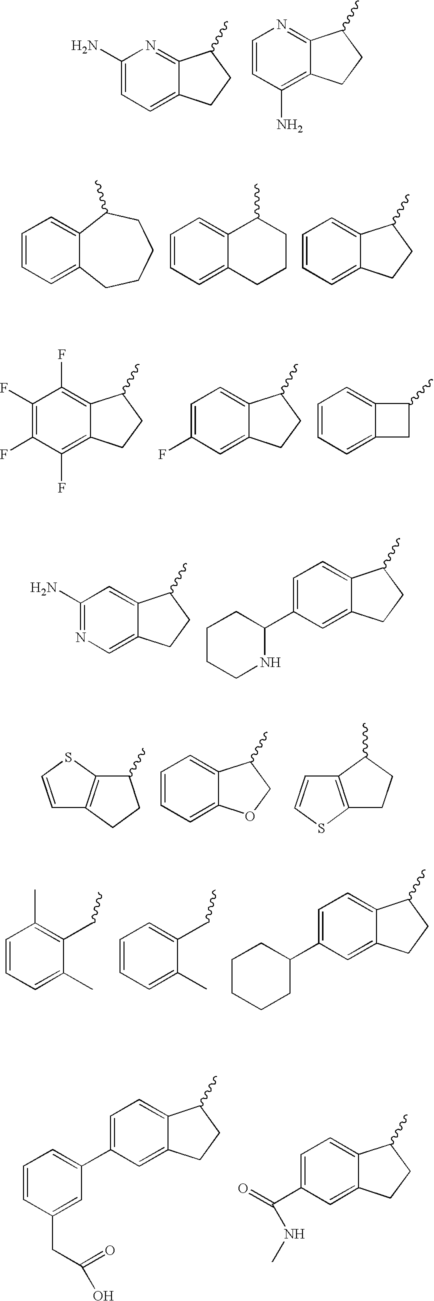

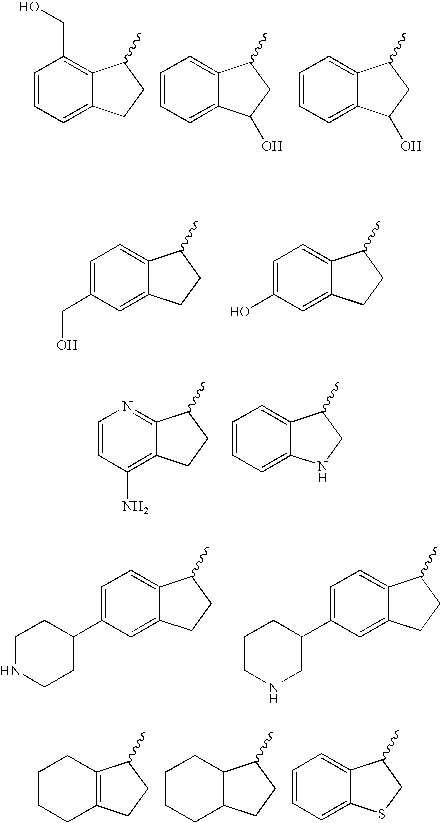

26. The compound of claim 1, wherein:
G₁ and/or G₂ and/or G₃ is

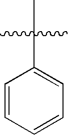

27. The compound of claim 1, wherein:
G₁ and/or G₂ and/or G₃ is

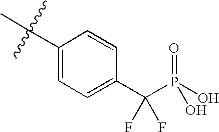

28. The compound of claim 1, wherein the compound is:

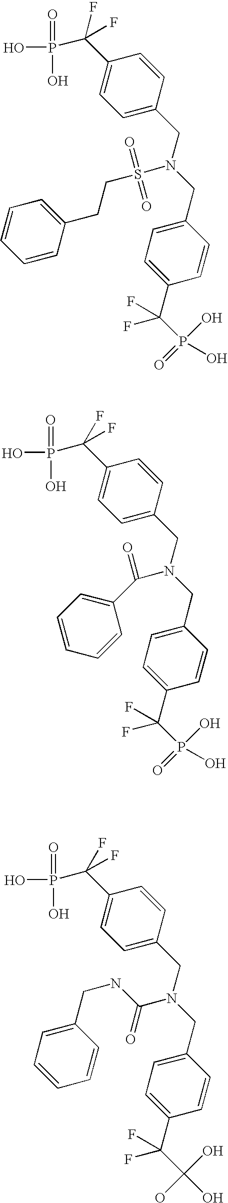

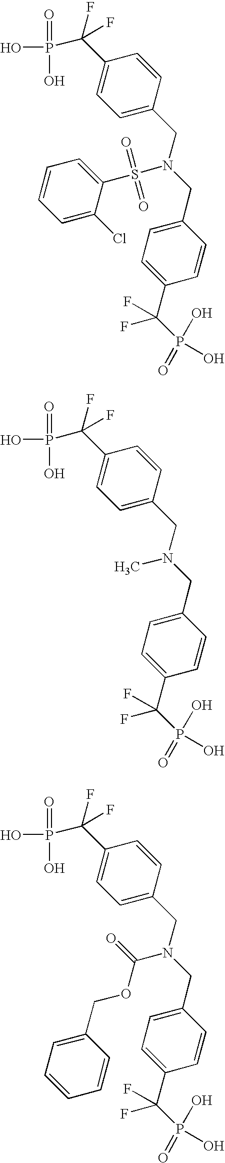

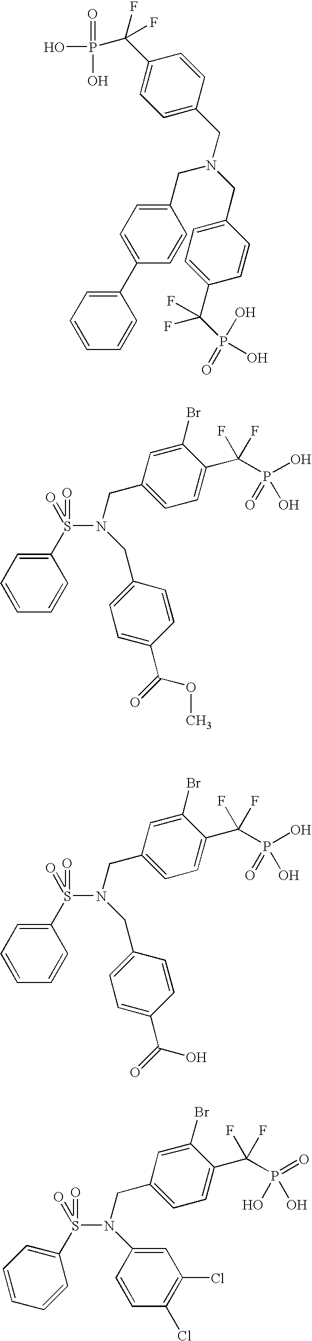

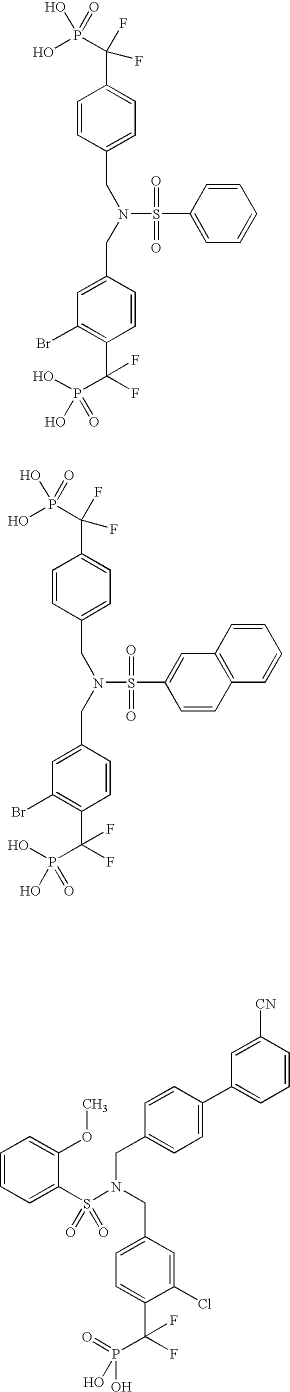

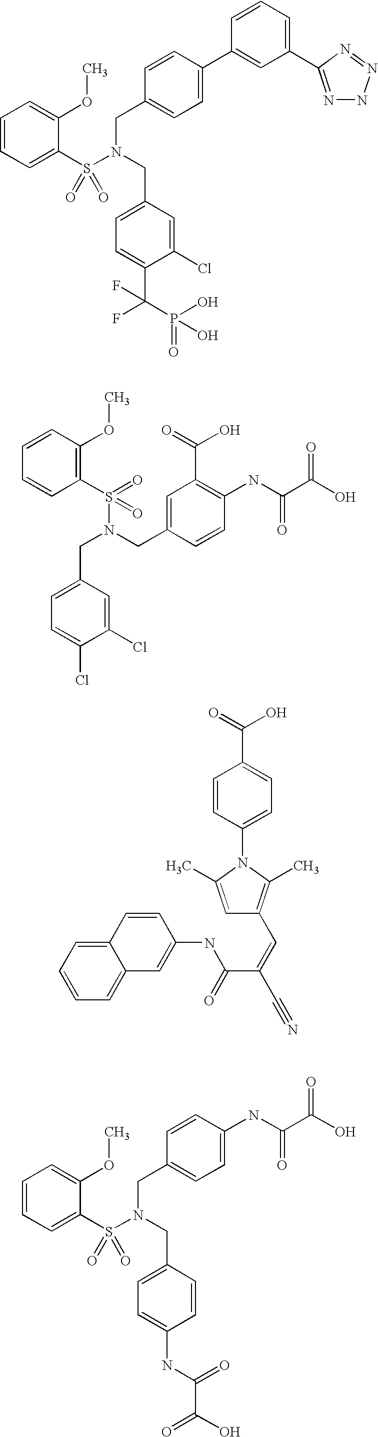

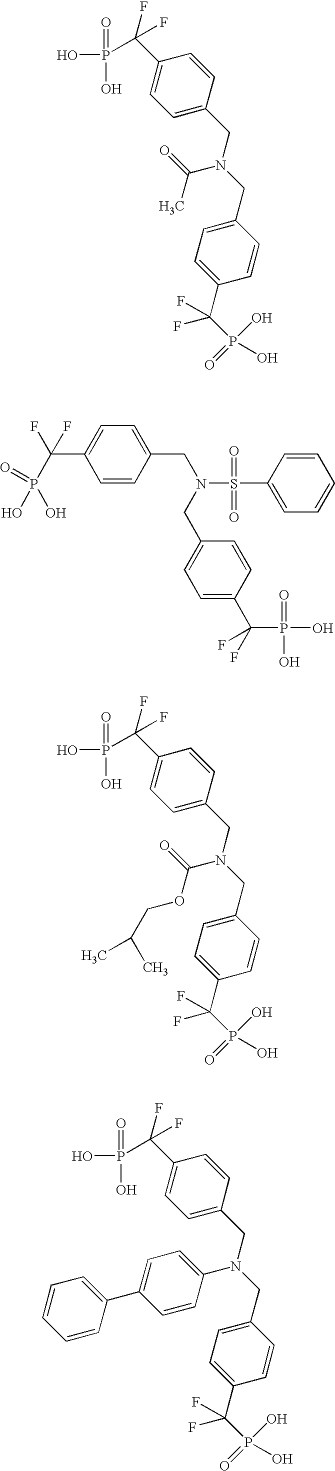

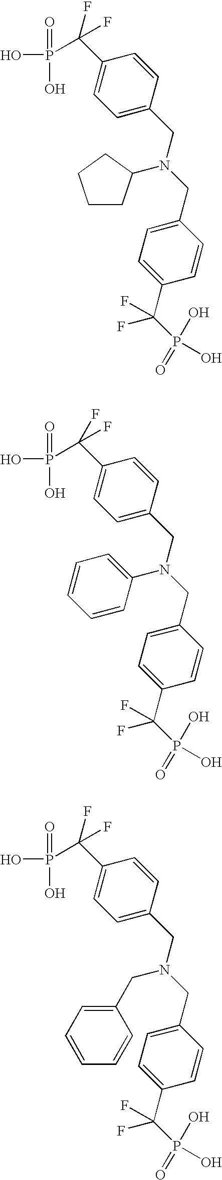

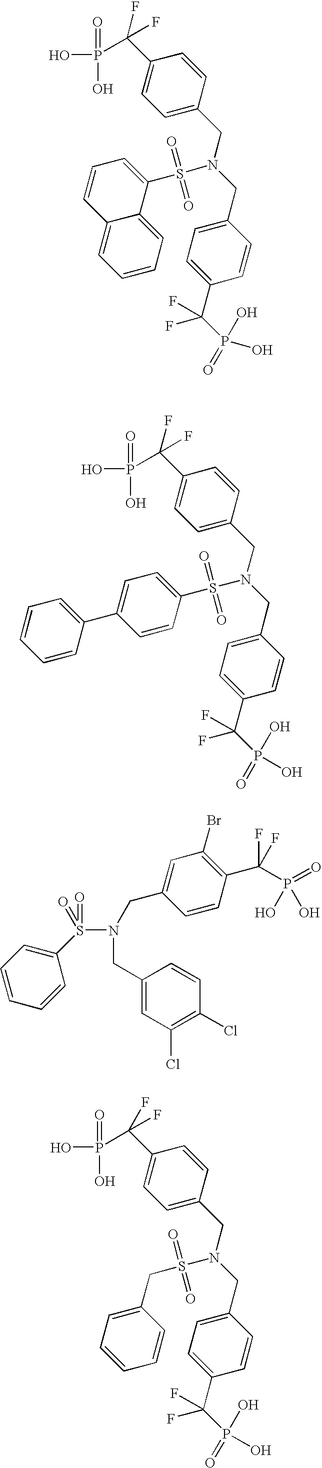

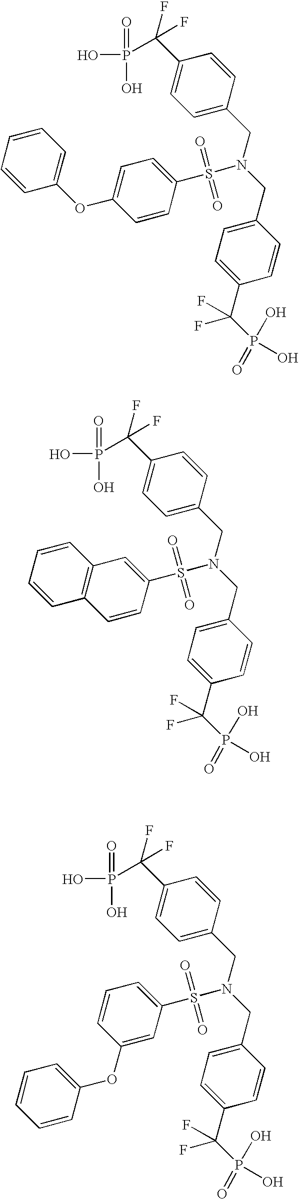

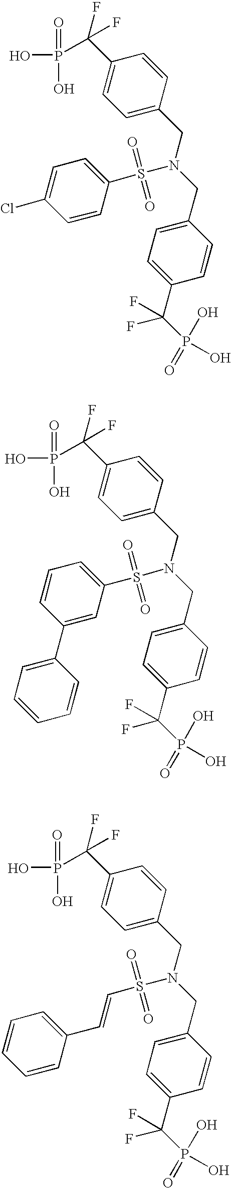

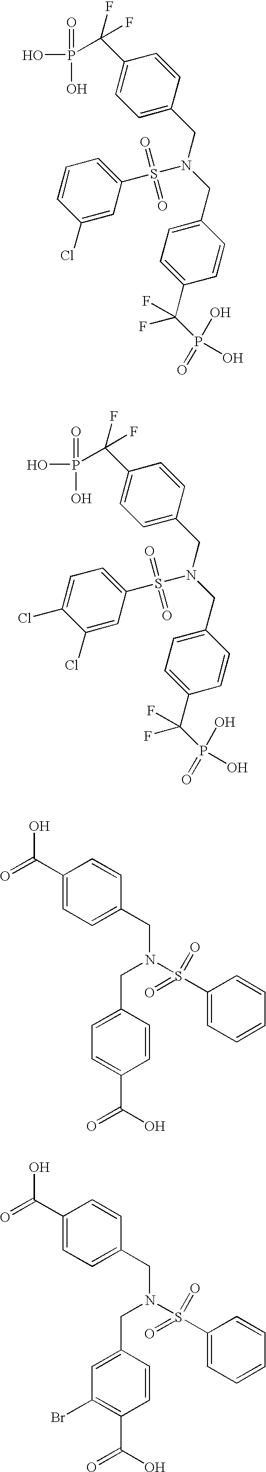

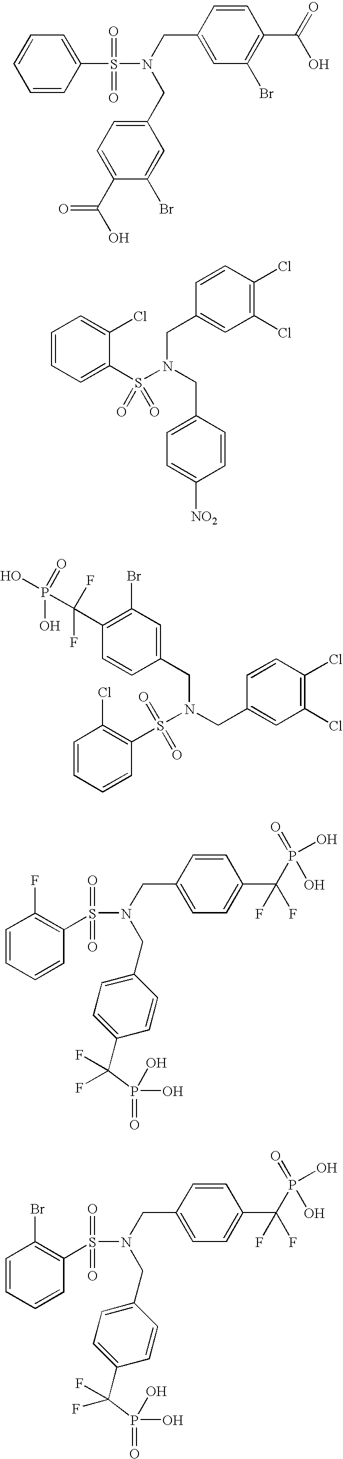

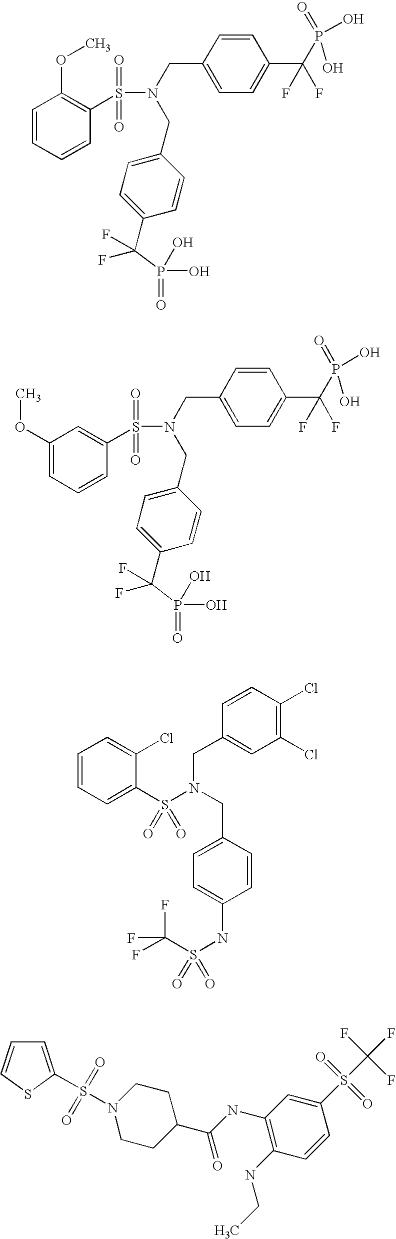

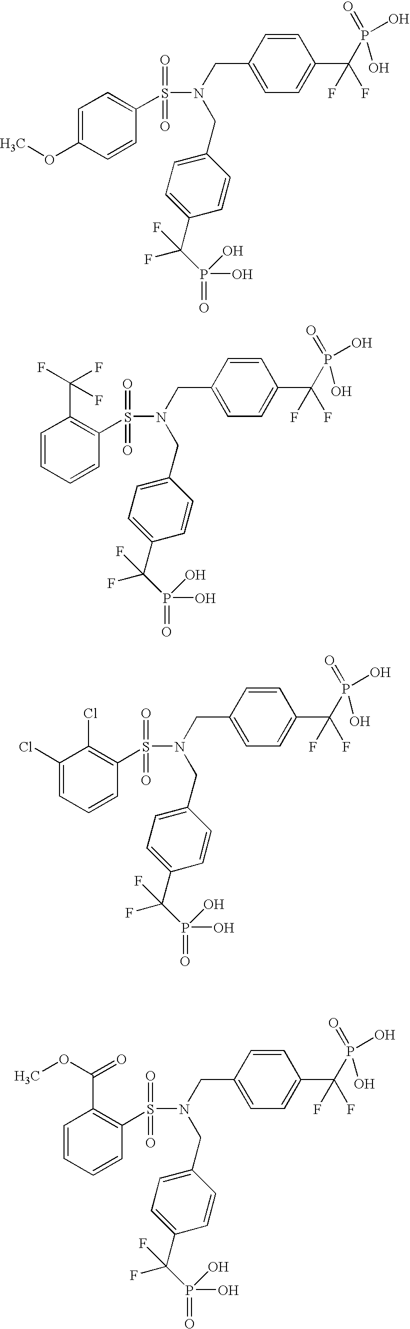

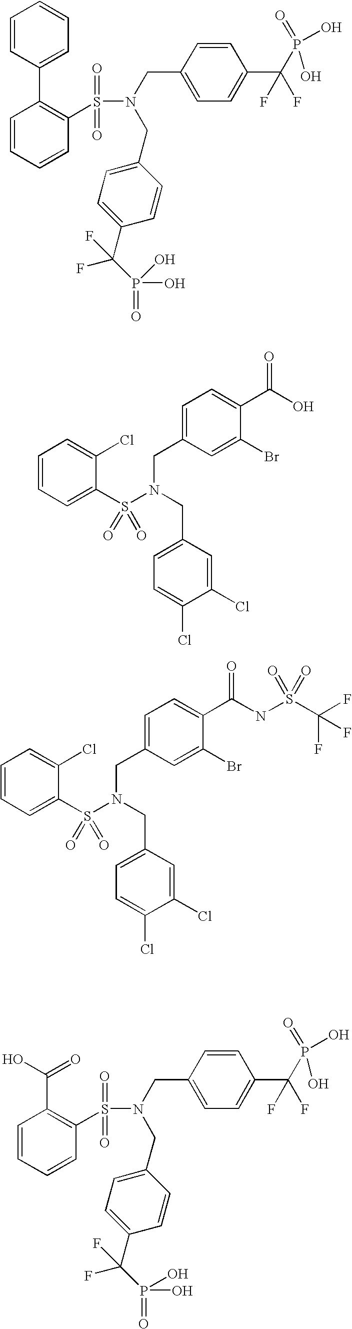

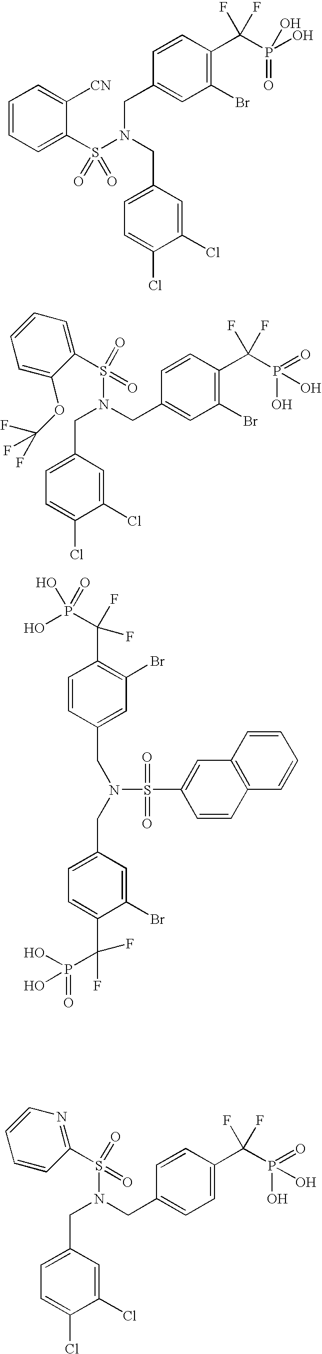

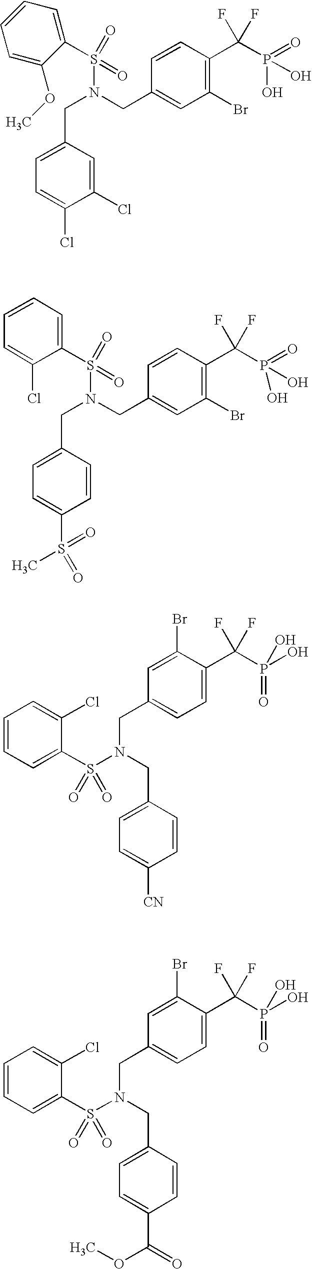

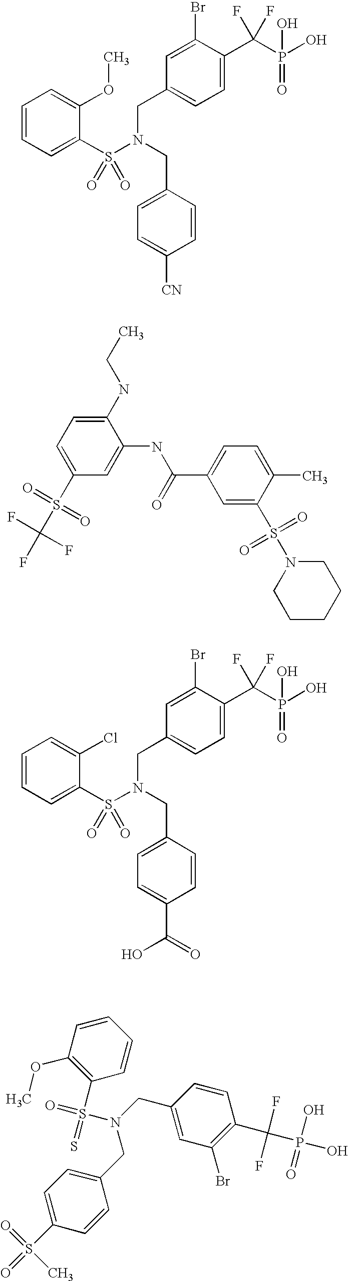

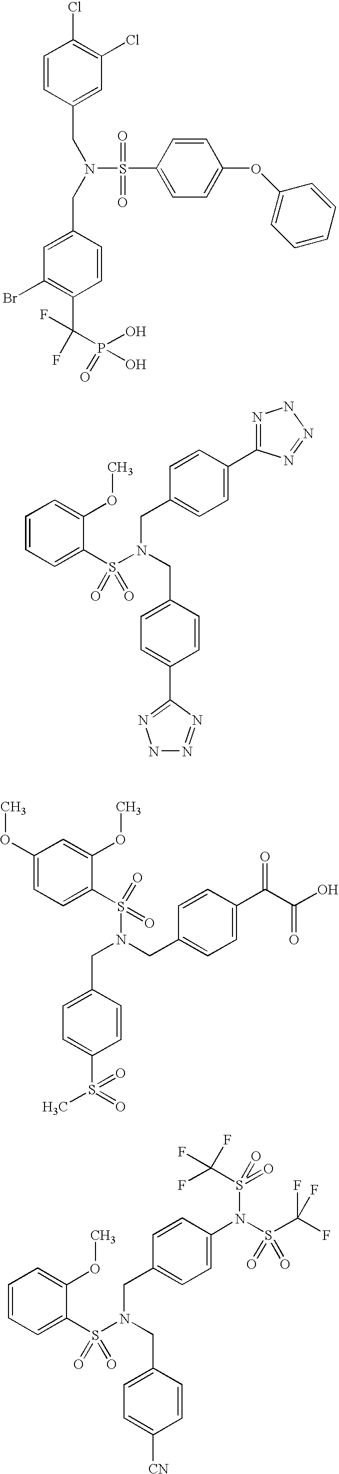

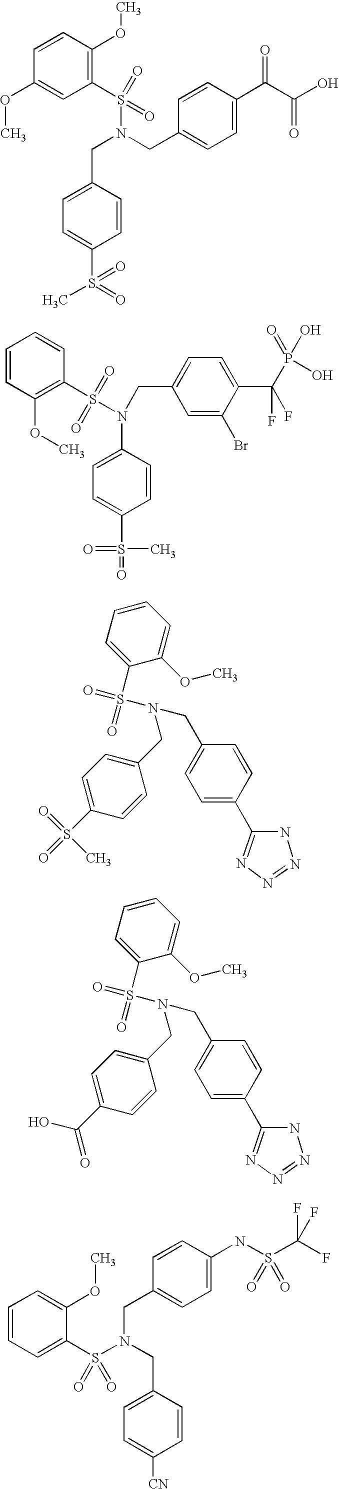

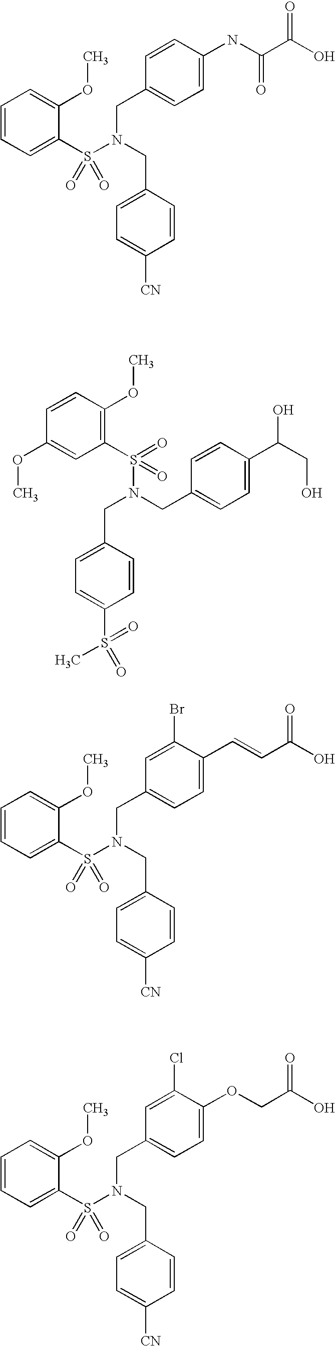

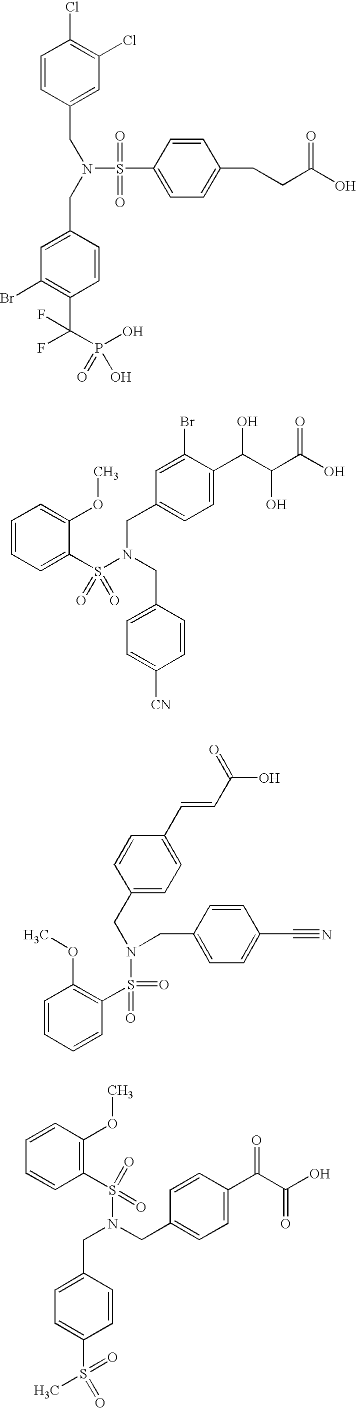

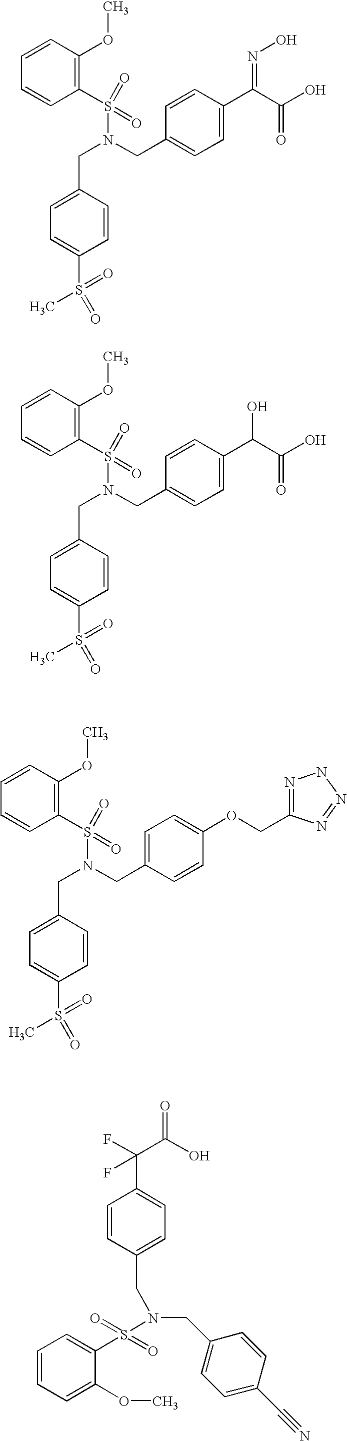

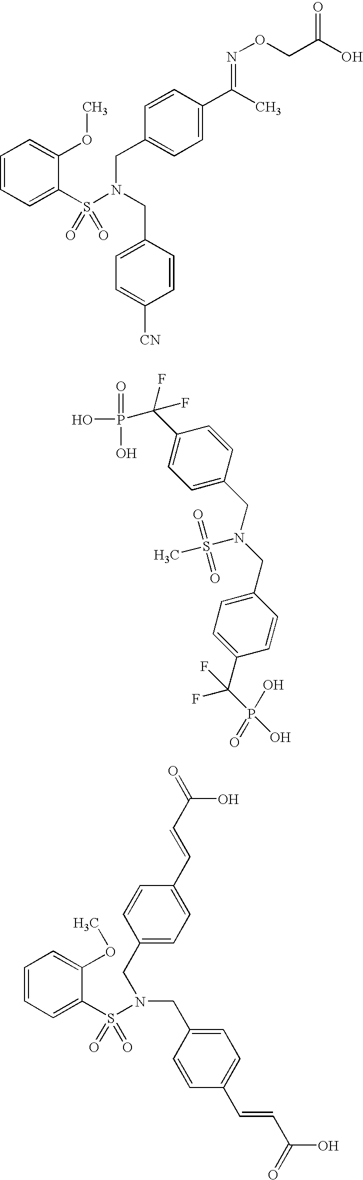

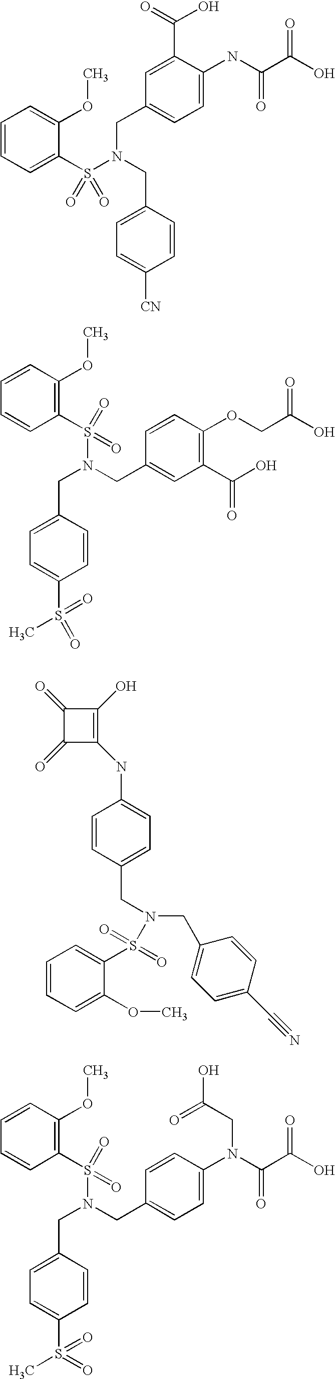

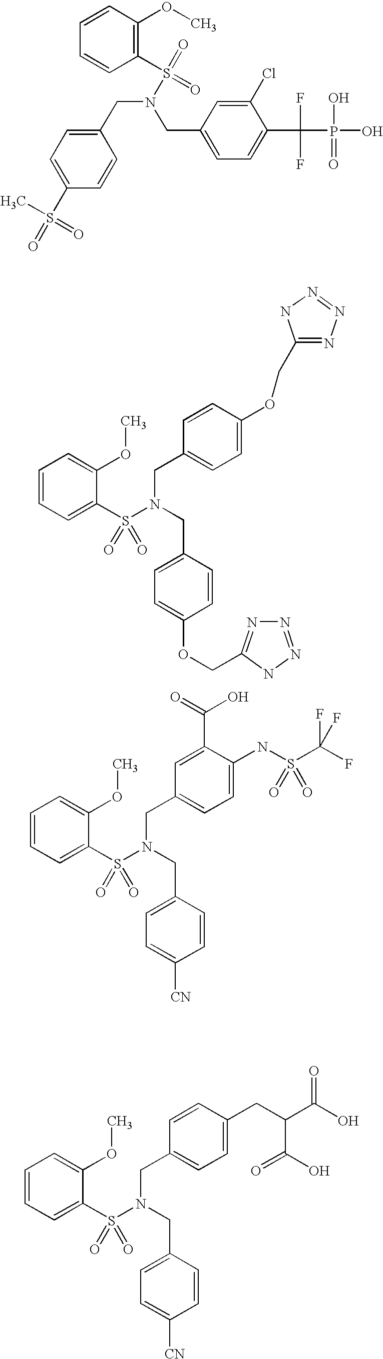

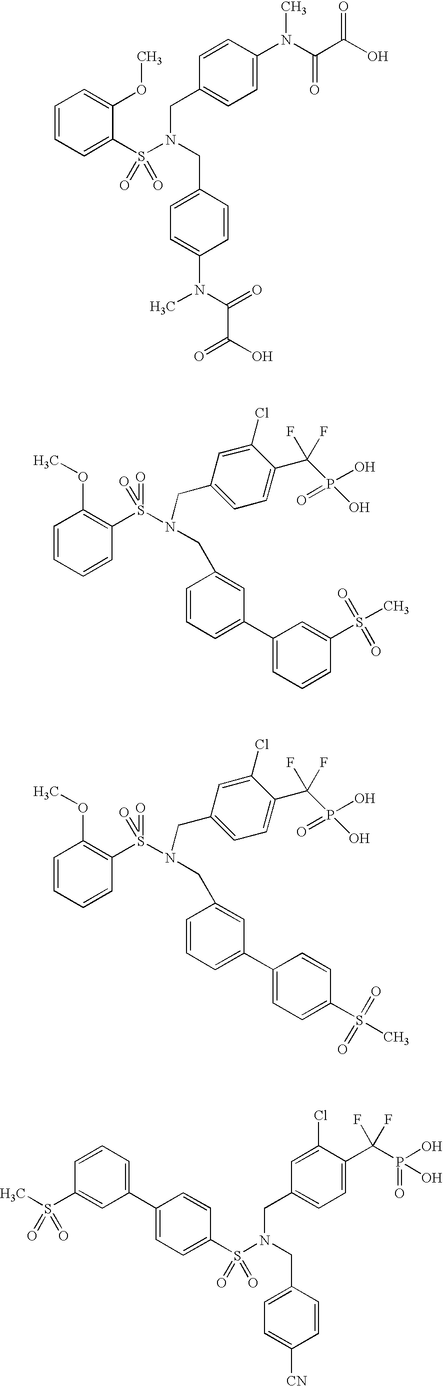

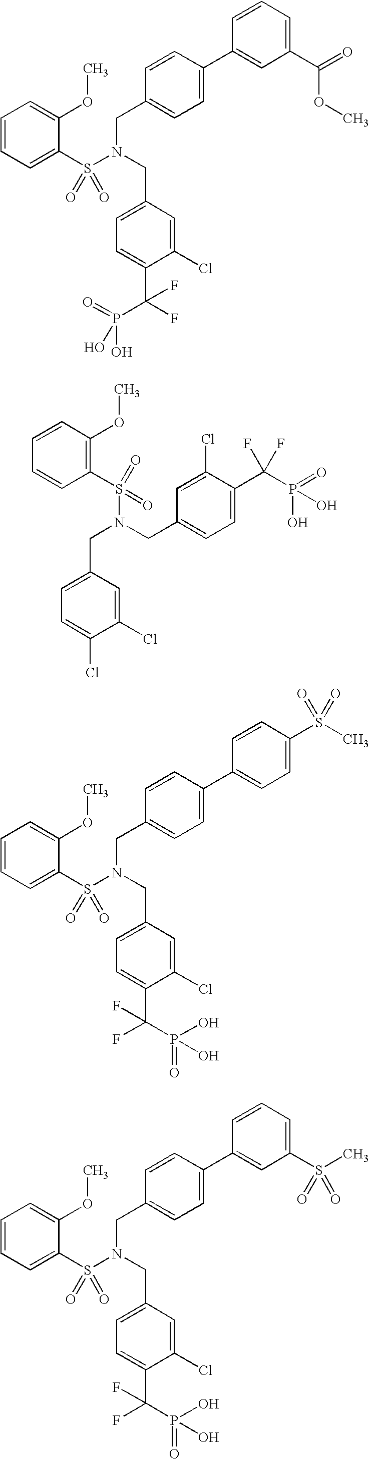

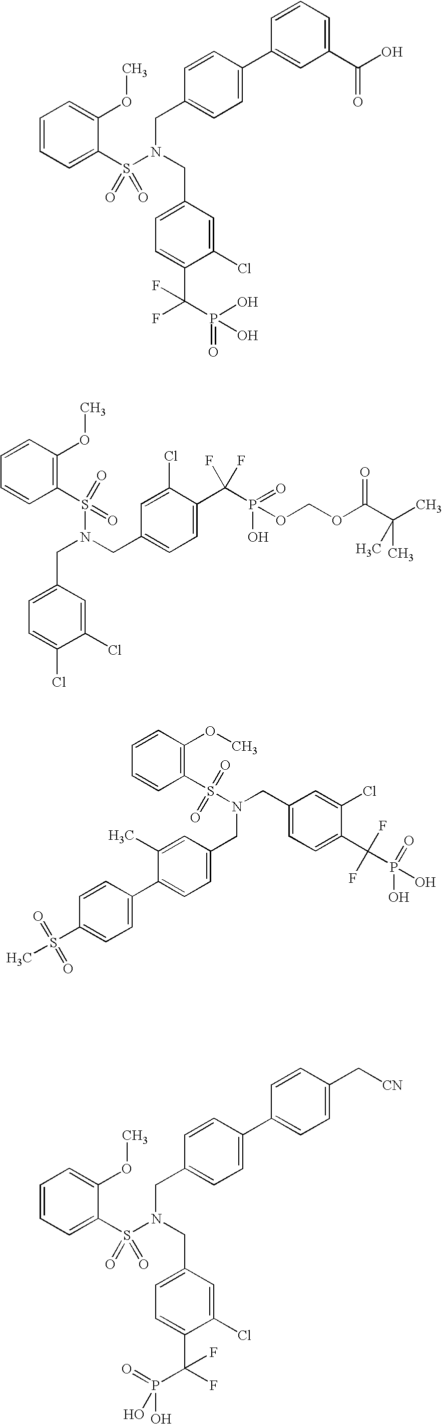

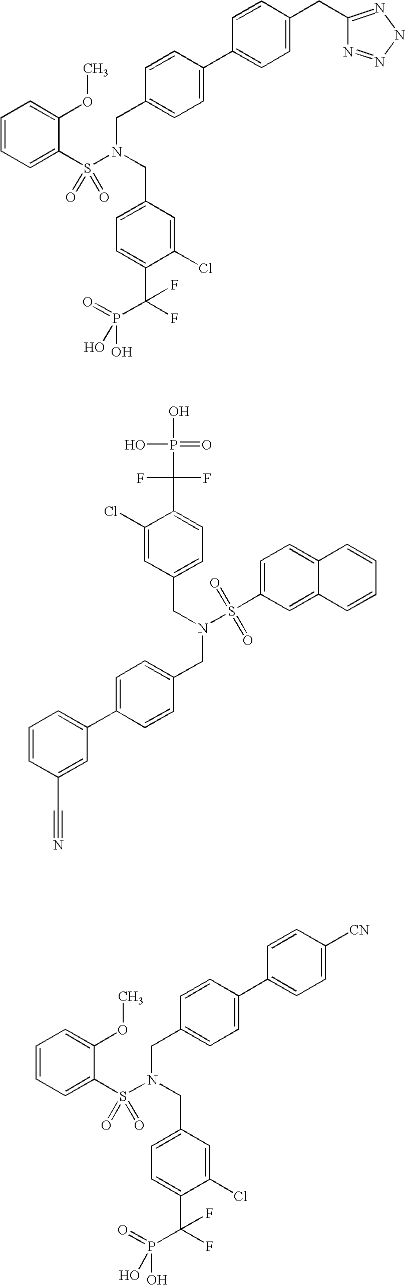

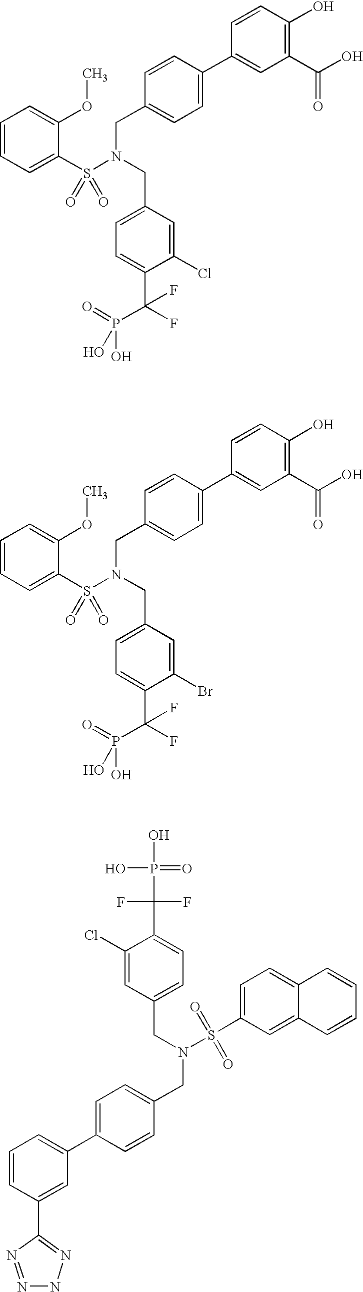

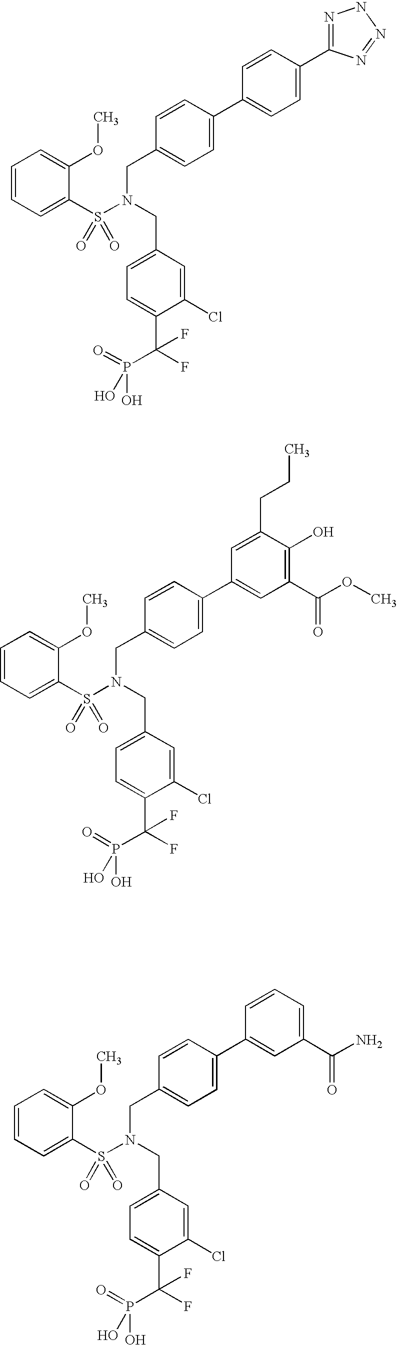

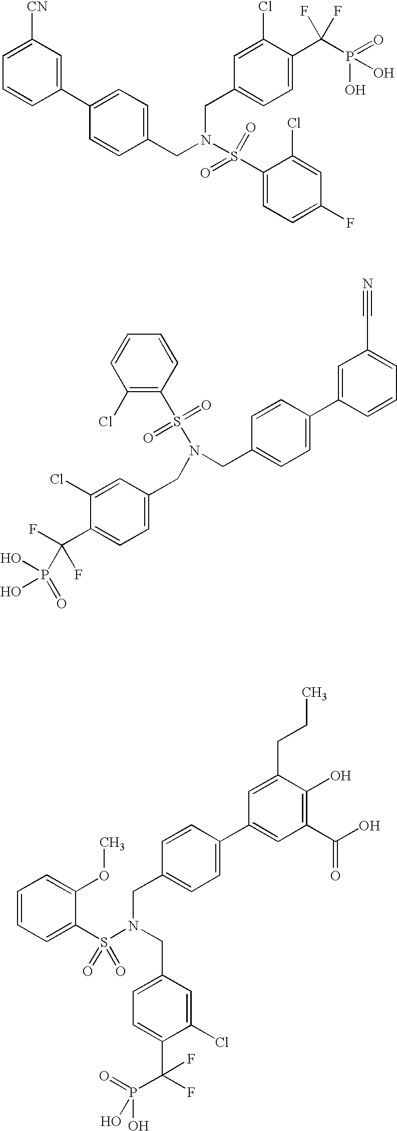

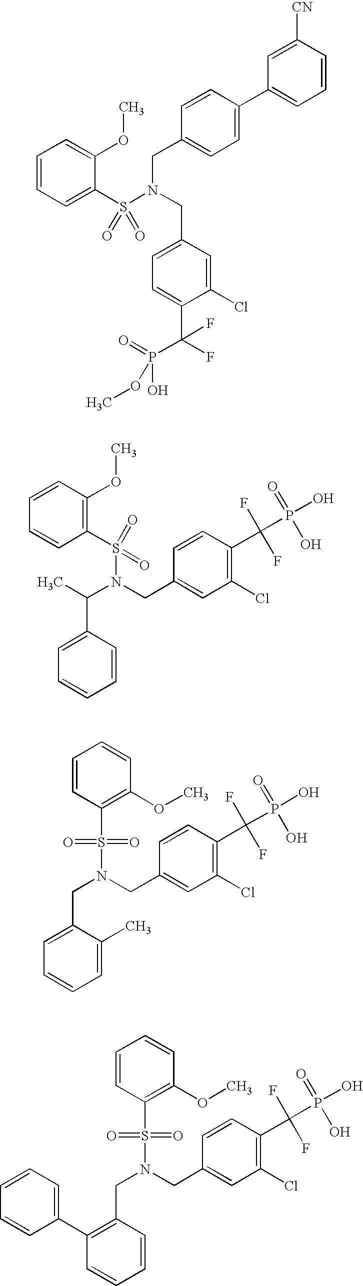

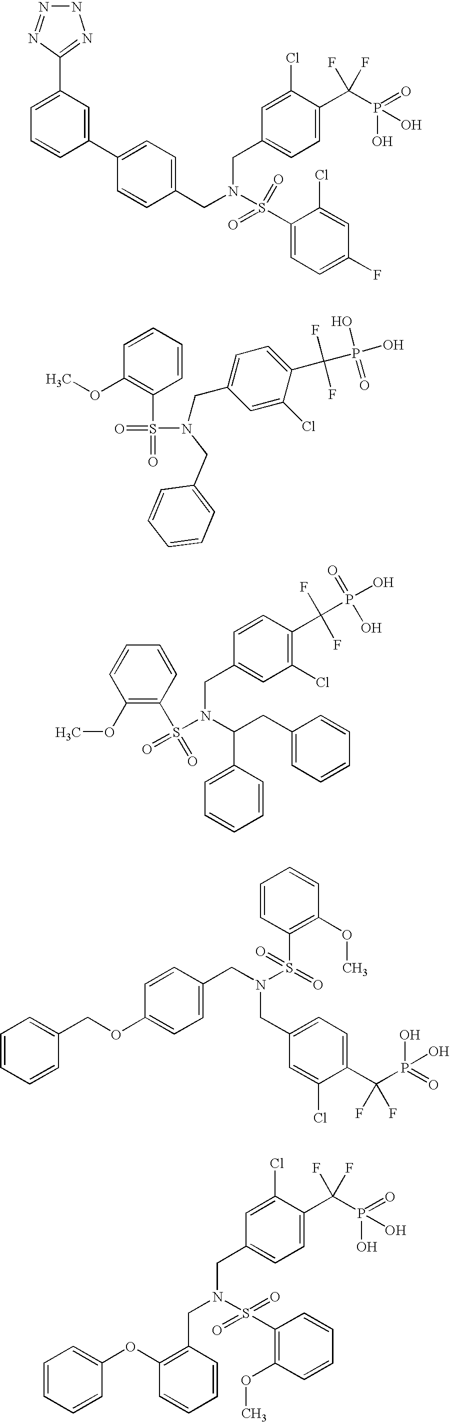

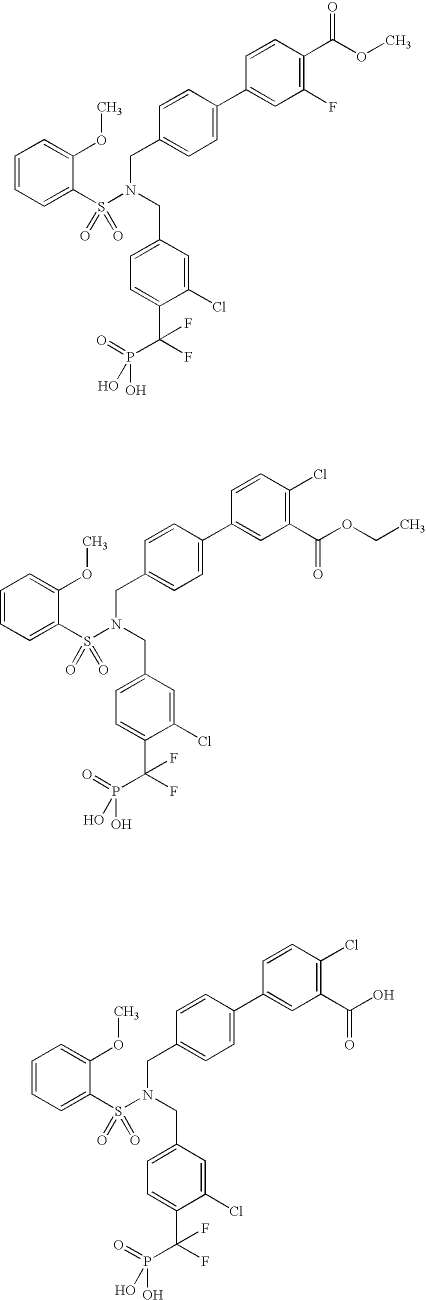

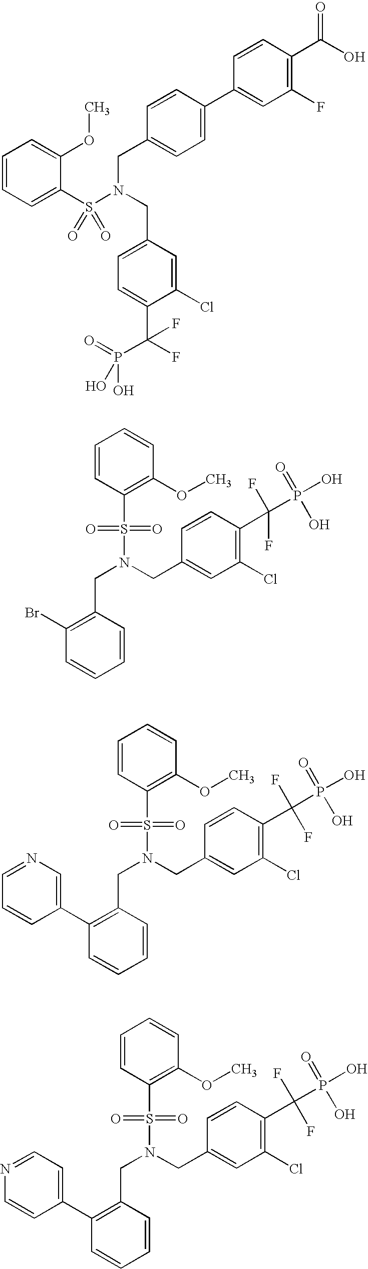

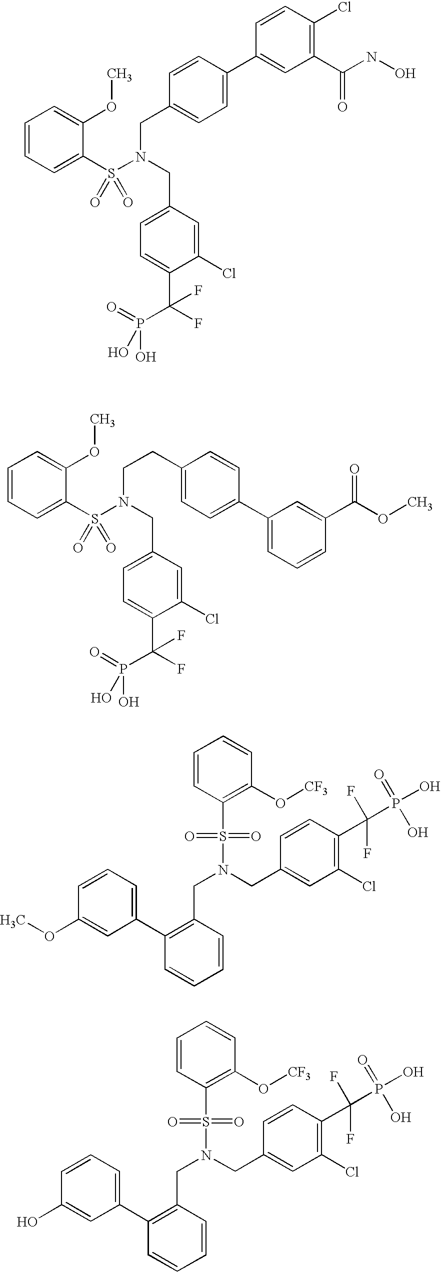

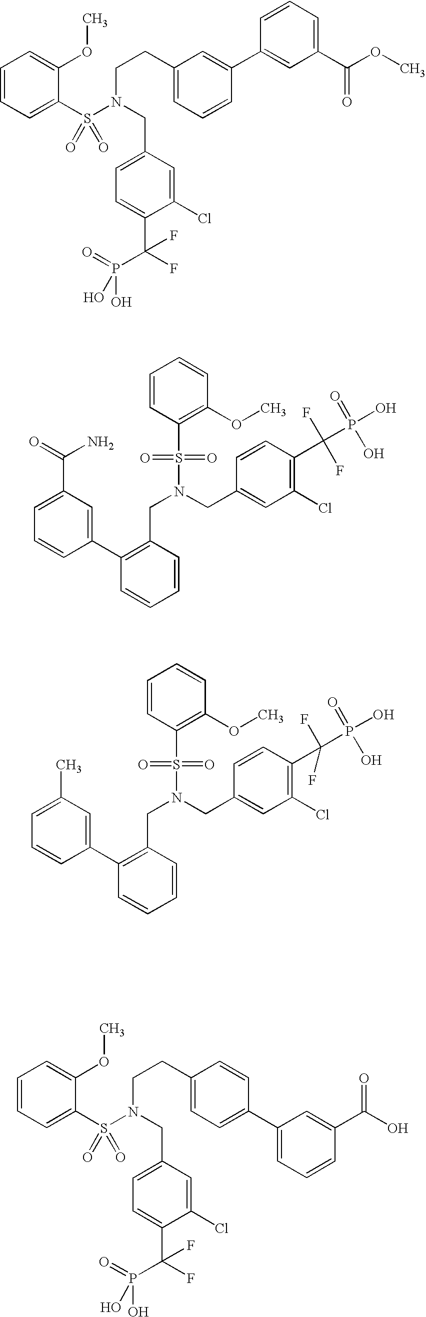

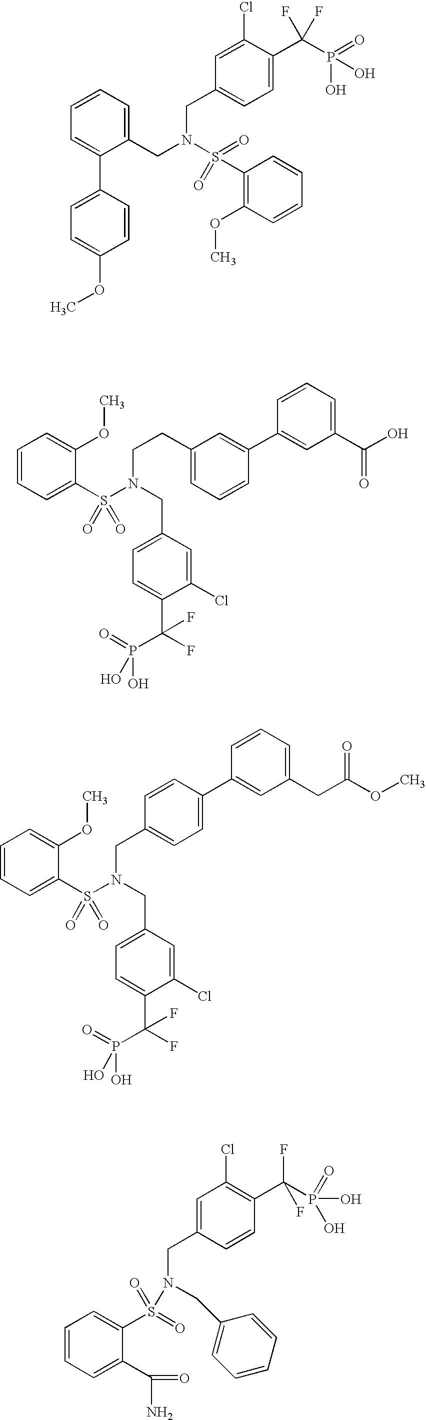

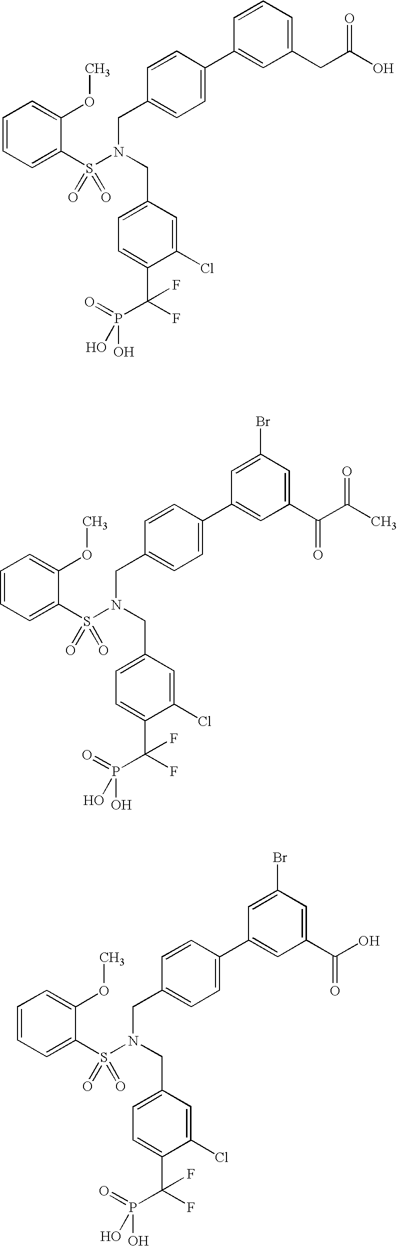

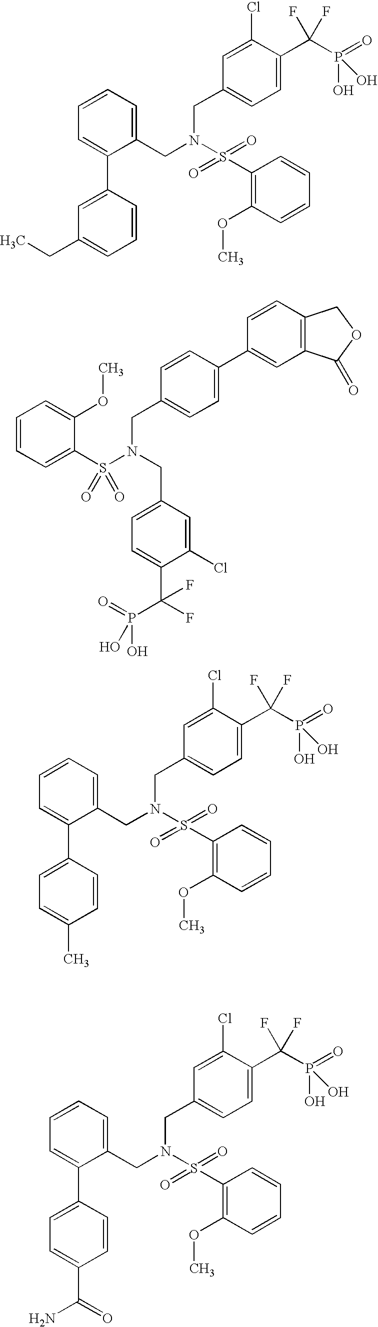

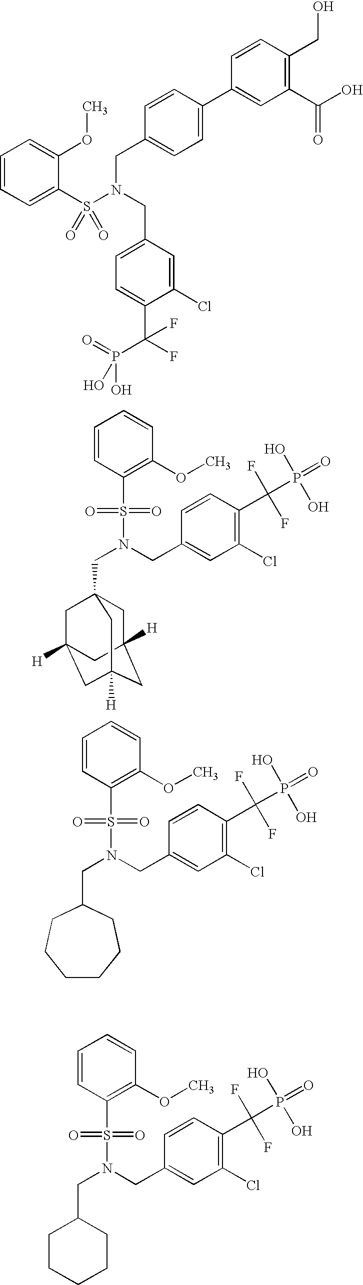

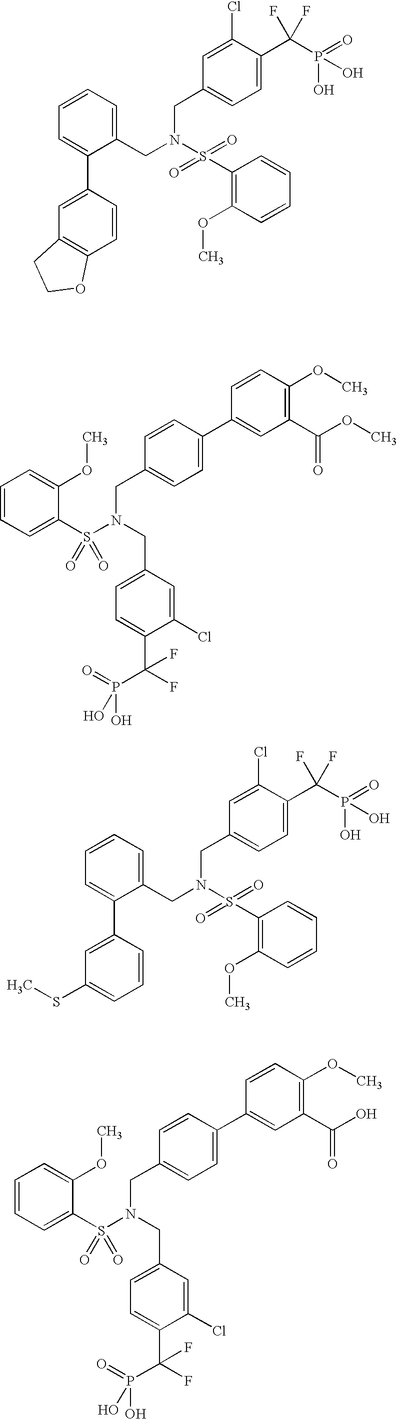

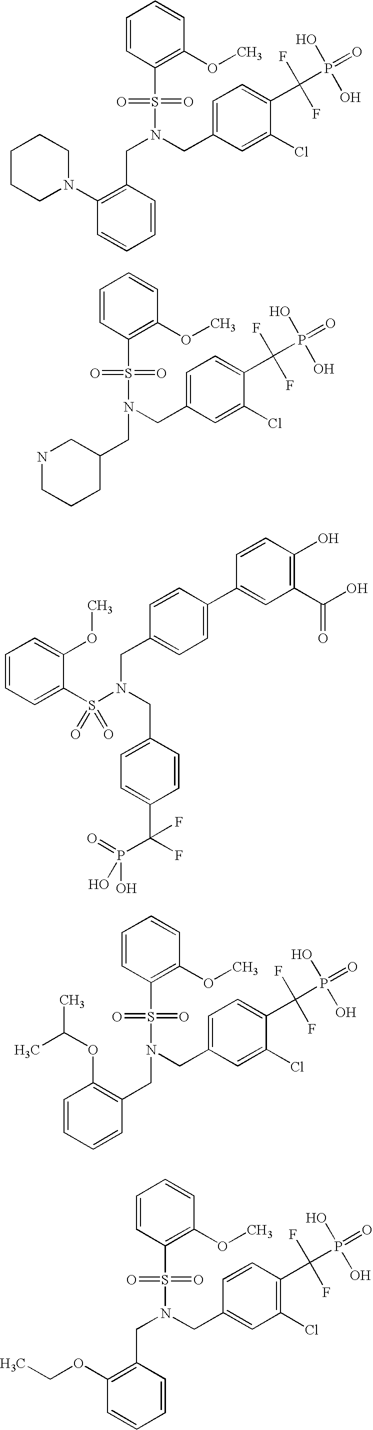

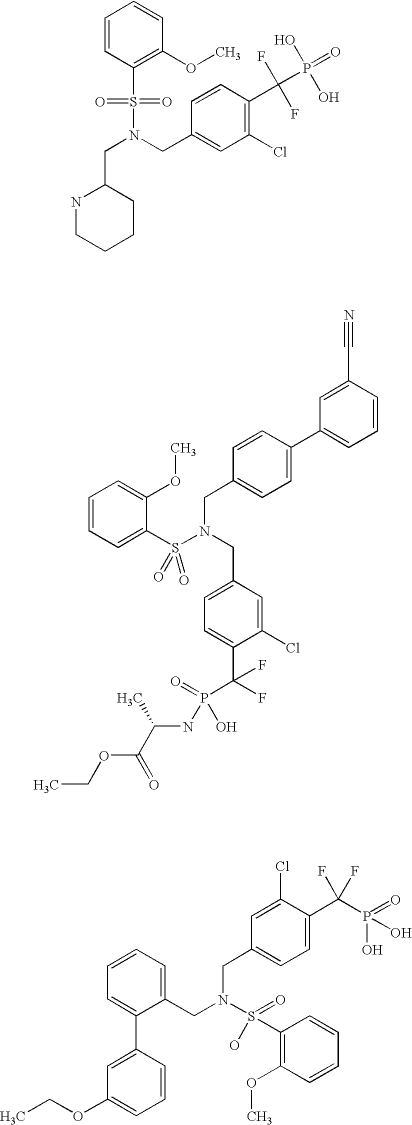

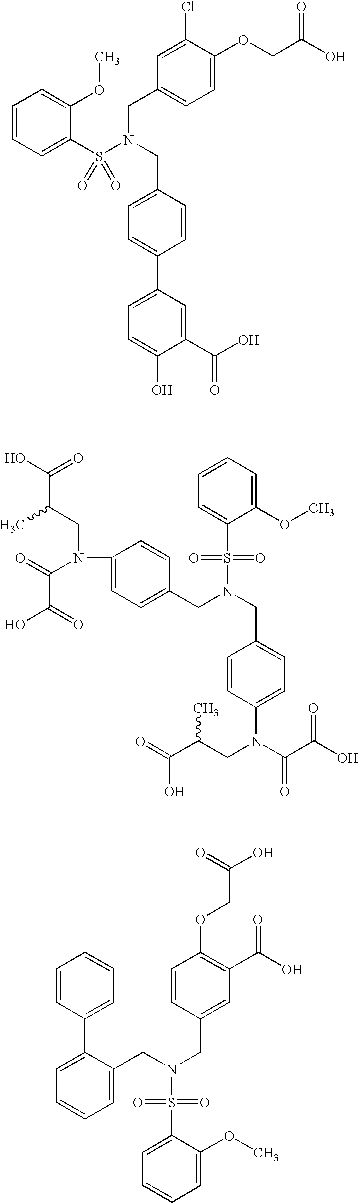

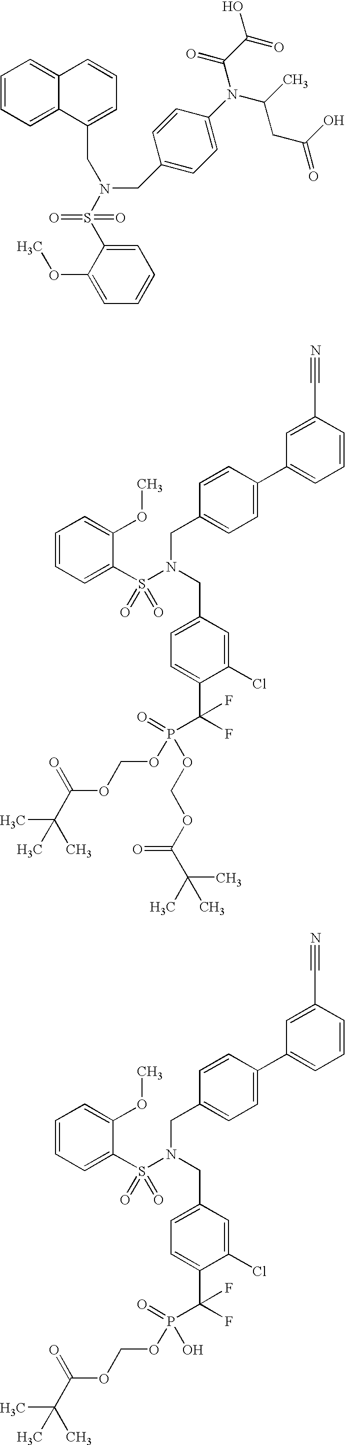

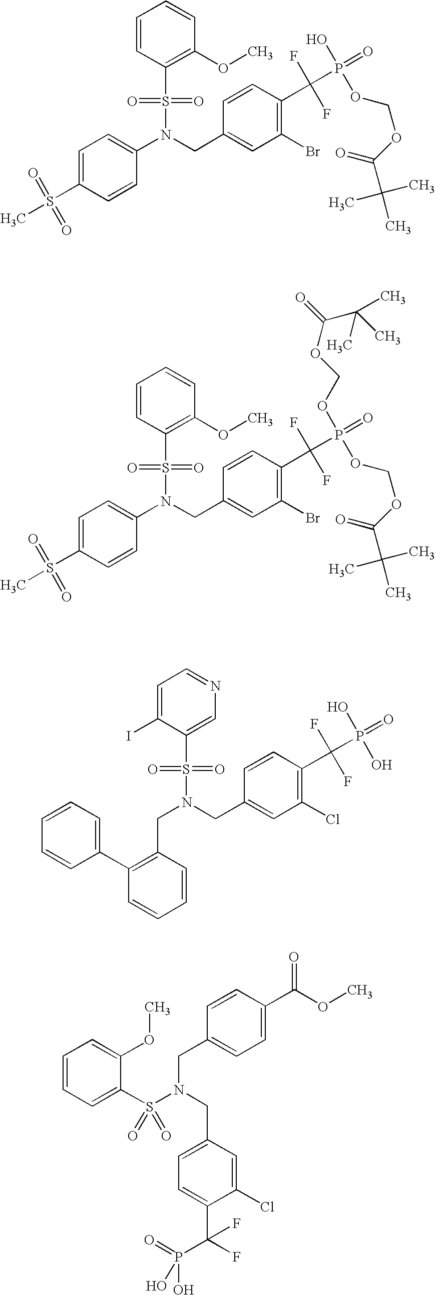

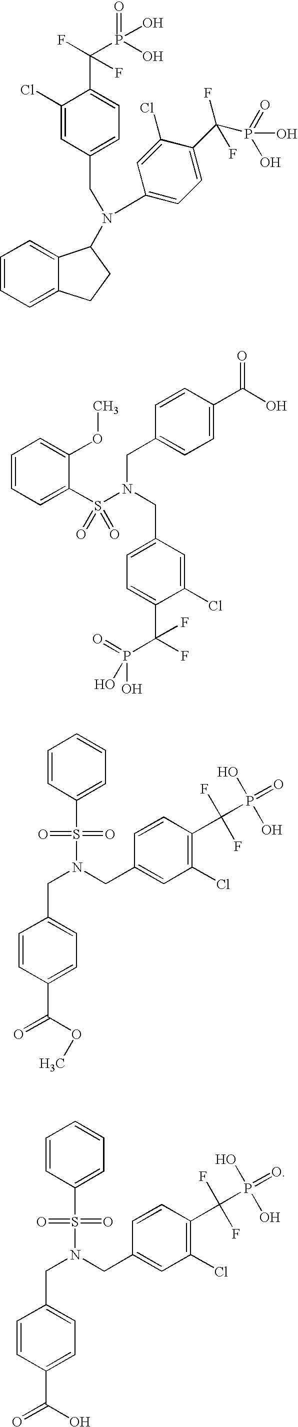

29. A prodrug of a compound of claim 1.

30. The prodrug of claim 29, wherein the prodrug is a mono- or di-ester of a phosphonic acid or an ester of a carboxylic acid, and has the formula —CH(H/Me)OC(═O)OiPr, —(CH(H/Me)OC(═O)tBu, or ROCH₂CHR′CH₂—, where R is C₁₄₋₂₀-n-alkyl and R′ is H, OH or OMe.

31. A prodrug of a compound of claim 28.

32. The prodrug of claim 31, wherein the prodrug is a mono- or di-ester of a phosphonic acid or an ester of a carboxylic acid, and has the formula —CH(H/Me)OC(═O)OiPr, —(CH(H/Me)OC(═O)tBu, or ROCH₂CHR′CH₂—, where R is C₁₄₋₂₀-n-alkyl and R′ is H, OH or OMe.

33. The prodrug claim 29 that is a mono- or bis-amidate prodrug, a mono- or di-lipid ester prodrug, a mono- or di-alpha-acyloxyalkyl ester or amide prodrug, a cytochrome P450 3A activated prodrug, a cyclic diester prodrug, a cyclic monoester monoamide prodrug, a cyclic diamide prodrug, or a carbohydrate prodrug.

34. The prodrug claim 31 that is a mono- or bis-amidate prodrug, a mono- or di-lipid ester prodrug, a mono- or di-alpha-acyloxyalkyl ester or amide prodrug, a cytochrome P450 3A activated prodrug, a cyclic diester prodrug, a cyclic monoester monoamide prodrug, a cyclic diamide prodrug, or a carbohydrate prodrug.

35. A pharmaceutical composition, comprising a compound of claim 1 and a pharmaceutically acceptable carrier.

36. The pharmaceutical composition of claim 35 that is formulated for single dosage administration.

37. A pharmaceutical composition, comprising a prodrug of claim 29 and a pharmaceutically acceptable carrier.

38. The pharmaceutical composition of claim 37 that is formulated for single dosage administration.

39. A method of treatment or amelioration of one or more symptoms of a disease or disorder that is modulated or otherwise affected by tyrosine phosphatase activity or in which tyrosine phosphatase activity is implicated, comprising administering a compound of claim 1 or a pharmaceutically acceptable derivative thereof.

40. The method of any of claim 39, wherein the disease or disorder is selected from metabolic disorders, autoimmune disease, and oncologic disease.

41. The method of claim 39, wherein the autoimmune disease is selected from glomeralonephritis, rheumatoid arthritis, systemic lupus erythematosus, scleroderma, chronic thyroiditis, Graves' disease, autoimmune gastritis, insulin-dependent diabetes mellitus (Type I), autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis and graft vs. host disease.

42. A method of treating or ameliorating one or more symptoms of an oncologic disease, comprising administering a compound of claim 1 or a pharmaceutically acceptable derivative thereof.

43. A method of treating or ameliorating one or more symptoms of cancer, comprising administering a compound of claim 1 or a pharmaceutically acceptable salt thereof.

44. The method of claim 43, wherein the disease is a solid tumor.

45. The method of claim 43, wherein the cancer is resistant to cytotoxic agents.

46. The method of claim 44, wherein the cancer is breast cancer, stomach cancer, cancer of the ovaries, cancer of the colon, lung cancer, brain cancer, cancer of the larynx, cancer of the lymphatic system, cancer of the genito-urinary tract including the bladder and the prostate, bone cancer and cancer of the pancreas.

47. A method of cancer chemotherapy, comprising administering a compound of claim 1 or a pharmaceutically acceptable salt thereof.

48. The method of claim 40, wherein the metabolic disorder is selected from diabetes mellitus and diabetes insipidus.

49. The method of claim 40, wherein the diabetes is selected from type 1 diabetes and type 2 diabetes.

50. A method for inhibiting tyrosine phosphatase activity, comprising contacting the tyrosine phosphatase with a compound of claim 1 or a pharmaceutically acceptable derivative thereof.

51. The method of claim 50, wherein the tyrosine phophatase is PTP-1B.
